SEQUENCE LISTING (4 sequences)

SEQ ID NO: 1 - DNA, 50 bp, Artificial Sequence
  Description of Artificial Sequence Synthetic polynucleotide
ggatatcatatgaaacatcaccatcaccatcacatcgtgggcggtgagag

SEQ ID NO: 2 - DNA, 33 bp, Artificial Sequence
  Description of Artificial Sequence Synthetic polynucleotide
ggatatgaattcttagccgtttgccttcatttg

SEQ ID NO: 3 - protein, 8 aa, Artificial Sequence
  Description of Artificial Sequence Synthetic polypeptide
MKHHHHHH

SEQ ID NO: 4 - protein, 6 aa, Artificial Sequence
  Description of Artificial Sequence Synthetic polypeptide
HHHHHH

(19) United States
(12) Patent Application Publication
(10) Pub. No.: US 2024/0132457 A1
(43) Pub. Date: Apr. 25, 2024

(54) 3-(2-(BENZO[D]THIAZOL-2-YL)-2-(PHENYLSUFONAMIDO)ETHYL)BENZIMIDAMIDE DERIVATIVES AND RELATED COMPOUNDS AS TMPRSS2 INHIBITORS FOR THE TREATMENT OF VIRAL INFECTIONS

(71) Applicant: INTEGERBIO, INC., North Bethesda, MD (US)

(72) Inventor: Stephen J. Shuttleworth, Buckinghamshire (GB)

(21) Appl. No.: 18/000,503

(22) PCT Filed: Jun. 2, 2021
(86) PCT No.: PCT/US2021/035487

§ 371 Date: Dec. 1, 2022

Related U.S. Application Data
(60) Provisional application No. 63/033,709, filed on Jun. 2, 2020.

(51) Int. Cl. C07D 277/64; A61P 31/16; C07D 271/10; C07D 513/04

(52) U.S. Cl. CPC C07D 277/64; A61P 31/16; C07D 271/10; C07D 513/04

(57) ABSTRACT

The present invention relates to compounds of formula I (I) as well as to the compounds of formula I for use as transmembrane serine protease 2 (TMPRSS2) inhibitors in the treatment of viral infections, such as e.g. corona virus infections. Exemplary compounds are e.g. 3-(2-(benzo[d]thiazol-2-yl)-2-(phenylsufonamido)ethyl)benzimidamide (example 1), as well as derivatives and related compounds thereof.

### CROSS-REFERENCE TO RELATED APPLICATION

This application is a U.S. National Phase Application, filed under 35 U.S.C. § 371, of International Application No. PCT/US2021/035487, filed on Jun. 2, 2021, which claims the benefit of and priority to U.S. Patent Application No. 63/033,709, filed on Jun. 2, 2020, the contents of which is incorporated by reference herein in its entirety.

### REFERENCE TO A SEQUENCE LISTING

This application contains a Sequence Listing in computer readable form. The computer readable form is incorporated herein by reference. Said ASCII copy, created on Aug. 7, 2023, is named TSP-001US_SL.txt and is 1,352 bytes in size.

### BACKGROUND

In the past two decades, severe acute respiratory syndrome coronavirus (SARS-CoV) and Middle East respiratory syndrome coronavirus (MERS-CoV) were transmitted from animals to humans, causing severe respiratory diseases SARS and MERS in endemic areas. In 2019, another coronavirus was discovered in patients with infectious respiratory disease in Wuhan, Hubei province, China, to have the ability for human-to-human transmission. The disease, now termed coronavirus disease 2019 (COVID-19), has spread rapidly all over the world, resulting in a pandemic. COVID-19 is induced by the pathogenic SARS-coronavirus 2 (SARS-CoV-2).

The serine protease, transmembrane serine protease 2 (TMPRSS2), a member of the type II transmembrane serine proteases (TTSPs), has been reported to be a host cell factor that is critical for the spread of several clinically relevant viruses, including coronaviruses and influenza A viruses. TMPRSS2 has been reported to cleave the surface glycoprotein haemagglutinin (HA) of influenza viruses with a monobasic cleavage site which is a prerequisite for virus fusion and propagation. It has been reported that host cell entry of coronaviruses depends upon binding of the viral spike (S) proteins to cellular receptors, and on S protein priming by host cell proteases: SARS-CoV-2 has been shown to use the SARS-CoV receptor angiotensin converting enzyme II (ACE2) for entry, and can be blocked by an inhibitor of TMPRSS2 which is employed by SARS-CoV-2 for S protein priming, and entry. TMPRSS2 has also been reported to be dispensable for development and homeostasis and thus constitutes an attractive drug target. Therefore, inhibition of TMPRSS2 would promote a blockade of the viral entry, thereby rendering TMPRSS2 inhibitors as promising candidates for the treatment of SARS-CoV-2 infection.

Considering the high mortality rate of COVID-19, other corona-viral and other serious viral infections, the development of effective therapeutics is an urgent issue and requires the identification of quality targets. TMPRSS2s are one of the key initiating factors of the SARS-CoV-2 infection. Thus, inhibitors of TMPRSS2 exhibit a differentiated mechanism of action for antiviral intervention of the SARS-CoV-2, and TMPRSS2 inhibitors provide a novel treatment for COVID-19 patients.

### SUMMARY

The disclosure is directed to, in part, inhibitors of transmembrane serine protease 2. Also provided are pharmaceutical compositions comprising at least one disclosed compound and a pharmaceutically acceptable carrier. Methods for using such compounds and compositions to treat disorders and/or diseases, for example, a viral infection are also described herein. Each of these different aspects can be described more particularly by the various embodiments described herein, which embodiments can be equally applicable to the different aspects.

In an embodiment, provided herein are compounds represented by Formula I:

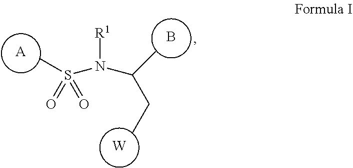

wherein: A is selected from the group consisting of phenyl, naphthyl, 5-6 membered heteroaryl, C₁₋₆alkyl, and C₁₋₆cycloalkyl, wherein A is optionally substituted by one, two, or three substituents each independently selected from the group consisting of R^{A}; R¹ is selected from hydrogen and C₁₋₆ alkyl; B is selected from the group consisting of 5-6 membered monocyclic heteroaryl, 8-10 membered bicyclic heteroaryl, and 8-10 membered bicyclic heterocyclyl, wherein B contains at least one nitrogen and B is optionally substituted on one, two, or three carbons by a substituent each independently selected from R^{B}; wherein when said heterocyclyl contains a —NH moiety, the nitrogen of —NH may optionally be substituted by one or more substituents each selected from R^{h}; W is selected from phenyl and heteroaryl, wherein W is substituted on a carbon by a warhead moiety R^{w}, wherein R^{w} is selected from the group consisting of amidine, amidoxime, guanidine, and N-hydroxyguanidine; R^{A} is selected from the group consisting of phenyl, 5-6 membered heteroaryl, and 4-10 membered heterocyclyl, wherein R^{A} may optionally be substituted by one or more substituents each selected from R^{g}; wherein when said heterocyclyl contains a —NH moiety, the nitrogen of —NH may optionally be substituted by one or more substituents each selected from R^{h}; or R^{A} is selected from the group consisting of halogen, cyano, hydroxyl, —N(RⁱR^{j}), C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}—, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆ alkylcarbonyl-N(R^{a})—C₁₋₆alkyl-, C₁₋₆alkyl-N(R^{a})-carbonyl-C₁₋₆alkyl-, and C₁₋₆alkoxyC₁₋₆alkyl-; wherein the C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}—, C₁₋₆alkyl-N(R^{a})—, C₁₋₆alkyl-N(R^{a})-carbonyl-, C₁₋₆ alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})C₁₋₆alkyl-, C₁₋₆alkyl-N(R^{a})-carbonyl-C₁₋₆alkyl-, or C₁₋₆alkoxy-C₁₋₆alkyl may optionally be substituted by R^{P}, phenyl, phenoxy, heteroaryl, heteroaryloxy, heteroaryl-(NR^{a})—, heterocyclyl, heterocyclyloxy or heterocyclyl-N(R^{a})—; wherein said phenyl or heteroaryl may optionally be substituted with one or more substituents selected from R^{f}; wherein said heterocyclyl may optionally be substituted by one or more substituents selected from R^{g}; and wherein when said heterocyclyl contains a —NH moiety, the nitrogen of —NH may optionally be substituted by one or more substituents each selected from R^{h}; R^{B} is selected from the group consisting of: halogen, cyano, hydroxyl, —N(RⁱR^{j}), phenyl, 5-6 membered heteroaryl, 5-6 membered heterocyclyl, —OR^{k}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}— (wherein w is 0, 1 or 2), C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})—C₁₋₆alkyl-, C₁₋₆alkyl-N(R^{a})-carbonyl-C₁₋₆alkyl-, and C₁₋₆alkoxyC₁₋₆alkyl-; wherein the C₁₋₆alkyl, C₂-6alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}—, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})C₁₋₆alkyl-, C₁₋₆alkyl-N(R^{a})-carbonyl-C₁₋₆alkyl-, and C₁₋₆alkoxy-C₁₋₆alkyl may optionally be substituted by one, two or three R^{P}, phenyl, phenoxy, 5-6 membered heteroaryl, heteroaryloxy, 5-6 membered heteroaryl-(NR^{a})—, 4-6 membered heterocyclyl, 4-6 membered heterocyclyloxy or 4-6 membered heterocyclyl-N(R^{a})—; and wherein said heteroaryl or phenyl may optionally be substituted with one or more substituents each selected from R^{f}; and wherein said heterocyclyl may optionally be substituted by one or more substituents each selected from R^{g}; and wherein when said heterocyclyl contains a —NH moiety, the nitrogen of —NH may optionally be substituted by one or more substituents each selected from R^{h}; R^{a} and R^{b} are independently selected, for each occurrence, from the group consisting of hydrogen and C₁₋₃alkyl, wherein the C₁₋₃alkyl may optionally be substituted by one or more substituents each selected from the group consisting of halogen, cyano, oxo, and hydroxyl; or R^{a} and R^{b}, together with the nitrogen to which they are attached, may form a 4-6 membered heterocyclic ring, which may have an additional heteroatom selected from O, S, and N; wherein the 4-6 membered heterocyclic ring may optionally be substituted by one or more substituents each selected from the group consisting of halogen, cyano, oxo, and hydroxyl; R^{f} is independently selected, for each occurrence, from the group consisting of R^{P}, hydrogen, C₁₋₆alkyl, C₃₋₆cycloalkyl, C₂₋₆alkenyl, C₂₋₆alkynyl, C₁₋₆alkoxy, C₁₋₆alkyl-S(O)_{w}—, C₁₋₆ alkylcarbonyl-N(R^{a})—, and C₁₋₆alkoxycarbonyl-N(R^{a})—; wherein the C₁₋₆alkyl, C₃₋₆cycloalkyl, C₂₋₆alkenyl, C₂₋₆alkynyl, C₁₋₆alkoxy, C₁₋₆alkyl-S(O)_{w}—, C₁₋₆alkylcarbonyl-N(R^{a})—, or C₁₋₆ alkoxycarbonyl-N(R^{a})— may optionally be substituted by one or more substituents each selected from R^{p}; R⁹ is independently selected, for each occurrence, from the group consisting of R^{P}, hydrogen, oxo, C₁₋₆alkyl, C₂₋₆alkenyl, C₂₋₆alkynyl, C₃₋₆cycloalkyl, C₁₋₆alkoxy, C₁₋₆alkyl-S(O)_{w}—, C₁₋₆ alkylcarbonyl-N(R^{a})—, C₁₋₆alkoxycarbonyl-N(R^{a})—, and —C(O)—NH(CH₃); wherein the C₁₋₆alkyl, C₂₋₆alkenyl, C₂₋₆alkynyl, C₃₋₆cycloalkyl, C₁₋₆alkoxy, C₁₋₆alkyl-S(O)_{w}—, C₁₋₆alkylcarbonyl-N(R^{a})—, or C₁₋₆alkoxycarbonyl-N(R^{a})— may optionally be substituted by one or more substituents each selected from R^{p}; R^{h} is independently selected, for each occurrence, from the group consisting of hydrogen, halogen, hydroxyl, cyano, C₁₋₆alkyl, C₃₋₆alkenyl, C₃₋₆alkynyl, C₃₋₆cycloalkyl, C₁₋₆alkoxy, (RⁱR^{j})N—, C₁₋₆alkyl-S(O)₂—, C₁₋₆alkoxycarbonyl-, (RⁱR^{j})N-carbonyl-, and (RⁱR^{j})N—SO₂—; wherein the C₁₋₆alkyl, C₁₋₆alkoxy, C₃₋₆alkenyl, C₃₋₆alkynyl C₃₋₆cycloalkyl, C₁₋₆alkyl-S(O)₂—, or C₁₋₆alkylcarbonyl- may optionally be substituted by one or more substituents each selected from R^{p}; Rⁱ and R^{j} are independently selected, for each occurrence, from the group consisting of hydrogen, C₁₋₆alkyl, and C₃₋₆cycloalkyl; wherein the C₁₋₆alkyl and C₃₋₆cycloalkyl may be optionally substituted by one or more substituents each selected from halogen, hydroxyl, cyano, (R^{a}R^{b})N—, (R^{a}R^{b})N-carbonyl-, (R^{a}R^{b})N-carbonyl-(R^{a})N—, and C₁₋₃alkoxy; or Rⁱ and R^{j} taken together with the nitrogen to which they are attached form a 4-7 membered heterocyclic ring, which may have an additional heteroatom selected from O, S, and N; wherein the 4-7 membered heterocyclic ring may optionally be substituted on carbon by one or more substituents each selected from the group consisting of halogen, hydroxyl, oxo, cyano, C₁₋₆alkyl, C₁₋₆alkoxy, (R^{a}R^{b})N—, (R^{a}R^{b})N—SO₂—, and (R^{a}R^{b})N-carbonyl-; wherein said C₁₋₆alkyl or C₁₋₆alkoxy may optionally be substituted by halogen, hydroxyl or cyano; wherein the 4-7 membered heterocyclic ring may be optionally substituted on nitrogen by one or more substituents each selected from the group consisting of C₁₋₆alkyl and (R^{a}R^{b})N-carbonyl-; and wherein said C₁₋₆alkyl may be optionally substituted by one or more substituents each selected from the group consisting of halogen, hydroxyl, and cyano; R^{p} is independently selected, for each occurrence, from the group consisting of halogen, hydroxyl, cyano, C₁₋₆alkoxy, (RⁱⁱR^{jj})N—, (RⁱⁱR^{jj})N-carbonyl-, (RⁱⁱR^{jj})N—SO₂—, (RⁱⁱR^{jj})N-carbonyl-N(Rⁱⁱ)—, (CH₃)C(O)N(Rⁱⁱ)—, —C(O)OH, and —C(O)N(RⁱⁱR^{jj}); Rⁱⁱ and R^{jj} are independently selected, for each occurrence, from the group consisting of hydrogen, C₁₋₆alkyl, and C₃₋₆cycloalkyl; R^{k} is selected from the group consisting of C₁₋₆alkyl, C₃₋₆ cycloalkyl, 4-6 membered heterocyclyl, C₁₋₆alkyl-(4-6 membered heterocyclyl), and C₁₋₆alkyl-(5-6 membered heteroaryl), wherein the C₁₋₆alkyl may optionally be substituted by one, two, or three R^{p}; w 0, 1, or 2; and pharmaceutically acceptable salts, stereoisomers, esters and prodrugs thereof.

In certain embodiments, provided herein are compounds represented by Formula I-a:

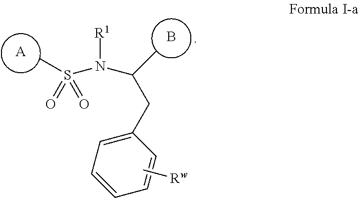

In certain embodiments, provided herein are compounds represented by Formula I-b:

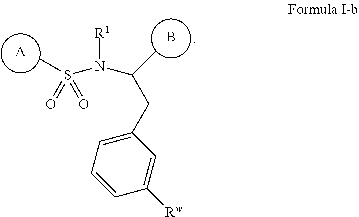

In some embodiments, provided herein are compounds represented by Formula I-c:

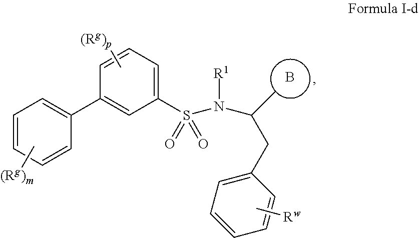

wherein R⁹ is independently selected, for each occurrence, from the group consisting of H, halogen, and C₁₋₆alkoxy; m is 1, 2, or 3; and p is 1, 2, or 3.

In an embodiment, provided herein are compounds represented by Formula II:

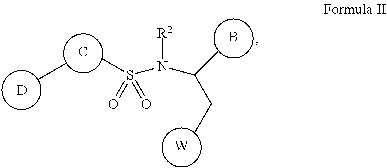

C and D are independently selected, for each occurrence, from the group consisting of phenyl and 5-6 membered monocyclic heteroaryl, wherein the phenyl or 5-6 membered monocyclic heteroaryl may optionally be substituted by one or more substituents each selected from Rg; R2 is selected from hydrogen and C1-6 alkyl; B is selected from the group consisting of 5-6 membered monocyclic heteroaryl, 8-10 membered bicyclic heteroaryl, and 8-10 membered bicyclic heterocyclyl, wherein B contains at least one nitrogen and B is optionally substituted on one, two, or three carbons by a substituent each independently selected from R^{B}; wherein when said heterocyclyl contains a —NH moiety, the nitrogen of —NH may optionally be substituted by one or more substituents each selected from R^{h}; W is selected from phenyl and heteroaryl, wherein W is substituted on a carbon by a warhead moiety R^{w}, wherein R^{w} is selected from the group consisting of amidine, amidoxime, guanidine, and N-hydroxyguanidine; R^{B} is selected from the group consisting of: halogen, cyano, hydroxyl, —N(RⁱR^{j}), phenyl, 5-6 membered heteroaryl, 5-6 membered heterocyclyl, —OR^{k}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}— (wherein w is 0, 1 or 2), C₁₋₆alkyl-N(R^{a})—, C₁₋₆ alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})—C₁₋₆alkyl-, C₁₋₆alkyl-N(R^{a})-carbonyl-C₁₋₆alkyl-, C₁₋₆alkoxyC₁₋₆alkyl-; wherein the C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}—, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})C₁₋₆alkyl-, C₁₋₆alkyl-N(R^{a})-carbonyl-C₁₋₆alkyl-, and C₁₋₆alkoxy-C₁₋₆alkyl may optionally be substituted by R^{P}, phenyl, phenoxy, 5-6 membered heteroaryl, heteroaryloxy, 5-6 membered heteroaryl-(NR^{a})—, 4-6 membered heterocyclyl, 4-6 membered heterocyclyloxy or 4-6 membered heterocyclyl-N(R^{a})—; and wherein said heteroaryl or phenyl may optionally be substituted with one or more substituents each selected from R^{f}; and wherein said heterocyclyl may optionally be substituted by one or more substituents each selected from R^{g}; and wherein when said heterocyclyl contains a —NH moiety, the nitrogen of —NH may optionally be substituted by one or more substituents each selected from R^{h}; R^{a} and R^{b} are independently selected, for each occurrence, from the group consisting of hydrogen and C₁₋₃alkyl, wherein the C₁₋₃alkyl may optionally be substituted by one or more substituents each selected from the group consisting of halogen, cyano, oxo, and hydroxyl; or R^{a} and R^{b}, together with the nitrogen to which they are attached, may form a 4-6 membered heterocyclic ring, which may have an additional heteroatom selected from 0, S, and N; wherein the 4-6 membered heterocyclic ring may optionally be substituted by one or more substituents each selected from the group consisting of halogen, cyano, oxo, and hydroxyl; R⁹ is independently selected, for each occurrence, from the group consisting of R^{P}, hydrogen, oxo, C₁₋₆alkyl, C₂₋₆alkenyl, C₂₋₆alkynyl, C₃₋₆cycloalkyl, C₁₋₆alkoxy, C₁₋₆alkyl-S(O)_{w}—, C₁₋₆alkylcarbonyl-N(R^{a})—, and C₁₋₆alkoxycarbonyl-N(R^{a})—; wherein the C₁₋₆alkyl, C₂₋₆alkenyl, C₂₋₆alkynyl, C₃₋₆cycloalkyl, C₁₋₆alkoxy, C₁₋₆alkyl-S(O)_{w}-, C₁₋₆alkylcarbonyl-N(R^{a})—, or C₁₋₆alkoxycarbonyl-N(R^{a})— may optionally be substituted by one or more substituents each selected from R^{p}; R^{h} is independently selected, for each occurrence, from the group consisting of hydrogen, halogen, hydroxyl, cyano, C₁₋₆alkyl, C₃₋₆alkenyl, C₃₋₆alkynyl, C₃₋₆cycloalkyl, C₁₋₆alkoxy, (RⁱR^{j})N—, C₁₋₆alkyl-S(O)₂—, C₁₋₆alkoxycarbonyl-, (RⁱR^{j})N-carbonyl-, and (RⁱR^{j})N—SO₂—; wherein the C₁₋₆alkyl, C₁₋₆alkoxy, C₃₋₆alkenyl, C₃₋₆alkynyl C₃₋₆cycloalkyl, C₁₋₆alkyl-S(O)₂—, or C₁₋₆alkylcarbonyl- may optionally be substituted by one or more substituents each selected from R^{p}; Rⁱ and R^{j} are independently selected, for each occurrence, from the group consisting of hydrogen, C₁₋₆alkyl, and C₃₋₆cycloalkyl; wherein the C₁₋₆alkyl and C₃₋₆cycloalkyl may be optionally substituted by one or more substituents each selected from halogen, hydroxyl, cyano, (R^{a}R^{b})N—, (R^{a}R^{b})N-carbonyl-, (R^{a}R^{b})N-carbonyl-(R^{a})N—, and C₁₋₃alkoxy; or Rⁱ and R^{j} taken together with the nitrogen to which they are attached form a 4-7 membered heterocyclic ring, which may have an additional heteroatom selected from O, S, and N; wherein the 4-7 membered heterocyclic ring may optionally be substituted on carbon by one or more substituents each selected from the group consisting of halogen, hydroxyl, oxo, cyano, C₁₋₆alkyl, C₁₋₆alkoxy, (R^{a}R^{b})N—, (R^{a}R^{b})N—SO₂—, and (R^{a}R^{b})N-carbonyl-; wherein said C₁₋₆alkyl or C₁₋₆alkoxy may optionally be substituted by halogen, hydroxyl or cyano; wherein the 4-7 membered heterocyclic ring may be optionally substituted on nitrogen by one or more substituents each selected from the group consisting of C₁₋₆alkyl and (R^{a}R^{b})N-carbonyl-; and wherein said C₁₋₆alkyl may be optionally substituted by one or more substituents each selected from the group consisting of halogen, hydroxyl, and cyano; R^{P} is independently selected, for each occurrence, from the group consisting of halogen, hydroxyl, cyano, C₁₋₆alkoxy, (RⁱⁱR^{jj})N—, (RⁱⁱR^{jj})N-carbonyl-, (RⁱⁱR^{jj})N—SO₂—, (RⁱⁱR^{jj})N-carbonyl-N(Rⁱⁱ)—, (CH₃)C(O)N(Rⁱⁱ)—, —C(O)OH, and —C(O)N(RⁱⁱR^{jj3}); Rⁱⁱ and R^{jj} are independently selected, for each occurrence, from the group consisting of hydrogen, C₁₋₆alkyl, and C₃₋₆cycloalkyl; R^{k} is selected from the group consisting of C₁₋₆alkyl, C₃₋₆cycloalkyl, 4-6 membered heterocyclyl, C₁₋₆alkyl-(4-6 membered heterocyclyl), and C₁₋₆alkyl-(5-6 membered heteroaryl), wherein the C₁₋₆alkyl may optionally be substituted by one, two, or three R^{p}; w 0, 1, or 2; and pharmaceutically acceptable salts, stereoisomers, esters and prodrugs thereof.

In some embodiments, provided herein are compounds represented by Formula II-a:

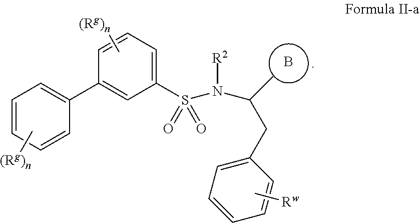

In certain embodiments, provided herein are compounds represented by Formula III:

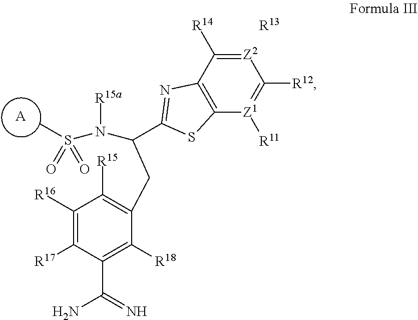

wherein: A is selected from the group consisting of phenyl, naphthyl, and 5-6 membered heteroaryl, wherein A is optionally substituted by one, two, or three substituents each independently selected from the group consisting of R^{A}; R¹¹, R¹², R¹³, and R¹⁴ are independently selected, for each occurrence, from the group consisting of H, —CN, —OH, —NO₂, —NH₂, halogen, C₁₋₆haloalkyl, C₁₋₆ alkyl, C₁₋₆alkoxy, and C₃₋₆cycloalkyl; R^{15a} is selected from the group consisting of H, C₁₋₆alkyl, and C₃₋₆cycloalkyl; R¹⁵, R¹⁶, R¹⁷, and R¹⁸ are independently selected, for each occurrence, from the group consisting of H, —NO₂, —NH₂, cyano, hydroxyl, halogen, C₁₋₆haloalkyl, C₁₋₆alkyl, C₁₋₆alkoxy, and C₃₋₆cycloalkyl; R^{A} is selected from the group consisting of —NO₂, —NH₂, cyano, hydroxyl, halogen, C₁₋₆haloalkyl, C₁₋₆alkyl, C₁₋₆alkoxy, and C₃₋₆cycloalkyl; Z¹ is C or N, wherein when Z¹ is N then R¹¹ is absent; Z² is C or N, wherein when Z² is N then R¹³ is absent; and pharmaceutically acceptable salts, stereoisomers, esters and prodrugs thereof.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### Definitions

“Treating” includes any effect, e.g., lessening, reducing, modulating, or eliminating, that results in the improvement of the condition, disease, disorder and the like.

The term “alkenyl” as used herein refers to an unsaturated straight or branched hydrocarbon having at least one carbon-carbon double bond. Exemplary alkenyl groups include, but are not limited to, a straight or branched group of 2-6 or 3-4 carbon atoms, referred to herein as C₂₋₆alkenyl, and C₃₋₄alkenyl, respectively. Exemplary alkenyl groups include, but are not limited to, vinyl, allyl, butenyl, pentenyl, etc.

The term “alkoxy” as used herein refers to a straight or branched alkyl group attached to oxygen (alkyl-O—). Exemplary alkoxy groups include, but are not limited to, alkoxy groups of 1-6 or 2-6 carbon atoms, referred to herein as C₁₋₆alkoxy, and C₂₋₆alkoxy, respectively. Exemplary alkoxy groups include, but are not limited to methoxy, ethoxy, isopropoxy, etc.

The term “alkoxyalkyl” as used herein refers to a straight or branched alkyl group attached to oxygen, attached to a second straight or branched alkyl group (alkyl-O-alkyl-). Exemplary alkoxyalkyl groups include, but are not limited to, alkoxyalkyl groups in which each of the alkyl groups independently contains 1-6 carbon atoms, referred to herein as C₁₋₆alkoxy-C₁₋₆alkyl. Exemplary alkoxyalkyl groups include, but are not limited to methoxymethyl, 2-methoxyethyl, 1-methoxyethyl, 2-methoxypropyl, ethoxymethyl, 2-isopropoxyethyl etc.

The term “alkyoxycarbonyl” as used herein refers to a straight or branched alkyl group attached to oxygen, attached to a carbonyl group (alkyl-O—C(O)—). Exemplary alkoxycarbonyl groups include, but are not limited to, alkoxycarbonyl groups of 1-6 carbon atoms, referred to herein as C₁₋₆alkoxycarbonyl. Exemplary alkoxycarbonyl groups include, but are not limited to, methoxycarbonyl, ethoxycarbonyl, t-butoxycarbonyl, etc.

The term “alkenyloxy” used herein refers to a straight or branched alkenyl group attached to oxygen (alkenyl-O—). Exemplary alkenyloxy groups include, but are not limited to, groups with an alkenyl group of 3-6 carbon atoms, referred to herein as C₃₋₆alkenyloxy. Exemplary “alkenyloxy” groups include, but are not limited to allyloxy, butenyloxy, etc.

The term “alkynyloxy” used herein refers to a straight or branched alkynyl group attached to oxygen (alkynyl-O). Exemplary alkynyloxy groups include, but are not limited to, groups with an alkynyl group of 3-6 carbon atoms, referred to herein as C₃₋₆alkynyloxy. Exemplary alkynyloxy groups include, but are not limited to, propynyloxy, butynyloxy, etc.

The term “alkyl” as used herein refers to a saturated straight or branched hydrocarbon. Exemplary alkyl groups include, but are not limited to, straight or branched hydrocarbons of 1-6, 1-4, or 1-3 carbon atoms, referred to herein as C₁₋₆alkyl, C₁₋₄alkyl, and C₁₋₃ alkyl, respectively. Exemplary alkyl groups include, but are not limited to, methyl, ethyl, propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, etc.

The term “alkylcarbonyl” as used herein refers to a straight or branched alkyl group attached to a carbonyl group (alkyl-C(O)—). Exemplary alkylcarbonyl groups include, but are not limited to, alkylcarbonyl groups of 1-6 atoms, referred to herein as C₁₋₆alkylcarbonyl groups. Exemplary alkylcarbonyl groups include, but are not limited to, acetyl, propanoyl, isopropanoyl, butanoyl, etc.

The term “alkynyl” as used herein refers to an unsaturated straight or branched hydrocarbon having at least one carbon-carbon triple bond. Exemplary alkynyl groups include, but are not limited to, straight or branched groups of 2-6, or 3-6 carbon atoms, referred to herein as C₂₋₆alkynyl, and C₃₋₆alkynyl, respectively. Exemplary alkynyl groups include, but are not limited to, ethynyl, propynyl, butynyl, pentynyl, hexynyl, methylpropynyl, etc.

The term “carbonyl” as used herein refers to the radical —C(O)—.

The term “cyano” as used herein refers to the radical —CN.

The term “cycloalkoxy” as used herein refers to a cycloalkyl group attached to oxygen (cycloalkyl-O—). Exemplary cycloalkoxy groups include, but are not limited to, cycloalkoxy groups of 3-6 carbon atoms, referred to herein as C₃₋₆cycloalkoxy groups. Exemplary cycloalkoxy groups include, but are not limited to, cyclopropoxy, cyclobutoxy, cyclohexyloxy, etc.

The terms “cycloalkyl” or a “carbocyclic group” as used herein refers to a saturated or partially unsaturated hydrocarbon group of, for example, 3-6, or 4-6 carbons, referred to herein as C₃₋₆cycloalkyl or C₄₋₆cycloalkyl, respectively. Exemplary cycloalkyl groups include, but are not limited to, cyclohexyl, cyclopentyl, cyclopentenyl, cyclobutyl or cyclopropyl.

The terms “halo” or “halogen” as used herein refer to F, Cl, Br, or I.

The term “haloalkyl” as used herein refers to an alkyl radical in which the alkyl group is substituted with one or more halogens. Typical haloalkyl groups include, but are not limited to, trifluoromethyl (i.e. CF₃), difluoromethyl, fluoromethyl, chloromethyl, dichloromethyl, dibromoethyl, tribromomethyl, tetrafluoroethyl, and the like. Exemplary haloalkyl groups include, but are not limited to, straight or branched hydrocarbons of 1-6, 1-4, or 1-3 carbon atoms substituted with a halogen (i.e. Cl, F, Br and I), referred to herein as C₁₋₆haloalkyl, C₁₋₄ haloalkyl, and C₁₋₃haloalkyl, respectively.

The terms “heteroaryl” or “heteroaromatic group” as used herein refers to a monocyclic aromatic 5-6 membered ring system or 8-10 membered bicyclic ring system containing one or more heteroatoms, for example one to three heteroatoms, such as nitrogen, oxygen, and sulfur. Where possible, said heteroaryl ring may be linked to the adjacent radical though carbon or nitrogen. Examples of heteroaryl rings include but are not limited to furan, thiophene, pyrrole, thiazole, oxazole, isothiazole, isoxazole, imidazole, pyrazole, triazole, pyridine or pyrimidine etc.

The terms “heterocyclyl,” “heterocycle,” or “heterocyclic group” are art-recognized and refer to saturated or partially unsaturated, mono cyclic or bicyclic 4-10 membered ring structures, including bridged or fused rings, and whose ring structures include one to three heteroatoms, such as nitrogen, oxygen, and sulfur. Where possible, heterocyclyl rings may be linked to the adjacent radical through carbon or nitrogen. Examples of heterocyclyl groups include, but are not limited to, pyrrolidine, piperidine, morpholine, thiomorpholine, piperazine, oxetane, azetidine, tetrahydrofuran or dihydrofuran etc.

The term “heterocyclyloxy” as used herein refers to a heterocyclyl group attached to oxygen (heterocyclyl-O—).

The term “heteroaryloxy” as used herein refers to a heteroaryl group attached to oxygen (heteroaryl-O—).

The terms “hydroxy” and “hydroxyl” as used herein refers to the radical —OH.

The term “oxo” as used herein refers to the radical ═O.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the invention can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the invention is desirably a mammal in which treatment of obesity or weight loss is desired. “Modulation” includes antagonism (e.g., inhibition), agonism, partial antagonism and/or partial agonism.

In the present specification, the term “therapeutically effective amount” means the amount of the subject compound that will elicit the biological or medical response of a tissue, system or animal, (e.g. mammal or human) that is being sought by the researcher, veterinarian, medical doctor or other clinician. The compounds of the invention are administered in therapeutically effective amounts to treat a disease. Alternatively, a therapeutically effective amount of a compound is the quantity required to achieve a desired therapeutic and/or prophylactic effect, such as an amount which results in weight loss.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The compounds of the disclosure may contain one or more chiral centers and, therefore, exist as stereoisomers. The term “stereoisomers” when used herein consist of all enantiomers or diastereomers. These compounds may be designated by the symbols “(+),” “(−),” “R” or “S,” depending on the configuration of substituents around the stereogenic carbon atom, but the skilled artisan will recognize that a structure may denote a chiral center implicitly. The present invention encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic rings may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present invention can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the invention embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The invention also embraces isotopically labeled compounds of the invention which are identical to those recited herein, except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine and chlorine, such as ²H ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the invention may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the invention can generally be prepared by following procedures analogous to those disclosed in the examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al, Nature Reviews Drug Discovery 2008, 7, 255). For example, if a compound of the invention or a pharmaceutically acceptable salt, hydrate or solvate of the compound contains a carboxylic acid functional group, a prodrug can comprise an ester formed by the replacement of the hydrogen atom of the acid group with a group such as (C₁₋₈)alkyl, (C₂₋₁₂)alkylcarbonyloxymethyl, 1-(alkylcarbonyloxy)ethyl having from 4 to 9 carbon atoms, 1-methyl-1-(alkylcarbonyloxy)-ethyl having from 5 to 10 carbon atoms, alkoxycarbonyloxymethyl having from 3 to 6 carbon atoms, 1-(alkoxycarbonyloxy)ethyl having from 4 to 7 carbon atoms, 1-methyl-1-(alkoxycarbonyloxy)ethyl having from 5 to 8 carbon atoms, N-(alkoxycarbonyl)aminomethyl having from 3 to 9 carbon atoms, 1-(N-(alkoxycarbonyl)amino)ethyl having from 4 to 10 carbon atoms, 3-phthalidyl, 4-crotonolactonyl, gamma-butyrolacton-4-yl, di-N,N—(C₁₋₂)alkylamino(C₂₋₃)alkyl (such as (3-dimethylaminoethyl), carbamoyl-(C₁₋₂)alkyl, N,N-di(C₁₋₂)alkylcarbamoyl-(C₁₋₂)alkyl and piperidino-, pyrrolidino- or morpholino(C₂₋₃)alkyl.

Similarly, if a compound of the invention contains an alcohol functional group, a prodrug can be formed by the replacement of the hydrogen atom of the alcohol group with a group such as (C₁₋₆)alkylcarbonyloxymethyl, 1-((C₁₋₆)alkylcarbonyloxy)ethyl, 1-methyl-1-((C₁₋₆)alkylcarbonyloxy)ethyl (C₁₋₆)alkoxycarbonyloxymethyl, N—(C₁₋₆)alkoxycarbonylaminomethyl, succinoyl, (C₁₋₆)alkylcarbonyl, α-amino(C₁₋₄)alkylcarbonyl, arylalkylcarbonyl and α-aminoalkylcarbonyl, or α-aminoalkylcarbonyl-α-aminoalkylcarbonyl, where each α-aminoalkylcarbonyl group is independently selected from the naturally occurring L-amino acids, P(O)(OH)₂, —P(O)(O(C₁₋₆)alkyl)₂ or glycosyl (the radical resulting from the removal of a hydroxyl group of the hemiacetal form of a carbohydrate).

If a compound of the invention incorporates an amine functional group, a prodrug can be formed, for example, by creation of an amide or carbamate, an N-alkylcarbonyloxyalkyl derivative, an (oxodioxolenyl)methyl derivative, an N-Mannich base, imine or enamine. In addition, a secondary amine can be metabolically cleaved to generate a bioactive primary amine, or a tertiary amine can metabolically cleaved to generate a bioactive primary or secondary amine. For examples, see Simplicio, et al., Molecules 2008, 13, 519 and references therein.

### I. Compounds

In certain embodiments, the present disclosure provides compounds of Formula I:

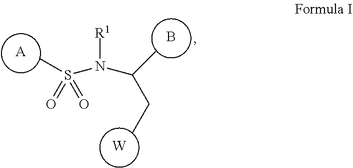

wherein: A is selected from the group consisting of phenyl, naphthyl, 5-6 membered heteroaryl, C₁₋₆ alkyl, and C₁₋₆cycloalkyl, wherein A is optionally substituted by one, two, or three substituents each independently selected from the group consisting of R^{A}; R¹ is selected from hydrogen and C₁₋₆ alkyl; B is selected from the group consisting of 5-6 membered monocyclic heteroaryl, 8-10 membered bicyclic heteroaryl, and 8-10 membered bicyclic heterocyclyl, wherein B contains at least one nitrogen and B is optionally substituted on one, two, or three carbons by a substituent each independently selected from R^{B}; wherein when said heterocyclyl contains a —NH moiety, the nitrogen of —NH may optionally be substituted by one or more substituents each selected from R^{h}; W is selected from phenyl and heteroaryl, wherein W is substituted on a carbon by a warhead moiety R^{w}, wherein R^{w} is selected from the group consisting of amidine, amidoxime, guanidine, and N-hydroxyguanidine; R^{A} is selected from the group consisting of phenyl, 5-6 membered heteroaryl, and 4-10 membered heterocyclyl, wherein R^{A} may optionally be substituted by one or more substituents each selected from R^{g}; wherein when said heterocyclyl contains a —NH moiety, the nitrogen of —NH may optionally be substituted by one or more substituents each selected from R^{h}; or R^{A} is selected from the group consisting of halogen, cyano, hydroxyl, —N(RⁱR^{j}), C₁₋₆alkyl, C₂-6alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}—, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})—C₁₋₆alkyl-, C₁₋₆alkyl-N(R^{a})-carbonyl-C₁₋₆alkyl-, and C₁₋₆alkoxyC₁₋₆alkyl-; wherein the C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}—, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})C₁₋₆alkyl-, C₁₋₆alkyl-N(R^{a})-carbonyl-C₁₋₆alkyl-, or C₁₋₆alkoxy-C₁₋₆alkyl may optionally be substituted by R^{P}, phenyl, phenoxy, heteroaryl, heteroaryloxy, heteroaryl-(NR^{a})—, heterocyclyl, heterocyclyloxy or heterocyclyl-N(R^{a})—; wherein said phenyl or heteroaryl may optionally be substituted with one or more substituents each selected from R^{f}; wherein said heterocyclyl may optionally be substituted by one or more substituents each selected from R^{g}; and wherein when said heterocyclyl contains a —NH moiety, the nitrogen of —NH may optionally be substituted by one or more substituents each selected from R^{h}; R^{B} is selected from the group consisting of: halogen, cyano, hydroxyl, —N(RⁱR^{j}), phenyl, 5-6 membered heteroaryl, 5-6 membered heterocyclyl, —OR^{k}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}— (wherein w is 0, 1 or 2), C₁₋₆ alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})—C₁₋₆alkyl-, C₁₋₆alkyl-N(R^{a})-carbonyl-C₁₋₆alkyl-, C₁₋₆alkoxyC₁₋₆alkyl-; wherein the C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆ cycloalkoxy, C₁₋₆alkyl-S(O)_{w}—, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})C₁₋₆alkyl-, C₁₋₆alkyl-N(R^{a})-carbonyl-C₁₋₆alkyl-, and C₁₋₆alkoxy-C₁₋₆alkyl may optionally be substituted by one, two or three R^{P}, phenyl, phenoxy, 5-6 membered heteroaryl, heteroaryloxy, 5-6 membered heteroaryl-(NR^{a})—, 4-6 membered heterocyclyl, 4-6 membered heterocyclyloxy or 4-6 membered heterocyclyl-N(R^{a})—; and wherein said heteroaryl or phenyl may optionally be substituted with one or more substituents each selected from R^{f}; and wherein said heterocyclyl may optionally be substituted by one or more substituents each selected from R^{g}; and wherein when said heterocyclyl contains a —NH moiety, the nitrogen of —NH may optionally be substituted by one or more substituents each selected from R^{h}; R^{a} and R^{b} are independently selected, for each occurrence, from the group consisting of hydrogen and C₁₋₃alkyl, wherein the C₁₋₃alkyl may optionally be substituted by one or more substituents each selected from the group consisting of halogen, cyano, oxo, and hydroxyl; or R^{a} and R^{b}, together with the nitrogen to which they are attached, may form a 4-6 membered heterocyclic ring, which may have an additional heteroatom selected from O, S, and N; wherein the 4-6 membered heterocyclic ring may optionally be substituted by one or more substituents each selected from the group consisting of halogen, cyano, oxo, and hydroxyl; R^{f} is independently selected, for each occurrence, from the group consisting of R^{P}, hydrogen, C₁₋₆alkyl, C₃₋₆cycloalkyl, C₂₋₆alkenyl, C₂₋₆alkynyl, C₁₋₆alkoxy, C₁₋₆alkyl-S(O)_{w}—, C₁₋₆alkylcarbonyl-N(R^{a})—, and C₁₋₆alkoxycarbonyl-N(R^{a})—; wherein the C₁₋₆alkyl, C₃₋₆cycloalkyl, C₂₋₆alkenyl, C₂₋₆alkynyl, C₁₋₆alkoxy, C₁₋₆alkyl-S(O)_{w}—, C₁₋₆alkylcarbonyl-N(R^{a})—, or C₁₋₆alkoxycarbonyl-N(R^{a})— may optionally be substituted by one or more substituents each selected from R^{p}; R⁹ is independently selected, for each occurrence, from the group consisting of R^{P}, hydrogen, oxo, C₁₋₆alkyl, C₂₋₆alkenyl, C₂₋₆alkynyl, C₃₋₆cycloalkyl, C₁₋₆alkoxy, C₁₋₆alkyl-S(O)_{w}—, C₁₋₆alkylcarbonyl-N(R^{a})—, C₁₋₆alkoxycarbonyl-N(R^{a})—, and —C(O)—NH(CH₃); wherein the C₁₋₆alkyl, C₂₋₆alkenyl, C₂₋₆alkynyl, C₃₋₆cycloalkyl, C₁₋₆alkoxy, C₁₋₆alkyl-S(O)_{w}—, C₁₋₆alkylcarbonyl-N(R^{a})—, or C₁₋₆ alkoxycarbonyl-N(R^{a})— may optionally be substituted by one or more substituents each selected from R^{p}; R^{h} is independently selected, for each occurrence, from the group consisting of hydrogen, halogen, hydroxyl, cyano, C₁₋₆alkyl, C₃₋₆alkenyl, C₃₋₆alkynyl, C₃₋₆cycloalkyl, C₁₋₆alkoxy, (RⁱR^{j})N—, C₁₋₆alkyl-S(O)₂—, C₁₋₆alkoxycarbonyl-, (RⁱR^{j})N-carbonyl-, and (RⁱR^{j})N—SO₂—; wherein the C₁₋₆alkyl, C₁₋₆alkoxy, C₃₋₆alkenyl, C₃₋₆alkynyl C₃₋₆cycloalkyl, C₁₋₆alkyl-S(O)₂—, or C₁₋₆alkylcarbonyl- may optionally be substituted by one or more substituents each selected from R^{p}; Rⁱ and R^{j} are independently selected, for each occurrence, from the group consisting of hydrogen, C₁₋₆alkyl, and C₃₋₆cycloalkyl; wherein the C₁₋₆alkyl and C₃₋₆cycloalkyl may be optionally substituted by one or more substituents each selected from halogen, hydroxyl, cyano, (R^{a}R^{b})N—, (R^{a}R^{b})N-carbonyl-, (R^{a}R^{b})N-carbonyl-(R^{a})N—, and C₁₋₃alkoxy; or Rⁱ and R^{j} taken together with the nitrogen to which they are attached form a 4-7 membered heterocyclic ring, which may have an additional heteroatom selected from O, S, and N; wherein the 4-7 membered heterocyclic ring may optionally be substituted on carbon by one or more substituents each selected from the group consisting of halogen, hydroxyl, oxo, cyano, C₁₋₆alkyl, C₁₋₆alkoxy, (R^{a}R^{b})N—, (R^{a}R^{b})N—SO₂—, and (R^{a}R^{b})N-carbonyl-; wherein said C₁₋₆alkyl or C₁₋₆alkoxy may optionally be substituted by halogen, hydroxyl or cyano; wherein the 4-7 membered heterocyclic ring may be optionally substituted on nitrogen by one or more substituents each selected from the group consisting of C₁₋₆alkyl and (R^{a}R^{b})N-carbonyl-; and wherein said C₁₋₆alkyl may be optionally substituted by one or more substituents each selected from the group consisting of halogen, hydroxyl, and cyano; R^{P} is independently selected, for each occurrence, from the group consisting of halogen, hydroxyl, cyano, C₁₋₆alkoxy, (RⁱⁱR^{jj})N—, (RⁱⁱR^{jj})N-carbonyl-, (RⁱⁱR^{jj})N—SO₂—, (RⁱⁱR^{jj})N-carbonyl-N(Rⁱⁱ)—, (CH₃)C(O)N(Rⁱⁱ)—, —C(O)OH, and —C(O)N(RⁱⁱR^{jj}); Rⁱⁱ and R^{jj} are independently selected, for each occurrence, from the group consisting of hydrogen, C₁₋₆alkyl, and C₃₋₆cycloalkyl; R^{k} is selected from the group consisting of C₁₋₆alkyl, C₃₋₆cycloalkyl, 4-6 membered heterocyclyl, C₁₋₆alkyl-(4-6 membered heterocyclyl), and C₁₋₆alkyl-(5-6 membered heteroaryl), wherein the C₁₋₆alkyl may optionally be substituted by one, two, or three R^{p}; w 0, 1, or 2; and pharmaceutically acceptable salts, stereoisomers, esters and prodrugs thereof.

In some embodiments, the disclosure provides compounds of Formula I:

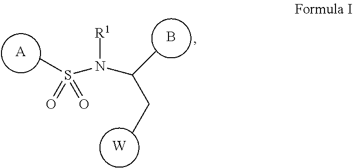

wherein: A is selected from the group consisting of phenyl, naphthyl, and 5-6 membered heteroaryl, wherein A is optionally substituted by one, two, or three substituents each independently selected from the group consisting of R^{A}; R¹ is selected from hydrogen and C₁₋₆ alkyl; B is selected from the group consisting of 5-6 membered monocyclic heteroaryl, 8-10 membered bicyclic heteroaryl, and 8-10 membered bicyclic heterocyclyl, wherein B contains at least one nitrogen and is optionally substituted on one, two, or three carbons by a substituent each independently selected from R^{B}; wherein when said heterocyclyl contains a —NH moiety, the nitrogen of —NH may optionally be substituted by one or more substituents each selected from R^{h}; W is selected from phenyl and heteroaryl, wherein W is substituted on a carbon by a warhead moiety R^{w}, wherein R^{w} is selected from the group consisting of amidine, amidoxime, guanidine, and N-hydroxyguanidine; R^{A} is selected from the group consisting of phenyl, 5-6 membered heteroaryl, and 4-10 membered heterocyclyl, wherein R^{A} may optionally be substituted by one or more substituents each selected from R^{g}; wherein when said heterocyclyl contains a —NH moiety, the nitrogen of —NH may optionally be substituted by one or more substituents each selected from R^{h}; or R^{A} is selected from the group consisting of halogen, cyano, hydroxyl, —N(RⁱR^{j}), C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}—, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})—C₁₋₆alkyl-, C₁₋₆alkyl-N(R^{a})-carbonyl-C₁₋₆alkyl-, and C₁₋₆alkoxyC₁₋₆alkyl-; wherein the C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃-6cycloalkoxy, C₁₋₆alkyl-S(O)_{w}—, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆ alkoxycarbonyl-N(R^{a})—, C₁₋₆alkylcarbonyl-N(R^{a})C₁₋₆alkyl-, C₁₋₆alkyl-N(R^{a})-carbonyl-C₁₋₆alkyl-, or C₁₋₆alkoxy-C₁₋₆alkyl may optionally be substituted by R^{P}, phenyl, phenoxy, heteroaryl, heteroaryloxy, heteroaryl-(NR^{a})—, heterocyclyl, heterocyclyloxy or heterocyclyl-N(R^{a})—; wherein said phenyl or heteroaryl may optionally be substituted with one or more substituents selected from R^{f}; wherein said heterocyclyl may optionally be substituted by one or more substituents selected from R^{g}; and wherein when said heterocyclyl contains a —NH moiety, the nitrogen of —NH may optionally be substituted by one or more substituents each selected from R^{h}; R^{B} is selected from the group consisting of: halogen, cyano, hydroxyl, —N(RⁱR^{j}), phenyl, heteroaryl, heterocyclyl, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}— (wherein w is 0, 1 or 2), C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})—C₁₋₆alkyl-, C₁₋₆alkyl-N(R^{a})-carbonyl-C₁₋₆alkyl-, C₁₋₆alkoxyC₁₋₆alkyl-; wherein C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}—, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})C₁₋₆alkyl-, C₁₋₆alkyl-N(R^{a})-carbonyl-C₁₋₆alkyl-, C₁₋₆alkoxy-C₁₋₆alkyl may optionally be substituted by R^{P}, phenyl, phenoxy, heteroaryl, heteroaryloxy, heteroaryl-(NR^{a})—, heterocyclyl, heterocyclyloxy or heterocyclyl-N(R^{a})—; and wherein said heteroaryl or phenyl may optionally be substituted with one or more substituents selected from R^{f}; and wherein said heterocyclyl may optionally be substituted by one or more substituents selected from R^{g}; and wherein when said heterocyclyl contains a —NH moiety, the nitrogen of —NH may optionally be substituted by one or more substituents each selected from R^{h}; R^{a} and R^{b} are independently selected, for each occurrence, from the group consisting of hydrogen and C₁₋₃alkyl, wherein the C₁₋₃alkyl may optionally be substituted by one or more substituents selected from the group consisting of halogen, cyano, oxo, and hydroxyl; or R^{a} and R^{b}, together with the nitrogen to which they are attached, may form a 4-6 membered heterocyclic ring, which may have an additional heteroatom selected from O, S, and N; wherein the 4-6 membered heterocyclic ring may optionally be substituted by one or more substituents selected from the group consisting of halogen, cyano, oxo, and hydroxyl; R^{f} is independently selected, for each occurrence, from the group consisting of R^{P}, hydrogen, C₁₋₆alkyl, C₃₋₆cycloalkyl, C₂₋₆alkenyl, C₂₋₆alkynyl, C₁₋₆alkoxy, C₁₋₆alkyl-S(O)_{w}—, C₁₋₆alkylcarbonyl-N(R^{a})—, and C₁₋₆alkoxycarbonyl-N(R^{a})—; wherein the C₁₋₆alkyl, C₃₋₆cycloalkyl, C₂₋₆alkenyl, C₂₋₆alkynyl, C₁₋₆alkoxy, C₁₋₆alkyl-S(O)_{w}—, C₁₋₆alkylcarbonyl-N(R^{a})—, or C₁₋₆alkoxycarbonyl-N(R^{a})— may optionally be substituted by one or more substituents selected from R^{p}; R⁹ is independently selected, for each occurrence, from the group consisting of R^{P}, hydrogen, oxo, C₁₋₆alkyl, C₂₋₆alkenyl, C₂₋₆alkynyl, C₃₋₆cycloalkyl, C₁₋₆alkoxy, C₁₋₆alkyl-S(O)_{w}—, C₁₋₆alkylcarbonyl-N(R^{a})—, and C₁₋₆alkoxycarbonyl-N(R^{a})—; wherein the C₁₋₆alkyl, C₂₋₆alkenyl, C₂₋₆alkynyl, C₃₋₆cycloalkyl, C₁₋₆alkoxy, C₁₋₆alkyl-S(O)_{w}—, C₁₋₆alkylcarbonyl-N(R^{a})—, or C₁₋₆ alkoxycarbonyl-N(R^{a})— may optionally be substituted by one or more substituents selected from R^{p}; R^{h} is independently selected, for each occurrence, from the group consisting of hydrogen, halogen, hydroxyl, cyano, C₁₋₆alkyl, C₃₋₆alkenyl, C₃₋₆alkynyl, C₃₋₆cycloalkyl, C₁₋₆alkoxy, (RⁱR^{j})N—, C₁₋₆alkyl-S(O)₂—, C₁₋₆alkoxycarbonyl-, (RⁱR^{j})N-carbonyl-, and (RⁱR^{j})N—SO₂—; wherein the C₁₋₆alkyl, C₁₋₆alkoxy, C₃₋₆alkenyl, C₃₋₆alkynyl C₃₋₆cycloalkyl, C₁₋₆alkyl-S(O)₂—, or C₁₋₆alkylcarbonyl- may optionally be substituted by one or more substituents selected from R^{p}; Rⁱ and R^{j} are independently selected, for each occurrence, from the group consisting of hydrogen, C₁₋₆alkyl, and C₃₋₆cycloalkyl; wherein the C₁₋₆alkyl and C₃₋₆cycloalkyl may be optionally substituted by one or more substituents selected from halogen, hydroxyl, cyano, (R^{a}R^{b})N—, (R^{a}R^{b})N-carbonyl-, and C₁₋₃alkoxy; or Rⁱ and R^{j} taken together with the nitrogen to which they are attached form a 4-7 membered heterocyclic ring, which may have an additional heteroatom selected from O, S, and N; wherein the 4-7 membered heterocyclic ring may optionally be substituted on carbon by one or more substituents selected from the group consisting of halogen, hydroxyl, oxo, cyano, C₁₋₆alkyl, C₁₋₆alkoxy, (R^{a}R^{b})N—, (R^{a}R^{b})N—SO₂—, and (R^{a}R^{b})N-carbonyl-; wherein said C₁₋₆alkyl or C₁₋₆alkoxy may optionally be substituted by halogen, hydroxyl or cyano; wherein the 4-7 membered heterocyclic ring may be optionally substituted on nitrogen by one or more substituents selected from the group consisting of C₁₋₆alkyl and (R^{a}R^{b})N-carbonyl-; and wherein said C₁₋₆alkyl may be optionally substituted by one or more substituents selected from the group consisting of halogen, hydroxyl, and cyano; R^{P} is independently selected, for each occurrence, from the group consisting of halogen, hydroxyl, cyano, C₁₋₆alkoxy, (RⁱR^{j})N—, (RⁱR^{j})N-carbonyl-, (RⁱR^{j})N—SO₂—, and (RⁱR^{j})N-carbonyl-N(R^{a})—; w 0, 1, or 2; and pharmaceutically acceptable salts, stereoisomers, esters and prodrugs thereof.

In some embodiments, A is selected from the group consisting of phenyl, naphthyl, 5-6 membered heteroaryl, C₁₋₆alkyl, and C₁₋₆cycloalkyl, wherein A is optionally substituted by one, two, or three substituents each independently selected from the group consisting of R^{A}; R¹ is selected from hydrogen and C₁₋₆ alkyl; B is selected from the group consisting of 5-6 membered monocyclic heteroaryl, 8-10 membered bicyclic heteroaryl, and 8-10 membered bicyclic heterocyclyl, wherein B contains at least one nitrogen and B is optionally substituted on one, two, or three carbons by a substituent each independently selected from R^{B}; wherein when said heterocyclyl contains a —NH moiety, the nitrogen of —NH may optionally be substituted by one or more substituents each selected from R^{h}; W is selected from phenyl and heteroaryl, wherein W is substituted on a carbon by a warhead moiety R^{w}, wherein R^{w} is selected from the group consisting of amidine, amidoxime, guanidine, and N-hydroxyguanidine; R^{A} is phenyl, wherein R^{A} may optionally be substituted by one or more substituents each selected from R^{g}; or R^{A} is selected from the group consisting of halogen, cyano, hydroxyl, —N(RⁱR^{j}), C₁₋₆alkyl, C₃₋₆cycloalkyl, C₁₋₆alkoxy, C₁₋₆ alkyl-N(R^{a})—, C₁₋₆alkyl-N(R^{a})-carbonyl-, C₁₋₆alkylcarbonyl-N(R^{a})—, and C₁₋₆alkyl-N(R^{a})-carbonyl-N(R^{a})—; wherein the C₁₋₆alkyl, C₃₋₆cycloalkyl, C₁₋₆alkoxy, C₁₋₆alkyl-N(R^{a})—, C₁₋₆alkyl-N(R^{a})— carbonyl-, C₁₋₆alkylcarbonyl-N(R^{a})—, and C₁₋₆alkyl-N(R^{a})-carbonyl-N(R^{a})— may optionally be substituted by R^{p}; R^{B} is selected from the group consisting of: halogen, cyano, hydroxyl, —N(RⁱR^{j}), phenyl, 5-6 membered heteroaryl, 5-6 membered heterocyclyl, —OR^{k}, C₁₋₆alkyl, C₃₋₆cycloalkyl, C₁₋₆ alkoxy, C₁₋₆alkyl-N(R^{a})—, C₁₋₆alkylcarbonyl-N(R^{a})—, and C₁₋₆alkyl-N(R^{a})-carbonyl-; wherein the —N(RⁱR^{j}), phenyl, 5-6 membered heteroaryl, 5-6 membered heterocyclyl, —OR^{k}, C₁₋₆alkyl, C₃₋₆ cycloalkyl, C₁₋₆alkoxy, C₁₋₆alkyl-N(R^{a})—, C₁₋₆alkylcarbonyl-N(R^{a})—, and C₁₋₆alkyl-N(R^{a})-carbonyl-, may optionally be substituted by one, two, or three R^{p}; R^{a} and R^{b} are independently selected, for each occurrence, from the group consisting of hydrogen and C₁₋₃alkyl, wherein the C₁₋₃alkyl may optionally be substituted by one or more substituents each selected from the group consisting of halogen, cyano, oxo, and hydroxyl; or R^{a} and R^{b}, together with the nitrogen to which they are attached, may form a 4-6 membered heterocyclic ring, which may have an additional heteroatom selected from O, S, and N; wherein the 4-6 membered heterocyclic ring may optionally be substituted by one or more substituents each selected from the group consisting of halogen, cyano, oxo, and hydroxyl; R^{f} is independently selected, for each occurrence, from the group consisting of R^{P}, hydrogen, C₁₋₆alkyl, C₃₋₆cycloalkyl, C₂₋₆alkenyl, C₂₋₆alkynyl, C₁₋₆alkoxy, C₁₋₆alkyl-S(O)_{w}—, C₁₋₆alkylcarbonyl-N(R^{a})—, and C₁₋₆alkoxycarbonyl-N(R^{a})—; wherein the C₁₋₆alkyl, C₃₋₆cycloalkyl, C₂₋₆alkenyl, C₂₋₆alkynyl, C₁₋₆alkoxy, C₁₋₆alkyl-S(O)_{w}—, C₁₋₆alkylcarbonyl-N(R^{a})—, or C₁₋₆ alkoxycarbonyl-N(R^{a})— may optionally be substituted by one or more substituents each selected from R^{p}; R⁹ is independently selected, for each occurrence, from the group consisting of R^{P}, hydrogen, oxo, C₁₋₆alkyl, C₃₋₆cycloalkyl, and C₁₋₆alkoxy; R^{h} is independently selected, for each occurrence, from the group consisting of hydrogen, halogen, hydroxyl, cyano, C₁₋₆alkyl, C₃₋₆cycloalkyl, C₁₋₆alkoxy, and (RⁱR^{j})N—; wherein the C₁₋₆alkyl, C₁₋₆alkoxy, or C₃₋₆cycloalkyl, may optionally be substituted by one or more substituents each selected from R^{p}; Rⁱ and R^{j} are independently selected, for each occurrence, from the group consisting of hydrogen, C₁₋₆alkyl, and C₃₋₆cycloalkyl; wherein the C₁₋₆alkyl and C₃₋₆cycloalkyl may be optionally substituted by one or more substituents each selected from halogen, hydroxyl, cyano, (R^{a}R^{b})N—, (R^{a}R^{b})N-carbonyl-, (R^{a}R^{b})N-carbonyl-(R^{a})N—, and C₁₋₃alkoxy; R^{P} is independently selected, for each occurrence, from the group consisting of halogen, hydroxyl, cyano, C₁₋₆alkoxy, (RⁱⁱR^{jj})N—, (RⁱⁱR^{jj})N-carbonyl-, (RⁱⁱR^{jj})N—SO₂—, (RⁱⁱR^{jj})N-carbonyl-N(Rⁱⁱ)—, (CH₃)C(O)N(Rⁱⁱ)—, —C(O)OH, and —C(O)N(RⁱⁱR^{jj}); Rⁱⁱ and R^{jj} are independently selected, for each occurrence, from the group consisting of hydrogen, C₁₋₆alkyl, and C₃₋₆cycloalkyl; R^{k} is selected from the group consisting of C₁₋₆alkyl, C₃₋₆cycloalkyl, 4-6 membered heterocyclyl, C₁₋₆alkyl-(4-6 membered heterocyclyl), and C₁₋₆alkyl-(5-6 membered heteroaryl), wherein the C₁₋₆alkyl may optionally be substituted by one, two, or three R^{p}; w 0, 1, or 2; and pharmaceutically acceptable salts, stereoisomers, esters and prodrugs thereof.

In some embodiments, R^{A} is selected from the group consisting of halogen, cyano, hydroxyl, —N(RⁱR^{j}), C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkoxycarbonyl-N(R^{a})—, C₁₋₆alkylcarbonyl-N(R^{a})—C₁₋₆ alkyl-, C₁₋₆alkyl-N(R^{a})-carbonyl-C₁₋₆alkyl-, and C₁₋₆alkoxyC₁₋₆alkyl-. In some embodiments, R^{A} is selected from the group consisting of halogen, cyano, hydroxyl, —N(RⁱR^{j}), C₁₋₆alkyl, C₃₋₆cycloalkyl, C₁₋₆alkoxy, C₁₋₆alkyl-N(R^{a})—, C₁₋₆alkyl-N(R^{a})-carbonyl-, C₁₋₆alkylcarbonyl-N(R^{a})—, and C₁₋₆alkyl-N(R^{a})-carbonyl-N(R^{a})—. In some embodiments, R^{A} may optionally be substituted by one, two, or three substituents each selected from the group consisting of —OH, NH₂, —N(Me)₂, —NH(Me), —OCH₃, —C(O)OH, —C(O)NH₂, —C(O)NH₂, —C(O)NHMe, —NH(CO)CH₃, —NH(CH₂)(CO)NH₂, —NH(CO)(CH₂)NH₂, —NH(CO)NH₂, —NH(CO)NHMe, and —NH(CO)NHEt.

In some embodiments, R^{B} is selected from the group consisting of: halogen, cyano, hydroxyl, —N(RⁱR^{j}), phenyl, 5-6 membered heteroaryl, 5-6 membered heterocyclyl, —OR^{k}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-SO₂—N(R^{a})—, C₁₋₆alkoxycarbonyl-N(R^{a})—, C₁₋₆alkylcarbonyl-N(R^{a})—C₁₋₆alkyl-, C₁₋₆alkyl-N(R^{a})-carbonyl-C₁₋₆alkyl-, and C₁₋₆alkoxyC₁₋₆alkyl-. In some embodiments, R^{B} is selected from the group consisting of: halogen, cyano, hydroxyl, —N(RⁱR^{j}), phenyl, 5-6 membered heteroaryl, 5-6 membered heterocyclyl, —OR^{k}, C₁₋₆alkyl, C₃₋₆cycloalkyl, C₁₋₆alkoxy, C₁₋₆alkyl-N(R^{a})—, and C₁₋₆alkyl-N(R^{a})-carbonyl-. In some embodiments, R^{B} may optionally be substituted by one, two, or three substituents each selected from the group consisting of —OH, NH₂, —N(Me)₂, —NH(Me), —OCH₃, —C(O)OH, —C(O)NH₂, —C(O)NH₂, —C(O)NHMe, —NH(CO)CH₃, —NH(CH₂)(CO)NH₂, —NH(CO)(CH₂)NH₂, —NH(CO)NH₂, —NH(CO)NHMe, and —NH(CO)NHEt. In some embodiments, R^{B} is selected from the group consisting of C₁₋₆alkyl, C₁₋₆alkoxy, C₁₋₆haloalkyl, (e.g. —CF₃), halogen, 5-6 membered heterocyclyl optionally substituted by C₁₋₆alkyl, and NH(C₁₋₆alkyl) optionally substituted by —NH₂, —N(CH₃)₂, and —OCH₃.

In some embodiments, R^{f} is independently selected, for each occurrence, from the group consisting of hydrogen, C₁₋₆alkyl, C₃₋₆cycloalkyl, halogen, hydroxyl, cyano, C₁₋₆alkoxy, (RⁱⁱR^{jj})N—, (RⁱⁱR^{jj})N-carbonyl-, (RⁱⁱR^{jj})N—SO₂—, (RⁱⁱR^{jj})N-carbonyl-N(Rⁱⁱ)—, (CH₃)C(O)N(Rⁱⁱ)—, —C(O)OH, and —C(O)N(RⁱⁱR^{jj}); and Rⁱⁱ and R^{jj} are independently selected, for each occurrence, from the group consisting of hydrogen, C₁₋₆alkyl, and C₃₋₆cycloalkyl; and wherein the alkyl or alkoxy may optionally be substituted by one, two, or three substituents each selected from the group consisting of —OH, NH₂, —N(Me)₂, —NH(Me), —OCH₃, —C(O)OH, —C(O)NH₂, —C(O)NH₂, —C(O)NHMe, —NH(CO)CH₃, —NH(CH₂)(CO)NH₂, —NH(CO)(CH₂)NH₂, —NH(CO)NH₂, —NH(CO)NHMe, and —NH(CO)NHEt.

In some embodiments, R⁹ is independently selected, for each occurrence, from the group consisting of hydrogen, oxo, C₁₋₆alkyl, C₃₋₆cycloalkyl, halogen, hydroxyl, cyano, C₁₋₆alkoxy, (RⁱⁱR^{jj})N—, (RⁱⁱR^{jj})N-carbonyl-, (RⁱⁱR^{jj})N—SO₂—, (RⁱⁱR^{jj})N-carbonyl-N(Rⁱⁱ)—, (CH₃)C(O)N(Rⁱⁱ)—, —C(O)OH, and —C(O)N(RⁱⁱR^{jj}); and Rⁱⁱ and R^{jj} are independently selected, for each occurrence, from the group consisting of hydrogen, C₁₋₆alkyl, and C₃₋₆cycloalkyl; and wherein the alkyl or alkoxy may optionally be substituted by one, two, or three substituents each selected from the group consisting of —OH, NH₂, —N(Me)₂, —NH(Me), —OCH₃, —C(O)OH, —C(O)NH₂, —C(O)NH₂, —C(O)NHMe, —NH(CO)CH₃, —NH(CH₂)(CO)NH₂, —NH(CO)(CH₂)NH₂, —NH(CO)NH₂, —NH(CO)NHMe, and —NH(CO)NHEt.

In some embodiments, R^{h} is selected from the group consisting of C₁₋₆alkyl and (RⁱR^{j})N—, wherein Rⁱ and R^{j} are independently, for each occurrence, are selected from the group consisting of hydrogen and C₁₋₆alkyl, wherein the C₁₋₆alkyl is substituted by one, two, or three substituent each selected from the group consisting of (R^{a}R^{b})N—, (R^{a}R^{b})N-carbonyl-, (R^{a}R^{b})N-carbonyl-(R^{a})N—, and R^{a} and R^{b} are independently selected, for each occurrence, from the group consisting of hydrogen and C₁₋₃alkyl.

In some embodiments, R^{k} is selected from the group consisting of C₁₋₆alkyl, C₃₋₆ cycloalkyl, 4-6 membered heterocyclyl, C₁₋₆alkyl-(4-6 membered heterocyclyl) (e.g.,

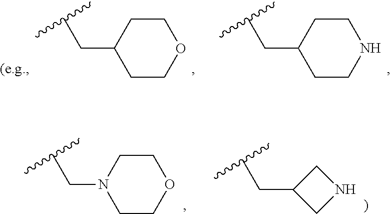 [SMILES: *CC1CCOCC1 ; *CC1CCNCC1 ; *CN1CCOCC1 ; *CC1CNC1]

and C₁₋₆alkyl-(5-6 membered heteroaryl) (e.g.,

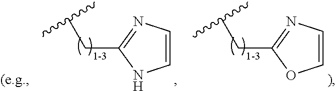

wherein the C₁₋₆alkyl may optionally be substituted by one, two, or three substituents each selected from the group consisting of —OH, NH₂, —N(Me)₂, —NH(Me), —OCH₃, —C(O)OH, —C(O)NH₂, —C(O)NH₂, —C(O)NHMe, —NH(CO)CH₃, —NH(CH₂)(CO)NH₂, —NH(CO)(CH₂)NH₂, —NH(CO)NH₂, —NH(CO)NHMe, and —NH(CO)NHEt.

In some embodiments, R^{w} is selected from the group consisting of:

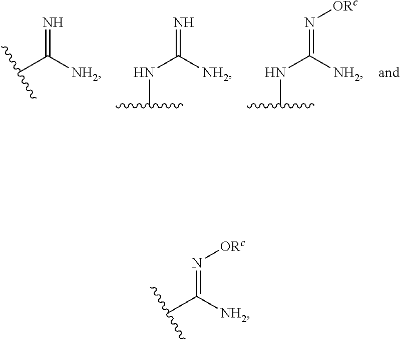

wherein R^{C} is hydrogen or C₁₋₆alkyl. In some embodiments, R^{C} is hydrogen. In some embodiments, R^{C} is C₁₋₆alkyl. In some embodiments, R^{w} is

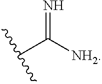 [SMILES: *C(=N)N]

In some embodiments, R^{w} is

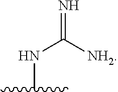 [SMILES: *NC(=N)N]

In some embodiments, R^{w} is

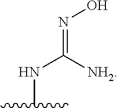 [SMILES: *N/C(N)=N/O]

In some embodiments, R^{w} is

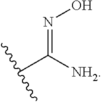 [SMILES: */C(N)=N/O]

In some embodiments, W is phenyl. In some embodiments, W is phenyl. In embodiments, W is unsubstituted phenyl. In embodiments, W is substituted phenyl.

In embodiments, the present disclosure provides compounds of Formula I-a:

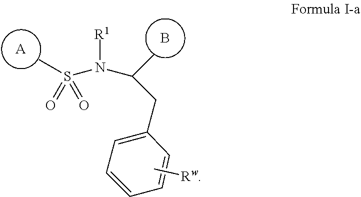

In embodiments, the present disclosure provides compounds of Formula I-b:

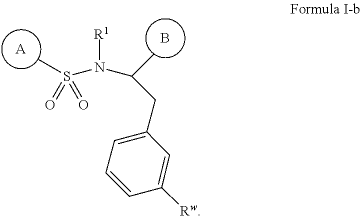

In certain embodiments, A is unsubstituted phenyl. In certain embodiments, A is substituted phenyl. In embodiments, A is a phenyl optionally substituted by one, two, or three R^{A} wherein R^{A} is selected from the group consisting of halogen, cyano, hydroxyl, —N(RⁱR^{j}), C₁₋₆alkyl, C₃₋₆cycloalkyl, C₁₋₆alkoxy, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, and phenyl, wherein the alkyl or phenyl may optionally be substituted by one, two or three substituent each selected from the group consisting of halogen, —NH₂, —OCH₃, and —OCH₂CH₃, and R^{a} is hydrogen or C₁₋₆alkyl. In certain embodiments, A is a phenyl optionally substituted by one, two or three substituent each selected from halogen and —NH—C(O)—(CH₂CH₂)NH₂. In embodiments, A is selected from the group consisting of

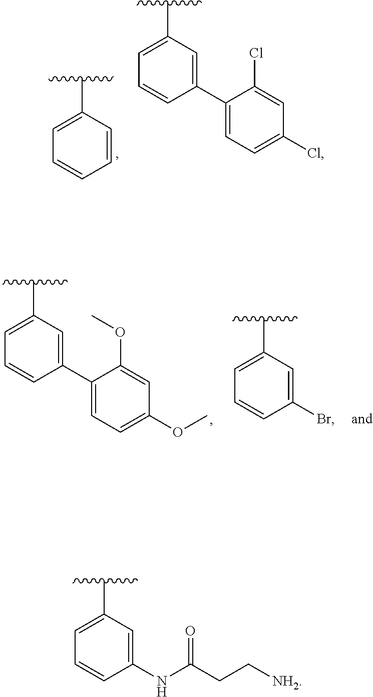

In some embodiments, A is bis-phenyl. In some embodiments, A is bis-phenyl optionally substituted by one, two or three substituent each selected from halogen, —OCH₃, and —OCH₂CH₃. In some embodiments, A is a monocyclic heteroaryl substituted by phenyl or monocyclic heteroaryl.

In embodiments, A is substituted C₁₋₆alkyl. In embodiments, A is unsubstituted C₁₋₆ alkyl. In certain embodiments, A is selected from the group consisting of —CH₂CH₂CH₃, —CH₂CH₂OCH₃ and cyclopropyl.

In embodiments, R^{A} is selected from the group consisting of halogen, —N(RⁱR^{j}), C₁₋₆alkyl, C₁₋₆alkoxy, C₃₋₆cycloalkyl, C₁₋₆alkyl-N(R^{a})—, C₁₋₆alkylcarbonyl-N(R^{a})—, and phenyl, wherein the alkyl and phenyl may optionally be substituted by one, two, or three substituents each independently selected from the group consisting of halogen, —NH₂, —OCH₃, and —OCH₂CH₃; R^{a} is selected from hydrogen and C₁₋₆alkyl, and Rⁱ and R^{j} are independently selected, for each occurrence, from the group consisting of hydrogen, C₁₋₆alkyl, and C₃₋₆cycloalkyl.

In embodiments, R^{B} is selected from the group consisting of halogen, cyano, hydroxyl, —OMe, —CHCl₂, —CCl₃, —CHF₂, —CF₃, —N(RⁱR^{j}), C₁₋₆alkyl-N(R^{a})—, C₁₋₆alkyl, —OR^{k}, and 5-6 membered heterocyclyl; wherein the alkyl and heterocyclyl may optionally be substituted by one, two, or three substituents each independently selected from the group consisting of R^{P}, and R^{k} is selected from the group consisting of C₁₋₆alkyl, C₃₋₆cycloalkyl, 4-6 membered heterocyclyl, C₁₋₆ alkyl-(4-6 membered heterocyclyl), and C₁₋₆alkyl-(5-6 membered heteroaryl), wherein the C₁₋₆alkyl may optionally be substituted by one, two, or three R^{p}; wherein R^{P} is selected from the group consisting of —OH, NH₂, —N(Me)₂, —NH(Me), —OCH₃, —C(O)OH, —C(O)NH₂, —C(O)NH₂, —C(O)NHMe, —NH(CO)CH₃, —NH(CH₂)(CO)NH₂, —NH(CO)(CH₂)NH₂, —NH(CO)NH₂, —NH(CO)NHMe, and —NH(CO)NHEt; R^{a} is selected from hydrogen and C₁₋₆alkyl; and Rⁱ and R^{j} are independently selected, for each occurrence, from the group consisting of hydrogen, C₁₋₆alkyl, and C₃₋₆cycloalkyl.

In some embodiments, the present disclosure provides compounds of Formula I-c:

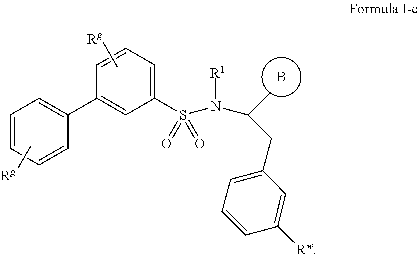

In some embodiments, the present disclosure provides compounds of Formula I-c:

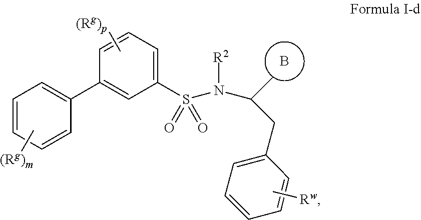

wherein R⁹ is independently selected, for each occurrence, from the group consisting of H, halogen, and C₁₋₆alkoxy; m is 1, 2, or 3; and p is 1, 2, or 3.

In embodiments, R⁹ is selected from the group consisting of hydrogen, halogen, C₁₋₆ alkyl, C₁₋₆haloalkyl, and C₁₋₆alkoxy. In embodiments, R⁹ is selected from the group consisting of hydrogen, —Cl, —OCH₃, and —OCH₂CH₃. In embodiments, R⁹ is hydrogen. In embodiments, R⁹ is halogen. In embodiments, R⁹ is C₁₋₆alkyl. In embodiments, R⁹ is C₁₋₆alkoxy.

In embodiments, B is optionally substituted 5-6 membered monocyclic heteroaryl, wherein B contains at least one nitrogen. In some embodiments, B is optionally substituted 8-10 membered bicyclic heteroaryl, wherein B contains at least one nitrogen. In some embodiments, B is optionally substituted 8-10 membered bicyclic heterocyclyl, wherein B contains at least one nitrogen. In some embodiments, B is optionally substituted by halogen, hydroxyl, C₁₋₆haloalkyl, C₁₋₆ alkoxy, C₁₋₆alkyl, (R^{a}R^{b})N—, N(R^{a}R^{b})—C₁₋₆alkyl-, (R^{a}R^{b})N—C(O)—C₁₋₆alkyl-, N(R^{a}R^{b})—C(O)—N(R^{a})—C₁-6alkyl-, C₁₋₆alkyl-N(R^{a})—C(O)—C₁₋₆alkyl-, C₁₋₆alkyl-C(O)—N(R^{a})—C₁₋₆alkyl-, C(O)OH—C₁₋₆alkyl-, N(R^{a}R^{b})—C₁₋₆alkyl-N(R^{a})—, C₁₋₆alkoxy-C₁₋₆alkyl-N(R^{a})—, N(R^{a}R^{b})—C(O)—C₁₋₆alkyl-N(R^{a})—, N(R^{a}R^{b})—C(O)—N(R^{a})—C₁₋₆alkyl-N(R^{a})—, C₁₋₆alkyl-N(R^{a})—C(O)—N(R^{a})—, N(R^{a}R^{b})-Cl₆alkyl-C(O)—N(R^{a})—, N(R^{a}R^{b})—C₁₋₆alkyl-O—, (R^{a})O—C₁₋₆alkyl-O—, N(R^{a}R^{b})—C(O)—N(R^{a})—C₁₋₆alkyl-O—, N(R^{a}R^{b})—C(O)—C₁₋₆alkyl-O—, (5-6 membered heterocyclyl)-C₁₋₆alkyl-O—, (5-6 membered heterocyclyl)-O—, (5-6 membered heteroaryl)-C₁₋₆alkyl-O—, (R^{a})C(O)—N(R^{a})—C₁₋₆alkyl-O—, and —N(RⁱR^{j}), wherein Rⁱ and R^{j} are taken together with the nitrogen to which they are attached form a 4-7 membered heterocyclic ring optionally be substituted by one, two or three substituent each selected from the group consisting of C₁₋₆alkyl, —NH₂, —C(O)NH(C₁₋₃alkyl), and Raand R^{b} are independently, for each occurrence, hydrogen or C₁₋₃alkyl. In certain embodiments, B is selected from the group consisting of pyridinyl, pyrimidinyl, pyrrole, thiazole, oxazole, isothiazole, isoxazole, imidazole, benzothiazole, benzoimidazole, and benzoxazole. In certain embodiments, B is selected from the group consisting of pyridinyl, pyrimidinyl, pyrrole, thiazole, oxazole, isothiazole, isoxazole, imidazole, benzothiazole, benzoimidazole, benzoxazole, tetrahydroimidazopyridine and tetrahydroimidazopyrazine.

In some embodiments, B is selected from the group consisting of

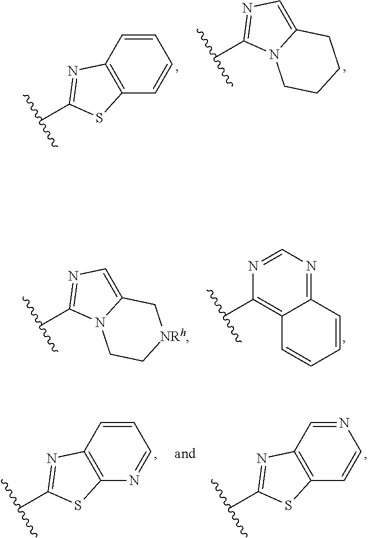

wherein B may optionally substituted on one, two, or three carbons by a substituent each independently selected from the group consisting of halogen, hydroxyl, C₁₋₆haloalkyl, C₁₋₆alkoxy, C₁₋₆alkyl, (R^{a}R^{b})N—, N(R^{a}R^{b})—C₁₋₆alkyl-, (R^{a}R^{b})N—C(O)—C₁₋₆alkyl-, N(R^{a}R^{b})—C(O)—N(R^{a})—C₁₋₆alkyl-, C₁₋₆alkyl-N(R^{a})—C(O)—C₁₋₆alkyl-, C₁₋₆alkyl-C(O)—N(R^{a})—C₁₋₆alkyl-, C(O)OH—C₁₋₆alkyl-, N(R^{a}R^{b})—C₁₋₆alkyl-N(R^{a})—, C₁₋₆alkoxy-C₁₋₆alkyl-N(R^{a})—, N(R^{a}R^{b})—C(O)—C₁₋₆alkyl-N(R^{a})—, N(R^{a}R^{b})—C(O)—N(R^{a})—C₁₋₆alkyl-N(R^{a})—, C₁₋₆alkyl-N(R^{a})—C(O)—N(R^{a})—, N(R^{a}R^{b})—C₁₋₆alkyl-C(O)—N(R^{a})—, N(R^{a}R^{b})—C₁₋₆alkyl-O—, (R^{a})O—C₁₋₆alkyl-O—, N(R^{a}R^{b})—C(O)—N(R^{a})—C₁₋₆alkyl-O—, N(R^{a}R^{b})—C(O)—C₁₋₆alkyl-O—, (5-6 membered heterocyclyl)-C₁₋₆alkyl-O—, (5-6 membered heterocyclyl)-O—, (5-6 membered heteroaryl)-C₁₋₆alkyl-O—, (R^{a})C(O)—N(R^{a})—C₁₋₆alkyl-O—, and —N(RⁱR^{j}), wherein Rⁱ and R^{j} are taken together with the nitrogen to which they are attached form a 4-7 membered heterocyclic ring optionally be substituted by one, two or three substituent each independently selected from the group consisting of C₁₋₆alkyl, —NH₂, —C(O)NH(C₁₋₃alkyl), and R^{a} and R^{b} are independently, for each occurrence, hydrogen or C₁₋₃alkyl; and R^{h} is selected from the group consisting of C₁₋₆alkyl and (RⁱR^{j})N—, wherein Rⁱ and R^{j} are independently, for each occurrence, are selected from the group consisting of hydrogen and C₁₋₆ alkyl, wherein the C₁₋₆alkyl is substituted by one, two, or three substituent each selected from the group consisting of (R^{a}R^{b})N—, (R^{a}R^{b})N-carbonyl-, (R^{a}R^{b})N-carbonyl-(R^{a})N—, and R^{a} and R^{b} are independently selected, for each occurrence, from the group consisting of hydrogen and C₁₋₃alkyl.

In some embodiments, B is selected from the group consisting of:

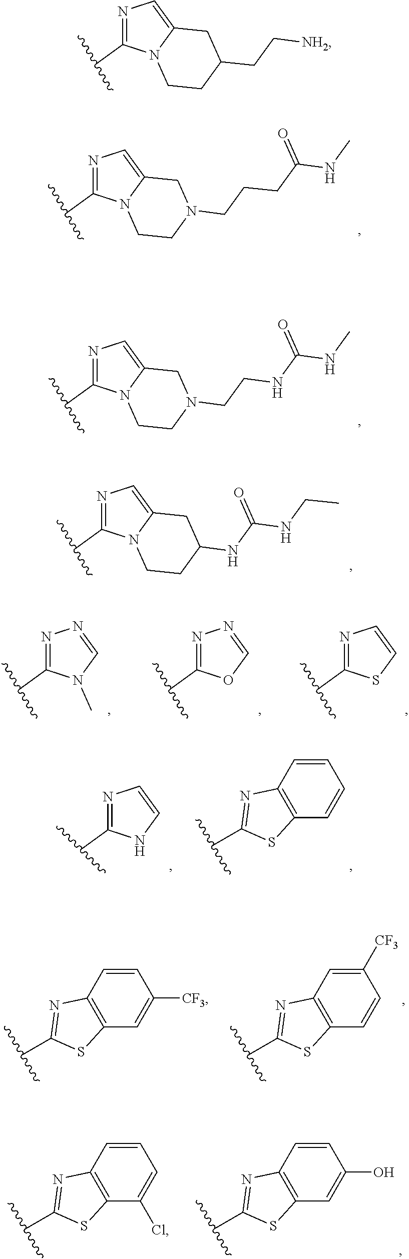

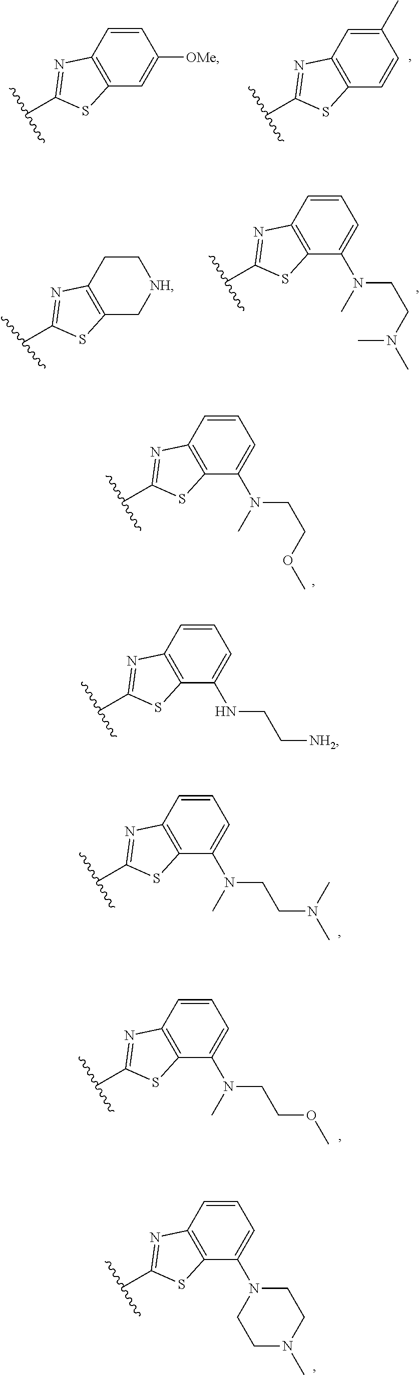

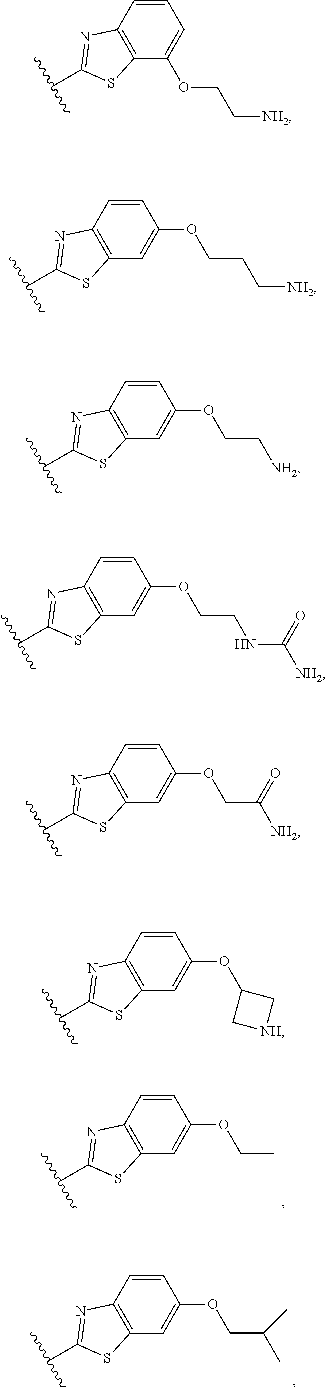

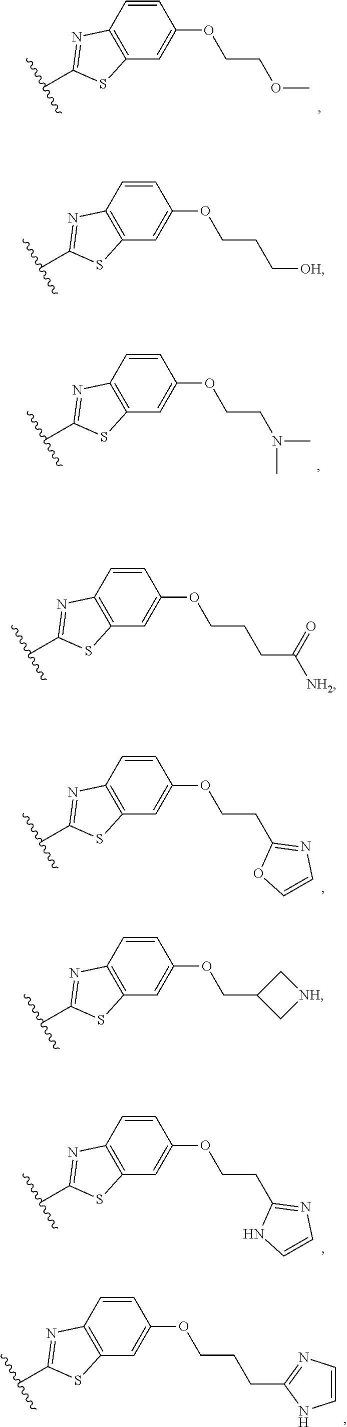

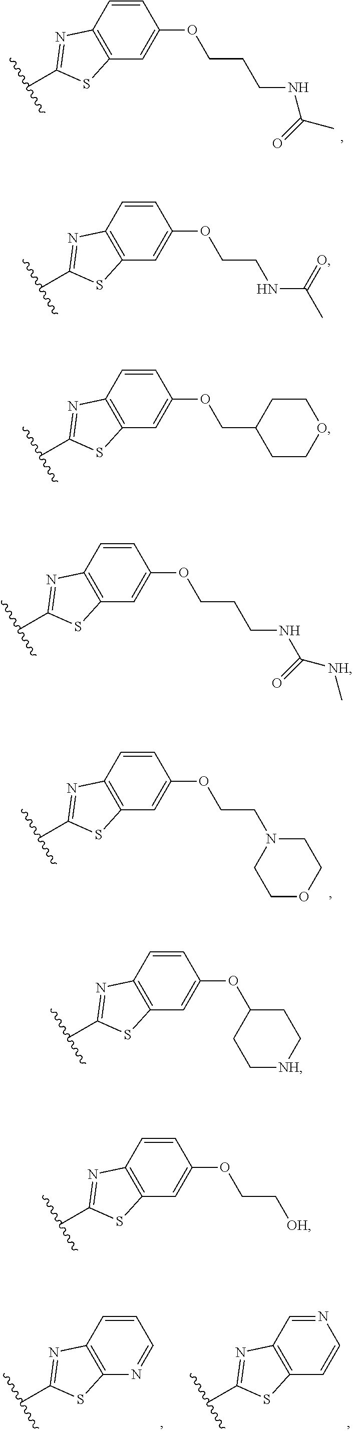

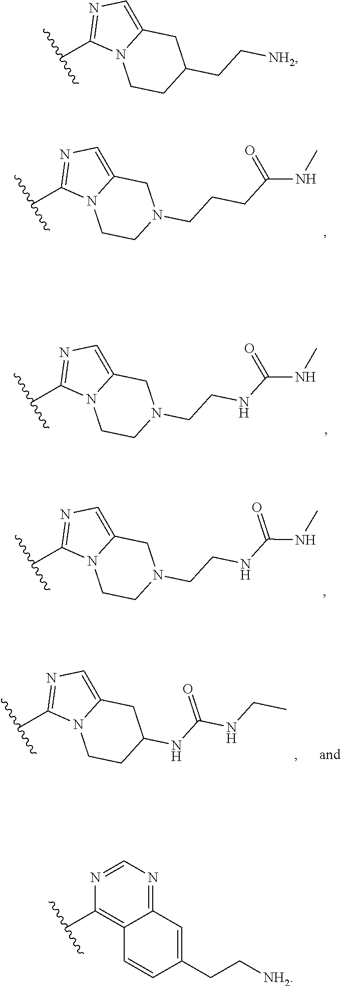 [SMILES: *c1ncc2n1CCC(CCN)C2 ; *c1ncc2n1CCN(CCCC(=O)NC)C2 ; *c1ncc2n1CCN(CCNC(=O)NC)C2 ; *c1ncc2n1CCN(CCNC(=O)NC)C2 ; *c1ncc2n1CCC(NC(=O)NCC)C2 ; *c1ncnc2cc(CCN)ccc12]

In some embodiments, B is selected from the group consisting of,

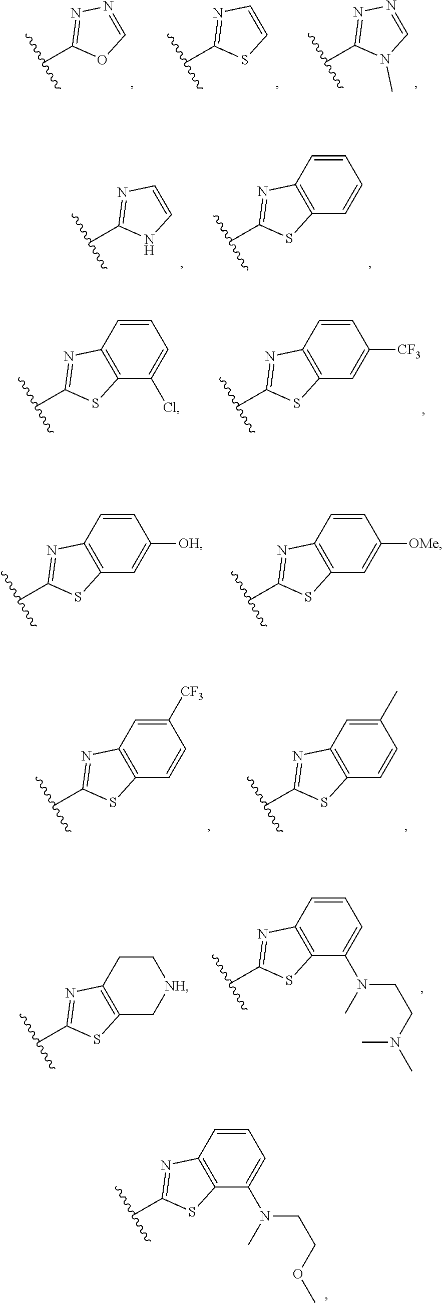

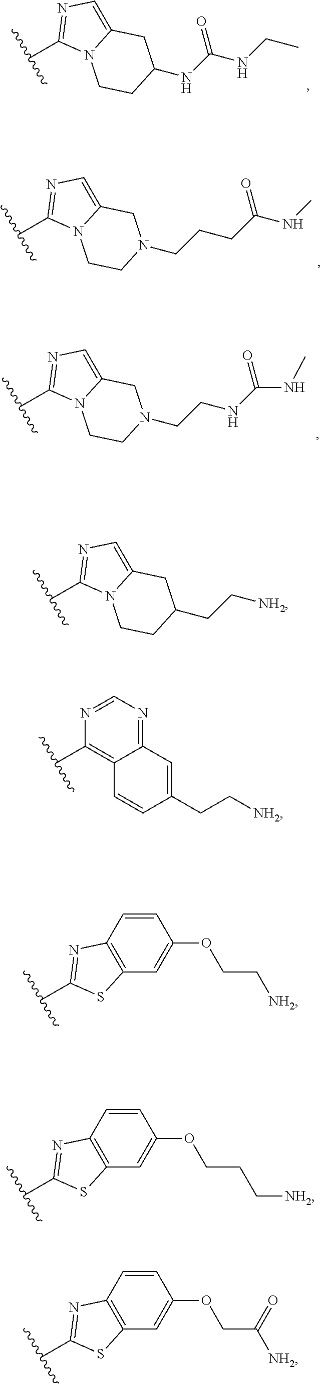

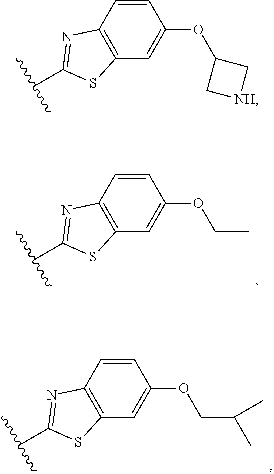 [SMILES: *c1nc2ccc(OC3CNC3)cc2s1 ; *c1nc2ccc(OCC)cc2s1 ; *c1nc2ccc(OCC(C)C)cc2s1]

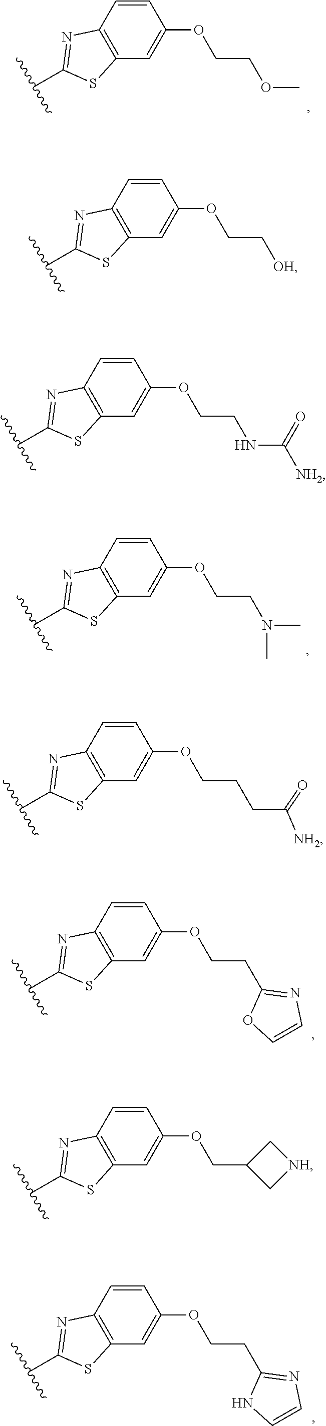

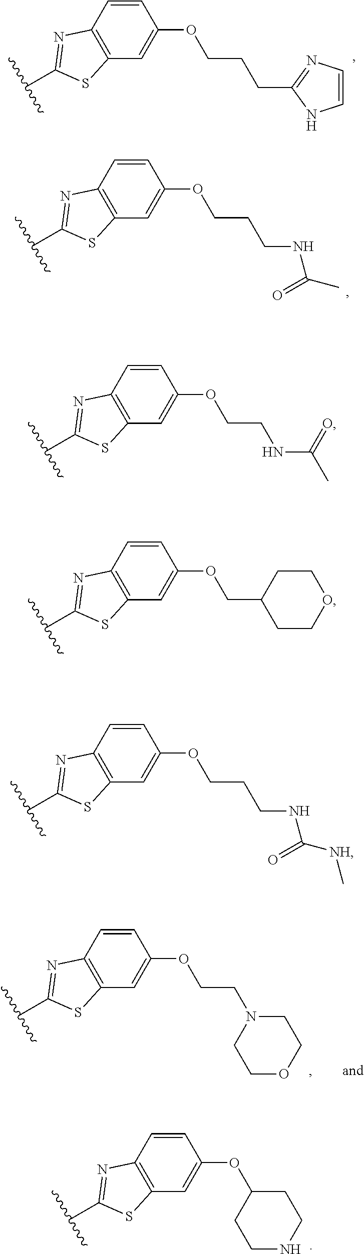

In some embodiments, R¹ is hydrogen. In certain embodiments, R¹ is C₁₋₆ alkyl.

In certain embodiments, the present disclosure provides compounds of Formula II:

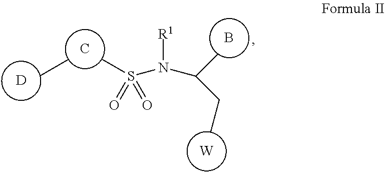

C and D are independently selected, for each occurrence, from the group consisting of phenyl and 5-6 membered monocyclic heteroaryl, wherein the phenyl or 5-6 membered monocyclic heteroaryl may optionally be substituted by one or more substituents each selected from Rg; R2 is selected from hydrogen and C1-6 alkyl; B is selected from the group consisting of 5-6 membered monocyclic heteroaryl, 8-10 membered bicyclic heteroaryl, and 8-10 membered bicyclic heterocyclyl, wherein B contains at least one nitrogen and B is optionally substituted on one, two, or three carbons by a substituent each independently selected from R^{B}; wherein when said heterocyclyl contains a —NH moiety, the nitrogen of —NH may optionally be substituted by one or more substituents each selected from R^{h}; W is selected from phenyl and heteroaryl, wherein W is substituted on a carbon by a warhead moiety R^{w}, wherein R^{w} is selected from the group consisting of amidine, amidoxime, guanidine, and N-hydroxyguanidine; R^{B} is selected from the group consisting of: halogen, cyano, hydroxyl, —N(RⁱR^{j}), phenyl, 5-6 membered heteroaryl, 5-6 membered heterocyclyl, —OR^{k}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}— (wherein w is 0, 1 or 2), C₁₋₆alkyl-N(R^{a})—, C₁₋₆ alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})—C₁₋₆alkyl-, C₁₋₆alkyl-N(R^{a})-carbonyl-C₁₋₆alkyl-, C₁₋₆alkoxyC₁₋₆alkyl-; wherein the C₁₋₆alkyl, C₂₋₆alkenyl, C₂-6alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}—, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})— carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})C₁₋₆alkyl-, C₁₋₆alkyl-N(R^{a})-carbonyl-C₁₋₆alkyl-, and C₁₋₆alkoxy-C₁₋₆alkyl may optionally be substituted by R^{P}, phenyl, phenoxy, 5-6 membered heteroaryl, heteroaryloxy, 5-6 membered heteroaryl-(NR^{a})—, 4-6 membered heterocyclyl, 4-6 membered heterocyclyloxy or 4-6 membered heterocyclyl-N(R^{a})—; and wherein said heteroaryl or phenyl may optionally be substituted with one or more substituents selected each from R^{f}; and wherein said heterocyclyl may optionally be substituted by one or more substituents each selected from R^{g}; and wherein when said heterocyclyl contains a —NH moiety, the nitrogen of —NH may optionally be substituted by one or more substituents each selected from R^{h}; R^{a} and R^{b} are independently selected, for each occurrence, from the group consisting of hydrogen and C₁₋₃alkyl, wherein the C₁₋₃alkyl may optionally be substituted by one or more substituents each selected from the group consisting of halogen, cyano, oxo, and hydroxyl; or R^{a} and R^{b}, together with the nitrogen to which they are attached, may form a 4-6 membered heterocyclic ring, which may have an additional heteroatom selected from O, S, and N; wherein the 4-6 membered heterocyclic ring may optionally be substituted by one or more substituents each selected from the group consisting of halogen, cyano, oxo, and hydroxyl; R⁹ is independently selected, for each occurrence, from the group consisting of R^{P}, hydrogen, oxo, C₁₋₆alkyl, C₂₋₆alkenyl, C₂₋₆alkynyl, C₃₋₆cycloalkyl, C₁₋₆alkoxy, C₁₋₆alkyl-S(O)_{w}—, C₁₋₆alkylcarbonyl-N(R^{a})—, and C₁₋₆alkoxycarbonyl-N(R^{a})—; wherein the C₁₋₆alkyl, C₂₋₆alkenyl, C₂₋₆alkynyl, C₃₋₆cycloalkyl, C₁₋₆alkoxy, C₁₋₆alkyl-S(O)_{w}—, C₁₋₆alkylcarbonyl-N(R^{a})—, or C₁₋₆alkoxycarbonyl-N(R^{a})— may optionally be substituted by one or more substituents each selected from R^{p}; R^{h} is independently selected, for each occurrence, from the group consisting of hydrogen, halogen, hydroxyl, cyano, C₁₋₆alkyl, C₃₋₆alkenyl, C₃₋₆alkynyl, C₃₋₆cycloalkyl, C₁₋₆alkoxy, (RⁱR^{j})N—, C₁₋₆alkyl-S(O)₂—, C₁₋₆alkoxycarbonyl-, (RⁱR^{j})N-carbonyl-, and (RⁱR^{j})N—SO₂—; wherein the C₁₋₆alkyl, C₁₋₆alkoxy, C₃₋₆alkenyl, C₃₋₆alkynyl C₃₋₆cycloalkyl, C₁₋₆alkyl-S(O)₂—, or C₁₋₆alkylcarbonyl- may optionally be substituted by one or more substituents each selected from R^{p}; Rⁱ and R^{j} are independently selected, for each occurrence, from the group consisting of hydrogen, C₁₋₆alkyl, and C₃₋₆cycloalkyl; wherein the C₁₋₆alkyl and C₃₋₆cycloalkyl may be optionally substituted by one or more substituents each selected from halogen, hydroxyl, cyano, (R^{a}R^{b})N—, (R^{a}R^{b})N-carbonyl-, (R^{a}R^{b})N-carbonyl-(R^{a})N—, and C₁₋₃alkoxy; or Rⁱ and R^{j} taken together with the nitrogen to which they are attached form a 4-7 membered heterocyclic ring, which may have an additional heteroatom selected from O, S, and N; wherein the 4-7 membered heterocyclic ring may optionally be substituted on carbon by one or more substituents each selected from the group consisting of halogen, hydroxyl, oxo, cyano, C₁₋₆alkyl, C₁₋₆alkoxy, (R^{a}R^{b})N—, (R^{a}R^{b})N—SO₂—, and (R^{a}R^{b})N-carbonyl-; wherein said C₁₋₆alkyl or C₁₋₆alkoxy may optionally be substituted by halogen, hydroxyl or cyano; wherein the 4-7 membered heterocyclic ring may be optionally substituted on nitrogen by one or more substituents each selected from the group consisting of C₁₋₆alkyl and (R^{a}R^{b})N-carbonyl-; and wherein said C₁₋₆alkyl may be optionally substituted by one or more substituents each selected from the group consisting of halogen, hydroxyl, and cyano; R^{P} is independently selected, for each occurrence, from the group consisting of halogen, hydroxyl, cyano, C₁₋₆alkoxy, (RⁱⁱR^{jj})N—, (RⁱⁱR^{jj})N-carbonyl-, (RⁱⁱR^{jj})N—SO₂—, (RⁱⁱR^{jj})N-carbonyl-N(Rⁱⁱ)—, (CH₃)C(O)N(Rⁱⁱ)—, —C(O)OH, and —C(O)N(RⁱⁱR^{jj}); Rⁱⁱ and R^{jj} are independently selected, for each occurrence, from the group consisting of hydrogen, C₁₋₆alkyl, and C₃₋₆cycloalkyl; R^{k} is selected from the group consisting of C₁₋₆alkyl, C₃₋₆cycloalkyl, 4-6 membered heterocyclyl, C₁₋₆alkyl-(4-6 membered heterocyclyl), and C₁₋₆alkyl-(5-6 membered heteroaryl), wherein the C₁₋₆alkyl may optionally be substituted by one, two, or three R^{p}; w 0, 1, or 2; and pharmaceutically acceptable salts, stereoisomers, esters and prodrugs thereof.

In some embodiments, the present disclosure provides compounds of Formula II:

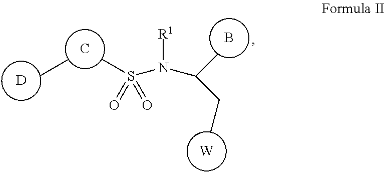

wherein: C and D are independently selected, for each occurrence, from the group consisting of phenyl and 5-6 membered monocyclic heteroaryl, wherein the phenyl or 5-6 membered monocyclic heteroaryl may optionally be substituted by one or more substituents selected from R^{g}; R² is selected from hydrogen and C₁₋₆ alkyl; B is selected from the group consisting of 5-6 membered monocyclic heteroaryl, 8-10 membered bicyclic heteroaryl, and 8-10 membered bicyclic heterocyclyl, wherein B contains at least one nitrogen and is optionally substituted on one, two, or three carbons by a substituent each independently selected from R^{B}; wherein when said heterocyclyl contains a —NH moiety, the nitrogen of —NH may optionally be substituted by one or more substituents each selected from R^{h}; W is selected from phenyl and heteroaryl, wherein W is substituted on a carbon by a warhead moiety R^{w}, wherein R^{w} is selected from the group consisting of amidine, amidoxime, guanidine, and N-hydroxyguanidine; R^{A} is selected from the group consisting of phenyl, 5-6 membered heteroaryl, and 4-10 membered heterocyclyl, wherein R^{A} may optionally be substituted by one or more substituents each selected from R^{g}; wherein when said heterocyclyl contains a —NH moiety, the nitrogen of —NH may optionally be substituted by one or more substituents each selected from R^{h}; or R^{A} is selected from the group consisting of halogen, cyano, hydroxyl, —N(RⁱR^{j}), C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃-6cycloalkoxy, C₁₋₆alkyl-S(O)_{w}—, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆ alkoxycarbonyl-N(R^{a})—, C₁₋₆alkylcarbonyl-N(R^{a})—C₁₋₆alkyl-, C₁₋₆alkyl-N(R^{a})-carbonyl-C₁₋₆alkyl-, and C₁₋₆alkoxyC₁₋₆alkyl-; wherein the C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}—, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})C₁₋₆alkyl-, C₁₋₆alkyl-N(R^{a})-carbonyl-C₁₋₆alkyl-, or C₁₋₆alkoxy-C₁₋₆alkyl may optionally be substituted by R^{P}, phenyl, phenoxy, heteroaryl, heteroaryloxy, heteroaryl-(NR^{a})—, heterocyclyl, heterocyclyloxy or heterocyclyl-N(R^{a})—; wherein said phenyl or heteroaryl may optionally be substituted with one or more substituents selected from R^{f}; wherein said heterocyclyl may optionally be substituted by one or more substituents selected from R^{g}; and wherein when said heterocyclyl contains a —NH moiety, the nitrogen of —NH may optionally be substituted by one or more substituents each selected from R^{h}; R^{B} is selected from the group consisting of: halogen, cyano, hydroxyl, —N(RⁱR^{j}), phenyl, heteroaryl, heterocyclyl, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}— (wherein w is 0, 1 or 2), C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})—C₁₋₆alkyl-, C₁₋₆alkyl-N(R^{a})-carbonyl-C₁₋₆alkyl-, C₁₋₆alkoxyC₁₋₆alkyl-; wherein C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}—, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})— carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})C₁₋₆alkyl-, C₁₋₆alkyl-N(R^{a})-carbonyl-C₁₋₆alkyl-, C₁₋₆alkoxy-C₁₋₆alkyl may optionally be substituted by R^{P}, phenyl, phenoxy, heteroaryl, heteroaryloxy, heteroaryl-(NR^{a})—, heterocyclyl, heterocyclyloxy or heterocyclyl-N(R^{a})—; and wherein said heteroaryl or phenyl may optionally be substituted with one or more substituents selected from R^{f}; and wherein said heterocyclyl may optionally be substituted by one or more substituents selected from R^{g}; and wherein when said heterocyclyl contains a —NH moiety, the nitrogen of —NH may optionally be substituted by one or more substituents each selected from R^{h}; R^{a} and R^{b} are independently selected, for each occurrence, from the group consisting of hydrogen and C₁₋₃alkyl, wherein the C₁₋₃alkyl may optionally be substituted by one or more substituents selected from the group consisting of halogen, cyano, oxo, and hydroxyl; or R^{a} and R^{b}, together with the nitrogen to which they are attached, may form a 4-6 membered heterocyclic ring, which may have an additional heteroatom selected from O, S, and N; wherein the 4-6 membered heterocyclic ring may optionally be substituted by one or more substituents selected from the group consisting of halogen, cyano, oxo, and hydroxyl; R^{f} is independently selected, for each occurrence, from the group consisting of R^{P}, hydrogen, C₁₋₆alkyl, C₃₋₆cycloalkyl, C₂₋₆alkenyl, C₂₋₆alkynyl, C₁₋₆alkoxy, C₁₋₆alkyl-S(O)_{w}—, C₁₋₆alkylcarbonyl-N(R^{a})—, and C₁₋₆alkoxycarbonyl-N(R^{a})—; wherein the C₁₋₆alkyl, C₃₋₆cycloalkyl, C₂₋₆alkenyl, C₂₋₆alkynyl, C₁₋₆alkoxy, C₁₋₆alkyl-S(O)_{w}—, C₁₋₆alkylcarbonyl-N(R^{a})—, or C₁₋₆alkoxycarbonyl-N(R^{a})— may optionally be substituted by one or more substituents selected from R^{p}; R⁹ is independently selected, for each occurrence, from the group consisting of R^{P}, hydrogen, oxo, C₁₋₆alkyl, C₂₋₆alkenyl, C₂₋₆alkynyl, C₃₋₆cycloalkyl, C₁₋₆alkoxy, C₁₋₆alkyl-S(O)_{w}—, C₁₋₆alkylcarbonyl-N(R^{a})—, and C₁₋₆alkoxycarbonyl-N(R^{a})—; wherein the C₁₋₆alkyl, C₂₋₆alkenyl, C₂₋₆alkynyl, C₃₋₆cycloalkyl, C₁₋₆alkoxy, C₁₋₆alkyl-S(O)_{w}—, C₁₋₆alkylcarbonyl-N(R^{a})—, or C₁₋₆alkoxycarbonyl-N(R^{a})— may optionally be substituted by one or more substituents selected from R^{p}; R^{h} is independently selected, for each occurrence, from the group consisting of hydrogen, halogen, hydroxyl, cyano, C₁₋₆alkyl, C₃₋₆alkenyl, C₃₋₆alkynyl, C₃₋₆cycloalkyl, C₁₋₆alkoxy, (RⁱR^{j})N—, C₁₋₆alkyl-S(O)₂—, C₁₋₆alkoxycarbonyl-, (RⁱR^{j})N-carbonyl-, and (RⁱR^{j})N—SO₂—; wherein the C₁₋₆alkyl, C₁₋₆alkoxy, C₃₋₆alkenyl, C₃₋₆alkynyl C₃₋₆cycloalkyl, C₁₋₆alkyl-S(O)₂—, or C₁₋₆alkylcarbonyl- may optionally be substituted by one or more substituents selected from R^{p}; Rⁱ and R^{j} are independently selected, for each occurrence, from the group consisting of hydrogen, C₁₋₆alkyl, and C₃₋₆cycloalkyl; wherein the C₁₋₆alkyl and C₃₋₆cycloalkyl may be optionally substituted by one or more substituents selected from halogen, hydroxyl, cyano, (R^{a}R^{b})N—, (R^{a}R^{b})N-carbonyl-, and C₁₋₃alkoxy; or Rⁱ and R^{j} taken together with the nitrogen to which they are attached form a 4-7 membered heterocyclic ring, which may have an additional heteroatom selected from O, S, and N; wherein the 4-7 membered heterocyclic ring may optionally be substituted on carbon by one or more substituents selected from the group consisting of halogen, hydroxyl, oxo, cyano, C₁₋₆alkyl, C₁₋₆alkoxy, (R^{a}R^{b})N—, (R^{a}R^{b})N—SO₂—, and (R^{a}R^{b})N-carbonyl-; wherein said C₁₋₆alkyl or C₁₋₆ alkoxy may optionally be substituted by halogen, hydroxyl or cyano; wherein the 4-7 membered heterocyclic ring may be optionally substituted on nitrogen by one or more substituents selected from the group consisting of C₁₋₆alkyl and (R^{a}R^{b})N-carbonyl-; and wherein said C₁₋₆alkyl may be optionally substituted by one or more substituents selected from the group consisting of halogen, hydroxyl, and cyano; R^{P} is independently selected, for each occurrence, from the group consisting of halogen, hydroxyl, cyano, C₁₋₆alkoxy, (RⁱR^{j})N—, (RⁱR^{j})N-carbonyl-, (RⁱR^{j})N—SO₂—, and (RⁱR^{j})N-carbonyl-N(R^{a})—; w 0, 1, or 2; and pharmaceutically acceptable salts, stereoisomers, esters and prodrugs thereof.

In certain embodiments, C is phenyl. In certain embodiments, C is a phenyl optionally substituted by one, two, or three substituents each selected from the group consisting of halogen, hydroxyl, cyano, C₁₋₆alkyl, C₁₋₆alkoxy, and C₃₋₆cycloalkyl. In certain embodiments, D is phenyl. In certain embodiments, D is a phenyl optionally substituted by one, two, or three substituents each selected from the group consisting of halogen, hydroxyl, cyano, C₁₋₆alkyl, C₁₋₆alkoxy, and C₃₋₆cycloalkyl. In embodiments, D is phenyl substituted by one, two, or three halogen.

In some embodiments, R^{f} is independently selected, for each occurrence, from the group consisting of hydrogen, C₁₋₆alkyl, C₃₋₆cycloalkyl, halogen, hydroxyl, cyano, C₁₋₆alkoxy, (RⁱⁱR^{jj})N—, (RⁱⁱR^{jj})N-carbonyl-, (RⁱⁱR^{jj})N—SO₂—, (RⁱⁱR^{jj})N-carbonyl-N(Rⁱⁱ)—, (CH₃)C(O)N(Rⁱⁱ)—, —C(O)OH, and —C(O)N(RⁱⁱR^{jj}); and Rⁱⁱ and R^{jj} are independently selected, for each occurrence, from the group consisting of hydrogen, C₁₋₆alkyl, and C₃₋₆cycloalkyl; and wherein the alkyl or alkoxy may optionally be substituted by one, two, or three substituents each selected from the group consisting of —OH, NH₂, —N(Me)₂, —NH(Me), —OCH₃, —C(O)OH, —C(O)NH₂, —C(O)NH₂, —C(O)NHMe, —NH(CO)CH₃, —NH(CH₂)(CO)NH₂, —NH(CO)(CH₂)NH₂, —NH(CO)NH₂, —NH(CO)NHMe, and —NH(CO)NHEt.

In some embodiments, R⁹ is independently selected, for each occurrence, from the group consisting of hydrogen, oxo, C₁₋₆alkyl, C₃₋₆cycloalkyl, halogen, hydroxyl, cyano, C₁₋₆alkoxy, (RⁱⁱR^{jj})N—, (RⁱⁱR^{jj})N-carbonyl-, (RⁱⁱR^{jj})N—SO₂—, (RⁱⁱR^{jj})N-carbonyl-N(Rⁱⁱ)—, (CH₃)C(O)N(Rⁱⁱ)—, —C(O)OH, and —C(O)N(RⁱⁱR^{jj}); and Rⁱⁱ and R^{jj} are independently selected, for each occurrence, from the group consisting of hydrogen, C₁₋₆alkyl, and C₃₋₆cycloalkyl; and wherein the alkyl or alkoxy may optionally be substituted by one, two, or three substituents each selected from the group consisting of —OH, NH₂, —N(Me)₂, —NH(Me), —OCH₃, —C(O)OH, —C(O)NH₂, —C(O)NH₂, —C(O)NHMe, —NH(CO)CH₃, —NH(CH₂)(CO)NH₂, —NH(CO)(CH₂)NH₂, —NH(CO)NH₂, —NH(CO)NHMe, and —NH(CO)NHEt.

In some embodiments, R^{h} is selected from the group consisting of C₁₋₆alkyl and (RⁱR^{j})N—, wherein Rⁱ and R^{j} are independently, for each occurrence, are selected from the group consisting of hydrogen and C₁₋₆alkyl, wherein the C₁₋₆alkyl is substituted by one, two, or three substituent each selected from the group consisting of (R^{a}R^{b})N—, (R^{a}R^{b})N-carbonyl-, (R^{a}R^{b})N-carbonyl-(R^{a})N—, and R^{a} and R^{b} are independently selected, for each occurrence, from the group consisting of hydrogen and C₁₋₃alkyl.

In some embodiments, R^{k} is selected from the group consisting of C₁₋₆alkyl, C₃₋₆cycloalkyl, 4-6 membered heterocyclyl, C₁₋₆alkyl-(4-6 membered heterocyclyl)

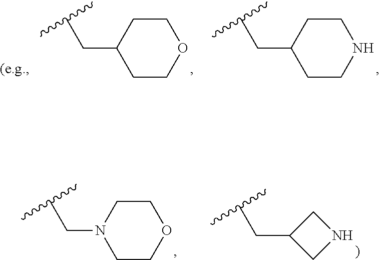 [SMILES: *CC1CCOCC1 ; *CC1CCNCC1 ; *CN1CCOCC1 ; *CC1CNC1]

and C₁₋₆alkyl-(5-6 membered heteroaryl) (e.g.,

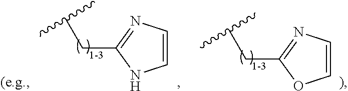

wherein the C₁₋₆alkyl may optionally be substituted by one, two, or three substituents each selected from the group consisting of —OH, NH₂, —N(Me)₂, —NH(Me), —OCH₃, —C(O)OH, —C(O)NH₂, —C(O)NH₂, —C(O)NHMe, —NH(CO)CH₃, —NH(CH₂)(CO)NH₂, —NH(CO)(CH₂)NH₂, —NH(CO)NH₂, —NH(CO)NHMe, and —NH(CO)NHEt.

In an embodiment, R^{w} is selected from the group consisting of:

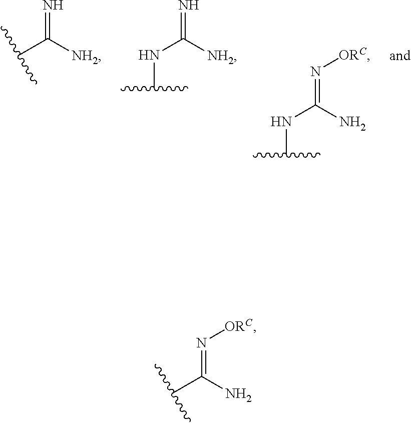

wherein R^{C} is hydrogen or C₁₋₆alkyl. In some embodiments, R^{C} is hydrogen. In some embodiments, R^{C} is C₁₋₆alkyl. In some embodiments, R^{w} is

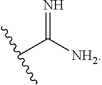 [SMILES: *C(=N)N]

In some embodiments, R^{w} is

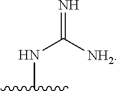 [SMILES: *NC(=N)N]

In some embodiments, R^{w} is

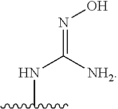 [SMILES: *N/C(N)=N/O]

In some embodiments, R^{w} is

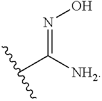 [SMILES: */C(N)=N/O]

In some embodiments, W is phenyl. In embodiments, W is unsubstituted phenyl. In embodiments, W is substituted phenyl.

In some embodiments, the present disclosure provides compounds of Formula I-c:

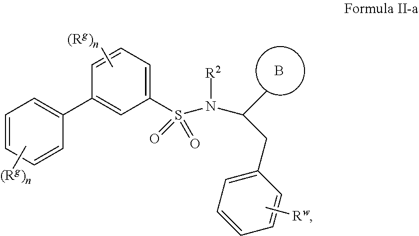

wherein n is independently, for each occurrence, 1, 2, or 3.

In some embodiments, n is independently, for each occurrence, 1 or 2. In some embodiments, n is 1. In some embodiments, n is 2. In other embodiments, n is 3.

In embodiments, R⁹ is selected from the group consisting of hydrogen, halogen, C₁₋₆alkyl, C₁₋₆haloalkyl, and C₁₋₆alkoxy. In embodiments, R⁹ is selected from the group consisting of hydrogen, —Cl, —OCH₃, and —OCH₂CH₃. In embodiments, R⁹ is hydrogen. In embodiments, R⁹ is halogen. In embodiments, R⁹ is C₁₋₆alkyl. In embodiments, R⁹ is C₁₋₆alkoxy.

In embodiments, R^{B} is selected from the group consisting of halogen, cyano, hydroxyl, —OMe, —CHCl₂, —CCl₃, —CHF₂, —CF₃, —N(RⁱR^{j}), C₁₋₆alkyl-N(R^{a})—, C₁₋₆alkyl, —OR^{k}, and 5-6 membered heterocyclyl; wherein the alkyl and heterocyclyl may optionally be substituted by one, two, or three substituents each independently selected from the group consisting of R^{P}, and R^{k} is selected from the group consisting of C₁₋₆alkyl, C₃₋₆cycloalkyl, 4-6 membered heterocyclyl, C₁₋₆alkyl-(4-6 membered heterocyclyl), and C₁₋₆alkyl-(5-6 membered heteroaryl), wherein the C₁₋₆alkyl may optionally be substituted by one, two, or three R^{p}; wherein R^{P} is selected from the group consisting of —OH, NH₂, —N(Me)₂, —NH(Me), —OCH₃, —C(O)OH, —C(O)NH₂, —C(O)NH₂, —C(O)NHMe, —NH(CO)CH₃, —NH(CH₂)(CO)NH₂, —NH(CO)(CH₂)NH₂, —NH(CO)NH₂, —NH(CO)NHMe, and —NH(CO)NHEt; R^{a} is selected from hydrogen and C₁₋₆alkyl; and Rⁱ and R^{j} are independently selected, for each occurrence, from the group consisting of hydrogen, C₁₋₆alkyl, and C₃₋₆cycloalkyl.

In embodiments, B is optionally substituted 5-6 membered monocyclic heteroaryl, wherein B contains at least one nitrogen. In some embodiments, B is optionally substituted 8-10 membered bicyclic heteroaryl, wherein B contains at least one nitrogen. In some embodiments, B is optionally substituted 8-10 membered bicyclic heterocyclyl, wherein B contains at least one nitrogen. In some embodiments, B is selected from the group consisting of pyridinyl, pyrimidinyl, pyrrole, thiazole, oxazole, isothiazole, isoxazole, imidazole, benzothiazole, benzoimidazole, and benzoxazole. In certain embodiments, B is selected from the group consisting of pyridinyl, pyrimidinyl, pyrrole, thiazole, oxazole, isothiazole, isoxazole, imidazole, benzothiazole, benzoimidazole, benzoxazole, tetrahydroimidazopyridine and tetrahydroimidazopyrazine. In embodiments, B is optionally substituted by one or two substituent selected from the group consisting of halogen, C₁₋₆alkyl, and C₁₋₆alkyl-N(R^{a})-carbonyl-N(R^{a})—, wherein the C₁₋₆alkyl may be optionally substituted by one or more substituents each selected from —NH₂, —C(O)NH(C₁₋₆alkyl), and —NH—C(O)—NH(C₁₋₆alkyl); and R^{a} is hydrogen or C₁₋₃alkyl.

In some embodiments, B is selected from the group consisting of

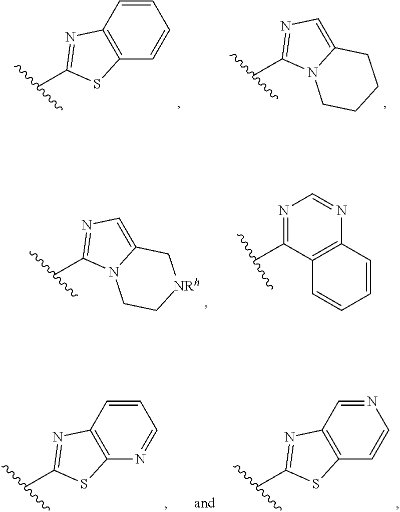

wherein B may optionally substituted on one, two, or three carbons by a substituent each independently selected from the group consisting of halogen, hydroxyl, C₁₋₆haloalkyl, C₁₋₆alkoxy, C₁₋₆alkyl, (R^{a}R^{b})N—, N(R^{a}R^{b})—C₁₋₆alkyl-, (R^{a}R^{b})N—C(O)—C₁₋₆alkyl-, N(R^{a}R^{b})—C(O)—N(R^{a})—C₁₋₆alkyl-, C₁₋₆alkyl-N(R^{a})—C(O)—C₁₋₆alkyl-, C₁₋₆alkyl-C(O)—N(R^{a})—C₁₋₆alkyl-, C(O)OH—C₁₋₆alkyl-, N(R^{a}R^{b})—C₁₋₆alkyl-N(R^{a})—, C₁₋₆alkoxy-C₁₋₆alkyl-N(R^{a})—, N(R^{a}R^{b})—C(O)—C₁₋₆alkyl-N(R^{a})—, N(R^{a}R^{b})—C(O)—N(R^{a})—C₁₋₆alkyl-N(R^{a})—, C₁₋₆alkyl-N(R^{a})—C(O)—N(R^{a})—, N(R^{a}R^{b})—C₁₋₆alkyl-C(O)—N(R^{a})—, N(R^{a}R^{b})—C₁₋₆alkyl-O—, (R^{a})O—C₁₋₆alkyl-O—, N(R^{a}R^{b})—C(O)—N(R^{a})—C₁₋₆alkyl-O—, N(R^{a}R^{b})—C(O)—C₁₋₆alkyl-O—, (5-6 membered heterocyclyl)-C₁₋₆alkyl-O—, (5-6 membered heterocyclyl)-O—, (5-6 membered heteroaryl)-C₁₋₆alkyl-O—, (R^{a})C(O)—N(R^{a})—C₁₋₆alkyl-O—, and —N(RⁱR^{j}), wherein Rⁱ and R^{j} are taken together with the nitrogen to which they are attached form a 4-7 membered heterocyclic ring optionally be substituted by one, two or three substituent each independently selected from the group consisting of C₁₋₆alkyl, —NH₂, —C(O)NH(C₁₋₃alkyl), and R^{a} and R^{b} are independently, for each occurrence, hydrogen or C₁₋₃ alkyl; and R^{h} is selected from the group consisting of C₁₋₆alkyl and (RⁱR^{j})N—, wherein Rⁱ and R^{j} are independently, for each occurrence, are selected from the group consisting of hydrogen and C₁₋₆alkyl, wherein the C₁₋₆alkyl is substituted by one, two, or three substituent each selected from the group consisting of (R^{a}R^{b})N—, (R^{a}R^{b})N-carbonyl-, (R^{a}R^{b})N-carbonyl-(R^{a})N—, and R^{a} and R^{b} are independently selected, for each occurrence, from the group consisting of hydrogen and C₁₋₃alkyl.

In some embodiments, B is selected from the group consisting of

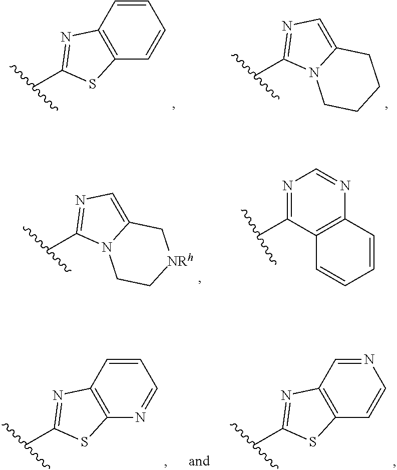

wherein B may optionally substituted on one, two, or three carbons by a substituent each independently selected from the group consisting of halogen, hydroxyl, C₁₋₆haloalkyl, C₁₋₆alkoxy, C₁₋₆alkyl, (R^{a}R^{b})N—, N(R^{a}R^{b})—C₁₋₆alkyl-, (R^{a}R^{b})N—C(O)—C₁₋₆alkyl-, N(R^{a}R^{b})—C(O)—N(R^{a})—C₁₋₆alkyl-, and C₁₋₆alkyl-N(R^{a})— C(O)—N(R^{a})—, wherein R^{a} and R^{b} are independently, for each occurrence, hydrogen or C₁₋₃alkyl; and R^{h} is selected from the group consisting of C₁₋₆alkyl and (RⁱR^{j})N—, wherein Rⁱ and R^{j} are independently, for each occurrence, are selected from the group consisting of hydrogen and C₁₋₆ alkyl, wherein the C₁₋₆alkyl is substituted by one, two, or three substituent each selected from the group consisting of (R^{a}R^{b})N—, (R^{a}R^{b})N-carbonyl-, (R^{a}R^{b})N-carbonyl-(R^{a})N—, and R^{a} and R^{b} are independently selected, for each occurrence, from the group consisting of hydrogen and C₁₋₃alkyl.

In embodiments, B is selected from the group consisting of:

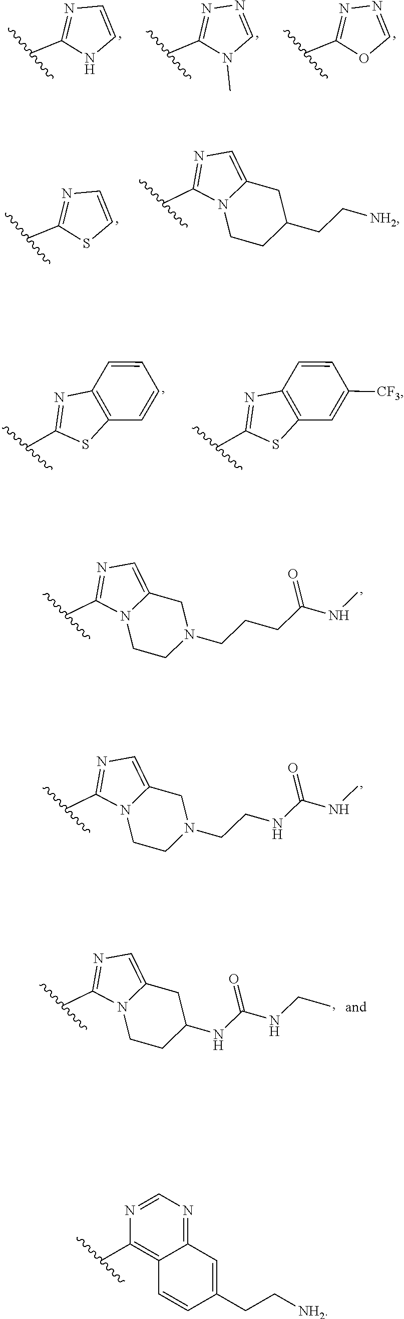

In certain embodiments, R² is hydrogen. In certain embodiments, R² is C₁₋₆ alkyl.

In certain embodiments, the present disclosure provides a compound selected from the group consisting of:

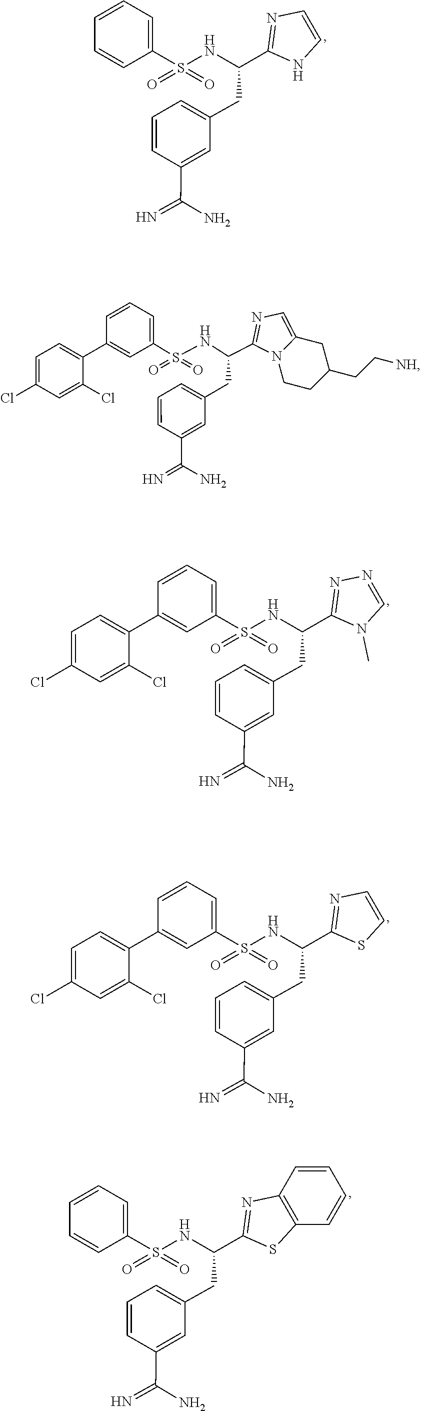

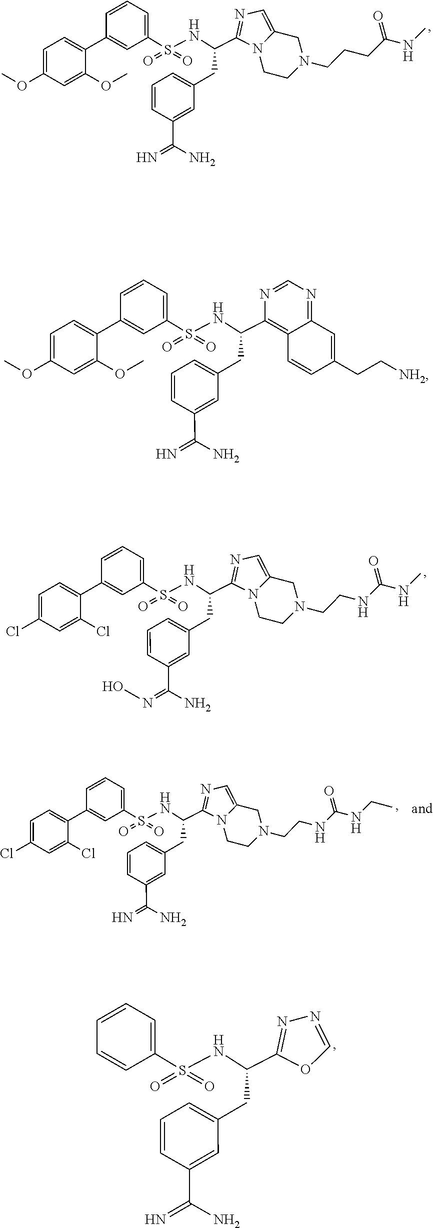

or a pharmaceutically acceptable salt thereof.

In certain embodiments, provided herein are compounds represented by Formula III:

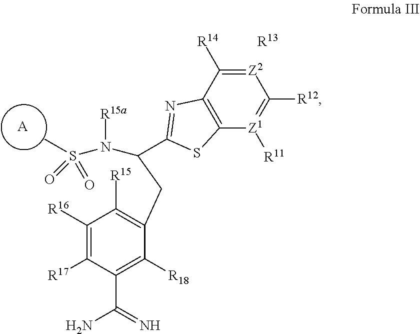

wherein: A is selected from the group consisting of phenyl, naphthyl, and 5-6 membered heteroaryl, wherein A is optionally substituted by one, two, or three substituents each independently selected from the group consisting of R^{A}; R¹¹, R¹², R¹³, and R¹⁴ are independently selected, for each occurrence, from the group consisting of H, —CN, —OH, —NO₂, —NH₂, halogen, C₁₋₆haloalkyl, C₁₋₆alkyl, C₁₋₆alkoxy, and C₃₋₆cycloalkyl; R^{15a} is selected from the group consisting of H, C₁₋₆alkyl, and C₃₋₆cycloalkyl; R¹⁵, R¹⁶, R¹⁷, and R¹⁸ are independently selected, for each occurrence, from the group consisting of H, —NO₂, —NH₂, cyano, hydroxyl, halogen, C₁₋₆haloalkyl, C₁₋₆alkyl, C₁₋₆alkoxy, and C₃₋₆cycloalkyl; R^{A} is selected from the group consisting of —NO₂, —NH₂, cyano, hydroxyl, halogen, C₁₋₆haloalkyl, C₁₋₆alkyl, C₁₋₆alkoxy, and C₃₋₆cycloalkyl; Z¹ is C or N, wherein when Z¹ is N then Rⁱⁱ is absent; Z² is C or N, wherein when Z² is N then R¹³ is absent; and pharmaceutically acceptable salts, stereoisomers, esters and prodrugs thereof.

In certain embodiments, A is an unsubstituted phenyl. In certain embodiments, A is a substituted phenyl. In embodiments, A is a phenyl substituted by one, two or three substituents each independently selected from the group consisting of R^{A}. In embodiments, A is a phenyl substituted by one, two or three substituents each independently selected from the group consisting of halogen and phenyl, wherein the phenyl is optionally substituted by one, two or three halogen. In certain embodiments, A is a phenyl optionally substituted by one, two or three halogen or one

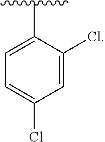 [SMILES: *c1ccc(Cl)cc1Cl]

In some embodiments, R¹¹ is selected from the group consisting of H, Cl,

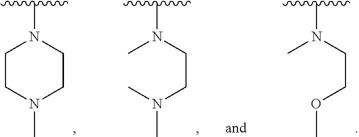

In some embodiments, R¹² is selected from the group consisting of H, —OH, —OCH₃, and CF₃.

In some embodiments, R¹³ is selected from the group consisting of H, CF₃, and CH₃.

In some embodiments, R¹⁴ is H. In some embodiments, R^{15a} is selected from H and C₁₋₆alkyl. In some embodiments, R¹⁵, R¹⁶, R¹⁷, and R¹⁸ are H.

In embodiments, Z¹ is C. In embodiments, Z¹ is N. In embodiments, Z² is C. In embodiments, Z² is N.

In certain embodiments, the present disclosure provides a compound selected from the group consisting of

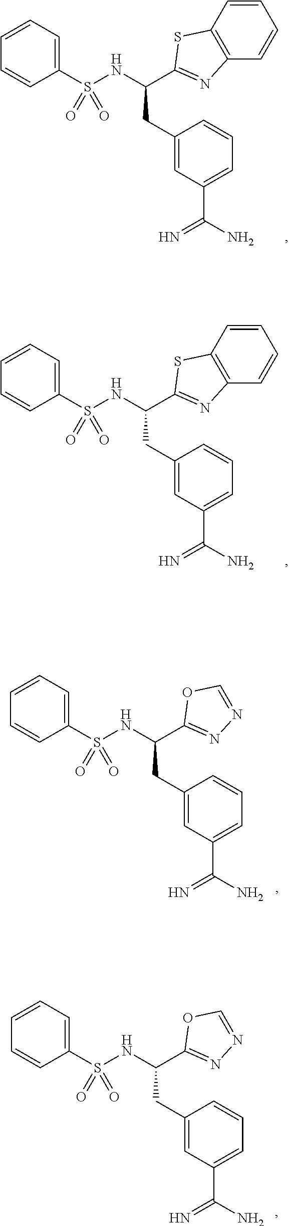 [SMILES: N=C(N)c1cccc(C[C@@H](NS(=O)(=O)c2ccccc2)c2nc3ccccc3s2)c1 ; N=C(N)c1cccc(C[C@H](NS(=O)(=O)c2ccccc2)c2nc3ccccc3s2)c1 ; N=C(N)c1cccc(C[C@@H](NS(=O)(=O)c2ccccc2)c2nnco2)c1 ; N=C(N)c1cccc(C[C@H](NS(=O)(=O)c2ccccc2)c2nnco2)c1]

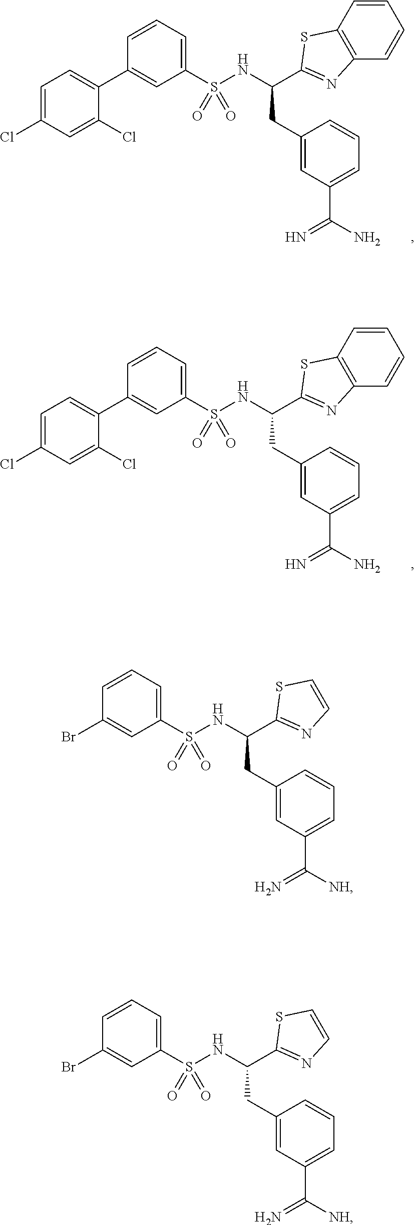

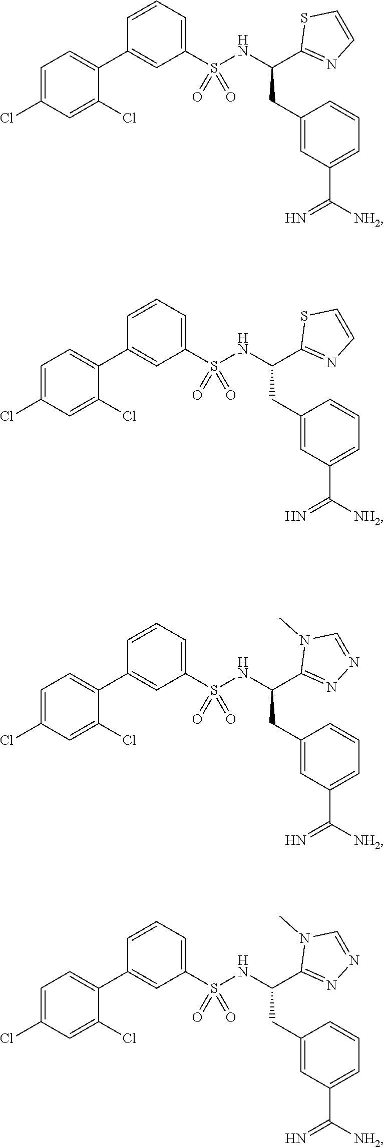 [SMILES: N=C(N)c1cccc(C[C@@H](NS(=O)(=O)c2cccc(-c3ccc(Cl)cc3Cl)c2)c2nccs2)c1 ; N=C(N)c1cccc(C[C@H](NS(=O)(=O)c2cccc(-c3ccc(Cl)cc3Cl)c2)c2nccs2)c1 ; Cn1cnnc1[C@@H](Cc1cccc(C(=N)N)c1)NS(=O)(=O)c1cccc(-c2ccc(Cl)cc2Cl)c1 ; Cn1cnnc1[C@H](Cc1cccc(C(=N)N)c1)NS(=O)(=O)c1cccc(-c2ccc(Cl)cc2Cl)c1]

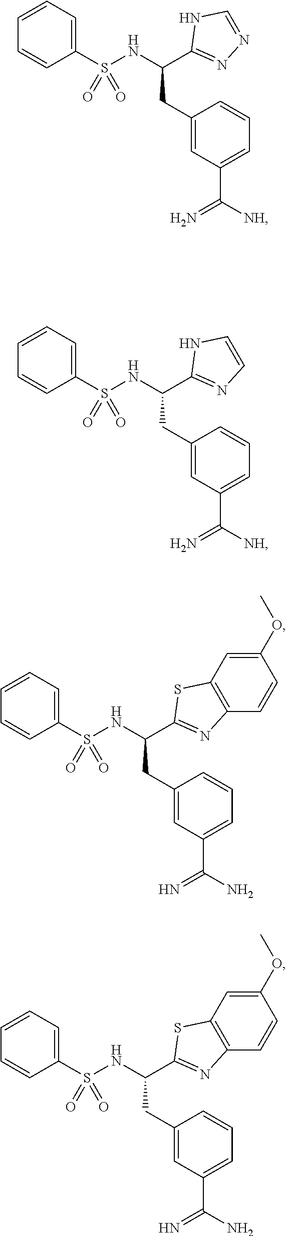

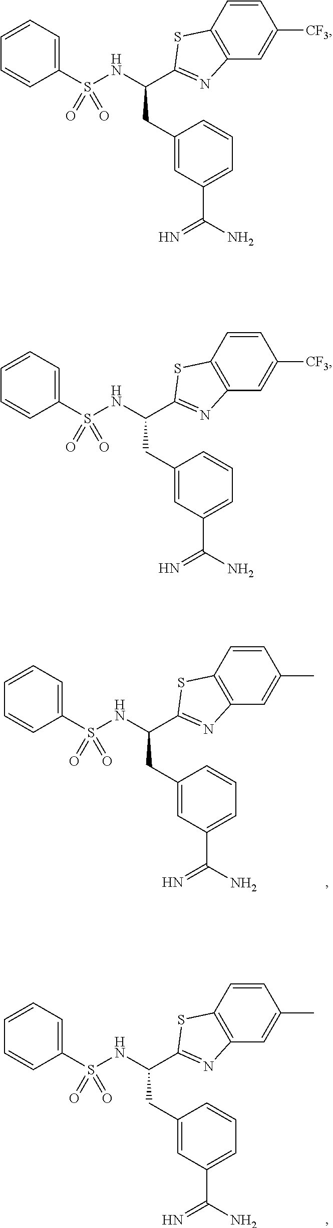 [SMILES: N=C(N)c1cccc(C[C@@H](NS(=O)(=O)c2ccccc2)c2nc3cc(C(F)(F)F)ccc3s2)c1 ; N=C(N)c1cccc(C[C@H](NS(=O)(=O)c2ccccc2)c2nc3cc(C(F)(F)F)ccc3s2)c1 ; Cc1ccc2sc([C@@H](Cc3cccc(C(=N)N)c3)NS(=O)(=O)c3ccccc3)nc2c1 ; Cc1ccc2sc([C@H](Cc3cccc(C(=N)N)c3)NS(=O)(=O)c3ccccc3)nc2c1]

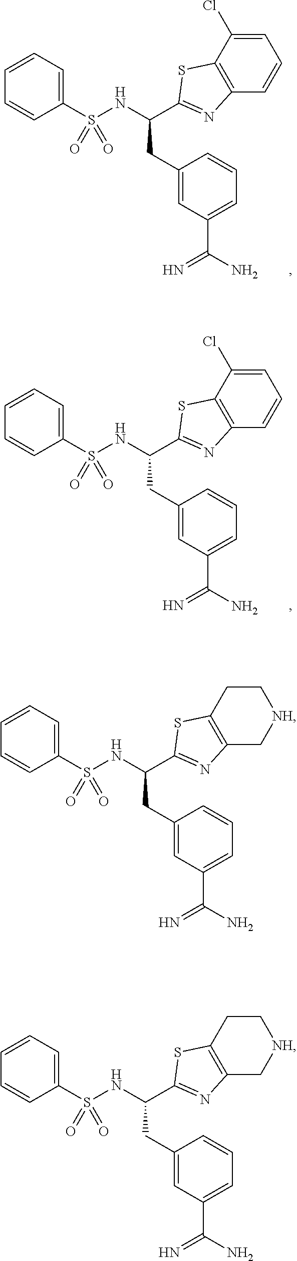 [SMILES: N=C(N)c1cccc(C[C@@H](NS(=O)(=O)c2ccccc2)c2nc3cccc(Cl)c3s2)c1 ; N=C(N)c1cccc(C[C@H](NS(=O)(=O)c2ccccc2)c2nc3cccc(Cl)c3s2)c1 ; N=C(N)c1cccc(C[C@@H](NS(=O)(=O)c2ccccc2)c2nc3c(s2)CCNC3)c1 ; N=C(N)c1cccc(C[C@H](NS(=O)(=O)c2ccccc2)c2nc3c(s2)CCNC3)c1]

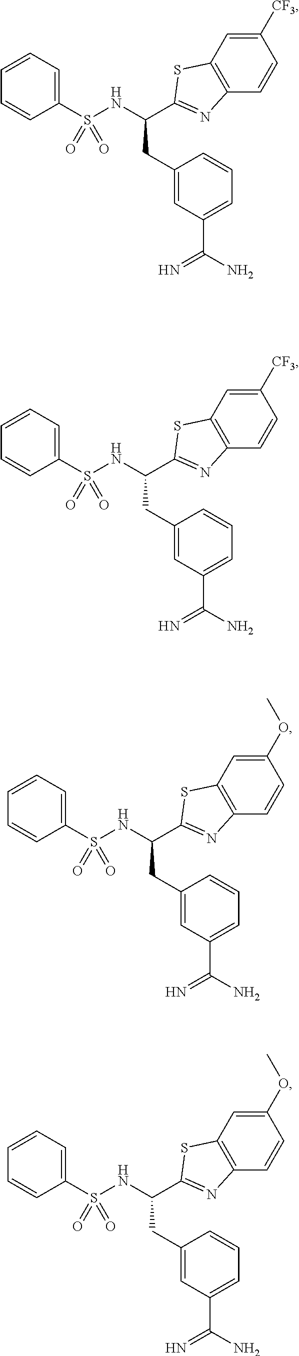 [SMILES: N=C(N)c1cccc(C[C@@H](NS(=O)(=O)c2ccccc2)c2nc3ccc(C(F)(F)F)cc3s2)c1 ; N=C(N)c1cccc(C[C@H](NS(=O)(=O)c2ccccc2)c2nc3ccc(C(F)(F)F)cc3s2)c1 ; COc1ccc2nc([C@@H](Cc3cccc(C(=N)N)c3)NS(=O)(=O)c3ccccc3)sc2c1 ; COc1ccc2nc([C@H](Cc3cccc(C(=N)N)c3)NS(=O)(=O)c3ccccc3)sc2c1]

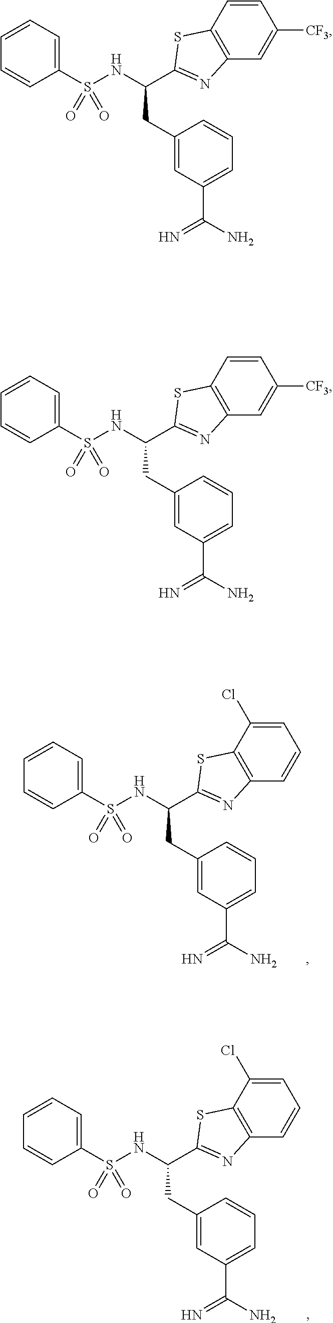 [SMILES: N=C(N)c1cccc(C[C@@H](NS(=O)(=O)c2ccccc2)c2nc3cc(C(F)(F)F)ccc3s2)c1 ; N=C(N)c1cccc(C[C@H](NS(=O)(=O)c2ccccc2)c2nc3cc(C(F)(F)F)ccc3s2)c1 ; N=C(N)c1cccc(C[C@@H](NS(=O)(=O)c2ccccc2)c2nc3cccc(Cl)c3s2)c1 ; N=C(N)c1cccc(C[C@H](NS(=O)(=O)c2ccccc2)c2nc3cccc(Cl)c3s2)c1]

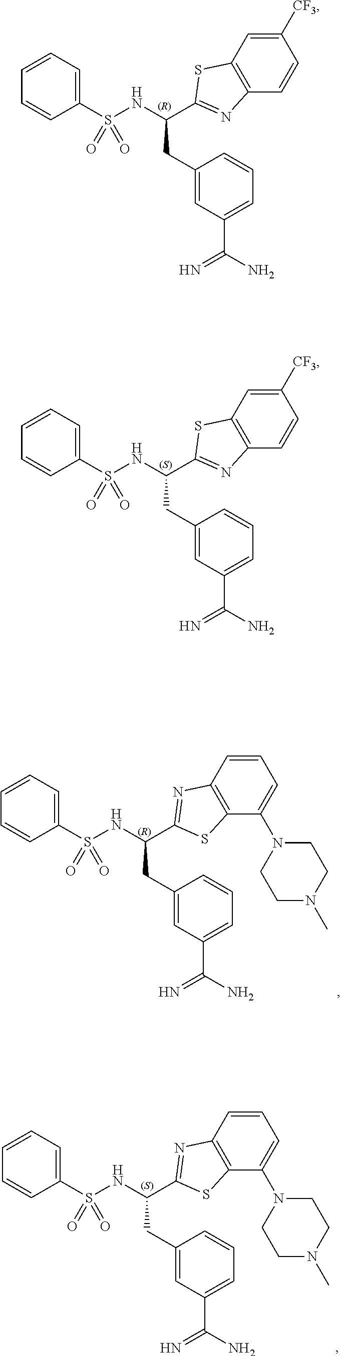

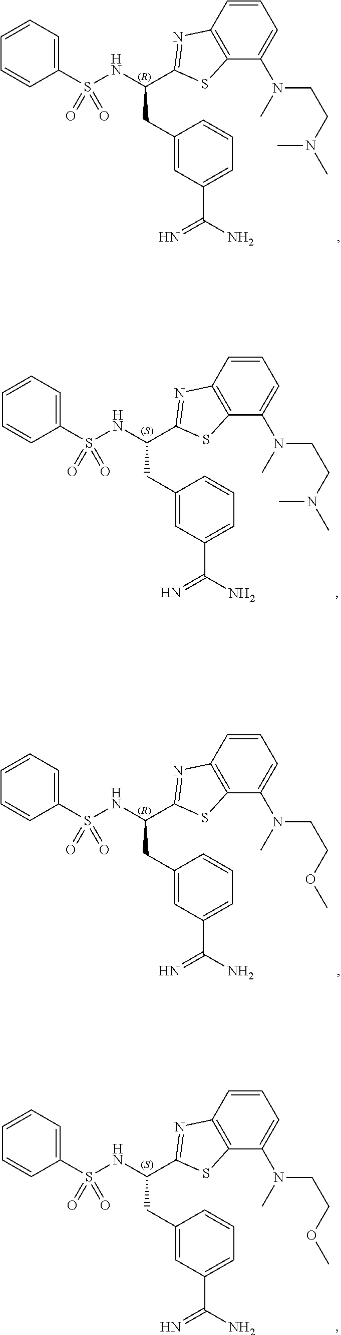

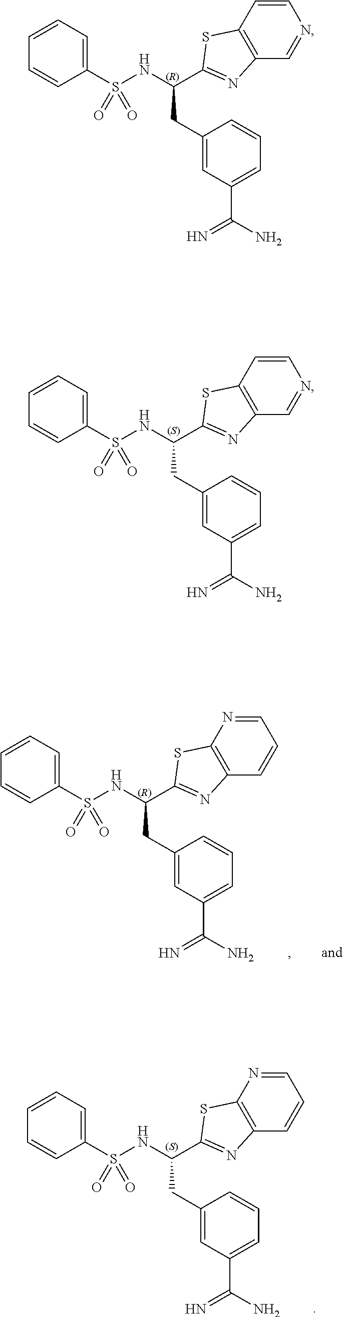

In some embodiments, the compound is selected from the group consisting of the compounds identified in Table 1 and Table 1a below:

TABLE 1
Exemplary compounds

| Compound No. | Structure |
|---|---|
| 1 | 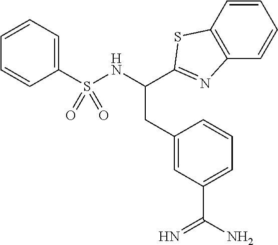 [SMILES: N=C(N)c1cccc(CC(NS(=O)(=O)c2ccccc2)c2nc3ccccc3s2)c1] |
| 2 | 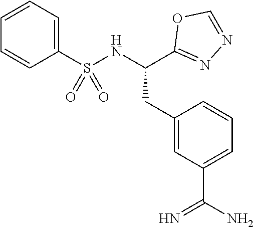 [SMILES: N=C(N)c1cccc(C[C@H](NS(=O)(=O)c2ccccc2)c2nnco2)c1] |
| 3 | 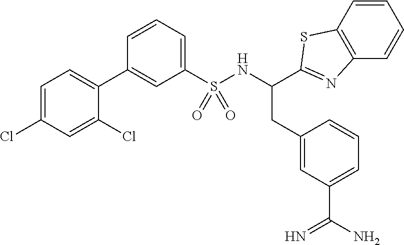 [SMILES: N=C(N)c1cccc(CC(NS(=O)(=O)c2cccc(-c3ccc(Cl)cc3Cl)c2)c2nc3ccccc3s2)c1] |
| 4 | 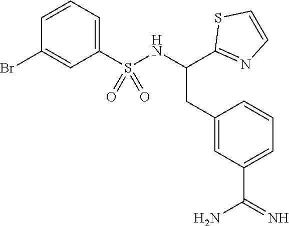 [SMILES: N=C(N)c1cccc(CC(NS(=O)(=O)c2cccc(Br)c2)c2nccs2)c1] |
| 5 | 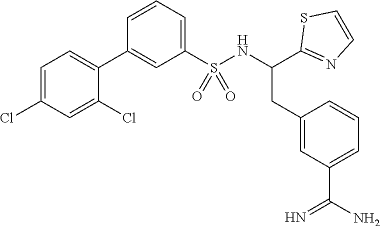 [SMILES: N=C(N)c1cccc(CC(NS(=O)(=O)c2cccc(-c3ccc(Cl)cc3Cl)c2)c2nccs2)c1] |
| 6 | 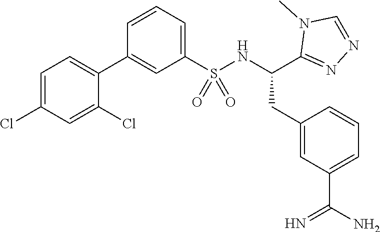 [SMILES: Cn1cnnc1[C@H](Cc1cccc(C(=N)N)c1)NS(=O)(=O)c1cccc(-c2ccc(Cl)cc2Cl)c1] |
| 7 | 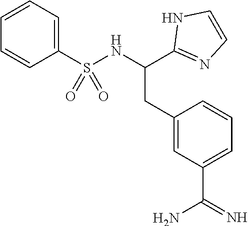 [SMILES: N=C(N)c1cccc(CC(NS(=O)(=O)c2ccccc2)c2ncc[nH]2)c1] |
| 8 | 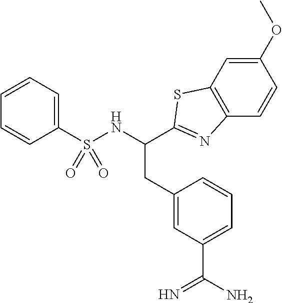 [SMILES: COc1ccc2nc(C(Cc3cccc(C(=N)N)c3)NS(=O)(=O)c3ccccc3)sc2c1] |
| 9 | 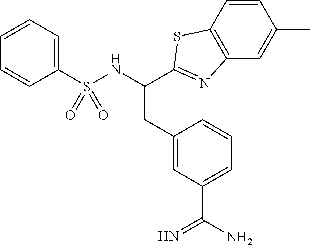 [SMILES: Cc1ccc2sc(C(Cc3cccc(C(=N)N)c3)NS(=O)(=O)c3ccccc3)nc2c1] |
| 10 | 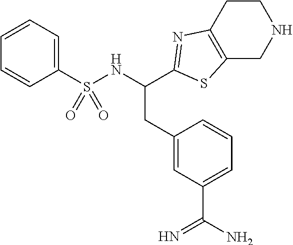 [SMILES: N=C(N)c1cccc(CC(NS(=O)(=O)c2ccccc2)c2nc3c(s2)CNCC3)c1] |
| 11 | 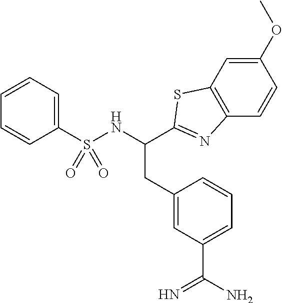 [SMILES: COc1ccc2nc(C(Cc3cccc(C(=N)N)c3)NS(=O)(=O)c3ccccc3)sc2c1] |
| 12 | 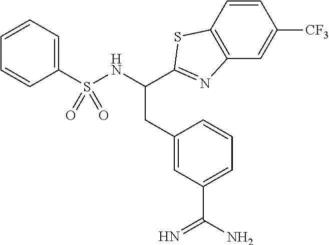 [SMILES: N=C(N)c1cccc(CC(NS(=O)(=O)c2ccccc2)c2nc3cc(C(F)(F)F)ccc3s2)c1] |
| 13 | 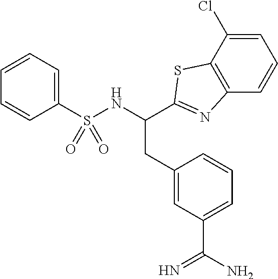 [SMILES: N=C(N)c1cccc(CC(NS(=O)(=O)c2ccccc2)c2nc3cccc(Cl)c3s2)c1] |
| 14 | 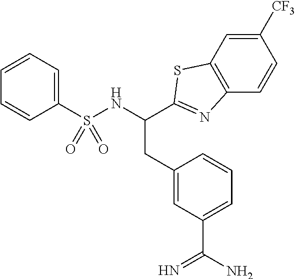 [SMILES: N=C(N)c1cccc(CC(NS(=O)(=O)c2ccccc2)c2nc3ccc(C(F)(F)F)cc3s2)c1] |
| 15 | 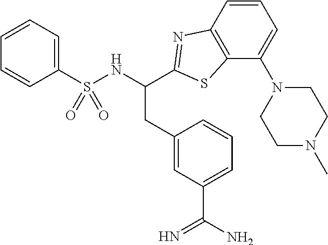 [SMILES: CN1CCN(c2cccc3nc(C(Cc4cccc(C(=N)N)c4)NS(=O)(=O)c4ccccc4)sc23)CC1] |
| 16 | 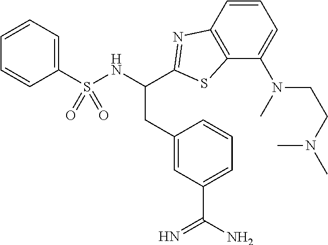 [SMILES: CN(C)CCN(C)c1cccc2nc(C(Cc3cccc(C(=N)N)c3)NS(=O)(=O)c3ccccc3)sc12] |
| 17 | 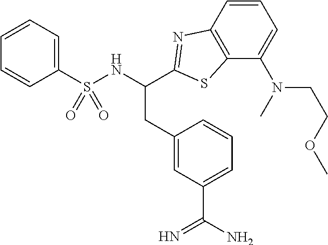 [SMILES: COCCN(C)c1cccc2nc(C(Cc3cccc(C(=N)N)c3)NS(=O)(=O)c3ccccc3)sc12] |
| 18 | 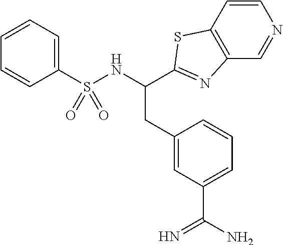 [SMILES: N=C(N)c1cccc(CC(NS(=O)(=O)c2ccccc2)c2nc3cnccc3s2)c1] |
| 19 | 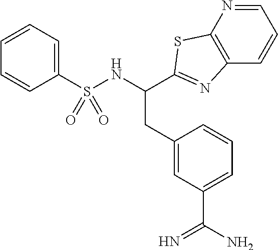 [SMILES: N=C(N)c1cccc(CC(NS(=O)(=O)c2ccccc2)c2nc3cccnc3s2)c1] |
| 20 | 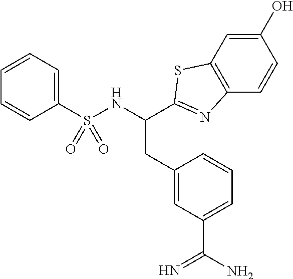 [SMILES: N=C(N)c1cccc(CC(NS(=O)(=O)c2ccccc2)c2nc3ccc(O)cc3s2)c1] |

TABLE 1a
Exemplary compounds

| Compound No. | Structure |
|---|---|
| A-1 | 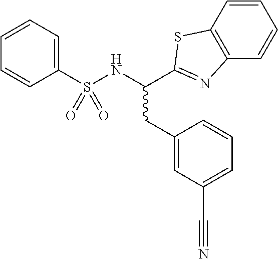 [SMILES: N#Cc1cccc(CC(NS(=O)(=O)c2ccccc2)c2nc3ccccc3s2)c1] |
| A-2 | 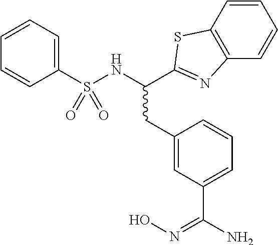 [SMILES: N/C(=N/O)c1cccc(CC(NS(=O)(=O)c2ccccc2)c2nc3ccccc3s2)c1] |
| A-3 | 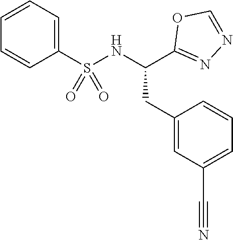 [SMILES: N#Cc1cccc(C[C@H](NS(=O)(=O)c2ccccc2)c2nnco2)c1] |
| A-4 | 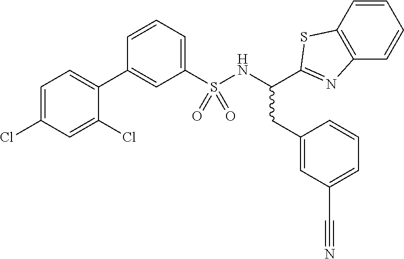 [SMILES: N#Cc1cccc(CC(NS(=O)(=O)c2cccc(-c3ccc(Cl)cc3Cl)c2)c2nc3ccccc3s2)c1] |
| A-5 | 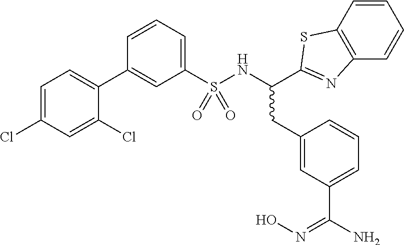 [SMILES: N/C(=N/O)c1cccc(CC(NS(=O)(=O)c2cccc(-c3ccc(Cl)cc3Cl)c2)c2nc3ccccc3s2)c1] |
| A-6 | 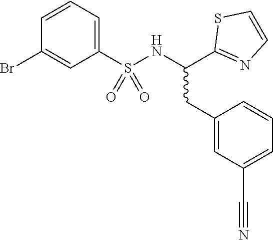 [SMILES: N#Cc1cccc(CC(NS(=O)(=O)c2cccc(Br)c2)c2nccs2)c1] |
| A-7 | 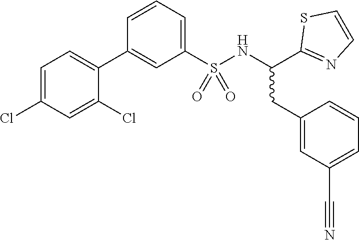 [SMILES: N#Cc1cccc(CC(NS(=O)(=O)c2cccc(-c3ccc(Cl)cc3Cl)c2)c2nccs2)c1] |
| A-8 | 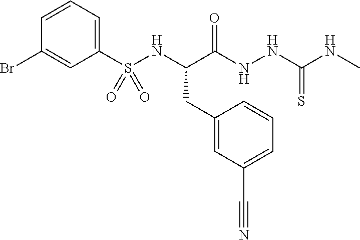 [SMILES: CNC(=S)NNC(=O)[C@H](Cc1cccc(C#N)c1)NS(=O)(=O)c1cccc(Br)c1] |
| A-9 | 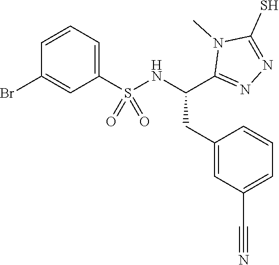 [SMILES: Cn1c(S)nnc1[C@H](Cc1cccc(C#N)c1)NS(=O)(=O)c1cccc(Br)c1] |
| A-10 | 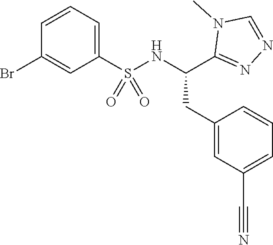 [SMILES: Cn1cnnc1[C@H](Cc1cccc(C#N)c1)NS(=O)(=O)c1cccc(Br)c1] |
| A-11 | 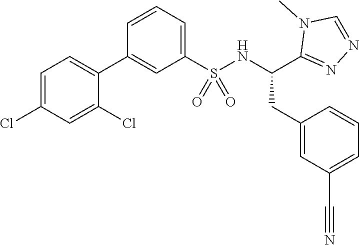 [SMILES: Cn1cnnc1[C@H](Cc1cccc(C#N)c1)NS(=O)(=O)c1cccc(-c2ccc(Cl)cc2Cl)c1] |
| A-12 | 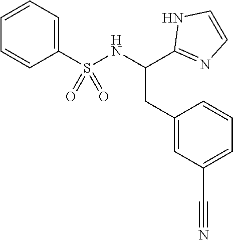 [SMILES: N#Cc1cccc(CC(NS(=O)(=O)c2ccccc2)c2ncc[nH]2)c1] |
| A-13 | 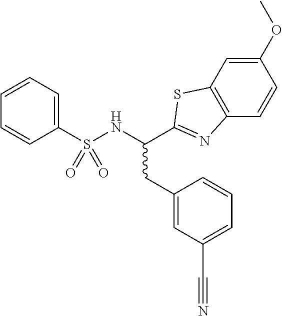 [SMILES: COc1ccc2nc(C(Cc3cccc(C#N)c3)NS(=O)(=O)c3ccccc3)sc2c1] |
| A-14 | 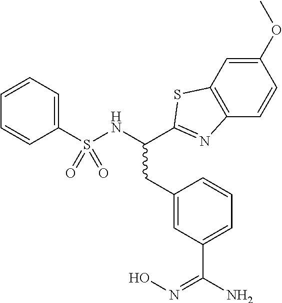 [SMILES: COc1ccc2nc(C(Cc3cccc(/C(N)=N\O)c3)NS(=O)(=O)c3ccccc3)sc2c1] |
| A-15 | 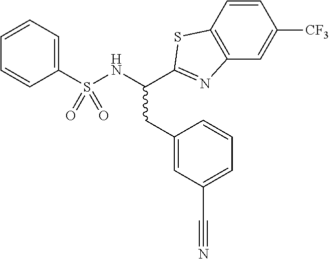 [SMILES: N#Cc1cccc(CC(NS(=O)(=O)c2ccccc2)c2nc3cc(C(F)(F)F)ccc3s2)c1] |
| A-16 | 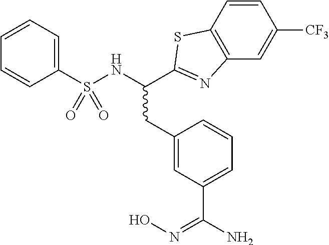 [SMILES: N/C(=N/O)c1cccc(CC(NS(=O)(=O)c2ccccc2)c2nc3cc(C(F)(F)F)ccc3s2)c1] |
| A-17 | 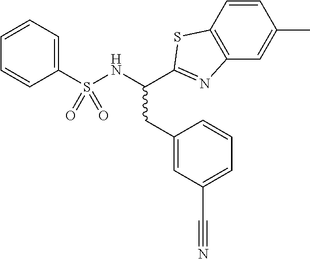 [SMILES: Cc1ccc2sc(C(Cc3cccc(C#N)c3)NS(=O)(=O)c3ccccc3)nc2c1] |
| A-18 | 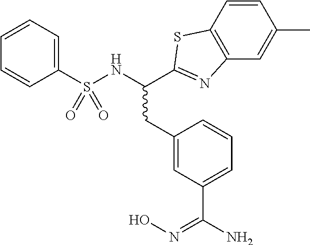 [SMILES: Cc1ccc2sc(C(Cc3cccc(/C(N)=N\O)c3)NS(=O)(=O)c3ccccc3)nc2c1] |
| A-19 | 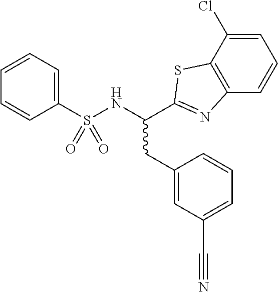 [SMILES: N#Cc1cccc(CC(NS(=O)(=O)c2ccccc2)c2nc3cccc(Cl)c3s2)c1] |
| A-20 | 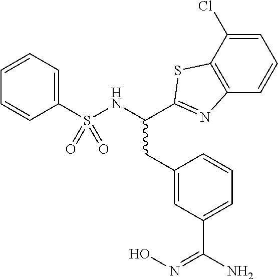 [SMILES: N/C(=N/O)c1cccc(CC(NS(=O)(=O)c2ccccc2)c2nc3cccc(Cl)c3s2)c1] |
| A-21 | 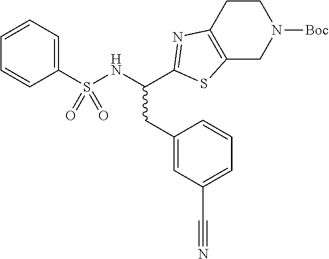 [SMILES: CC(C)(C)OC(=O)N1CCc2nc(C(Cc3cccc(C#N)c3)NS(=O)(=O)c3ccccc3)sc2C1] |
| A-22 | 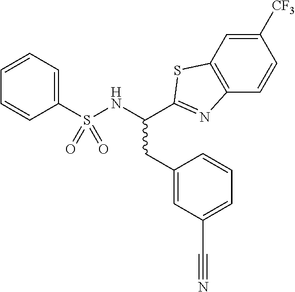 [SMILES: N#Cc1cccc(CC(NS(=O)(=O)c2ccccc2)c2nc3ccc(C(F)(F)F)cc3s2)c1] |
| A-23 | 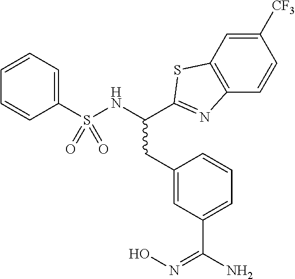 [SMILES: N/C(=N/O)c1cccc(CC(NS(=O)(=O)c2ccccc2)c2nc3ccc(C(F)(F)F)cc3s2)c1] |
| A-24 | 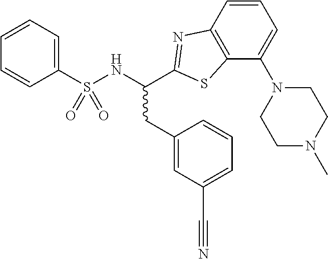 [SMILES: CN1CCN(c2cccc3nc(C(Cc4cccc(C#N)c4)NS(=O)(=O)c4ccccc4)sc23)CC1] |
| A-25 | 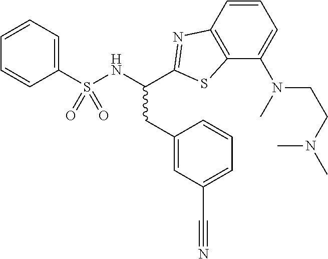 [SMILES: CN(C)CCN(C)c1cccc2nc(C(Cc3cccc(C#N)c3)NS(=O)(=O)c3ccccc3)sc12] |
| A-26 | 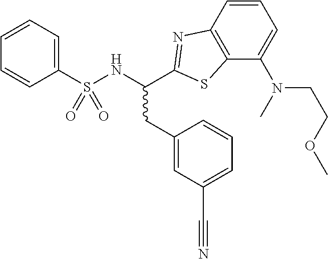 [SMILES: COCCN(C)c1cccc2nc(C(Cc3cccc(C#N)c3)NS(=O)(=O)c3ccccc3)sc12] |
| A-27 | 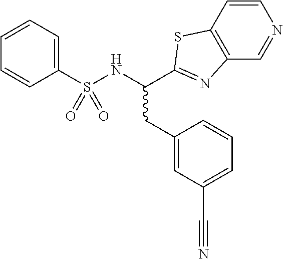 [SMILES: N#Cc1cccc(CC(NS(=O)(=O)c2ccccc2)c2nc3cnccc3s2)c1] |
| A-28 | 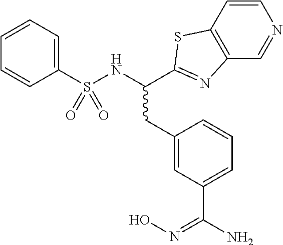 [SMILES: N/C(=N/O)c1cccc(CC(NS(=O)(=O)c2ccccc2)c2nc3cnccc3s2)c1] |
| A-29 | 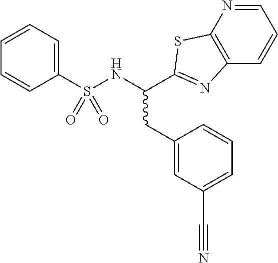 [SMILES: N#Cc1cccc(CC(NS(=O)(=O)c2ccccc2)c2nc3cccnc3s2)c1] |
| A-30 | 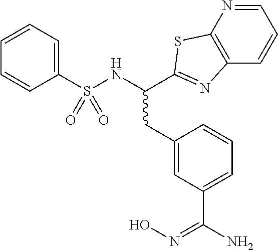 [SMILES: N/C(=N/O)c1cccc(CC(NS(=O)(=O)c2ccccc2)c2nc3cccnc3s2)c1] |
| A-31 | 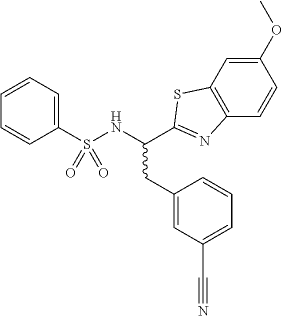 [SMILES: COc1ccc2nc(C(Cc3cccc(C#N)c3)NS(=O)(=O)c3ccccc3)sc2c1] |
| A-32 | 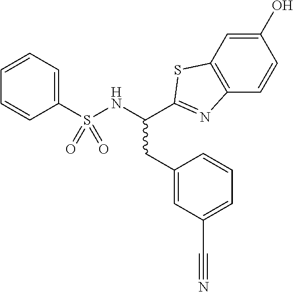 [SMILES: N#Cc1cccc(CC(NS(=O)(=O)c2ccccc2)c2nc3ccc(O)cc3s2)c1] |

### II. Methods

Another aspect of the disclosure provides methods of treating patients suffering from a viral infection, e.g., a coronaviral infection. In particular, in certain embodiments, the disclosure provides a method of treating the below medical indications comprising administering to a subject in need thereof a therapeutically effective amount of a compound described herein, such as a compound of Formula I, I-a, I-b, I-c, I-d, II, II-a or III. In one aspect, the compounds described herein are contemplated as a TMPRSS2 inhibitor. In certain embodiments, the disclosure provides a method of treating a viral infection in a patient in need thereof, comprising inhibiting TMPRSS2 by administering a compound of Formula I, I-a, I-b, I-c, I-d, II, II-a or III.

In some embodiments, the disease or disorder is caused by a virus. In certain embodiments, the virus is selected from the group consisting of a retrovirus (e.g., human immunodeficiency virus (HIV), simian immunodeficiency virus (SIV), human T-cell lymphotropic virus (HTLV)-1, HTLV-2, HTLV-3, HTLV-4), Ebola virus, hepatitis A virus, hepatitis B virus, hepatitis C virus, a herpes simplex virus (HSV) (e.g., HSV-1, HSV-2, varicella zoster virus, cytomegalovirus), an adenovirus, an orthomyxovirus (e.g., influenza virus A, influenza virus B, influenza virus C, influenza virus D, thogotovirus), a flavivirus (e.g., dengue virus, Zika virus), West Nile virus, Rift Valley fever virus, an arenavirus, Crimean-Congo hemorrhagic fever virus, an echovirus, a rhinovirus, coxsackie virus, a coronavirus (e.g., Severe acute respiratory syndrome coronavirus 2 (SARS-CoV-2)), a respiratory syncytial virus, a mumps virus, a rotavirus, measles virus, rubella virus, a parvovirus (e.g., an adeno-associated virus), a vaccinia virus, a variola virus, a molluscum virus, bovine leukemia virus, a poliovirus, a rabies virus, a polyomavirus (e.g., JC virus, BK virus), an alphavirus, and a rubivirus (e.g., rubella virus).

In certain embodiments, a PEGylated-arginase of the described herein is used for treating a disease or disorder caused by a viral infection, e.g., a disease or disorder selected from the group consisting of acquired immune deficiency syndrome (AIDS), HTLV-1 associated myelopathy/tropical spastic paraparesis, Ebola virus disease, hepatitis A, hepatitis B, hepatitis C, herpes, herpes zoster, acute varicella, mononucleosis, respiratory infections, pneumonia, influenza, dengue fever, encephalitis (e.g., Japanese encephalitis), West Nile fever, Rift Valley fever, Crimean-Congo hemorrhagic fever, Kyasanur Forest disease, Yellow fever, Zika fever, aseptic meningitis, myocarditis, common cold, lung infections, molloscum contagiosum, enzootic bovine leucosis, coronavirus disease 2019 (COVID-19), mumps, gastroenteritis, measles, rubella, slapped-cheek disease, smallpox, warts (e.g., genital warts), molluscum contagiosum, polio, rabies, and Pityriasis rosea.

In some embodiments, the viral disease or disorder is caused by a human immunodeficiency virus (HIV). HIV refers to two species of retrovirus (HIV-1, HIV-2) that infect cells of the immune system, e.g., CD4+ T cells, macrophages, and microglial cells. HIV can progress to acquired immunodeficiency syndrome (AIDS). In some embodiments, the viral disease or disorder is caused by a human papillomavirus (HPV). HPV is a sexually transmitted infection that may result in warts, e.g., genital warts. In some embodiments, the viral disease or disorder is caused by a herpesvirus, e.g., hepatitis C virus (HCV), or cytomegalovirus (CMV). Hepatitis C primarily affects the liver and often leads to liver disease and/or cirrhosis. Cytomegalovirus (CMV), e.g., human cytomegalovirus, is associated with pneuomia and mononucleosis. In some embodiments, the viral disease or disorder is caused by a flavivirus, e.g., Ebola virus, Zika virus, or West Nile virus. Ebola virus causes Ebola virus disease (EVD), a viral haemorrhagic fever.

In some embodiments, the virus is an RNA virus (having a genome that is composed of RNA). RNA viruses may be single-stranded RNA (ssRNA) or double-stranded RNA (dsRNA). RNA viruses have high mutation rates compared to DNA viruses, as RNA polymerase lacks proofreading capability (see Steinhauer D A, Holland J J (1987). “Rapid evolution of RNA viruses”. Annu. Rev. Microbiol. 41: 409-33). Exemplary RNA viruses include, without limitation, bunyaviruses (e.g., hantavirus), coronaviruses (e.g., MERS-CoV, SARS-CoV, SARS-CoV-2), flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses. In some embodiments, the RNA virus is an influenza virus, e.g., influenza A. In some embodiments, the RNA virus is RSV. In some embodiments, the RNA virus is MERS-CoV. In some embodiments, the RNA virus is SARS-CoV2. In some embodiments, the RNA virus is ZIKA.

RNA viruses are classified by the type of genome (double-stranded, negative (−), or positive (+) single-stranded). Double-stranded RNA viruses contain a number of different RNA molecules, each coding for one or more viral proteins. Positive-sense ssRNA viruses utilize their genome directly as mRNA; ribosomes within the host cell translate mRNA into a single protein that is then modified to form the various proteins needed for viral replication. One such protein is RNA-dependent RNA polymerase (RNA replicase), which copies the viral RNA in order to form a double-stranded, replicative form. Negative-sense ssRNA viruses have their genome copied by an RNA replicase enzyme to produce positive-sense RNA for replication. Therefore, the virus comprises an RNA replicase enzyme. The resultant positive-sense RNA then acts as viral mRNA and is translated by the host ribosomes. In some embodiments, the virus is a dsRNA virus. In some embodiments, the virus is a negative ssRNA virus. In some embodiments, the virus is a positive ssRNA virus. In some embodiments, the positive ssRNA virus is a coronavirus.

SARS-CoV2, also sometimes referred to as the novel coronavirus of 2019 or 2019-nCoV, is a positive-sense single-stranded RNA virus. SARS-CoV2 has four structural proteins, known as the S (spike), E (envelope), M (membrane), and N (nucleocapsid) proteins. The N protein holds the RNA genome; together, the S, E, and M proteins form the viral envelope. Spike allows the virus to attach to the membrane of a host cell, such as the ACE2 receptor in human cells (Kruse R. L. (2020), Therapeutic strategies in an outbreak scenario to treat the novel coronavirus originating in Wuhan, China (version 2). F1000Research, 9:72). SARS-CoV2 is the highly contagious, causative viral agent of coronavirus disease 2019 (COVID19), a global pandemic.

In some embodiments, the virus is a DNA virus (having a genome that is composed of DNA). Exemplary DNA viruses include, without limitation, parvoviruses (e.g., adeno-associated viruses), adenoviruses, asfarviruses, herpesviruses (e.g., herpes simplex virus 1 and 2 (HSV-1 and HSV-2), epstein-barr virus (EBV), cytomegalovirus (CMV)), papillonoviruses (e.g., HPV), polyoraviruses (e.g., simian vacuolating virus 40 (SV40)), and poxviruses (e.g., vaccinia virus, cowpox virus, smallpox virus, fowlpox virus, sheeppox virus, myxoma virus). In certain embodiments, the DNA virus is an adenovirus, e.g., AdV5. In certain embodiments, the DNA virus is an enterovirus, e.g., EV71. In certain embodiments, the DNA virus is a herpesvirus, e.g., HSV-1.

In some embodiments, the infection is localized, e.g., to an organ or, e.g., to a tissue. In some embodiments, infection is localized to an organ including but not limited to the eye, the ear, the inner ear, the lungs, trachea, bronchus, bronchioli, the liver, the gall bladder, the bile duct, the kidney, the bladder, the testis, the cervix, the ovary, the uterus, the skin, or the brain. In certain embodiments, the infection is a viral infection (e.g., an HSV-1, an HSV-2, a VZV, a CMV) and is localized to the eye. In certain embodiments, the infection is an adenoviral infection and is localized to the eye. In certain embodiments, the infection is a bacterial infection (e.g., Chlamydia) and is localized to the eye.

In some embodiments, the infection is chronic. As used herein, “chronic” refers to an infection that persists for an extended period of time, or recurs. In some embodiments, the infection is acute. As used herein, “acute” refers to an infection that is of short duration.

Methods to quantify viral replication are known in the art. In some embodiments, viral count is determined using a plaque assay. In some embodiments, viral count is determined using a focus forming assay (FFA). In some embodiments, viral count is determined using an endpoint dilution assay. In some embodiments, viral count is determined using an enzyme-linked immunosorbent assay (ELISA). In some embodiments, viral count is determined using Tunable resistive pulse sensing (TRPS) to detect individual virus particles. In some embodiments, viral replication is determined by quantifying the amount or percentage of host cell death, e.g., in vitro, for example, using propidium iodide (PI) to identify dead cells, quantifying the amount of morphologically rounded cells, or by immunofluorescence microscopy for apoptotic markers. In some embodiments, viral count is determined by measuring viral titer or multiplicity of infection (MOI) or by performing a plaque assay, a focus forming assay, and endpoint dilution assay, a viral protein quantification assay (for example, a hemagglutination assay, a bicinchoninic acid assay (BCA), or a single radial immunodiffusion assay (SRID) assay), transmission electron microscopy analysis, a tunable resistive pulse sensing (TRPS) assay, a flow cytometry assay, a quantitative PCR (qPCR) assay, or an Enzyme-linked immunosorbent assay (ELISA). In some embodiments, viral replication is determined by quantification of viral nucleic acid (for example, viral DNA or viral RNA) content.

Methods to quantify viral transmission are known in the art. In some embodiments, viral transmission is quantified using epidemiological modeling (see, e.g., Graw F. et al., (2016) Modeling Viral Spread. Annu Rev Virol, 3(1)). In some embodiments, viral transmission is assessed in vitro, e.g., in cell culture, e.g., using microscopy, e.g., using transmission electron microscopy (TEM).

Methods to quantify viral assembly are known in the art. In some embodiments, viral assembly is determined using statistical modeling (see, e.g., Clement N et al., (2018) Viral Capsid Assembly: A Quantified Uncertainty Approach. JComp Biol, 25(1)). In some embodiments, viral assembly is determined using biochemical techniques to determine capsid complex formation, e.g., co-immunoprecipitation, e.g., western blotting. In some embodiments, viral assembly is determined by flow cytometry for detection of colocalized viral protein (see, e.g., Stoffel, C L. et al (2005). “Rapid Determination of Baculovirus Titer by a Dual Channel Virus Counter” American Biotechnologv Laboratory. 37 (22): 24-25).

Viral genes encode elements necessary for the process of viral infection, a multi-step process, including, for example, attachment to the host cell, penetration, de-envelopment, viral gene transcription cascade, viral protein expression, viral genome replication, viral packaging and assembly, envelopment, transport and maturation, release and egress, and host cell-to-cell transmission. R genes are those genes corresponding to early steps of viral infection, e.g., viral genome replication. γ genes are those genes corresponding to late steps of viral infection, e.g., egress. Methods to quantify viral gene expression are known in the art. In some embodiments, viral gene expression is determined using reverse transcriptase and quantitative polymerase chain reaction (RT-qPCR). In some embodiments, RNA sequencing (RNA-Seq) is used to determine viral gene expression. In some embodiments, viral DNA is quantified using a Southern blot. In some embodiments, p gene expression is quantified. In some embodiments, γ gene expression is quantified. In some embodiments, p gene expression and γ gene expression are quantified. In some embodiments, expression of the entire viral genome is quantified.

Methods to quantify virus release are known in the art. In some embodiments, viral release is determined by biochemical assay, e.g., western blotting, e.g., metabolic labeling (see, e.g., Yadav et al., (2012). “A facile quantitative assay for viral particle genesis reveals cooperativity in virion assembly and saturation of an antiviral protein.” Virology. 429(2): 155-162). In some embodiments, viral release is determined by ELISA. In some embodiments, viral release is determined using electron microscopy, e.g., transmission electron microscopy (TEM). In some embodiments, viral release is determined by infectivity measurements for the detection of virions in a sample, e.g., serum. In some embodiments, viral release is determined by quantification of viral DNA or viral RNA in serum in vivo or culture supernatant in vitro.

Methods of treatment of the present invention can be used as a monotherapy or in combination with one or more other therapies (for example, anti-infective agents) that can be used to treat a disease or disorder, for example, an infection. The term “combination,” as used herein, is understood to mean that two or more different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Accordingly, in certain embodiments, the subject has received, is receiving, or is scheduled to receive one or more other therapies suitable for use in treating the disease or disorder. In certain embodiments, the method of treatment of the present invention further comprises administering to the subject one or more other therapies suitable for use in treating a disease or disorder, for example, an infection. In certain embodiments, the one or more other therapies comprise an agent that ameliorates one or more symptoms of infection with an intracellular pathogen. In certain embodiments, the one or more other therapies comprise surgical removal of an infected tissue.

Accordingly, in certain embodiments, the subject has received, is receiving, or is scheduled to receive one or more other therapies suitable for use in treating the disease or disorder. In certain embodiments, the method of treatment of the present invention further comprises administering to the subject one or more other therapies suitable for use in treating a disease or disorder, for example, an infection. In certain embodiments, the one or more other therapies comprise an agent that ameliorates one or more symptoms of infection with an intracellular pathogen. In certain embodiments, the one or more other therapies comprise surgical removal of an infected tissue.

It is understood that a method of use disclosed herein can be used in combination with an agent, for example, an anti-infective agent that ameliorates one or more symptoms of a disease or disorder associated with an intracellular pathogen. For example, a method of use disclosed herein can be used in combination with another antiviral agent.

In some embodiments, methods described herein further comprise administering an additional anti-viral agent. In some embodiments, the anti-viral agent is selected from the group consisting of ribavirin, favipiravir, ST-193, oseltamivir, zanamivir, peramivir, danoprevir, ritonavir, and remdesivir. In some embodiments, the another agent is selected from the group consisting of protease inhibitors (e.g., nafamostat, camostat, gabexate, epsilon-aminocapronic acid and aprotinin), fusion inhibitors (e.g., BMY-27709, CL 61917, and CL 62554), M2 proton channel blockers (e.g., amantadine and rimantadine), polymerase inhibitors (e.g., 2-deoxy-2′fluoroguanosides (2′-fluoroGuo), 6-endonuclease inhibitors (e.g., L-735,822 and flutamide) neuraminidase inhibitors (e.g., zanamivir (Relenza), oseltamivir, peramivir and ABT-675 (A-315675), reverse transcriptase inhibitor (e.g., abacavir, adefovir, delavirdine, didanosine, efavirenz, emtricitabine, lamivudine, nevirapine, stavudine, tenofovir, tenofovir disoproxil, and zalcitabine), acyclovir, acyclovir, protease inhibitors (e.g., amprenavir, indinavir, nelfinavir, ritonavir, and saquinavir), arbidol, atazanavir, atripla, boceprevir, cidofovir, combivir, darunavir, docosanol, edoxudine, entry inhibitors (e.g., enfuvirtide and maraviroc), entecavir, famciclovir, fomivirsen, fosamprenavir, foscarnet, fosfonet, ganciclovir, ibacitabine, immunovir, idoxuridine, imiquimod, inosine, integrase inhibitor (e.g., raltegravir), interferons (e.g., types I, II, and III), lopinavir, loviride, moroxydine, nexavir, nucleoside analogues (e.g., aciclovir), penciclovir, pleconaril, podophyllotoxin, ribavirin, tipranavir, trifluridine, trizivir, tromantadine, truvada, valaciclovir, valganciclovir, vicriviroc, vidarabine, viramidine, and zodovudine. In some embodiments, the additional anti-viral agent is selected from the group consisting of lamivudine, an interferon alpha, a VAP anti-idiotypic antibody, enfuvirtide, amantadine, rimantadine, pleconaril, aciclovir, zidovudine, fomivirsen, a morpholino, a protease inhibitor, double-stranded RNA activated caspase oligomerizer (DRACO), rifampicin, zanamivir, oseltamivir, danoprevir, ritonavir, and remdesivir. In some embodiments, the another agent is selected from the group consisting of quinine (optionally in combination with clindamycin), chloroquine, amodiaquine, artemisinin and its derivatives (e.g., artemether, artesunate, dihydroartemisinin, arteether), doxycycline, pyrimethamine, mefloquine, halofantrine, hydroxychloroquine, eflornithine, nitazoxanide, ornidazole, paromomycin, pentamidine, primaquine, pyrimethamine, proguanil (optionally in combination with atovaquone), a sulfonamide (e.g., sulfadoxine, sulfamethoxypyridazine), tafenoquine, tinidazole and a PPT1 inhibitor (including Lys05 and DC661). In some embodiments, the another agent is an antibiotic. In some embodiments, the antibiotic is a penicillin antibiotic, a quinolone antibiotic, a tetracycline antibiotic, a macrolide antibiotic, a lincosamide antibiotic, a cephalosporin antibiotic, or an RNA synthetase inhibitor. In some embodiments, the antibiotic is selected from the group consisting of azithromycin, vancomycin, metronidazole, gentamicin, colistin, fidaxomicin, telavancin, oritavancin, dalbavancin, daptomycin, cephalexin, cefuroxime, cefadroxil, cefazolin, cephalothin, cefaclor, cefamandole, cefoxitin, cefprozil, ceftobiprole, cipro, Levaquin, floxin, tequin, avelox, norflox, tetracycline, minocycline, oxytetracycline, doxycycline, amoxicillin, ampicillin, penicillin V, dicloxacillin, carbenicillin, methicillin, ertapenem, doripenem, imipenem/cilastatin, meropenem, amikacin, kanamycin, neomycin, netilmicin, tobramycin, paromomycin, cefixime, cefdinir, cefditoren, cefoperazone, cefotaxime, ceftazidime, ceftibuten, ceftizoxime, ceftriaxone, cefoxotin, and streptomycin. In some embodiments, the antibiotic is azithromycin.

In some embodiments, the additional therapeutic agents can be kinase inhibitors including but not limited to erlotinib, gefitinib, neratinib, afatinib, osimertinib, lapatanib, crizotinib, brigatinib, ceritinib, alectinib, lorlatinib, everolimus, temsirolimus, abemaciclib, LEE011, palbociclib, cabozantinib, sunitinib, pazopanib, sorafenib, regorafenib, sunitinib, axitinib, dasatinib, imatinib, nilotinib, ponatinib, idelalisib, ibrutinib, Loxo 292, larotrectinib, and quizartinib.

In some embodiments, the additional therapeutic agents can be therapeutic anti-viral vaccines.

In some embodiments, the additional therapeutic agents can be immunomodulatory agents including but not limited to anti-PD-lor anti-PDL-1 therapeutics including pembrolizumab, nivolumab, atezolizumab, durvalumab, BMS-936559, or avelumab, anti-TIM3 (anti-HAVcr2) therapeutics including but not limited to TSR-022 or MBG453, anti-LAG3 therapeutics including but not limited to relatlimab, LAG525, or TSR-033, anti-4-1BB (anti-CD37, anti-TNFRSF9), CD40 agonist therapeutics including but not limited to SGN-40, CP-870,893 or R07009789, anti-CD47 therapeutics including but not limited to Hu5F9-G4, anti-CD20 therapeutics, anti-CD38 therapeutics, STING agonists including but not limited to ADU-S100, MK-1454, ASA404, or amidobenzimidazoles, anthracyclines including but not limited to doxorubicin or mitoxanthrone, hypomethylating agents including but not limited to azacytidine or decitabine, other immunomodulatory therapeutics including but not limited to epidermal growth factor inhibitors, statins, metformin, angiotensin receptor blockers, thalidomide, lenalidomide, pomalidomide, prednisone, or dexamethasone.

Another aspect of the disclosure provides methods of treating patients suffering from a disorder such as a tumor, e.g., a solid tumor, and cancer. In particular, in certain embodiments, the disclosure provides a method of treating a tumor or cancer comprising administering to a subject in need thereof a therapeutically effective amount of a compound described herein, such as a compound of Formula I, I-a, I-b, I-c, I-d, II, II-a or III. Exemplary disorders include, but not limited to, gastrointestinal stromal tumors, esophageal cancer, gastric cancer, melanomas, gliomas, glioblastomas, ovarian cancer, bladder cancer, pancreatic cancer, prostate cancer, lung cancers, breast cancers, renal cancers, hepatic cancers, osteosarcomas, multiple myelomas, cervical carcinomas, cancers that are metastatic to bone, papillary thyroid carcinoma, non-small cell lung cancer, colorectal cancers, cancer of the thyroid, endocrine system, brain, breast, cervix, colon, head & neck, liver, kidney, lung, non-small cell lung, melanoma, mesothelioma, ovary, sarcoma, stomach, uterus, Medulloblastoma, colorectal cancer, pancreatic cancer. Additional examples may include, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer. A cancer treated by the methods described herein may be a metastatic cancer. In preferable embodiments, the cancer is a prostate cancer. In embodiments, the prostate cancer is a metastatic prostate cancer.

Yet in another aspect of the disclosure provides methods of treating patients suffering from a disorder such as a blood disorder (e.g., blood clots, blood coagulation disorders, bleeding disorders, hemophilia), cardiovascular disease (e.g., ischaemic heart disease (ID), angina pectoris, coronary heart disease, stroke, transient ischaemic attacks, cerebrovascular disease, hypertensive disease, aortic aneurysm, peripheral arterial disease, retinal arterial disease), inflammatory disease (e.g., rheumatoid or rheumatic inflammatory disease, especially arthritis (including rheumatoid arthritis), or other chronic inflammatory disorders, such as chronic asthma, arterial or post-transplantational atherosclerosis, endometriosis) and chronic obstructive pulmonary disease.

As described herein, the compound of the disclosure (e.g., a compound of Formula I, I-a, I-b, I-c, I-d, II, II-a, or III) are contemplated as a protease inhibitor, wherein the protease is TMPRSS2, ACE2, Cathepsin B, Cathepsin L, Elastase, FVIIa, Fxa, FXIa, Furin, Kallikrein 1, Kallikrein 5, Kallikrein 7, Kallikrein 12, Kallikrein 13, Kallikrein 14, Matriptase 2, MMP 1, MMP 2, MMP 7, MMP 10, MMP 13, MMP 14, Mpro, Plasma Kallikrein, Plasmin, Plpro, TACE, Thrombin a, Trypsin, Tryptase b2, Tryptase g1, or Urokinase. The disclosure provides methods of treating patients suffering from a disorder comprising inhibiting a protease by administering a compound of Formula I, I-a, I-b, I-c, I-d, II, II-a, or III. In various embodiments, the protease is selected from the group consisting of ACE2, Cathepsin B, Cathepsin L, Elastase, FVIIa, Fxa, FXIa, Furin, Kallikrein 1, Kallikrein 5, Kallikrein 7, Kallikrein 12, Kallikrein 13, Kallikrein 14, Matriptase 2, MMP 1, MMP 2, MMP 7, MMP 10, MMP 13, MMP 14, Mpro, Plasma Kallikrein, Plasmin, Plpro, TACE, Thrombin a, Trypsin, Tryptase b2, Tryptase g1, and Urokinase. In some embodiments, the protease is selected from the group consisting of Kallikrein 1, Matriptase 2 and Urokinase. In some embodiments, the protease is selected from the group consisting of Kallikrein 1 and Urokinase. In some embodiments, the protease is selected from the group consisting of FXa, Kallikrein 1, MMP 1, MMP 2, MMP 10, MMP 14, Mpro, Plasma Kallikrein, Thrombin a, Trypsin, Tryptase b2, Tryptase g1, and Urokinase. In embodiments, the disorder is a viral infection (e.g., a coronaviral infection). In embodiments, the disorder is a cancer (e.g., prostate cancer, metastatic prostate cancer). In some embodiments, the disorder is a blood disorder or a cardiovascular disease.

### III. Pharmaceutical Compositions and Kits

Another aspect of the disclosure provides pharmaceutical compositions comprising compounds as disclosed herein formulated together with a pharmaceutically acceptable carrier. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous) rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions of this disclosure may be used in the form of a pharmaceutical preparation, for example, in solid, semisolid or liquid form, which contains one or more of the compound of the disclosure, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof, and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well-known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants

In another aspect, the disclosure provides enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e. g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present disclosure.

Advantageously, the disclosure also provides kits for use by a e.g. a consumer in need of 3CL inhibitor. Such kits include a suitable dosage form such as those described above and instructions describing the method of using such dosage form to mediate, reduce or prevent inflammation. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well-known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

Also contemplated herein are methods and compositions that include a second active agent or administering a second active agent. For example, in addition to having a viral infection, a subject or patient can further have viral infection- or virus-related co-morbidities, i.e., diseases and other adverse health conditions associated with, exacerbated by, or precipitated by being infected by a virus. Contemplated herein are disclosed compounds in combination with at least one other agent that has previously been shown to treat these virus-related conditions.

### EXAMPLES

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

At least some of the compounds identified as “Intermediates” herein are contemplated as compounds of the disclosure.

¹H NMR spectra are recorded at ambient temperature using e.g., a Varian Unity Inova (400 MHz) spectrometer with a triple resonance 5 mm probe for Example compounds, and either a Bruker Avance DRX (400 MHz) spectrometer or a Bruker Avance DPX (300 MHz) spectrometer for Intermediate compounds. Chemical shifts are expressed in ppm relative to tetramethylsilane. The following abbreviations have been used: br=broad signal, s=singlet, d=doublet, dd=double doublet, dt=double triplet, ddd=double double doublet, t=triplet, td=triple doublet, tdd=triple double doublet, q=quartet, m=multiplet.

The following abbreviations are used in this disclosure and have the following definitions: “AcOH” is acetic acid, “BOP” is (benzotriazol-1-yloxytris(dimethylamino)phosphonium hexafluorophosphate), “DCM” is dichloromethane, “DIPEA” is N,N-diisopropylethylamine, “DMSO” is dimethyl sulfoxide, “EtOH” is ethanol, “EtOAc” is ethyl acetate, “MeOH” is methanol, “MeCN” is acetonitrile, “MTBE” is methyl tert-butyl ether, “RT” is room temperature, “T3P” is propanephosphonic acid anhydride, “Pd(PPh₃)₄” is tetrakis(triphenylphosphine)palladium(0), “UPLC” is ultra performance liquid chromatography, “HPLC” is high-performance liquid chromatography, “NH₄OAc” is ammonium acetate, “TEA” is triethylamine, and “TFA” is trifluoroacetic acid.

General Chemistry

Exemplary compounds described herein are available by the general synthetic methods illustrated in the Schemes below, Intermediate preparations, and the accompanying Examples.

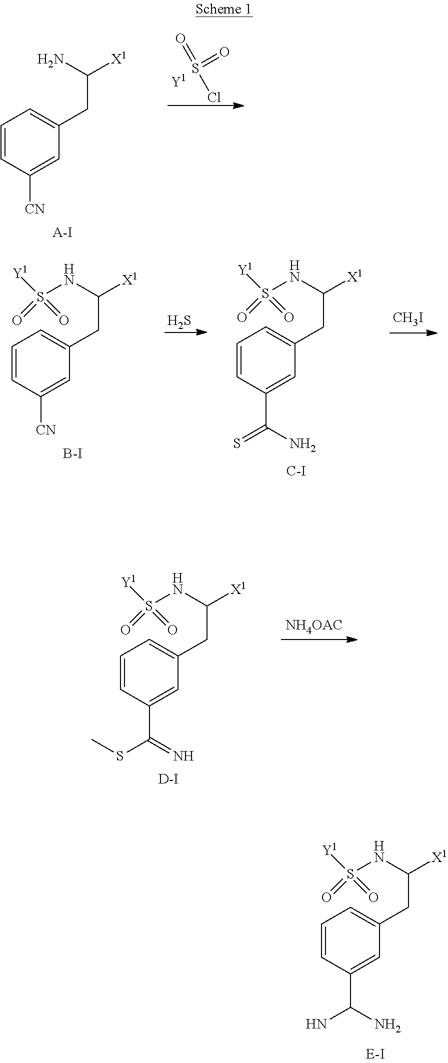

Scheme 1 illustrates an exemplary preparation of amidine E-I. Treatment of A-I with a sulfonyl chloride, which can be aryl sulfonyl chloride or heteroaryl sulfonyl chloride, in the presence of base (e.g. triethylamine) affords compound B-I. Further treatment of B-I with hydrogen sulfide affords compound C-I. After methylating intermediate C-I to afford D-I in the presence of methyl iodide, intermediate D-I is converted to amidine E-I with using ammonium acetate.

In Scheme 1, examples of X¹ include optionally substituted aryl, optionally substituted heteroaryl, and optionally substituted heterocyclyl, and examples of Y¹ include optionally substituted aryl, optionally substituted heteroaryl, and optionally substituted heterocyclyl. The optional substituents of X¹ and Y¹ are exemplified by the tables of intermediates disclosed herein.

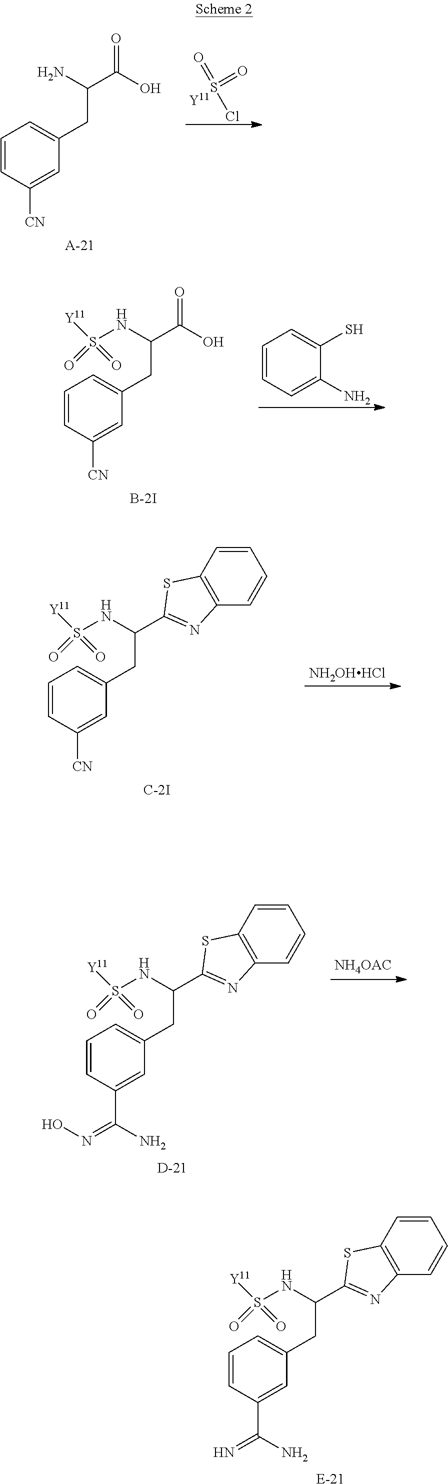

Scheme 2 illustrates an exemplary preparation for amidine E-2I. Reacting the nitrile A-2I with a sulfonyl chloride (e.g., aryl sulfonyl chloride, heteroaryl sulfonyl chloride) in the presence of a base (e.g., Na₂CO₃) affords B-2I. Treating B-2I with 2-aminobenzenethiol in the presence of a base (e.g., N,N-diisopropylethylamine) affords benzothiazole C-2I. Upon reacting C-2I with hydroxylamine hydrochloride affords hydroxybenzimidamide D-2I, which can further be treated with ammonium acetate to afford amidine E-2I. Examples of variable Y¹¹ include, but not limited to, optionally substituted phenyl or naphthyl, optionally substituted heteroaryl, optionally substituted cycloalkyl, and optionally substituted heterocyclyl.

The compounds disclosed herein can be prepared by methods known from the literature, purified at the end by preparative HPLC and are present at TFA salts (Sturzebecher et al., Bioorg. Med Chem. Lett., 9, 3147-3152 (1999); Steinmetzer et al., J. Med. Chem. 49, 4116-4126, (2006); Steinmetzer et al., Bioorg. Med. Chem. Lett. 67-73, (2009); Schweinitz et al., Bioorg. Med. Chem. Lett 19, 1960-1965, (2009); Sturzebecher et al., J. Med. Chem., 40, 3091-3099, (1997); Hammami et al MedChemComm 3, 807-813 (2012); Steinmetzer et al., PCT Int. App. (2013) WO2013014074 A1)). Compounds with a 3-amidinoazaphenylalanine as a central building block are also synthesized by literature methods (Zega et al., Bioorg Med Chem 14 (2004) 1563-1567, Zega et al Bioorg Med Chem 9 (2001) 2745-2756).

### Example 1: Exemplary Synthesis of Amidine Compound

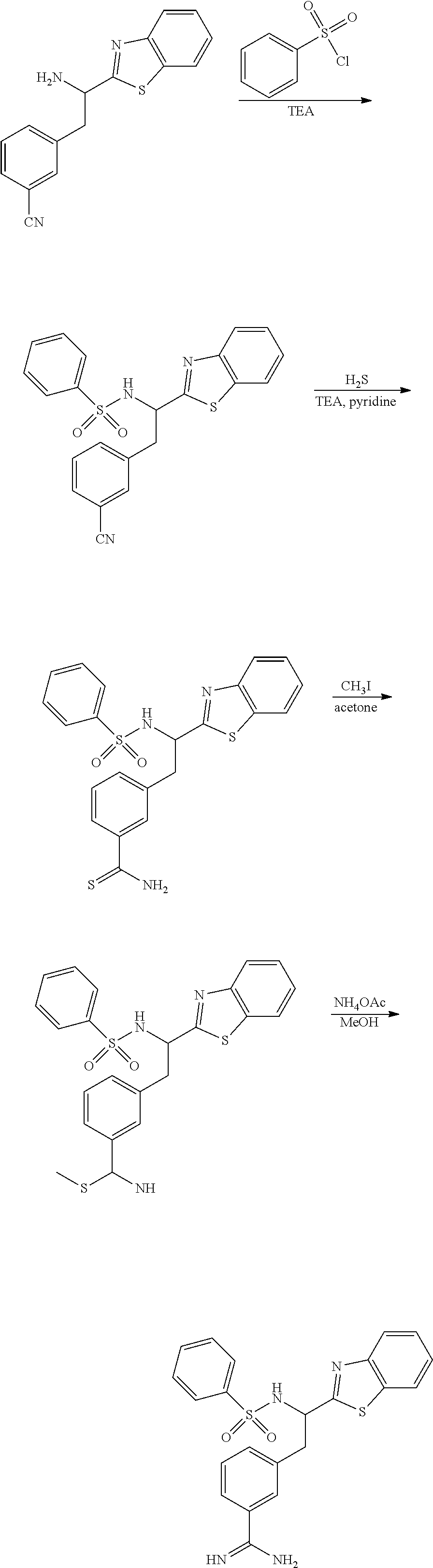

A solution of 3-(2-amino-2-(benzo[d]thiazol-2-yl)ethyl)benzonitrile was added with benzenesulfonyl chloride in the presence of triethylamine. The resulting residue was dissolved in pyridine, triethylamine was added, and then added with gaseous hydrogen sulfide. The resulting thioamide was treated with methyl iodide in acetone to produce methyl 3-(2-(benzo[d]thiazol-2-yl)-2-(phenylsulfonamido)ethyl)benzimidothioate, which was treated with ammonium acetate in methanol to afford 3-(2-(benzo[d]thiazol-2-yl)-2-(phenylsulfonamido)ethyl)benzimidamide.

### Example 2: Exemplary Cloning of the Catalytic Domain of TMPRSS2, and Expression, Purification, Refolding and Activation of the Catalytic Domain of TMPRSS2

Cloning of the Catalytic Domain of TMPRSS2

The nucleotide sequence of the serine protease domain was amplified from the plasmid pCAGGS-TMPRSS2 by PCR using 5′-GGATATCATATGAAACATCACCATCACCATCACATCGTGGGCGGTGAGAG-3′ (SEQ ID NO: 1) and 5′-GGATATGAATTCTTAGCCGTTTGCCTTCATTTG-3′ (SEQ ID NO: 2) as sense and antisense primers respectively. The primers were chosen to introduce a sequence coding for Met-Lys-(His)₆ (SEQ ID NO: 3) at the 5′-end of the cDNA encoding the protease domain. The approximately 750-bp long amplification product was purified and subcloned into a pET24(b) vector (Novagen, Merck Bioscience) for expression into E. coli.

The catalytic domain of TMPRSS2 was expressed in the form of inclusion bodies, as described below, then denatured, purified, refolded and activated.

Expression, Purification, Refolding and Activation of the Catalytic Domain of TMPRSS2

The expression vector, encoding the protease domain, was transformed into E. coli BL21 (DE3) CodonPlus competent cells. The cells were incubated in LB (Luria-Bertani) medium containing 30 μg/ml kanamycin at 37-C for 3 h and 220 rev./min. The expression of the catalytic domain was induced by the addition of 1 mM IPTG (isopropyl β-D-thiogalactopyranoside) at D600=1 and the incubation was continued for 10 h at 5-C. The cells were harvested and suspended in buffer (50 mM Tris/HCl and 0.9% NaCl, pH 7.5) and lysed via ultrasound. After DNA depletion with Benzonase (25 units/g of cell pellet, Novagen), the inclusion bodies were washed and denatured in denaturation buffer (8 M urea, 10 mM Tris and 100 mM sodium phosphate, pH 8.0). The denatured protein was freed from insoluble constituents by centrifugation and filtration (0.2 μm) and the His-tagged TMPRSS2 was purified by metal chelate chromatography (Ni2+-nitrilotriacetate agarose, Qiagen). TMPRSS2-containing fractions were pooled and renatured by rapid dilution in 50-fold volume refolding buffer (50 mM Tris, pH 7.5, 0.5 M L-arginine, 20 mM CaCl2), 1 mM EDTA, 100 mM NaCl, 0.05% Brij 58, 0.05 mM GSSG and 0.5 mM GSH). After 3 days of incubation at 8° C., the refolding solution was concentrated by tangential filtration (Vivaflow 200, 10 kDa cut-off, Sartorius) and the buffer was exchanged to activation buffer (50 mM Tris, pH 7.5, 1 M NaCl and 0.05% Brij 58). The refolded TMPRSS2 was activated by removal of the N-terminal Met-Lys-(His)6 sequence (SEQ ID NO: 3), because a free isoleucine residue in position 16 at the N-terminus of the protease domain was required for activity. This was obtained by incubating the protease for 5 h with 2.5 m-units/ml of activated DAPase (Qiagen) at room temperature (˜20-C). The activated protease was separated from non-activated protease and His-tagged DAPase by metal chelate chromatography and was later designated as active TMPRSS2.

A yield of this protocol was about 0.6 mg of active catalytic domain per 2 L cell culture. For analysis of the purified protein, an SDS-side followed by Western blotting was performed using TMPRSS2-specific antibodies.

### Example 3. Exemplary Enzyme Kinetic Studies for the Determination of TMPRSS2

All measurements were performed at room temperature in 50 mM Tris/HCl buffer (pH 8.0; containing 154 mM NaCl). All substrate stock solutions (2 mM) were prepared in ultrapure water containing 10% DMSO and further diluted by water to the appropriate concentrations.

Measurements with Chromogenic pNa Substrates

The cleavage of the pNa substrates was measured at 405 nm using a microplate IEMS Reader MF 1401 (Labsystems). The initial screening was performed with a single substrate concentration of 200 μM in the assay. For the five best substrates, the enzyme kinetic parameters Kₘ and Vₘₐₓ were determined from two independent experiments.

Measurements with Fluorogenic AMC Substrates

The determination of the TMPRSS2-inhibitory effect was carried out using a Satire² fluorescence plate reader (Tecan; λ_{Ex}=380 nm and λ_{Em}=460 nm) and H-dCha-Pro-Arg-AMC×2 TFA as a substrate. The enzyme used was the recombinant protease domain of TMPRSS2. For the determination of the inhibition constants, the measurement buffer was combined with substrate with different inhibitor concentrations, which were varied at least over the range of one order of magnitude. After enzyme addition, the steady-state rates were determined by linear regression. The Kₘ and Vₘₐₓ values (in unit: ARFU/s) were calculated as the average of two independent measurements. The Kᵢ values were calculated by adapting the determined rates as a function of the inhibitor and substrate concentrations to the rate equation for completely reversible binding inhibitors:

$$v = \frac{{V}_{max} \cdot [ S ]}{{K}_{m} \cdot ( 1 + \frac{[ l ]}{{K}_{i}} ) + [ S ]}$$

Inhibitor Measurements

The Kᵢ determinations were performed according to the method of Dixon (Dixon, M. (1953). The determination of enzyme inhibitor constants. Biochem. J. 55, 170-171) using the fluorogenic substrate H-D-cyclohexylalanine-Pro-Arg-AMC (200, 100 and 50 μM). The Kᵢ values were calculated as the average of two independent measurements.

### Example 4. Exemplary Inhibition of TMPRSS2-Mediated Virus Propagation in the Presence of Synthetic Serine Protease Inhibitors

MDCK-TMPRSS2 cells with inducible expression of the protease TMPRSS2 were used for the experiments. MDCK-TMPRSS2 cells were isolated by stable transfection of MDCK cells (Madin Darby Canine Kidney) with the plasmids pcEFTet-On/NEO and pTRE2pur-TMPRSS2-FLAG (Bottcher et al., Vaccine 27, 62324-6329 (2009); Bottcher et J Viral 84, 5605-5614 (2010)). The expression of TMPRSS2 in these cells can be induced by adding doxycycline (Dox) to the culture medium (Tet-On expression system, Gossen and Bujard, Science 1995).

To analyze the efficacy of synthetic serine protease inhibitors on the inhibition of influenza virus proteolytic activation by TMPRSS2, multicyclic replication and virus spread in MDCK-TMPRSS2 cells in the presence of the inhibitors was examined. MDCK-TMPRSS2 cells were first cultured in 24-well plates for 24 h in the presence and absence of 0.2 μg/mL doxycycline. The cells were subsequently infected with the human influenza isolate A/Memphis/14/96 (H1N1) and incubated for 24 h in the presence or absence of various inhibitors at 37° C. and 5% CO₂. Subsequently, infected cells were immunohistochemically stained against the viral nucleoprotein. A concentration-dependent inhibition of virus proliferation and spread by the synthetic inhibitors used was demonstrated.

### Example 5. Exemplary Inhibition of TMPRSS2-Mediated Virus Propagation by Synthetic Serine Protease Inhibitors in Human Airway Epithelial Cells

Calu-3 cells (human respiratory epithelial cells, endogenous expression of TMPRSS2) were used to demonstrate the inhibitory effect of TMPRSS2-mediated viral spread. For this purpose, the cells were cultured in 6-well plates and infected with the human influenza virus isolate A/Memphis/14/96 (H1N1) in the presence and absence of inhibitor 2 (50 μM) for 72 h and at various times the virus titers in cell culture supernatant by means of Plaque test (determination of infectious virus per ml, pfu: plaque forming units) determined. A significant delay in virus replication and a 1000-fold decrease in the virus titer in the presence of inhibitor 2 compared to the control without inhibitor (w/o inhibitor) was observed.

### Example 6. Inhibition of TMPRSS2-Mediated Viral Replication by Synthetic Serine Protease Inhibitors in Human Respiratory Epithelial Cells after Treatment with the Inhibitors at Different Times

These experiments were carried out analogously to Example 5, but the inhibitors were not until 14 or 24 hours after infection of the cells with the influenza viral A/Memphis/14/96 (H1N1) or A/Hamburg/5/09 (H1N1) to the cell culture. Even in these cases, significant inhibition of virus replication and 50-100 fold reduction in virus titers were still observed.

### Example 7. Inhibition of TMPRSS2-Mediated Viral Replication by Combined Treatment with a Synthetic Serine Protease Inhibitor and a Neuraminidase Inhibitor in Human Respiratory Epithelial Cells

This experiment was carried out analogously to Example 5. For this purpose, Calu-3 cells were infected with the isolate A/Aichi/2/68 (H3N2) and then in the presence or absence of inhibitor 2 (20 μM) or the neuraminidase inhibitor oseltamivir carboxylate (0.1 μM) or in the presence of both inhibitors (same concentrations) for 72 h. At various times, the virus titer was determined by plaque assay. It was observed that inhibitor-2 and oseltamivir carboxylate act synergistically and that an almost complete inhibition of viral replication by combination of both inhibitors were achieved.

### Example 8. Exemplary Infection and Multicycle Viral Replication in Inhibitor-Treated Cells

All infection experiments were performed using infection medium. For analysis of influenza virus multicycle replication in Calu-3 cells in the presence of inhibitors, cells were seeded in six-well plates and grown to confluence. The cells were then inoculated with virus at a low MOI (multiplicity of infection) of 0.0001 for 1 h in the absence of inhibitors, washed with PBS and replenished with fresh infection medium containing inhibitors at the indicated concentrations. The cells were incubated for 72 h and at 24, 48 and 72 h postinfection, supernatants were collected and viral titres were determined as pfu (plaque-forming units) by plaque assay as described previously. Briefly, MDCK cells grown in 24-well plates were inoculated with 10-fold serial dilutions of each virus sample for 1 h. The inoculum was then removed and replaced by Avicel overlay containing 1 g/ml TPCK-treated trypsin. The cells were incubated for 48 h and subsequently immunostained using virus-specific antibodies, HRP-conjugated secondary antibodies and the peroxidase substrate TrueBlue® (KPL).

To analyse cleavage of HA of progeny virus, Calu-3 cells were infected at a 100-fold higher MOI of 0.01 and incubated for 24 h (for A/Aichi/H3N2) or 48 h (for A/Hamburg/H1N1). Virus-containing cell supernatants were cleared from cell debris by low-speed centrifugation (4100 g, 5 min) and then pelleted by ultracentrifugation (Beckman Coulter rotor SW 41 Ti, 30000 rev./min, 2 h, 4° C.). Pellets were resuspended in reducing SDS sample buffer, heated at 95° C. for 5 min and subjected to SDS/PAGE and Western blot analysis using antibodies against H1 or H3 as described above.

### Example 9. Exemplary Cytotoxicity Assay

To determine the viability of inhibitor-treated cells, a quantitative colorimetric MTT [3-(4,5-dimethylthiazol-2-yl)-2,5-diphenyl-2H-tetrazolium bromide] assay (Sigma) was used. Calu-3 cells grown in 96-well plates were treated with PBS or the different inhibitors at the indicated concentrations in infection medium (total volume 100 μl per well) for 48 h at 37° C. Then, 20 l of MTT stock solution (2 mg/ml in PBS) was added to each well and the cells were further incubated for 2-3 h at 37° C., until purple formazan crystals are visible. Finally, the MTT-containing medium was removed and the formazan dissolved in 200 μl of DMSO, after which the absorbance was measured at 562 nm on a microplate ELISA reader.

### Example 10. Exemplary Examination of Coronaviruses Employing TMPRSS2 for S Protein Priming in Human Cell Lines

The examination of coronaviruses can be done using methods known from the literature (Hoffmann, M. et al. 2020 Apr. 16; 181(2):271-280).

Calu-3 cells were pre-incubated with TMPRSS2 inhibitor and subsequently inoculated with pseudoparticles harboring MERS-S, SARS-S and SARS-2-S viral glycoproteins (coronaviruses employing TMPRSS2 for S protein priming in human cell lines).

Calu-3 cells were pre-incubated with TMPRSS2 inhibitor and infected with SARS-CoV-2. Subsequently, the cells were washed and genome equivalents in culture supernatants are determined by quantitative RT-PCR.

### Example 11. Synthesis of Compound 1

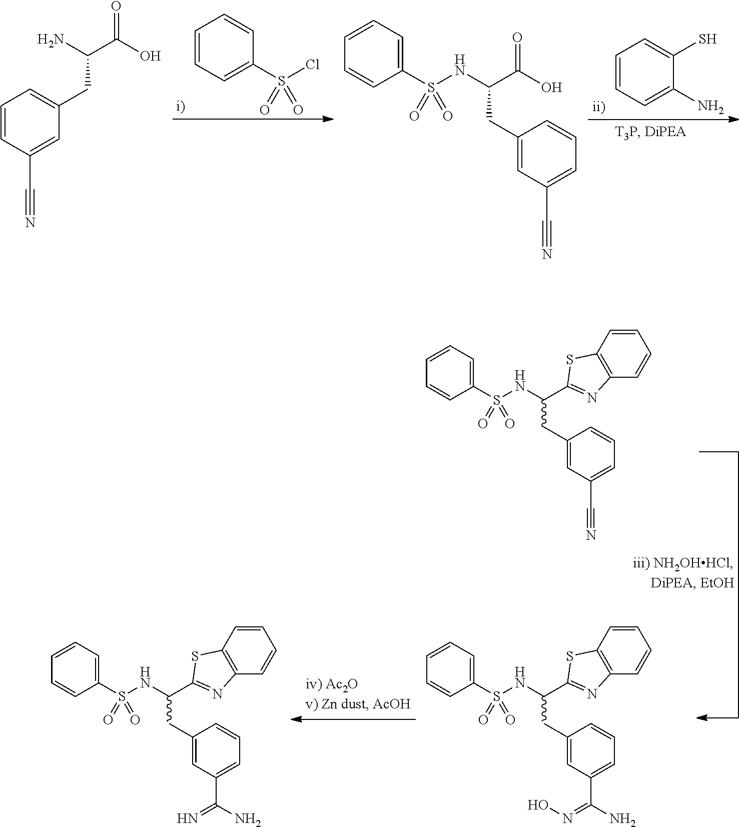

Benzenesulfonyl chloride (1.0 eq.), Na₂CO₃ (1.2 eq.), H₂O, −5° C. to RT, 3 h. ii) 2-Aminothiophenol (1.005 eq.), DIPEA (1.5 eq.), T3P (50% w/w in EtOAc) (1.009 eq.), EtOAc, RT to reflux, 3 h. iii) Hydroxylamine hydrochloride (2.5 eq.), DIPEA (3.0 eq.), EtOH (5 mL), reflux, 1 h. iv) Acetic anhydride (1.05 eq.), AcOH (2 mL), 0° C., 10 mins. v) Zn dust (10.0 eq.), AcOH (5 mL), RT, 18 h

### Step 1: (2S)-2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[1-(1,3-Benzothiazol-2-yl)-2-(3-cyanophenyl)ethyl]benzenesulfonamide

To a magnetically stirred solution of (2S)-2-benzenesulfonamido-3-(3-cyanophenyl)propanoic acid (0.250 g, 0.757 mmol, 1.0 eq.) in EtOAc (5.5 mL) were added DIPEA (0.201 mL, 1.15 mmol, 1.5 eq.) and 2-aminothiophenol (0.081 mL, 0.760 mmol, 1.005 eq.). The mixture was cooled in an ice/water bath and placed under a nitrogen atmosphere before adding T3P (50% w/w in EtOAc) (0.486 g, 0.763 mmol, 1.009 eq.) dropwise over 10 minutes. The reaction mixture was allowed to warm to RT for 40 mins and then stirred under reflux for 3 h. The reaction mixture was cooled to RT and then diluted with EtOAc (25 mL). The mixture was washed with aq. saturated NaHCO₃ solution and the organic layer dried over anhydrous sodium sulfate before filtering and concentrating to dryness. The residue was purified by Flash Column Chromatography (Silica, 10-50% EtOAc, heptane) to afford product as a white solid (0.191 g, 0.451 mmol, 60% yield). ¹H NMR (DMSO-d6) δ: 9.03 (s, 1H), 8.19-8.12 (m, 1H), 8.05-7.98 (m, 1H), 7.67 (t, J=1.7 Hz, 1H), 7.63-7.45 (m, 7H), 7.41-7.31 (m, 3H), 5.09 (dd, J=10.2, 4.5 Hz, 1H), 3.46 (dd, J=13.9, 4.6 Hz, 1H), 3.08 (dd, J=13.9, 10.5 Hz, 1H). UPLC-MS (basic 4 min) Rt=1.88 min. m/z=420.1 for [M+H]⁺

### Step 3: 3-[2-Benzenesulfonamido-2-(1,3-benzothiazol-2-yl)ethyl]-N-hydroxybenzene-1-carboximidamide)

To a magnetically stirred solution of N-[1-(1,3-benzothiazol-2-yl)-2-(3-cyanophenyl)ethyl] benzenesulfonamide (0.305 g, 0.727 mmol, 1.0 eq.) in EtOH (5 mL) were added hydroxylamine hydrochloride (0.126 g, 1.82 mmol, 2.5 eq.) and DIPEA (0.38 mL, 2.18 mmol, 3.0 eq.). The reaction mixture was stirred under reflux for 1 h. The reaction mixture was cooled down to RT and then concentrated to dryness to afford product as a yellow oil (0.329 g, 0.727 mmol, 100% yield) which was used in the next step without further purification. UPLC-MS (acidic 2 min) Rt=0.98 min. m/z=453.2 for [M+H]⁺

### Step 4: 3-[2-Benzenesulfonamido-2-(1,3-benzothiazol-2-yl)ethyl]benzene-1-carboximidamide (compound 1 (racemic mixture, batch 1))

To a magnetically stirred solution of 3-[2-benzenesulfonamido-2-(1,3-benzothiazol-2-yl)ethyl]-N-hydroxybenzene-1-carboximidamide (0.330 g, 0.729 mmol, 1.0 eq) in acetic acid (5.0 mL) was added acetic anhydride (0.072 mL, 0.766 mmol, 1.05 eq.) dropwise at 0° C. The resulting mixture was stirred at RT for 10 mins. and then zinc (0.477 g, 7.29 mmol, 10.0 eq.) was added. The reaction mixture was stirred at room temperature for 18 h. The reaction mixture was filtered through a plug of celite and then concentrated to dryness. The residue was dissolved in EtOAc (10 mL) and then washed with aqueous saturated sodium bicarbonate solution (10 mL). The combined organic layers were dried over anhydrous sodium sulfate and the concentrated to dryness. The residue was purified by Reverse Phase column chromatography on a 120 g C18 cartridge eluting with a 5-95% H₂O:MeCN eluent (0.1% ammonia) to afford product as a white solid (0.031 g, 0.073 mmol, 10% yield). ¹H NMR (400 MHz, DMSO-d₆): δ 9.32 (s, 2H), 9.15 (s, 2H), 9.03 (d, J=8.2 Hz, 1H), 8.18-8.11 (m, 1H), 8.03-7.96 (m, 1H), 7.81 (t, J=1.8 Hz, 1H), 7.69-7.61 (m, 1H), 7.59 (dd, J=7.2, 1.3 Hz, 1H), 7.58-7.52 (m, 3H), 7.52-7.37 (m, 2H), 7.32 (dt, J=8.2, 7.0 Hz, 2H), 5.14 (ddd, J=10.0, 8.1, 4.9 Hz, 1H), 3.46 (dd, J=13.9, 4.9 Hz, 1H), 3.13 (dd, J=13.9, 10.0 Hz, 1H). UPLC-MS (basic 4 min): Rt=1.41 min; m/z=437.3 for [M]⁺, 99% purity. Chiral analysis performed by Reach Separations showed 63.6% chiral purity.

Following the procedure used for the preparation of compound 1 (racemic mixture, batch 1), compound 1 (racemic mixture, batch 3) was isolated with 100% purity by UPLC-MS. Chiral analysis performed by Reach Separations showed 55.4% chiral purity for compound 1 (racemic mixture, batch 3).

Compound 1 (enantiomer 1, batch 1) was isolated by preparative chiral HPLC of compound 1 (racemic mixture, batch 2), the acetate salt form (98% pure by UPLC-MS)). Product purified by SFC using Lux C₁ (21.2 mm×250 mm, 5 um) with a 20:80 MeOH:CO₂ (0.2% v/v NH₃) eluent to afford product as a white solid (0.007 g).

Compound 1 (enantiomer 1, batch 1) was isolated in 98% purity by UPLC-MS. Chiral analysis performed by Reach Separations showed 93% chiral purity. Rt=6.659 minutes.

Compound 1 (enantiomer 1, batch 2) was isolated by preparative chiral HPLC of compound 1 (racemic mixture, batch 3). Product purified by SFC using Lux C1 (21.2 mm×250 mm, Sum) with a 20:80 MeOH:CO₂ (0.2% v/v NH₃) eluent to afford product as a white solid (0.040 g). Compound 1 (enantiomer 1, batch 2) was isolated in 95% purity by UPLC-MS. Chiral analysis performed by Reach Separations showed 99.6% chiral purity. Rt=8.749 minutes.

Compound 1 (enantiomer 2, batch 1) was isolated by preparative chiral HPLC of compound 1 (racemic mixture, batch 2), the acetate salt form (98% pure by UPLC-MS)). Product purified by SFC using Lux C1 (21.2 mm×250 mm, Sum) with a 20:80 MeOH:CO₂ (0.2% v/v NH₃) eluent to afford product as a white solid (0.004 g). Compound 1 (enantiomer 2, batch 1) was isolated in 99% purity by UPLC-MS. Chiral analysis performed by Reach Separations showed 93.3% chiral purity. Rt=8.113 minutes.

Compound 1 (enantiomer 2, batch 2) was isolated by preparative chiral HPLC of compound 1 (racemic mixture, batch 3). Product purified by SFC using Lux C1 (21.2 mm×250 mm, 5 um) with a 20:80 MeOH:CO₂ (0.2% v/v NH₃) eluent to afford product as a white solid (0.027 g). Compound 1 (enantiomer 2, batch 2) was isolated in 98% purity by UPLC-MS. Chiral analysis performed by Reach Separations showed 98.4% chiral purity. Rt=10.822 minutes.

### Example 12. Synthesis of Compound 2

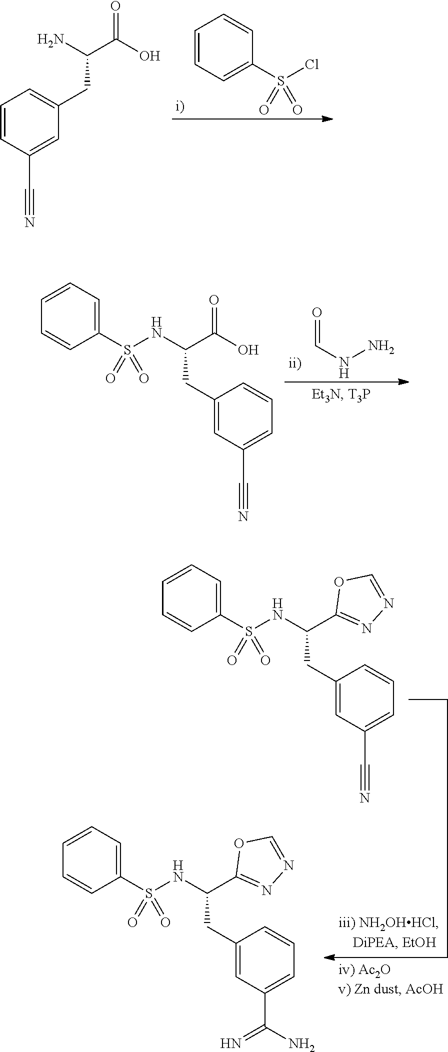

Benzenesulfonyl chloride (1.0 eq.), Na₂CO₃ (1.2 eq.), H₂O, −5° C. to RT, 3 h. ii) formic hydrazide (1.05 eq.), triethylamine (4.0 eq.), T3P (50% w/w in EtOAc) (2.5 eq.), toluene, 110° C., 4 h. iii) hydroxylamine hydrochloride (2.5 eq.), DIPEA (3.0 eq.), EtOH (5 mL), reflux, 1 h. iv) acetic anhydride (1.05 eq.), AcOH (2 mL), 0° C., 10 mins. v) Zn dust (10.0 eq.), AcOH (5 mL), RT, 18 h

### Step 1: (2S)-2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[(1S)-2-(3-Cyanophenyl)-1-(1,3,4-oxadiazol-2-yl)ethyl]benzenesulfonamide

To a magnetically stirred solution of (2S)-2-benzenesulfonamido-3-(3-cyanophenyl)propanoic acid in toluene (9.3 mL) were added formic hydrazide (0.095 g, 1.59 mmol, 1.05 eq.) and triethylamine (0.842 mL, 6.05 mmol, 4.0 eq.). A solution of T3P (50% w/w in EtOAc) (2.3 mL, 3.78 mmol, 2.5 eq.) was added dropwise over 5 minutes and the reaction mixture was stirred at 110° C. for 4 h. The reaction mixture was cooled down to RT and then diluted with EtOAc (25 mL). The mixture was washed with aq. sat. NaHCO₃ solution (10 mL) followed by aq. 1M HCl solution (10 mL). The organic layer was dried over anhydrous sodium sulfate before filtering and concentrating to dryness. The residue was purified by Reverse Phase column chromatography on a C18 cartridge eluting with a 5-95% H₂O:MeCN eluent (0.1% ammonia) to afford product as a white solid (0.130 g, 0.337 mmol, 22% yield). ¹H NMR (DMSO-d6) δ: 9.06 (s, 1H), 8.83 (d, J=8.4 Hz, 1H), 7.61 (dt, J=6.9, 1.5 Hz, 2H), 7.58-7.47 (m, 4H), 7.46-7.31 (m, 3H), 5.01-4.91 (m, 1H), 3.21 (dd, J=13.8, 6.0 Hz, 1H), 3.09 (dd, J=13.8, 9.6 Hz, 1H). UPLC-MS (basic 2 min) Rt=0.91 min. m/z=355.3 for [M+H]⁺

### Step 3: 3-[(2S)-2-Benzenesulfonamido-2-(1,3,4-oxadiazol-2-yl)ethyl]-N′-hydroxybenzene-1-carboximidamide

To a magnetically stirred solution of N-[(1S)-2-(3-cyanophenyl)-1-(1,3,4-oxadiazol-2-yl)ethyl] benzenesulfonamide (0.315 g, 0.889 mmol, 1.0 eq.) in EtOH (6.1 mL) were added hydroxylamine hydrochloride (0.124 g, 1.78 mmol, 2.0 eq.) and DIPEA (0.464 mL, 2.67 mmol, 3.0 eq.). The reaction mixture was stirred under reflux for 1 h. The reaction mixture was cooled down to RT and then concentrated to dryness to afford product as a yellow oil (0.340 g, 0.878 mmol, 99% yield) which was used in the next step without further purification. UPLC-MS (basic 4 min) Rt=1.01 min. m/z=388.3 for [M+H]⁺

### Step 4: Amino({3-[(2S)-2-benzenesulfonamido-2-(1,3,4-oxadiazol-2-yl)ethyl]phenyl})methylidene]amino acetate

To a magnetically stirred solution of 3-[(2S)-2-benzenesulfonamido-2-(1,3,4-oxadiazol-2-yl)ethyl]-N′-hydroxybenzene-1-carboximidamide (0.340 g, 0.878 mmol, 1.0 eq) in acetic acid (6.1 mL) was added acetic anhydride (0.087 mL, 0.922 mmol, 1.05 eq.) dropwise at 0° C. The resulting mixture was stirred at RT for 60 mins. and then concentrated to dryness. The residue was purified by Normal Phase column chromatography eluting with a 10-90% EtOAc, heptane eluent to afford product as a white solid (0.230 g, 0.514 mmol, 59% yield). ¹H NMR (400 MHz, DMSO-d₆): δ 9.07 (s, 1H), 8.97 (s, 1H), 7.66-7.55 (m, 5H), 7.54-7.44 (m, 2H), 7.36-7.25 (m, 2H), 6.84 (s, 2H), 4.97 (t, J=7.8 Hz, 1H), 3.23 (dd, J=13.8, 7.3 Hz, 1H), 3.15 (dd, J=13.7, 8.4 Hz, 1H), 2.21 (s, 3H), 1.33-1.21 (m, 1H). UPLC-MS (basic 4 min): Rt=1.13 min; m/z=430.0 for [M]⁺

### Step 5: 3-[2-Benzenesulfonamido-2-(1,3,4-oxadiazol-2-yl)ethyl]benzene-1-carboximidamide), (compound 2 (enantiomer 1, batch 1))

To a magnetically stirred solution of amino({3-[(2S)-2-benzenesulfonamido-2-(1,3,4-oxadiazol-2-yl)ethyl]phenyl})methylidene]amino acetate (0.230 g, 0.514 mmol, 1.0 eq) in acetic acid (3.5 mL) was added zinc (0.336 g, 5.14 mmol, 10.0 eq.) and the reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford product as a white solid (0.082 g, 43% yield). ¹H NMR (400 MHz, DMSO-d₆): δ 8.95-8.92 (m, 1H), 8.10 (s, 2H), 7.60 (d, J=1.8 Hz, 1H), 7.56 (dt, J=7.6, 1.7 Hz, 3H), 7.52-7.46 (m, 2H), 7.44-7.38 (m, 1H), 7.38-7.30 (m, 2H), 7.29-7.21 (m, 2H), 4.80 (t, J=7.6 Hz, 1H), 3.14-3.00 (m, 2H). UPLC-MS (basic 4 min): Rt=0.80 min; m/z=372.3 for [M+H]⁺, 99% purity. Chiral analysis performed by Reach Separations showed 87.5% chiral purity.

Compound 2 (enantiomer 1, batch 1) was purified by SFC using Lux iA3 (21.2 mm×250 mm, 5 um) with a 25:75 MeOH:CO₂ (0.2% v/v NH₃) eluent to afford compound 2 (enantiomer 1, batch 2) as a white solid (0.027 g). ¹H NMR (400 MHz, DMSO-d₆): δ 8.95-8.92 (m, 1H), 8.10 (s, 2H), 7.60 (d, J=1.8 Hz, 1H), 7.56 (dt, J=7.6, 1.7 Hz, 3H), 7.52-7.46 (m, 2H), 7.44-7.38 (m, 1H), 7.38-7.30 (m, 2H), 7.29-7.21 (m, 2H), 4.80 (t, J=7.6 Hz, 1H), 3.14-3.00 (m, 2H). UPLC-MS (acidic 4 min): Rt=0.76 min; m/z=372.1 for [M+H]+, 100% purity. Chiral analysis performed by Reach Separations showed 98.5% chiral purity. Rt=4.37 min.

### Example 13. Synthesis of Compound 3

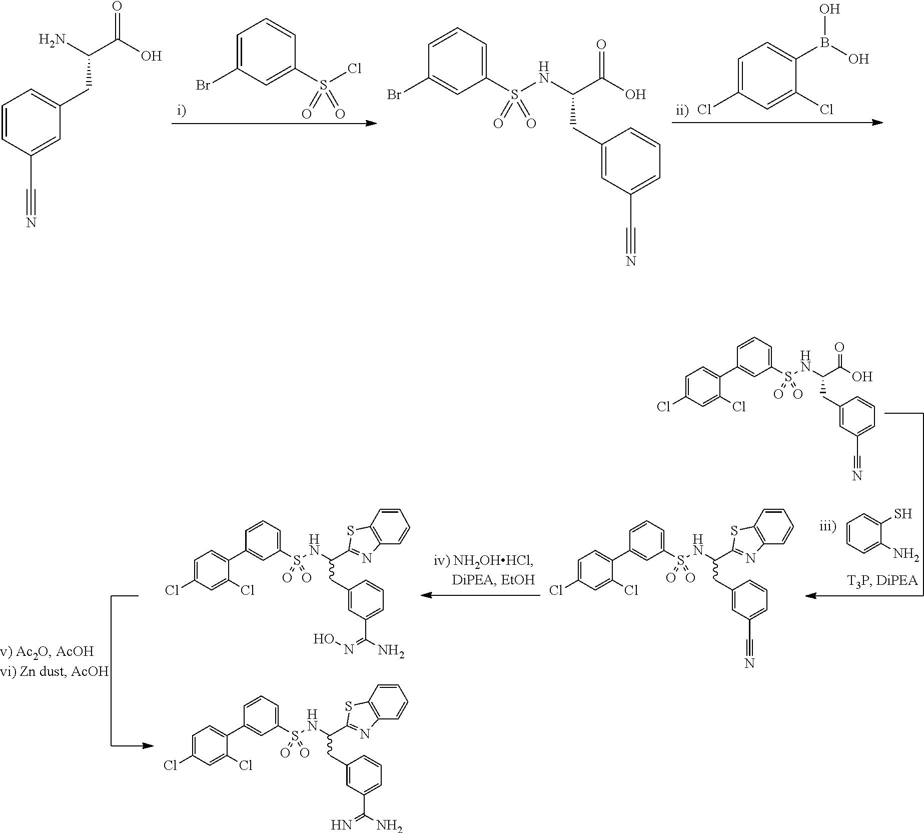

3-Bromobenzene-1-sulfonyl chloride (1.0 eq.), Na₂CO₃ (1.4 eq.), H₂O, −5° C. to RT, 2 h. ii) 2,4-Dichlorophenyl)boronic acid (2.0 eq.), Pd(PPh₃)₄ (0.1 eq.), K₂CO₃ (3.0 eq.), toluene, EtOH, RT, 24 h. iii) 2-Aminothiophenol (1.005 eq.), DIPEA (1.5 eq.), T3P (50% w/w in EtOAc) (1.2 eq.), EtOAc, RT to reflux, 4 h. iv) Hydroxylamine hydrochloride (2.0 eq.), DIPEA (3.0 eq.), EtOH (2 mL), reflux, 1 h. v) Acetic anhydride (3.0 eq.), AcOH (2 mL). vi) Zn dust (10.0 eq.), AcOH (2 mL), RT, 24 h.

### Step 1: (2S)-2-(3-Bromobenzenesulfonamido)-3-(3-cyanophenyl)propanoic acid)

To a magnetically stirred solution of sodium bicarbonate (3.34 g, 31.5 mmol, 1.4 eq.) in water (28 mL) was added 3-bromobenzene-1-sulfonyl chloride (5.85 g, 22.9 mmol, 1.0 eq.) and the resulting solution was cooled to −5° C. (2S)-2-amino-3-(3-cyanophenyl)propanoic acid (5.00 g, 26.3 mmol, 1.1 eq.) was added in 4 portions over a period of 40 mins. The resulting slurry was allowed to warm to RT and stirred for 2 h. The reaction mixture was acidified with 20% HCl and then filtered under vacuum. The solid was washed with water (2×50 mL) and then dried to afford product as a white solid (7.00 g, 17.1 mmol, 75% yield). ¹H NMR (DMSO-d6) δ: 8.51 (d, J=9.1 Hz, 1H), 7.75-7.70 (m, 1H), 7.65-7.43 (m, 5H), 7.41-7.29 (m, 2H), 4.04 (ddd, J=10.3, 9.1, 4.5 Hz, 1H), 3.07 (dd, J=13.8, 4.5 Hz, 1H), 2.76 (dd, J=13.8, 10.4 Hz, 1H). UPLC-MS (basic 2 min) Rt=0.80 min. m/z=410.0 for [M+H]⁺

### Step 2: (2S)-3-(3-Cyanophenyl)-2-{2′,4′-dichloro-[1,1′-biphenyl]-3-sulfonamido}propanoic acid)

To a degassed solution of (2S)-2-(3-bromobenzenesulfonamido)-3-(3-cyanophenyl)propanoic acid) (2.00 g, 4.89 mmol, 1.0 eq.), 2,4-dichlorophenyl)boronic acid (1.87 g, 9.77 mmol, 2.0 eq.) and K₂CO₃ (2.03 g, 14.7 mmol, 3.0 eq.) in toluene (20 mL) and EtOH (20 mL) was added Pd(PPh₃)₄ (0.056 g, 0.049 mmol, 0.1 eq.) and the reaction mixture was stirred under reflux for 24 h. The reaction mixture was cooled down to RT and then concentrated to dryness. The residue was taken up in water (100 mL) and then extracted with EtOAc (2×20 mL). The combined organic phase was washed with brine (50 mL) and then dried over anhydrous sodium sulfate before filtering and concentrating to dryness. The residue was triturated with MTBE and heptane to afford product as a beige solid (1.90 g, 4.00 mmol, 82% yield). ¹H NMR (DMSO-d6) δ: 7.82-7.72 (m, 3H), 7.72-7.53 (m, 6H), 7.53-7.47 (m, 2H), 7.41 (t, J=7.6 Hz, 1H), 6.82 (s, 1H), 3.30 (d, J=4.5 Hz, 1H), 3.17-3.07 (m, 1H), 2.96 (dd, J=13.4, 5.3 Hz, 1H). UPLC-MS (basic 2 min) Rt=0.98 min. m/z=476.0 for [M+H]⁺

### Step 3: N-[1-(1,3-benzothiazol-2-yl)-2-(3-cyanophenyl)ethyl]-2′,4′-dichloro-[1,1′-biphenyl]-3-sulfonamide)

To a magnetically stirred solution of (2S)-3-(3-cyanophenyl)-2-{2′,4′-dichloro-[1,1′-biphenyl]-3-sulfonamido}propanoic acid (0.500 g, 1.05 mmol, 1.0 eq.) in EtOAc (7.6 mL) were added DIPEA (0.279 mL, 1.56 mmol, 1.5 eq.) and 2-aminothiophenol (0.110 mL, 1.06 mmol, 1.005 eq.). The mixture was cooled in an ice/water bath and placed under a nitrogen atmosphere before adding T3P (50% w/w in EtOAc) (0.803 g, 1.26 mmol, 1.2 eq.) dropwise over 10 minutes. The reaction mixture was allowed to warm to RT for 40 mins and then stirred under reflux for 4 h. The reaction mixture was cooled to RT and then diluted with EtOAc (25 mL). The mixture was washed with aq. saturated NaHCO₃ solution and the organic layer dried over anhydrous sodium sulfate before filtering and concentrating to dryness. The residue was purified by Flash Column Chromatography (Silica, 10-50% EtOAc, heptane) to afford product as a white solid (0.400 g, 0.709 mmol, 59% yield). ¹H NMR (DMSO-d6) δ: 9.12 (s, 1H), 8.20-8.09 (m, 1H), 8.05-7.93 (m, 1H), 7.81 (d, J=2.1 Hz, 1H), 7.74 (d, J=1.7 Hz, 1H), 7.66-7.42 (m, 9H), 7.38 (d, J=7.8 Hz, 1H), 7.37-7.26 (m, 1H), 5.18 (dd, J=10.2, 4.7 Hz, 1H), 3.45 (dd, J=13.9, 4.8 Hz, 1H), 3.12 (dd, J=13.9, 10.4 Hz, 1H). UPLC-MS (basic 4 min) Rt=2.38 min. m/z=564.3 for [M]⁺

### Step 4: 3-[2-(1,3-Benzothiazol-2-yl)-2-{2′,4′-dichloro-[1,1′-biphenyl]-3-sulfonamido}ethyl]-N′-hydroxybenzene-1-carboximidamide

To a magnetically stirred solution of N-[1-(1,3-benzothiazol-2-yl)-2-(3-cyanophenyl)ethyl]-2′,4′-dichloro-[1,1′-biphenyl]-3-sulfonamide) (0.265 g, 0.469 mmol, 1.0 eq.) in EtOH (3.1 mL) were added hydroxylamine hydrochloride (0.065 g, 0.939 mmol, 2.0 eq.) and DIPEA (0.245 mL, 1.41 mmol, 3.0 eq.). The reaction mixture was stirred under reflux for 1 h. The reaction mixture was cooled down to RT and then concentrated to dryness to afford product as a yellow oil (0.280 g, 0.371 mmol, 79% yield) which was used in the next step without further purification. UPLC-MS (basic 4 min) Rt=2.11 min. m/z=597.3 for [M]⁺

### Step 5: Amino({3-[2-(1,3-benzothiazol-2-yl)-2-{2′,4′-dichloro-[1,1′-biphenyl]-3-sulfonamido}ethyl]phenyl})methylidene]amino acetate

To a magnetically stirred solution of 3-[2-(1,3-benzothiazol-2-yl)-2-{2′,4′-dichloro-[1,1′-biphenyl]-3-sulfonamido}ethyl]-N′-hydroxybenzene-1-carboximidamide (0.280 g, 0.469 mmol, 1.0 eq) in acetic acid (3.2 mL) was added acetic anhydride (0.046 mL, 0.492 mmol, 1.05 eq.) dropwise at 0° C. The resulting mixture was stirred at RT for 40 mins. and then concentrated to dryness. The residue was purified by Reverse Phase column chromatography on a 120 g C18 cartridge eluting with a 5-95% H₂O:MeCN eluent (0.1% ammonia) to afford product as a white solid (0.185 g, 0.249 mmol, 53% yield). ¹H NMR (400 MHz, DMSO-d₆): δ 81 (s, 2H), 5.83 (s, 2H), 5.12 (dd, J=9.7, 5.2 Hz, 1H), 3.42-3.34 (m, 1H), 3.11 (dd, J=13.9, 9.7 Hz, 1H), 2.21 (s, 3H). UPLC-MS (basic 4 min): Rt=2.19 min; m/z=639.0 for [M]⁺

### Step 6: 3-[2-(1,3-Benzothiazol-2-yl)-2-{2′,4′-dichloro-[1,1′-biphenyl]-3-sulfonamido}ethyl]benzene-1-carboximidamide) (racemic)

To a magnetically stirred solution of amino({3-[2-(1,3-benzothiazol-2-yl)-2-{2′,4′-dichloro-[1,1′-biphenyl]-3-sulfonamido}ethyl]phenyl})methylidene]amino acetate (0.139 g, 0.217 mmol, 1.0 eq) in acetic acid (1.5 mL) was added zinc (0.142 g, 2.17 mmol, 10.0 eq.) and the reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford product as a white solid (0.056 g, 0.093 mmol, 43% yield). ¹H NMR (400 MHz, DMSO-d₆): δ 8.01 (d, J=8.0 Hz, 1H), 7.89 (d, J=8.0 Hz, 1H), 7.77 (dd, J=6.6, 2.1 Hz, 2H), 7.60-7.53 (m, 3H), 7.49 (ddd, J=8.2, 6.4, 1.7 Hz, 2H), 7.45-7.34 (m, 3H), 7.35 (d, J=1.6 Hz, 1H), 7.26 (t, J=7.7 Hz, 1H), 7.18 (d, J=8.3 Hz, 1H), 4.94 (dd, J=8.5, 4.8 Hz, 1H), 3.29 (dd, J=13.4, 4.8 Hz, 1H), 3.10 (dd, J=13.4, 8.6 Hz, 1H). UPLC-MS (basic 6 min): Rt=3.63 min; m/z=580.9 for [M]⁺, 97% purity. Chiral analysis performed by Reach Separations showed 64.3% chiral purity.

### Example 14. Synthesis of Compound 3 (Enantiomer 1)

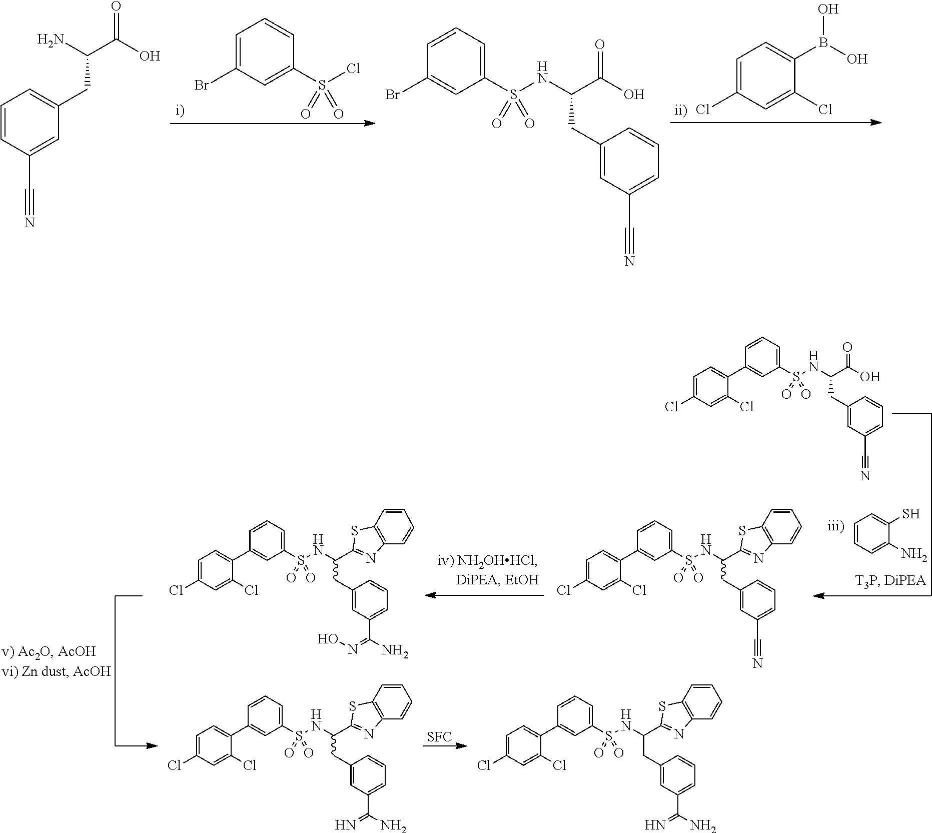

### Step 1: (2S)-2-(3-Bromobenzenesulfonamido)-3-(3-cyanophenyl)propanoic acid)

To a magnetically stirred solution of sodium bicarbonate (3.34 g, 31.5 mmol, 1.4 eq.) in water (28 mL) was added 3-bromobenzene-1-sulfonyl chloride (5.85 g, 22.9 mmol, 1.0 eq.) and the resulting solution was cooled to −5° C. (2S)-2-amino-3-(3-cyanophenyl)propanoic acid (5.00 g, 26.3 mmol, 1.1 eq.) was added in 4 portions over a period of 40 mins. The resulting slurry was allowed to warm to RT and stirred for 2 h. The reaction mixture was acidified with 20% HCl and then filtered under vacuum. The solid was washed with water (2×50 mL) and then dried to afford product as a white solid (7.00 g, 17.1 mmol, 75% yield). ¹H NMR (DMSO-d6) δ: 8.51 (d, J=9.1 Hz, 1H), 7.75-7.70 (m, 1H), 7.65-7.43 (m, 5H), 7.41-7.29 (m, 2H), 4.04 (ddd, J=10.3, 9.1, 4.5 Hz, 1H), 3.07 (dd, J=13.8, 4.5 Hz, 1H), 2.76 (dd, J=13.8, 10.4 Hz, 1H). UPLC-MS (basic 2 min) Rt=0.80 min. m/z=410.0 for [M+H]⁺ Step 2: (2S)-3-(3-Cyanophenyl)-2-{2′,4′-dichloro-[1,1′-biphenyl]-3-sulfonamido}propanoic acid)

To a degassed solution of (2S)-2-(3-bromobenzenesulfonamido)-3-(3-cyanophenyl)propanoic acid) (2.00 g, 4.89 mmol, 1.0 eq.), 2,4-dichlorophenyl)boronic acid (1.87 g, 9.77 mmol, 2.0 eq.) and K₂CO₃ (2.03 g, 14.7 mmol, 3.0 eq.) in toluene (20 mL) and EtOH (20 mL) was added Pd(PPh₃)₄ (0.056 g, 0.049 mmol, 0.1 eq.) and the reaction mixture was stirred under reflux for 24 h. The reaction mixture was cooled down to RT and then concentrated to dryness. The residue was taken up in water (100 mL) and then extracted with EtOAc (2×20 mL). The combined organic phase was washed with brine (50 mL) and then dried over anhydrous sodium sulfate before filtering and concentrating to dryness. The residue was triturated with MTBE and heptane to afford product as a beige solid (1.90 g, 4.00 mmol, 82% yield). ¹H NMR (DMSO-d6) δ: 7.82-7.72 (m, 3H), 7.72-7.53 (m, 6H), 7.53-7.47 (m, 2H), 7.41 (t, J=7.6 Hz, 1H), 6.82 (s, 1H), 3.30 (d, J=4.5 Hz, 1H), 3.17-3.07 (m, 1H), 2.96 (dd, J=13.4, 5.3 Hz, 1H). UPLC-MS (basic 2 min) Rt=0.98 min. m/z=476.0 for [M+H]⁺

### Step 3: N-[1-(1,3-Benzothiazol-2-yl)-2-(3-cyanophenyl)ethyl]-2′,4′-dichloro-[1,1′-biphenyl]-3-sulfonamide)

To a magnetically stirred solution of (2S)-3-(3-cyanophenyl)-2-{2′,4′-dichloro-[1,1′-biphenyl]-3-sulfonamido}propanoic acid (0.500 g, 1.05 mmol, 1.0 eq.) in EtOAc (7.6 mL) were added DIPEA (0.279 mL, 1.56 mmol, 1.5 eq.) and 2-aminothiophenol (0.110 mL, 1.06 mmol, 1.005 eq.). The mixture was cooled in an ice/water bath and placed under a nitrogen atmosphere before adding T3P (50% w/w in EtOAc) (0.803 g, 1.26 mmol, 1.2 eq.) dropwise over 10 minutes. The reaction mixture was allowed to warm to RT for 40 mins and then stirred under reflux for 4 h. The reaction mixture was cooled to RT and then diluted with EtOAc (25 mL). The mixture was washed with aq. saturated NaHCO₃ solution and the organic layer dried over anhydrous sodium sulfate before filtering and concentrating to dryness. The residue was purified by Flash Column Chromatography (Silica, 10-50% EtOAc, heptane) to afford product as a white solid (0.400 g, 0.709 mmol, 59% yield). ¹H NMR (DMSO-d6) δ: 9.12 (s, 1H), 8.20-8.09 (m, 1H), 8.05-7.93 (m, 1H), 7.81 (d, J=2.1 Hz, 1H), 7.74 (d, J=1.7 Hz, 1H), 7.66-7.42 (m, 9H), 7.38 (d, J=7.8 Hz, 1H), 7.37-7.26 (m, 1H), 5.18 (dd, J=10.2, 4.7 Hz, 1H), 3.45 (dd, J=13.9, 4.8 Hz, 1H), 3.12 (dd, J=13.9, 10.4 Hz, 1H). UPLC-MS (basic 4 min) Rt=2.38 min. m/z=564.3 for [M]⁺ Step 4: 3-[2-(1,3-Benzothiazol-2-yl)-2-{2′,4′-dichloro-[1,1′-biphenyl]-3-sulfonamido}ethyl]-N′-hydroxybenzene-1-carboximidamide

To a magnetically stirred solution of N-[1-(1,3-benzothiazol-2-yl)-2-(3-cyanophenyl)ethyl]-2′,4′-dichloro-[1,1′-biphenyl]-3-sulfonamide) (0.265 g, 0.469 mmol, 1.0 eq.) in EtOH (3.1 mL) were added hydroxyl amine hydrochloride (0.065 g, 0.939 mmol, 2.0 eq.) and DIPEA (0.245 mL, 1.41 mmol, 3.0 eq.). The reaction mixture was stirred under reflux for 1 h. The reaction mixture was cooled down to RT and then concentrated to dryness to afford product as a yellow oil (0.280 g, 0.371 mmol, 79% yield) which was used in the next step without further purification. UPLC-MS (basic 4 min) Rt=2.11 min. m/z=597.3 for [M]⁺

### Step 5: Amino({3-[2-(1,3-benzothiazol-2-yl)-2-{2′,4′-dichloro-[1,1′-biphenyl]-3-sulfonamido}ethyl]phenyl})methylidene]amino acetate

To a magnetically stirred solution of 3-[2-(1,3-benzothiazol-2-yl)-2-{2′,4′-dichloro-[1,1′-biphenyl]-3-sulfonamido}ethyl]-N′-hydroxybenzene-1-carboximidamide (0.280 g, 0.469 mmol, 1.0 eq) in acetic acid (3.2 mL) was added acetic anhydride (0.046 mL, 0.492 mmol, 1.05 eq.) dropwise at 0° C. The resulting mixture was stirred at RT for 40 mins. and then concentrated to dryness. The residue was purified by Reverse Phase column chromatography on a 120 g C18 cartridge eluting with a 5-95% H₂O:MeCN eluent (0.1% ammonia) to afford product as a white solid (0.185 g, 0.249 mmol, 53% yield). ¹H NMR (400 MHz, DMSO-d₆): δ 81 (s, 2H), 5.83 (s, 2H), 5.12 (dd, J=9.7, 5.2 Hz, 1H), 3.42-3.34 (m, 1H), 3.11 (dd, J=13.9, 9.7 Hz, 1H), 2.21 (s, 3H). UPLC-MS (basic 4 min): Rt=2.19 min; m/z=639.0 for [M]⁺

### Step 6: 3-[2-(1,3-Benzothiazol-2-yl)-2-{2′,4′-dichloro-[1,1′-biphenyl]-3-sulfonamido}ethyl]benzene-1-carboximidamide)

To a magnetically stirred solution of amino({3-[2-(1,3-benzothiazol-2-yl)-2-{2′,4′-dichloro-[1,1′-biphenyl]-3-sulfonamido}ethyl]phenyl})methylidene]amino acetate (0.139 g, 0.217 mmol, 1.0 eq) in acetic acid (1.5 mL) was added zinc (0.142 g, 2.17 mmol, 10.0 eq.) and the reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford product as a white solid (0.056 g, 0.093 mmol, 43% yield). ¹H NMR (400 MHz, DMSO-d₆): δ 8.01 (d, J=8.0 Hz, 1H), 7.89 (d, J=8.0 Hz, 1H), 7.77 (dd, J=6.6, 2.1 Hz, 2H), 7.60-7.53 (m, 3H), 7.49 (ddd, J=8.2, 6.4, 1.7 Hz, 2H), 7.45-7.34 (m, 3H), 7.35 (d, J=1.6 Hz, 1H), 7.26 (t, J=7.7 Hz, 1H), 7.18 (d, J=8.3 Hz, 1H), 4.94 (dd, J=8.5, 4.8 Hz, 1H), 3.29 (dd, J=13.4, 4.8 Hz, 1H), 3.10 (dd, J=13.4, 8.6 Hz, 1H). UPLC-MS (basic 6 min): Rt=3.63 min; m/z=580.9 for [M]⁺, 96% purity. Chiral analysis performed by Reach Separations showed 64.3% chiral purity.

Product purified by SFC using Lux C4 (21.2 mm×250 mm, Sum) with a 60:40 MeOH:CO₂ (0.2% v/v NH₃) eluent to afford product as a white solid (0.009 g). ¹H NMR (400 MHz, DMSO-d₆): δ 8.01 (d, J=8.0 Hz, 1H), 7.89 (d, J=8.0 Hz, 1H), 7.77 (dd, J=6.6, 2.1 Hz, 2H), 7.60-7.53 (m, 3H), 7.49 (ddd, J=8.2, 6.4, 1.7 Hz, 2H), 7.45-7.34 (m, 3H), 7.35 (d, J=1.6 Hz, 1H), 7.26 (t, J=7.7 Hz, 1H), 7.18 (d, J=8.3 Hz, 1H), 4.94 (dd, J=8.5, 4.8 Hz, 1H), 3.29 (dd, J=13.4, 4.8 Hz, 1H), 3.10 (dd, J=13.4, 8.6 Hz, 1H). UPLC-MS (acidic 4 min): Rt=1.45 min; m/z=581.1 for [M+H]⁺, 99% purity. Chiral analysis performed by Reach Separations showed 100% chiral purity. Rt=2.29 min.

### Example 15. Synthesis of Compound 3 (Enantiomer 2)

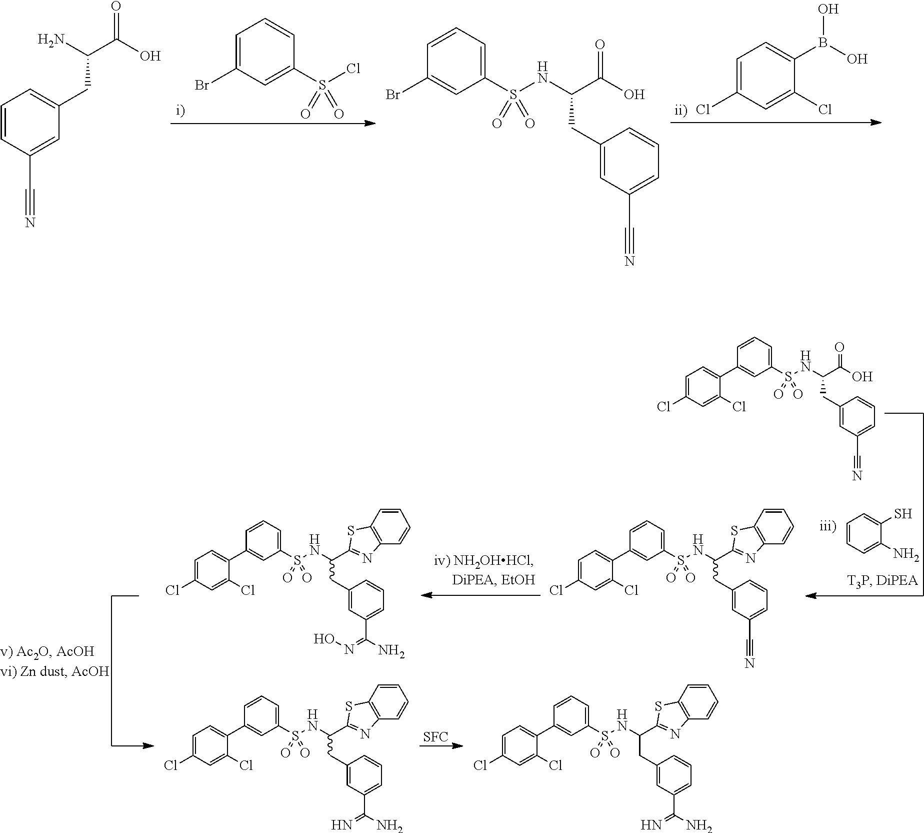

### Step 1: (2S)-2-(3-Bromobenzenesulfonamido)-3-(3-cyanophenyl)propanoic acid)

To a magnetically stirred solution of sodium bicarbonate (3.34 g, 31.5 mmol, 1.4 eq.) in water (28 mL) was added 3-bromobenzene-1-sulfonyl chloride (5.85 g, 22.9 mmol, 1.0 eq.) and the resulting solution was cooled to −5° C. (2S)-2-amino-3-(3-cyanophenyl)propanoic acid (5.00 g, 26.3 mmol, 1.1 eq.) was added in 4 portions over a period of 40 mins. The resulting slurry was allowed to warm to RT and stirred for 2 h. The reaction mixture was acidified with 20% HCl and then filtered under vacuum. The solid was washed with water (2×50 mL) and then dried to afford product as a white solid (7.00 g, 17.1 mmol, 75% yield). ¹H NMR (DMSO-d6) δ: 8.51 (d, J=9.1 Hz, 1H), 7.75-7.70 (m, 1H), 7.65-7.43 (m, 5H), 7.41-7.29 (m, 2H), 4.04 (ddd, J=10.3, 9.1, 4.5 Hz, 1H), 3.07 (dd, J=13.8, 4.5 Hz, 1H), 2.76 (dd, J=13.8, 10.4 Hz, 1H). UPLC-MS (basic 2 min) Rt=0.80 min. m/z=410.0 for [M+H]⁺

### Step 2: (2S)-3-(3-Cyanophenyl)-2-{2′,4′-dichloro-[1,1′-biphenyl]-3-sulfonamido}propanoic acid)

To a degassed solution of (2S)-2-(3-bromobenzenesulfonamido)-3-(3-cyanophenyl)propanoic acid) (2.00 g, 4.89 mmol, 1.0 eq.), 2,4-dichlorophenyl)boronic acid (1.87 g, 9.77 mmol, 2.0 eq.) and K₂CO₃ (2.03 g, 14.7 mmol, 3.0 eq.) in toluene (20 mL) and EtOH (20 mL) was added Pd(PPh₃)₄ (0.056 g, 0.049 mmol, 0.1 eq.) and the reaction mixture was stirred under reflux for 24 h. The reaction mixture was cooled down to RT and then concentrated to dryness. The residue was taken up in water (100 mL) and then extracted with EtOAc (2×20 mL). The combined organic phase was washed with brine (50 mL) and then dried over anhydrous sodium sulfate before filtering and concentrating to dryness. The residue was triturated with MTBE and heptane to afford product as a beige solid (1.90 g, 4.00 mmol, 82% yield). ¹H NMR (DMSO-d6) δ: 7.82-7.72 (m, 3H), 7.72-7.53 (m, 6H), 7.53-7.47 (m, 2H), 7.41 (t, J=7.6 Hz, 1H), 6.82 (s, 1H), 3.30 (d, J=4.5 Hz, 1H), 3.17-3.07 (m, 1H), 2.96 (dd, J=13.4, 5.3 Hz, 1H). UPLC-MS (basic 2 min) Rt=0.98 min. m/z=476.0 for [M+H]⁺

### Step 3: N-[1-(1,3-Benzothiazol-2-yl)-2-(3-cyanophenyl)ethyl]-2′,4′-dichloro-[1,1′-biphenyl]-3-sulfonamide)

To a magnetically stirred solution of (2S)-3-(3-cyanophenyl)-2-{2′,4′-dichloro-[1,1′-biphenyl]-3-sulfonamido}propanoic acid (0.500 g, 1.05 mmol, 1.0 eq.) in EtOAc (7.6 mL) were added DIPEA (0.279 mL, 1.56 mmol, 1.5 eq.) and 2-aminothiophenol (0.110 mL, 1.06 mmol, 1.005 eq.). The mixture was cooled in an ice/water bath and placed under a nitrogen atmosphere before adding T3P (50% w/w in EtOAc) (0.803 g, 1.26 mmol, 1.2 eq.) dropwise over 10 minutes. The reaction mixture was allowed to warm to RT for 40 mins and then stirred under reflux for 4 h. The reaction mixture was cooled to RT and then diluted with EtOAc (25 mL). The mixture was washed with aq. saturated NaHCO₃ solution and the organic layer dried over anhydrous sodium sulfate before filtering and concentrating to dryness. The residue was purified by Flash Column Chromatography (Silica, 10-50% EtOAc, heptane) to afford product as a white solid (0.400 g, 0.709 mmol, 59% yield). ¹H NMR (DMSO-d6) δ: 9.12 (s, 1H), 8.20-8.09 (m, 1H), 8.05-7.93 (m, 1H), 7.81 (d, J=2.1 Hz, 1H), 7.74 (d, J=1.7 Hz, 1H), 7.66-7.42 (m, 9H), 7.38 (d, J=7.8 Hz, 1H), 7.37-7.26 (m, 1H), 5.18 (dd, J=10.2, 4.7 Hz, 1H), 3.45 (dd, J=13.9, 4.8 Hz, 1H), 3.12 (dd, J=13.9, 10.4 Hz, 1H). UPLC-MS (basic 4 min) Rt=2.38 min. m/z=564.3 for [M]⁺

### Step 4: 3-[2-(1,3-Benzothiazol-2-yl)-2-{2′,4′-dichloro-[1,1′-biphenyl]-3-sulfonamido}ethyl]-N′-hydroxybenzene-1-carboximidamide

To a magnetically stirred solution of N-[1-(1,3-benzothiazol-2-yl)-2-(3-cyanophenyl)ethyl]-2′,4′-dichloro-[1,1′-biphenyl]-3-sulfonamide) (0.265 g, 0.469 mmol, 1.0 eq.) in EtOH (3.1 mL) were added hydroxyl amine hydrochloride (0.065 g, 0.939 mmol, 2.0 eq.) and DIPEA (0.245 mL, 1.41 mmol, 3.0 eq.). The reaction mixture was stirred under reflux for 1 h. The reaction mixture was cooled down to RT and then concentrated to dryness to afford product as a yellow oil (0.280 g, 0.371 mmol, 79% yield) which was used in the next step without further purification. UPLC-MS (basic 4 min) Rt=2.11 min. m/z=597.3 for [M]⁺

### Step 5: Amino({3-[2-(1,3-benzothiazol-2-yl)-2-{2′,4′-dichloro-[1,1′-biphenyl]-3-sulfonamido}ethyl]phenyl})methylidene]amino acetate

To a magnetically stirred solution of 3-[2-(1,3-benzothiazol-2-yl)-2-{2′,4′-dichloro-[1,1′-biphenyl]-3-sulfonamido}ethyl]-N′-hydroxybenzene-1-carboximidamide (0.280 g, 0.469 mmol, 1.0 eq) in acetic acid (3.2 mL) was added acetic anhydride (0.046 mL, 0.492 mmol, 1.05 eq.) dropwise at 0° C. The resulting mixture was stirred at RT for 40 mins. and then concentrated to dryness. The residue was purified by Reverse Phase column chromatography on a 120 g C18 cartridge eluting with a 5-95% H₂O:MeCN eluent (0.1% ammonia) to afford product as a white solid (0.185 g, 0.249 mmol, 53% yield). ¹H NMR (400 MHz, DMSO-d₆): δ 81 (s, 2H), 5.83 (s, 2H), 5.12 (dd, J=9.7, 5.2 Hz, 1H), 3.42-3.34 (m, 1H), 3.11 (dd, J=13.9, 9.7 Hz, 1H), 2.21 (s, 3H). UPLC-MS (basic 4 min): Rt=2.19 min; m/z=639.0 for [M]⁺

### Step 6: 3-[2-(1,3-Benzothiazol-2-yl)-2-{2′,4′-dichloro-[1,1′-biphenyl]-3-sulfonamido}ethyl]benzene-1-carboximidamide)

To a magnetically stirred solution of amino({3-[2-(1,3-benzothiazol-2-yl)-2-{2′,4′-dichloro-[1,1′-biphenyl]-3-sulfonamido}ethyl]phenyl})methylidene]amino acetate (0.139 g, 0.217 mmol, 1.0 eq) in acetic acid (1.5 mL) was added zinc (0.142 g, 2.17 mmol, 10.0 eq.) and the reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford product as a white solid (0.056 g, 0.093 mmol, 43% yield). ¹H NMR (400 MHz, DMSO-d₆): δ 8.01 (d, J=8.0 Hz, 1H), 7.89 (d, J=8.0 Hz, 1H), 7.77 (dd, J=6.6, 2.1 Hz, 2H), 7.60-7.53 (m, 3H), 7.49 (ddd, J=8.2, 6.4, 1.7 Hz, 2H), 7.45-7.34 (m, 3H), 7.35 (d, J=1.6 Hz, 1H), 7.26 (t, J=7.7 Hz, 1H), 7.18 (d, J=8.3 Hz, 1H), 4.94 (dd, J=8.5, 4.8 Hz, 1H), 3.29 (dd, J=13.4, 4.8 Hz, 1H), 3.10 (dd, J=13.4, 8.6 Hz, 1H). UPLC-MS (basic 6 min): Rt=3.63 min; m/z=580.9 for [M]⁺, 96% purity. Chiral analysis performed by Reach Separations showed 64.3% chiral purity.

Product purified by SFC using Lux C₄ (21.2 mm×250 mm, Sum) with a 60:40 MeOH:CO₂ (0.2% v/v NH₃) eluent to afford product as a white solid (0.003 g).

¹H NMR (400 MHz, DMSO-d₆): δ 8.01 (d, J=8.0 Hz, 1H), 7.89 (d, J=8.0 Hz, 1H), 7.77 (dd, J=6.6, 2.1 Hz, 2H), 7.60-7.53 (m, 3H), 7.49 (ddd, J=8.2, 6.4, 1.7 Hz, 2H), 7.45-7.34 (m, 3H), 7.35 (d, J=1.6 Hz, 1H), 7.26 (t, J=7.7 Hz, 1H), 7.18 (d, J=8.3 Hz, 1H), 4.94 (dd, J=8.5, 4.8 Hz, 1H), 3.29 (dd, J=13.4, 4.8 Hz, 1H), 3.10 (dd, J=13.4, 8.6 Hz, 1H). UPLC-MS (acidic 4 min): Rt=1.45 min; m/z=581.1 for [M+H]⁺, 100% purity. Chiral analysis performed by Reach Separations showed 100% chiral purity. Rt=3.92 min.

### Example 16. Synthesis of Compound 4

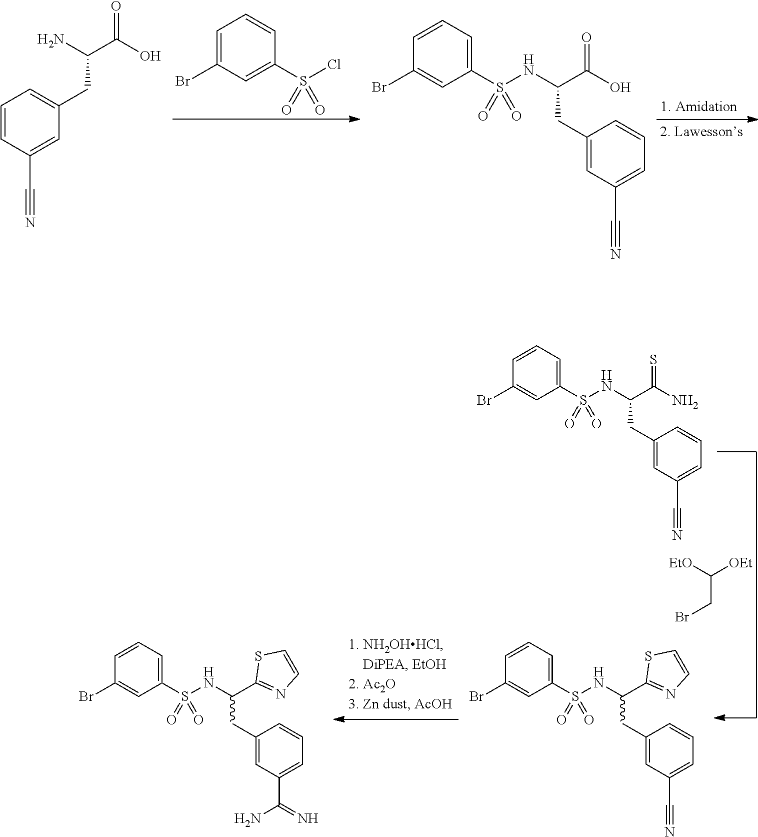

### Step 1: (2S)-2-(3-Bromobenzenesulfonamido)-3-(3-cyanophenyl)propanoic acid)

To a magnetically stirred solution of sodium bicarbonate (3.34 g, 31.5 mmol, 1.4 eq.) in water (28 mL) was added 3-bromobenzene-1-sulfonyl chloride (5.85 g, 22.9 mmol, 1.0 eq.) and the resulting solution was cooled to −5° C. (2S)-2-amino-3-(3-cyanophenyl)propanoic acid (5.00 g, 26.3 mmol, 1.1 eq.) was added in 4 portions over a period of 40 mins. The resulting slurry was allowed to warm to RT and stirred for 2 h. The reaction mixture was acidified with 20% HCl and then filtered under vacuum. The solid was washed with water (2×50 mL) and then dried to afford product as a white solid (7.00 g, 17.1 mmol, 75% yield). ¹H NMR (DMSO-d6) δ: 8.51 (d, J=9.1 Hz, 1H), 7.75-7.70 (m, 1H), 7.65-7.43 (m, 5H), 7.41-7.29 (m, 2H), 4.04 (ddd, J=10.3, 9.1, 4.5 Hz, 1H), 3.07 (dd, J=13.8, 4.5 Hz, 1H), 2.76 (dd, J=13.8, 10.4 Hz, 1H). UPLC-MS (basic 2 min) Rt=0.80 min. m/z=410.0 for [M+H]⁺

### Step 2: (2S)-2-(3-Bromobenzenesulfonamido)-3-(3-cyanophenyl) propenamide

To a magnetically stirred solution of (2S)-2-(3-bromobenzenesulfonamido)-3-(3-cyanophenyl)propanoic acid) (0.799 g, 1.95 mmol, 1.0 eq.) in DCM (20.0 mL) were added DIPEA (0.80 mL, 4.59 mmol, 2.4 eq.) and BOP (0.986 g, 2.23 mmol, 1.14 eq.). The mixture was stirred at RT for 15 minutes before adding conc. ammonia (0.60 mL, 10.8 mmol, 5.5 eq.). The reaction mixture was stirred at RT for 24 h before concentrating to dryness. The residue was purified by Reverse Phase column chromatography eluting with a 5-95% H₂O:MeCN eluent (0.1% ammonia) to afford product as a white solid (0.366 g, 0.0.896 mmol, 46% yield). ¹H NMR (DMSO-d6) δ: 7.71 (ddd, J=8.0, 1.9, 1.0 Hz, 1H), 7.40-7.63 (m, 7H), 7.34-7.40 (m, 2H), 7.09 (s, 1H), 3.97 (dd, J=10.3, 4.3 Hz, 1H), 2.91 (dd, J=13.7, 4.3 Hz, 1H), 2.63-2.73 (m, 1H). UPLC-MS (acidic 2 min) Rt=0.95 min. m/z=406.0 for [M]⁺

### Step 3: (2S)-2-(3-Bromobenzenesulfonamido)-3-(3-cyanophenyl)propanethioamide)

To a magnetically stirred solution of (2S)-2-(3-bromobenzenesulfonamido)-3-(3-cyanophenyl) propenamide (0.623 g, 1.53 mmol, 1.0 eq.) in MeCN (15.0 mL) was added Lawesson's Reagent (1.01 g, 2.50 mmol, 1.6 eq.) and the reaction mixture was stirred at RT for 24 h. The reaction mixture was diluted with DCM (25 mL) and washed with aq. saturated NaHCO₃ solution (25 mL). The organic layer was dried over anhydrous sodium sulfate before filtering and concentrating to dryness. The residue was purified by Reverse Phase column chromatography eluting with a 5-95% H₂O:MeCN eluent (0.1% formic acid) to afford product as a yellow solid (0.163 g, 0.384 mmol, 25% yield). ¹H NMR (400 MHz, DMSO-d₆): δ 9.63 (br s, 1H), 9.30 (br s, 1H), 7.79-8.52 (m, 1H), 7.70 (ddd, J=8.0, 1.9, 1.0 Hz, 1H), 7.54-7.61 (m, 3H), 7.50-7.54 (m, 1H), 7.48 (d, J=7.9 Hz, 1H), 7.28-7.42 (m, 2H), 4.28 (dd, J=9.8, 4.5 Hz, 1H), 2.92 (dd, J=13.7, 4.5 Hz, 1H), 2.78 (dd, J=13.6, 9.9 Hz, 1H). UPLC-MS (acidic 2 min): Rt=1.05 min; m/z=422.0 for [M]⁺

### Step 4: 3-Bromo-N-[(1S)-2-(3-cyanophenyl)-1-(1,3-thiazol-2-yl)ethyl]benzene-1-sulfonamide

To a magnetically stirred solution of (2S)-2-(3-bromobenzenesulfonamido)-3-(3-cyanophenyl) propanethioamide) (0.290 g, 0.683 mmol, 1.0 eq.) in THE (6.0 mL) were added 2-bromo-1,1-dimethoxyethane (0.250 mL, 2.12 mmol, 3.0 eq.) and 4N HCl in 1,4-Dioxane (0.700 mL, 2.80 mmol, 4.0 eq.). The reaction mixture was stirred under reflux for 1 h. The reaction mixture was cooled down to RT and then concentrated to dryness. The residue was purified by Reverse Phase column chromatography eluting with a 5-95% H₂O:MeCN eluent (0.1% ammonia) to afford product as a white solid (0.152 g, 0.339 mmol, 50% yield). ¹H NMR (400 MHz, DMSO-d₆): δ 8.98 (br s, 1H), 7.71 (d, J=3.3 Hz, 1H), 7.64-7.68 (m, 1H), 7.62-7.64 (m, 1H), 7.56-7.59 (m, 1H), 7.44-7.54 (m, 4H), 7.27-7.34 (m, 2H), 4.91-5.05 (m, 1H), 3.27-3.33 (m, 1H), 2.93 (dd, J=13.9, 10.6 Hz, 1H). UPLC-MS (acidic 2 min): Rt=1.11 min; m/z=477.8 for [M]⁺

### Step 5: 3-[(2S)-2-(3-Bromobenzenesulfonamido)-2-(1,3-thiazol-2-yl)ethyl]-N′-hydroxybenzene-1-carboximidamide

To a magnetically stirred solution of 3-bromo-N-[(1S)-2-(3-cyanophenyl)-1-(1,3-thiazol-2-yl)ethyl]benzene-1-sulfonamide) (0.050 g, 0.112 mmol, 1.0 eq.) in EtOH (1.0 mL) were added hydroxylamine hydrochloride (0.015 g, 0.223 mmol, 2.0 eq.) and DIPEA (0.058 mL, 0.335 mmol, 3.0 eq.). The reaction mixture was stirred under reflux for 1 h. The reaction mixture was cooled down to RT and then concentrated to dryness to afford product as a yellow oil (0.054 g, 0.112 mmol, 100% yield) which was used in the next step without further purification. UPLC-MS (basic 2 min) Rt=0.95 min. m/z=480.8 for [M]⁻

### Step 6: Amino({3-[(2S)-2-(3-bromobenzenesulfonamido)-2-(1,3-thiazol-2-yl)ethyl]phenyl})methylidene] amino acetate

To a magnetically stirred solution of 3-[(2S)-2-(3-bromobenzenesulfonamido)-2-(1,3-thiazol-2-yl)ethyl]-N′-hydroxybenzene-1-carboximidamide) (0.054 g, 0.112 mmol, 1.0 eq) in acetic acid (0.5 mL) was added acetic anhydride (0.032 mL, 0.337 mmol, 3.0 eq.). The resulting mixture was stirred at RT for 30 mins. and then concentrated to dryness. The residue was used in the next step without further purification. UPLC-MS (basic 2 min): Rt=0.99 min; m/z=522.8 for [M]⁺

### Step 7: 3-[2-(3-Bromobenzenesulfonamido)-2-(1,3-thiazol-2-yl)ethyl]benzene-1-carboximidamide)

To a magnetically stirred solution of amino({3-[(2S)-2-(3-bromobenzenesulfonamido)-2-(1,3-thiazol-2-yl)ethyl]phenyl})methylidene]amino acetate) (0.024 g, 0.020 mmol, 1.0 eq) in acetic acid (1.5 mL) was added zinc (0.013 g, 0.202 mmol, 10.0 eq.) and the reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford product as a white solid (0.004 g, 0.009 mmol, 43% yield). ¹H NMR (400 MHz, DMSO-d₆): δ 8.18 (s, 2H), 7.63-7.39 (m, 8H), 7.33-7.11 (m, 4H), 4.78-4.67 (m, 1H), 3.16-3.11 (m, 1H), 2.98-2.88 (m, 1H). UPLC-MS (basic 2 min): Rt=1.08 min; m/z=466.9 for [M+H]⁺, 98% purity. Chiral analysis performed by Reach Separations showed 50% chiral purity.

### Example 17. Synthesis of Compound 5

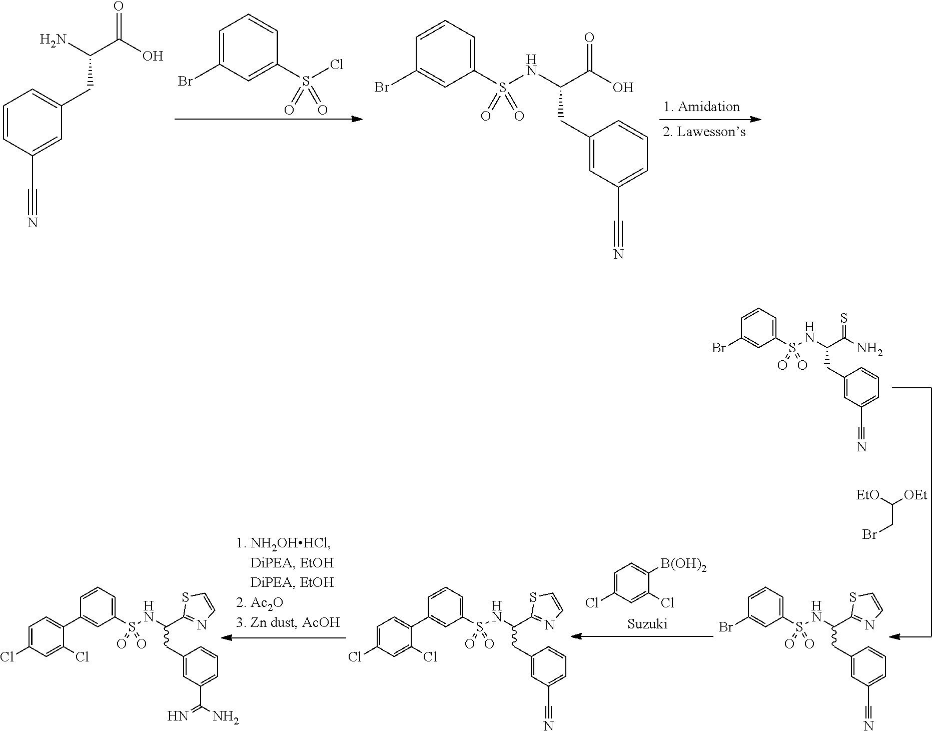

### Step 1: (2S)-2-(3-Bromobenzenesulfonamido)-3-(3-cyanophenyl)propanoic acid)

To a magnetically stirred solution of sodium bicarbonate (3.34 g, 31.5 mmol, 1.4 eq.) in water (28 mL) was added 3-bromobenzene-1-sulfonyl chloride (5.85 g, 22.9 mmol, 1.0 eq.) and the resulting solution was cooled to −5° C. (2S)-2-amino-3-(3-cyanophenyl)propanoic acid (5.00 g, 26.3 mmol, 1.1 eq.) was added in 4 portions over a period of 40 mins. The resulting slurry was allowed to warm to RT and stirred for 2 h. The reaction mixture was acidified with 20% HCl and then filtered under vacuum. The solid was washed with water (2×50 mL) and then dried to afford product as a white solid (7.00 g, 17.1 mmol, 75% yield). ¹H NMR (DMSO-d6) δ: 8.51 (d, J=9.1 Hz, 1H), 7.75-7.70 (m, 1H), 7.65-7.43 (m, 5H), 7.41-7.29 (m, 2H), 4.04 (ddd, J=10.3, 9.1, 4.5 Hz, 1H), 3.07 (dd, J=13.8, 4.5 Hz, 1H), 2.76 (dd, J=13.8, 10.4 Hz, 1H). UPLC-MS (basic 2 min) Rt=0.80 min. m/z=410.0 for [M+H]⁺

### Step 2: (2S)-2-(3-Bromobenzenesulfonamido)-3-(3-cyanophenyl) propenamide

To a magnetically stirred solution of (2S)-2-(3-bromobenzenesulfonamido)-3-(3-cyanophenyl)propanoic acid) (0.799 g, 1.95 mmol, 1.0 eq.) in DCM (20.0 mL) were added DIPEA (0.80 mL, 4.59 mmol, 2.4 eq.) and BOP (0.986 g, 2.23 mmol, 1.14 eq.). The mixture was stirred at RT for 15 minutes before adding conc. ammonia (0.60 mL, 10.8 mmol, 5.5 eq.). The reaction mixture was stirred at RT for 24 h before concentrating to dryness. The residue was purified by Reverse Phase column chromatography eluting with a 5-95% H₂O:MeCN eluent (0.1% ammonia) to afford product as a white solid (0.366 g, 0.0.896 mmol, 46% yield). ¹H NMR (DMSO-d6) δ: 7.71 (ddd, J=8.0, 1.9, 1.0 Hz, 1H), 7.40-7.63 (m, 7H), 7.34-7.40 (m, 2H), 7.09 (s, 1H), 3.97 (dd, J=10.3, 4.3 Hz, 1H), 2.91 (dd, J=13.7, 4.3 Hz, 1H), 2.63-2.73 (m, 1H). UPLC-MS (acidic 2 min) Rt=0.95 min. m/z=406.0 for [M]⁺

### Step 3: (2S)-2-(3-Bromobenzenesulfonamido)-3-(3-cyanophenyl)propanethioamide)

To a magnetically stirred solution of (2S)-2-(3-bromobenzenesulfonamido)-3-(3-cyanophenyl) propenamide (0.623 g, 1.53 mmol, 1.0 eq.) in MeCN (15.0 mL) was added Lawesson's Reagent (1.01 g, 2.50 mmol, 1.6 eq.) and the reaction mixture was stirred at RT for 24 h. The reaction mixture was diluted with DCM (25 mL) and washed with aq. saturated NaHCO₃ solution (25 mL). The organic layer was dried over anhydrous sodium sulfate before filtering and concentrating to dryness. The residue was purified by Reverse Phase column chromatography eluting with a 5-95% H₂O:MeCN eluent (0.1% formic acid) to afford product as a yellow solid (0.163 g, 0.384 mmol, 25% yield). ¹H NMR (400 MHz, DMSO-d₆): δ 9.63 (br s, 1H), 9.30 (br s, 1H), 7.79-8.52 (m, 1H), 7.70 (ddd, J=8.0, 1.9, 1.0 Hz, 1H), 7.54-7.61 (m, 3H), 7.50-7.54 (m, 1H), 7.48 (d, J=7.9 Hz, 1H), 7.28-7.42 (m, 2H), 4.28 (dd, J=9.8, 4.5 Hz, 1H), 2.92 (dd, J=13.7, 4.5 Hz, 1H), 2.78 (dd, J=13.6, 9.9 Hz, 1H). UPLC-MS (acidic 2 min): Rt=1.05 min; m/z=422.0 for [M]⁺

### Step 4: 3-Bromo-N-[2-(3-cyanophenyl)-1-(1,3-thiazol-2-yl)ethyl]benzene-1-sulfonamide

To a magnetically stirred solution of (2S)-2-(3-bromobenzenesulfonamido)-3-(3-cyanophenyl) propanethioamide) (0.290 g, 0.683 mmol, 1.0 eq.) in THE (6.0 mL) were added 2-bromo-1,1-dimethoxyethane (0.250 mL, 2.12 mmol, 3.0 eq.) and 4N HCl in 1,4-Dioxane (0.700 mL, 2.80 mmol, 4.0 eq.). The reaction mixture was stirred under reflux for 1 h. The reaction mixture was cooled down to RT and then concentrated to dryness. The residue was purified by Reverse Phase column chromatography eluting with a 5-95% H₂O:MeCN eluent (0.1% ammonia) to afford product as a white solid (0.152 g, 0.339 mmol, 50% yield). ¹H NMR (400 MHz, DMSO-d₆): δ 8.98 (br s, 1H), 7.71 (d, J=3.3 Hz, 1H), 7.64-7.68 (m, 1H), 7.62-7.64 (m, 1H), 7.56-7.59 (m, 1H), 7.44-7.54 (m, 4H), 7.27-7.34 (m, 2H), 4.91-5.05 (m, 1H), 3.27-3.33 (m, 1H), 2.93 (dd, J=13.9, 10.6 Hz, 1H). UPLC-MS (acidic 2 min): Rt=1.11 min; m/z=477.8 for [M]⁺

### Step 5: 2′,4′-Dichloro-N-[2-(3-cyanophenyl)-1-(1,3-thiazol-2-yl)ethyl]-[1,1′-biphenyl]-3-sulfonamide

To a magnetically stirred solution of 3-bromo-N-[2-(3-cyanophenyl)-1-(1,3-thiazol-2-yl)ethyl]benzene-1-sulfonamide (0.054 g, 0.120 mmol, 1.0 eq.) in toluene (2.0 mL), EtOH (2.0 mL) and water (0.1 mL) were added (2,4-dichlorophenyl)boronic acid (0.051 g, 0.267 mmol, 2.2 eq.), potassium carbonate (0.070 g, 0.506 mmol, 4.2 eq.) and tetrakis(triphenylphosphane) palladium (0.016 g, 0.014 mmol, 0.1 eq.). The reaction mixture was stirred under reflux for 24 h. The reaction mixture was cooled down to RT and then concentrated to dryness. The residue was purified by Reverse Phase column chromatography eluting with a 5-95% H₂O:MeCN eluent (0.1% ammonia) to afford product as a white solid (0.045 g, 0.087 mmol, 73% yield). UPLC-MS (acidic 2 min): Rt=1.28 min; m/z=513.8 for [M]⁺

### Step 6: 3-[2-{2′,4′-Dichloro-[1,1′-biphenyl]-3-sulfonamido}-2-(1,3-thiazol-2-yl)ethyl]-N′-hydroxybenzene-1-carboximidamide

To a magnetically stirred solution of 2′,4′-dichloro-N-[2-(3-cyanophenyl)-1-(1,3-thiazol-2-yl)ethyl]-[1,1′-biphenyl]-3-sulfonamide (0.045 g, 0.087 mmol, 1.0 eq.) in EtOH (1.0 mL) were added hydroxylamine hydrochloride (0.012 g, 0.175 mmol, 2.0 eq.) and DIPEA (0.046 mL, 0.262 mmol, 3.0 eq.). The reaction mixture was stirred under reflux for 1 h. The reaction mixture was cooled down to RT and then concentrated to dryness to afford product as a yellow oil (0.048 g, 0.088 mmol, 100% yield) which was used in the next step without further purification. UPLC-MS (basic 2 min) Rt=1.14 min. m/z=546.8 for [M]⁺

### Step 7: Amino({3-[2-{2′,4′-dichloro-[1,1′-biphenyl]-3-sulfonamido}-2-(1,3-thiazol-2-yl)ethyl]phenyl})methylidene]amino acetate

To a magnetically stirred solution of 3-[2-{2′,4′-dichloro-[1,1′-biphenyl]-3-sulfonamido}-2-(1,3-thiazol-2-yl)ethyl]-N′-hydroxybenzene-1-carboximidamide (0.049 g, 0.090 mmol, 1.0 eq) in acetic acid (0.5 mL) was added acetic anhydride (0.025 mL, 0.269 mmol, 3.0 eq.). The resulting mixture was stirred at RT for 30 mins. and then concentrated to dryness. The residue was used in the next step without further purification. UPLC-MS (basic 2 min): Rt=1.18 min; m/z=588.9 for [M]⁺

### Step 8: 3-[2-{2′,4′-Dichloro-[1,1′-biphenyl]-3-sulfonamido}-2-(1,3-thiazol-2-yl)ethyl]benzene-1-carboximidamide

To a magnetically stirred solution of amino({3-[2-{2′,4′-dichloro-[1,1′-biphenyl]-3-sulfonamido}-2-(1,3-thiazol-2-yl)ethyl]phenyl})methylidene]amino acetate (0.014 g, 0.018 mmol, 1.0 eq) in acetic acid (1.0 mL) was added zinc (0.012 g, 0.180 mmol, 10.0 eq.) and the reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford compound 5 (Isomer 1, batch 1) as a white solid (0.005 g, 0.010 mmol, 56% yield). ¹H NMR (400 MHz, DMSO-d₆): δ 7.75 (d, J=2.1 Hz, 1H), 7.61-7.32 (m, 11H), 7.18 (dt, J=15.1, 7.6 Hz, 2H), 4.88-4.80 (m, 1H), 3.18-3.15 (m, 1H), 2.99-2.93 (m, 1H). UPLC-MS (basic 6 min): Rt=3.22 min; m/z=530.9 for [M+H]⁺, 98% purity. Chiral analysis performed by Reach Separations showed 50.5% chiral purity.

Compound 5 (Isomer 2) was isolated by preparative chiral HPLC of compound 5 (Isomer 1, batch 2), 96% pure by UPLC-MS, and 50.3% pure by chiral HPLC]. Product purified by SFC using Lux C₂ (20 mm×250 mm, Sum) with a 50:50 MeOH:CO₂ (0.2% v/v NH₃) eluent to afford product as a white solid (0.036 g). Compound 5 (Isomer 2) was isolated in 98% purity by UPLC-MS. Chiral analysis performed by Reach Separations showed 100% chiral purity. Rt=3.678 minutes.

Compound 5 (Isomer 3) was isolated by preparative chiral HPLC of compound 5 (Isomer 1, batch 2), 96% pure by UPLC-MS, and 50.3% pure by chiral HPLC]. Product purified by SFC using Lux C2 (20 mm×250 mm, Sum) with a 50:50 MeOH:CO₂ (0.2% v/v NH₃) eluent to afford product as a white solid (0.036 g). Compound 5 (Isomer 3) was isolated in 99% purity by UPLC-MS. Chiral analysis performed by Reach Separations showed 100% chiral purity. Rt=5.385 minutes.

### Example 18. Synthesis of Compound 6

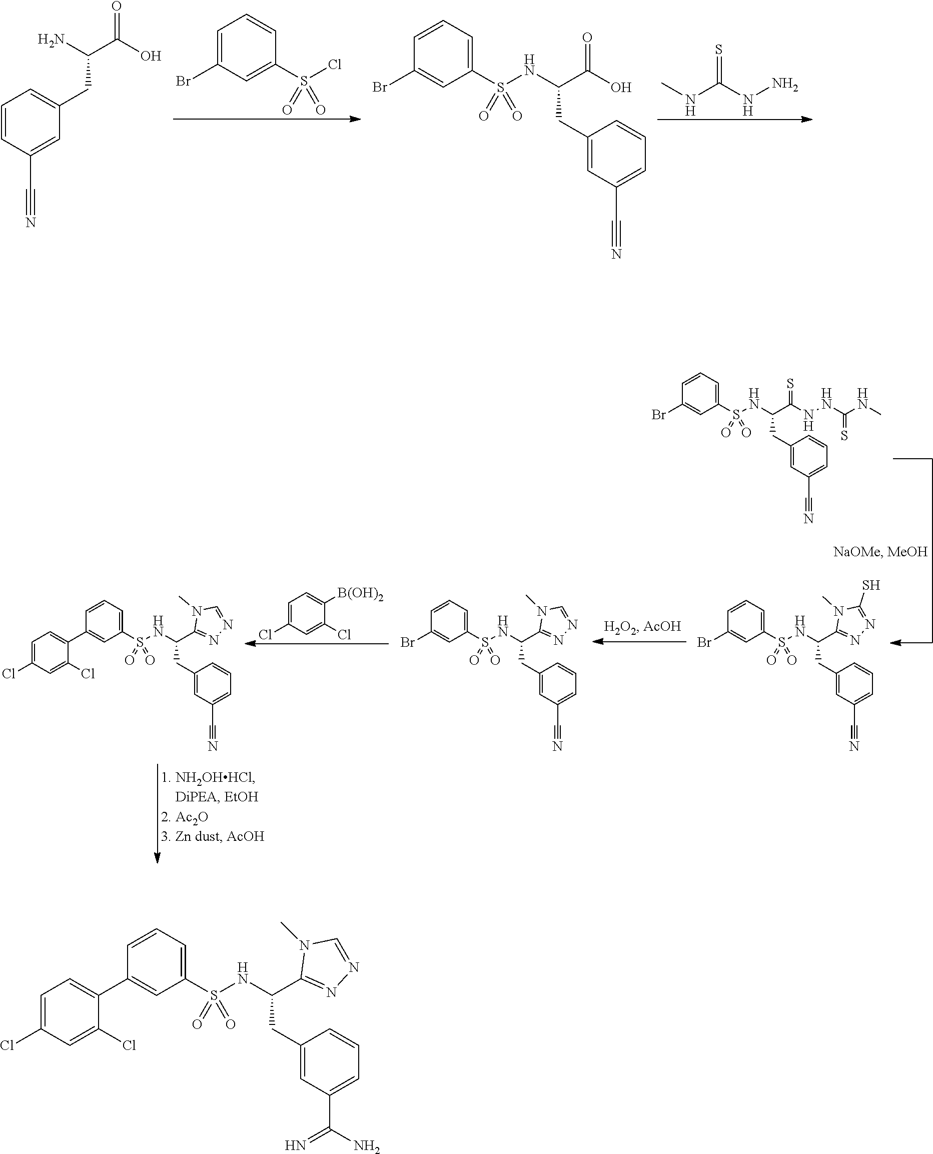

### Step 1: (2S)-2-(3-Bromobenzenesulfonamido)-3-(3-cyanophenyl)propanoic acid)

To a magnetically stirred solution of sodium bicarbonate (3.34 g, 31.5 mmol, 1.4 eq.) in water (28 mL) was added 3-bromobenzene-1-sulfonyl chloride (5.85 g, 22.9 mmol, 1.0 eq.) and the resulting solution was cooled to −5° C. (2S)-2-amino-3-(3-cyanophenyl)propanoic acid (5.00 g, 26.3 mmol, 1.1 eq.) was added in 4 portions over a period of 40 mins. The resulting slurry was allowed to warm to RT and stirred for 2 h. The reaction mixture was acidified with 20% HCl and then filtered under vacuum. The solid was washed with water (2×50 mL) and then dried to afford product as a white solid (7.00 g, 17.1 mmol, 75% yield). ¹H NMR (DMSO-d6) δ: 8.51 (d, J=9.1 Hz, 1H), 7.75-7.70 (m, 1H), 7.65-7.43 (m, 5H), 7.41-7.29 (m, 2H), 4.04 (ddd, J=10.3, 9.1, 4.5 Hz, 1H), 3.07 (dd, J=13.8, 4.5 Hz, 1H), 2.76 (dd, J=13.8, 10.4 Hz, 1H). UPLC-MS (basic 2 min) Rt=0.80 min. m/z=410.0 for [M+H]⁺

### Step 2: (2S)-2-(3-Bromobenzenesulfonamido)-3-(3-cyanophenyl)-N-[(methylcarbamothioyl)amino]propanamide)

To a magnetically stirred solution of (2S)-2-(3-bromobenzenesulfonamido)-3-(3-cyanophenyl)propanoic acid (1.00 g, 2.44 mmol, 1.0 eq.) in pyridine (20.0 mL) were added EDCI·HCl (0.620 g, 3.23 mmol, 1.3 eq.). The mixture was stirred at RT for 15 minutes before adding 3-amino-1-methylthiourea (0.60 mL, 10.8 mmol, 5.5 eq.). The reaction mixture was stirred at RT for 24 h before concentrating to dryness. The residue was purified by Reverse Phase column chromatography eluting with a 5-95% H₂O:MeCN eluent (0.1% ammonia) to afford product as a pale yellow solid (0.812 g, 1.636 mmol, 67% yield). ¹H NMR (DMSO-d6) δ: 10.13 (br s, 1H), 9.40 (s, 1H), 8.40-8.69 (m, 1H), 7.64-7.77 (m, 1H), 7.53-7.63 (m, 2H), 7.46-7.53 (m, 3H), 7.38-7.45 (m, 1H), 7.35 (t, J=7.8 Hz, 2H), 4.01 (br s, 1H), 3.06-3.16 (m, 1H), 2.86-2.96 (m, 3H), 2.70 (dd, J=14.0, 10.6 Hz, 1H). UPLC-MS (acidic 2 min) Rt=0.95 min. m/z=498.0 for [M]⁺

### Step 3: 3-Bromo-N-[(1S)-2-(3-cyanophenyl)-1-(4-methyl-5-sulfanyl-4H-1,2,4-triazol-3-yl)ethyl]benzene-1-sulfonamide

To a magnetically stirred solution of (2S)-2-(3-bromobenzenesulfonamido)-3-(3-cyanophenyl)-N-[(methylcarbamothioyl)amino]propenamide (0.501 g, 1.01 mmol, 1.0 eq.) in MeOH (10.0 mL) were added sodium methoxide (1.2 mL, 5.4 M in MeOH, 6.48 mmol, 6.4 eq.) and the reaction mixture was stirred at RT for 24 h. The reaction mixture was concentrated to dryness and the residue was purified by Reverse Phase column chromatography eluting with a 5-95% H₂O:MeCN eluent (0.1% formic acid) to afford product as a white solid (0.232 g, 0.485 mmol, 48% yield). ¹H NMR (400 MHz, DMSO-d₆): δ 7.69-7.75 (m, 1H), 7.65 (s, 1H), 7.60 (dt, J=7.7, 1.3 Hz, 1H), 7.57 (t, J=1.8 Hz, 1H), 7.48-7.55 (m, 2H), 7.38 (td, J=7.8, 4.5 Hz, 2H), 4.78 (dd, J=9.6, 5.7 Hz, 1H), 3.40 (s, 3H), 3.17 (dd, J=14.1, 5.6 Hz, 1H), 3.02 (dd, J=13.9, 9.7 Hz, 1H). UPLC-MS (acidic 2 min): Rt=1.03 min; m/z=477.9 for [M]⁺

### Step 4: 3-Bromo-N-[(1S)-2-(3-cyanophenyl)-1-(4-methyl-4H-1,2,4-triazol-3-yl)ethyl]benzene-1-sulfonamide

To a magnetically stirred solution of 3-bromo-N-[(1S)-2-(3-cyanophenyl)-1-(4-methyl-5-sulfanyl-4H-1,2,4-triazol-3-yl)ethyl]benzene-1-sulfonamide (0.232 g, 0.485 mmol, 1.0 eq.) in DCM (3.0 mL) were added hydrogen peroxide (1.0 mL, 12.9 mmol, 27.0 eq.) and acetic acid (2.0 mL, 34.8 mmol, 72.0 eq.). The reaction mixture was stirred at room temperature for 18 h and then concentrated to dryness. The residue was purified by Reverse Phase column chromatography eluting with a 5-95% H₂O:MeCN eluent (0.1% ammonia) to afford product as a white solid (0.069 g, 0.155 mmol, 32% yield). ¹H NMR (400 MHz, DMSO-d₆): δ 8.86 (br s, 1H), 8.27 (s, 1H), 7.71 (ddd, J=8.0, 1.9, 1.0 Hz, 1H), 7.63 (d, J=1.4 Hz, 1H), 7.58 (dt, J=7.7, 1.3 Hz, 1H), 7.53-7.56 (m, 1H), 7.51-7.53 (m, 1H), 7.50 (q, J=1.7 Hz, 1H), 7.37 (t, J=7.9 Hz, 2H), 4.84 (br s, 1H), 3.55 (s, 3H), 3.02-3.24 (m, 2H). UPLC-MS (acidic 2 min): Rt=0.96 min; m/z=445.8 for [M]⁺

### Step 5: 2′,4′-Dichloro-N-[(1S)-2-(3-cyanophenyl)-1-(1,3-thiazol-2-yl)ethyl]-[1,1′-biphenyl]-3-sulfonamide

To a magnetically stirred solution of 3-bromo-N-[(1S)-2-(3-cyanophenyl)-1-(4-methyl-4H-1,2,4-triazol-3-yl)ethyl]benzene-1-sulfonamide (0.069 g, 0.155 mmol, 1.0 eq.) in toluene (1.0 mL), EtOH (1.0 mL) and water (0.1 mL) were added (2,4-dichlorophenyl)boronic acid (0.059 g, 0.309 mmol, 2.0 eq.), potassium carbonate (0.064 g, 0.464 mmol, 3.0 eq.) and tetrakis(triphenylphosphane) palladium (0.002 g, 0.002 mmol, 0.01 eq.). The reaction mixture was stirred under reflux for 24 h. The reaction mixture was cooled down to RT and then concentrated to dryness. The residue was purified by Reverse Phase column chromatography eluting with a 5-95% H₂O:MeCN eluent (0.1% ammonia) to afford product as a white solid (0.050 g, 0.098 mmol, 63% yield). UPLC-MS (basic 2 min): Rt=1.12 min; m/z=511.9 for [M]+¹H NMR (400 MHz, DMSO-d₆): δ 8.20 (s, 1H), 7.79 (d, J=2.1 Hz, 1H), 7.68-7.53 (m, 35H), 7.50 (d, J=8.6 Hz, 2H), 7.47 (d, J=8.3 Hz, 2H), 7.37 (t, J=7.7 Hz, 2H), 4.37 (t, J=5.0 Hz, 1H), 3.46 (s, 3H), 3.21 (dd, J=14.4, 6.9 Hz, 2H), 3.14-3.07 (m, 2H).

### Step 6: 3-[(2S)-2-{2′,4′-Dichloro-[1,1′-biphenyl]-3-sulfonamido}-2-(4-methyl-4H-1,2,4-triazol-3-yl)ethyl]-N′-hydroxybenzene-1-carboximidamide

To a magnetically stirred solution of 2′,4′-dichloro-N-[(1S)-2-(3-cyanophenyl)-1-(4-methyl-4H-1,2,4-triazol-3-yl)ethyl]-[1,1′-biphenyl]-3-sulfonamide (0.050 g, 0.098 mmol, 1.0 eq.) in EtOH (1.0 mL) were added hydroxylamine hydrochloride (0.007 g, 0.103 mmol, 1.05 eq.) and DIPEA (0.051 mL, 0.293 mmol, 3.0 eq.). The reaction mixture was stirred under reflux for 1 h. The reaction mixture was cooled down to RT and then concentrated to dryness to afford product as a yellow oil (0.053 g, 0.097 mmol, 100% yield) which was used in the next step without further purification. UPLC-MS (basic 2 min) Rt=1.02 min. m/z=545.0 for [M]⁺

### Step 7: Amino({3-[(2S)-2-{2′,4′-dichloro-[1,1′-biphenyl]-3-sulfonamido}-2-(4-methyl-4H-1,2,4-triazol-3-yl)ethyl]phenyl})methylidene]amino acetate

To a magnetically stirred solution of 3-[(2S)-2-{2′,4′-dichloro-[1,1′-biphenyl]-3-sulfonamido}-2-(4-methyl-4H-1,2,4-triazol-3-yl)ethyl]-N′-hydroxybenzene-1-carboximidamide (0.053 g, 0.097 mmol, 1.0 eq) in acetic acid (1.0 mL) was added acetic anhydride (0.028 mL, 0.291 mmol, 3.0 eq.). The resulting mixture was stirred at RT for 30 mins. and then concentrated to dryness. The residue was used in the next step without further purification. UPLC-MS (basic 4 min): Rt=1.70 min; m/z=587.0 for [M]⁺

### Step 8: 3-[(2S)-2-{2′,4′-Dichloro-[1,1′-biphenyl]-3-sulfonamido}-2-(4-methyl-4H-1,2,4-triazol-3-yl)ethyl]benzene-1-carboximidamide

To a magnetically stirred solution of amino({3-[(2S)-2-{2′,4′-dichloro-[1,1′-biphenyl]-3-sulfonamido}-2-(4-methyl-4H-1,2,4-triazol-3-yl)ethyl]phenyl})methylidene]amino acetate (0.057 g, 0.097 mmol, 1.0 eq) in acetic acid (1.0 mL) was added zinc (0.063 g, 0.970 mmol, 10.0 eq.) and the reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford product as a white solid (0.006 g, 0.011 mmol, 12% yield). ¹H NMR (400 MHz, DMSO-d₆): δ 8.12 (s, 1H), 7.78 (d, J=2.1 Hz, 1H), 7.64-7.49 (m, 8H), 7.46 (d, J=8.3 Hz, 1H), 7.30-7.23 (m, 2H), 4.79 (t, J=7.6 Hz, 1H), 3.38 (s, 3H), 3.20 (dd, 1H), 3.07 (dd, J=13.5, 7.3 Hz, 1H). UPLC-MS (basic 4 min): Rt=1.22 min; m/z=529.1 for [M]+, 95% purity. Chiral analysis performed by Reach Separations showed 100% chiral purity.

### Example 19. Synthesis of Compound 7

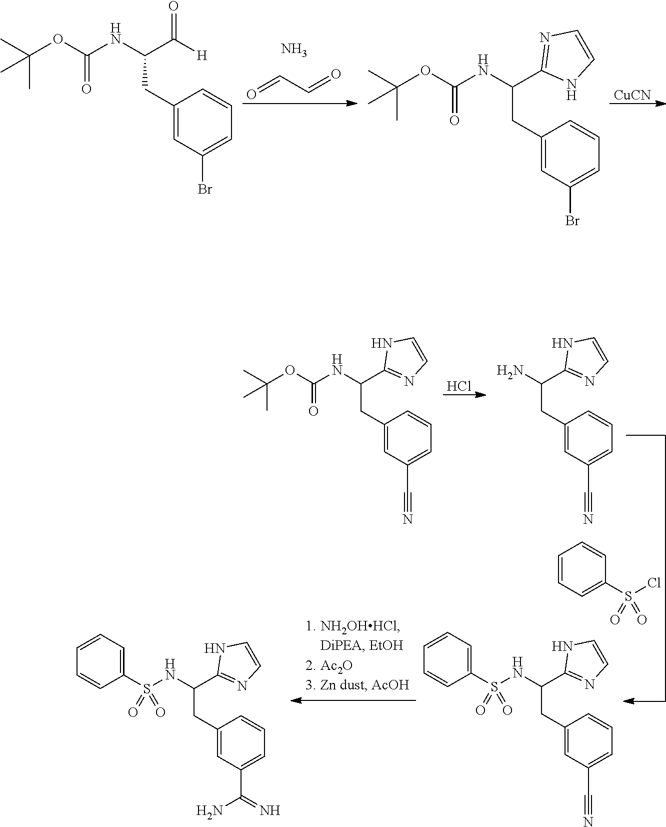

### Step 1: Tert-butyl N-[(1S)-2-(3-bromophenyl)-1-(1H-imidazol-2-yl)ethyl]carbamate)

To a magnetically stirred solution of tert-butyl N-[(2S)-1-(3-bromophenyl)-3-oxopropan-2-yl]carbamate (4.80 g, 14.6 mmol, 1.0 eq.) in MeCN (100 mL) were added 40% aq. solution of oxaldehyde (12.9 mL, 113 mmol, 7.6 eq.) and conc. ammonia (13.5 mL, 243 mmol, 16.5 eq.). The reaction mixture was stirred at RT for 48 h. Aq. sat. NaHCO₃ solution (200 mL) was added and the product was extracted with DCM (2×200 mL). The combined organic phase was washed with water (200 mL), dried over anhydrous sodium sulfate, filtered and concentrated to dryness to afford product as a pale orange solid (3.30 g, 9.01 mmol, 62% yield). ¹H NMR (DMSO-d6) δ: 11.71 (br s, 1H), 7.32-7.43 (m, 2H), 7.11-7.28 (m, 3H), 6.99 (s, 1H), 6.80 (s, 1H), 4.78 (td, J=9.2, 5.5 Hz, 1H), 3.19 (dd, J=13.6, 5.4 Hz, 1H), 2.94 (dd, J=13.6, 9.5 Hz, 1H), 1.20-1.37 (m, 9H). UPLC-MS (basic 2 min) Rt=1.07 min. m/z=365.9 for [M]⁺

### Step 2: Tert-butyl N-[(1S)-2-(3-cyanophenyl)-1-(1H-imidazol-2-yl)ethyl]carbamate

To a magnetically stirred solution of tert-butyl N-[(1S)-2-(3-bromophenyl)-1-(1H-imidazol-2-yl)ethyl]carbamate (1.080 g, 2.949 mmol, 1.0 eq.) in NMP (20.0 mL) were added CuCN (0.410 g, 4.58 mmol, 1.6 eq.) and tetrakis(triphenylphosphane) palladium (0.937 g, 0.811 mmol, 0.28 eq.). The reaction mixture was stirred at 100° C. for 1 h. The reaction mixture was allowed to cool to RT and then aq. sat. NH₄Cl solution (50 mL) was added. The product was extracted with DCM (3×50 mL). The combined organic phase was washed with water (50 mL), dried over anhydrous sodium sulfate, filtered and concentrated to dryness. The residue was purified by Reverse Phase column chromatography eluting with a 5-95% H₂O:MeCN eluent (0.1% formic acid) to afford product as a pale green glass (0.564 g, 1.81 mmol, 61% yield). UPLC-MS (basic 2 min) Rt=0.96 min. m/z=313.0 for [M+H]⁺

### Step 3: N-[(1S)-2-(3-Cyanophenyl)-1-(1H-imidazol-2-yl)ethyl]benzenesulfonamide

To a magnetically stirred solution of tert-butyl N-[(1S)-2-(3-cyanophenyl)-1-(1H-imidazol-2-yl)ethyl]carbamate (0.564 g, 1.81 mmol, 1.0 eq.) in diethyl ether (20.0 mL) and MeOH (5.0 mL) was added 4N HCl in dioxane (10 mL, 40 mmol, 22.0 eq.) and the reaction mixture was stirred at RT for 24 h. The reaction mixture was concentrated to dryness to afford product as a brown solid (0.466 g, 1.63 mmol, 91% yield) which was used in the next step without further purification. UPLC-MS (basic 2 min) Rt=0.72 min. m/z=213.0 for [M+H]⁺

### Step 4: N-[(1S)-2-(3-Cyanophenyl)-1-(1H-imidazol-2-yl)ethyl]benzenesulfonamide

To a magnetically stirred solution of 2-[(1S)-1-azaniumyl-2-(3-cyanophenyl)ethyl]-1H-imidazol-1-ium dichloride (0.233 g, 0.817 mmol, 1.0 eq.) in pyridine (5.0 mL) was added benzenesulfonyl chloride (0.124 mL, 0.968 mmol, 1.2 eq.) and the reaction mixture was stirred at RT for 4 d. The reaction mixture was concentrated to dryness and the residue was purified by Reverse Phase column chromatography on a C18 cartridge eluting with a 5-95% H₂O:MeCN eluent (0.1% ammonia) to afford product as a colorless glass (0.140 g, 0.397 mmol, 49% yield). ¹H NMR (DMSO-d6) δ: 11.47-11.76 (m, 1H), 8.04-8.77 (m, 1H), 7.54-7.59 (m, 1H), 7.49-7.54 (m, 2H), 7.45-7.49 (m, 1H), 7.34-7.45 (m, 3H), 7.30-7.34 (m, 2H), 6.85 (br s, 1H), 6.70 (br s, 1H), 4.54 (t, J=7.5 Hz, 1H), 3.11 (dd, J=13.6, 7.0 Hz, 1H), 2.90-3.02 (m, 1H). UPLC-MS (basic 2 min) Rt=0.91 min. m/z=352.9 for [M+H]⁺

### Step 5: 3-[(2S)-2-Benzenesulfonamido-2-(1H-imidazol-2-yl)ethyl]-N′-hydroxybenzene-1-carboximidamide

To a magnetically stirred solution of N-[(1S)-2-(3-cyanophenyl)-1-(1H-imidazol-2-yl)ethyl]benzenesulfonamide (0.140 g, 0.397 mmol, 1.0 eq.) in EtOH (4.0 mL) were added hydroxylamine hydrochloride (0.091 g, 1.31 mmol, 3.3 eq.) and DIPEA (0.300 mL, 1.72 mmol, 4.0 eq.). The reaction mixture was stirred under reflux for 6 h. The reaction mixture was cooled down to RT and then concentrated to dryness to afford product as a pale brown solid (0.060 g, 0.156 mmol, 39% yield) which was used in the next step without further purification. UPLC-MS (basic 2 min) Rt=0.79 min. m/z=386.0 for [M+H]⁺

### Step 6: [Amino({3-[(2S)-2-benzenesulfonamido-2-(1H-imidazol-2-yl)ethyl]phenyl})methylidene]amino acetate

To a magnetically stirred solution of 3-[(2S)-2-benzenesulfonamido-2-(1H-imidazol-2-yl)ethyl]-N′-hydroxybenzene-1-carboximidamide (0.060 g, 0.156 mmol, 1.0 eq) in acetic acid (2.0 mL) was added acetic anhydride (0.020 mL, 0.212 mmol, 1.36 eq.) dropwise. The resulting mixture was stirred at RT for 60 mins. and then concentrated to dryness to afford product as a white solid (0.073 g, 0.171 mmol, 100% yield) which was used in the next step without further purification. UPLC-MS (basic 2 min): Rt=0.84 min; m/z=428.0 for [M+H]⁺

### Step 7: 2-[2-{3-[Amino(iminiumyl)methyl]phenyl}-1-benzenesulfonamidoethyl]-1H-imidazol-3-ium dichloride

To a magnetically stirred solution of [amino({3-[(2S)-2-benzenesulfonamido-2-(1H-imidazol-2-yl)ethyl]phenyl})methylidene]amino acetate) (0.073 g, 0.171 mmol, 1.0 eq) in acetic acid (2.0 mL) was added zinc (0.257 g, 3.93 mmol, 23.0 eq.) and the reaction mixture was stirred at room temperature for 48 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% formic acid) to afford the formate salt as a white solid. The formate salt was passed through a SAX cartridge eluting with 1M HCl/MeOH and then concentrated to afford product as a white solid (0.008 g, 10% yield). ¹H NMR (400 MHz, MeOH-d₄): δ 7.60-7.71 (m, 4H), 7.52-7.60 (m, 1H), 7.45 (d, J=7.6 Hz, 2H), 7.35-7.42 (m, 4H), 5.02 (br t, J=7.7 Hz, 1H), 3.32-3.37 (m, 1H), 3.18-3.30 (m, 1H). UPLC-MS (basic 6 min): Rt=1.28 min; m/z=370.0 for [M+H]⁺, 100% purity. Chiral analysis performed by Reach Separations showed 82.1% chiral purity.

### Example 20. Synthesis of Compound 8

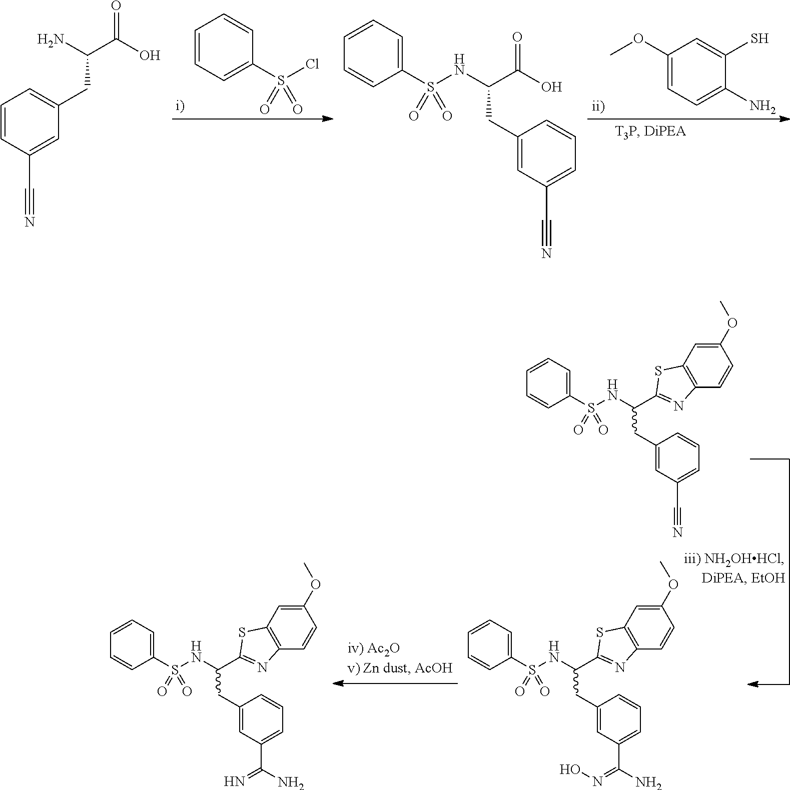

### Step 1: 2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[2-(3-Cyanophenyl)-1-(6-methoxy-1,3-benzothiazol-2-yl)ethyl]benzenesulfonamide

2-benzenesulfonamido-3-(3-cyanophenyl)propanoic acid (0.300 g, 0.908 mmol, 1.0 eq.) was dissolved in T3P (50% w/w in EtOAc) (1.1 mL, 1.82 mmol, 2.0 eq.). DIPEA (0.237 mL, 1.36 mmol, 1.5 eq.) and 2-amino-5-methoxybenzene-1-thiol (0.169 g, 1.09 mmol, 1.2 eq.) were added and the reaction mixture was heated at 100° C. under microwave irradiation for 10 mins. The reaction mixture was cooled to RT and then concentrated to dryness. The residue was purified by Reverse Phase column chromatography eluting with a 5-95% H₂O:MeCN eluent (0.1% ammonia) to afford product as a white solid (0.197 g, 0.429 mmol, 47% yield). ¹H NMR (DMSO-d6) δ: 8.93 (s, 1H), 7.82 (d, J=8.9 Hz, 1H), 7.65 (d, J=2.6 Hz, 1H), 7.59 (t, J=1.7 Hz, 1H), 7.52 (dq, J=8.2, 2.1, 1.7 Hz, 2H), 7.44 (td, J=7.5, 1.4 Hz, 3H), 7.33-7.26 (m, 3H), 7.10 (dd, J=8.9, 2.6 Hz, 1H), 4.97 (d, J=8.7 Hz, 1H), 2.99 (dd, J=13.9, 10.5 Hz, 1H). UPLC-MS (basic 2 min) Rt=1.10 min. m/z=448.9 for [M−H]⁺

### Step 3: 3-[2-Benzenesulfonamido-2-(6-methoxy-1,3-benzothiazol-2-yl)ethyl]-N′-hydroxybenzene-1-carboximidamide

To a magnetically stirred solution of N-[2-(3-cyanophenyl)-1-(6-methoxy-1,3-benzothiazol-2-yl)ethyl] benzenesulfonamide (0.197 g, 0.438 mmol, 1.0 eq.) in EtOH (2 mL) were added hydroxylamine hydrochloride (0.061 g, 0.876 mmol, 2.0 eq.) and DIPEA (0.229 mL, 1.32 mmol, 3.0 eq.). The reaction mixture was stirred under reflux for 1 h. The reaction mixture was cooled down to RT and then concentrated to dryness to afford product as a yellow oil (0.212 g, 0.439 mmol, 100% yield) which was used in the next step without further purification. UPLC-MS (basic 2 min) Rt=1.00 min. m/z=483.9 for [M+H]⁺

### Step 4: [Amino({3-[2-benzenesulfonamido-2-(6-methoxy-1,3-benzothiazol-2-yl)ethyl]phenyl})methylidene] amino acetate

To a magnetically stirred solution of 3-[2-benzenesulfonamido-2-(6-methoxy-1,3-benzothiazol-2-yl)ethyl]-N′-hydroxybenzene-1-carboximidamide) (0.212 g, 0.439 mmol, 1.0 eq) in acetic acid (2.0 mL) was added acetic anhydride (0.124 mL, 1.32 mmol, 3.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% ammonia) to afford product as a white solid (0.076 g, 0.145 mmol, 33% yield). UPLC-MS (basic 2 min): Rt=1.04 min; m/z=524.9 for [M+H]⁺

### Step 5: 3-[2-Benzenesulfonamido-2-(6-methoxy-1,3-benzothiazol-2-yl)ethyl]benzene-1-carboximidamide

To a magnetically stirred solution of [amino({3-[2-benzenesulfonamido-2-(6-methoxy-1,3-benzothiazol-2-yl)ethyl]phenyl})methylidene]amino acetate (0.076 g, 0.145 mmol, 1.0 eq) in acetic acid (1.0 mL) was added zinc (0.095 g, 1.45 mmol, 10.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% formic acid) to afford the formate salt as a white solid. The formate salt was passed through an SCX column to afford product as a white solid (0.023 g, 0.048 mmol, 34% yield). ¹H NMR (400 MHz, DMSO-d₆): δ 7.75 (d, J=8.9 Hz, 1H), 7.66 (s, 1H), 7.56 (d, J=2.6 Hz, 1H), 7.51 (d, J=7.7 Hz, 1H), 7.46 (d, 2H), 7.34-7.28 (m, 1H), 7.27-7.20 (m, 3H), 7.17 (t, J=7.6 Hz, 1H), 7.04 (dd, J=8.9, 2.6 Hz, 1H), 4.82 (dd, J=8.8, 5.1 Hz, 1H), 3.81 (s, 3H), 3.24 (dd, J=13.6, 5.1 Hz, 1H), 3.01 (dd, J=13.6, 8.9 Hz, 1H). UPLC-MS (basic 4 min): Rt=1.39 min; m/z=466.9 for [M]+, 100% purity. Chiral analysis performed by Reach Separations showed 50% chiral purity.

### Example 21. Synthesis of Compound 12

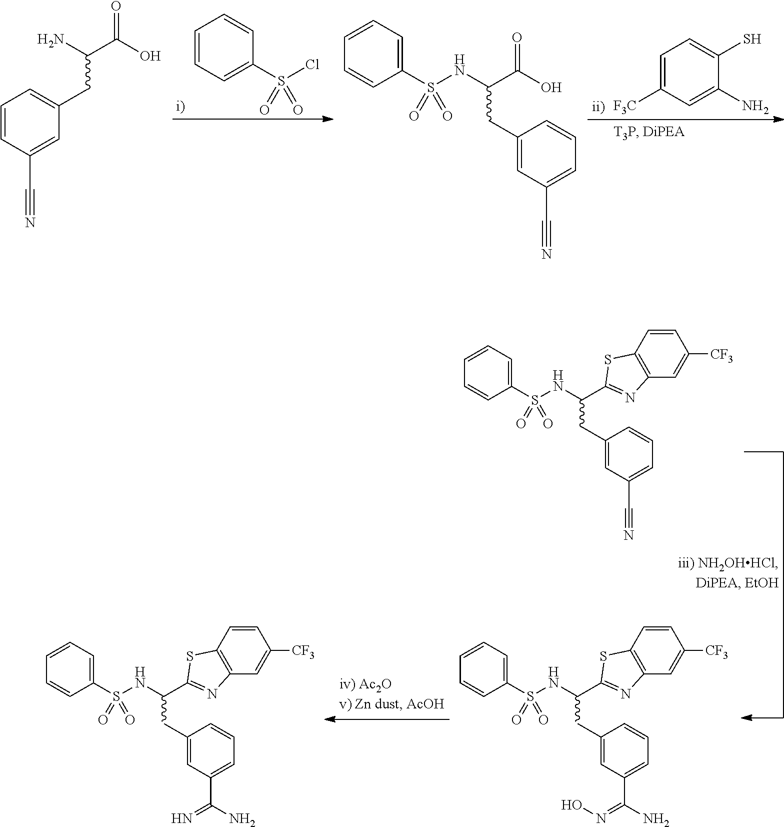

### Step 1: 2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[2-(3-Cyanophenyl)-1-[5-(trifluoromethyl)-1,3-benzothiazol-2-yl]ethyl]benzenesulfonamide

2-Benzenesulfonamido-3-(3-cyanophenyl)propanoic acid (0.300 g, 0.908 mmol, 1.0 eq.) was dissolved in T3P (50% w/w in EtOAc) (1.1 mL, 1.82 mmol, 2.0 eq.). DIPEA (0.395 mL, 2.27 mmol, 2.5 eq.) and 2-amino-4-(trifluoromethyl)benzene-1-thiol hydrochloride (0.250 g, 1.09 mmol, 1.2 eq.) were added and the reaction mixture was heated at 100° C. under microwave irradiation for 10 mins. The reaction mixture was cooled to RT and then concentrated to dryness. The residue was purified by Reverse Phase column chromatography eluting with a 5-95% H₂O:MeCN eluent (0.1% ammonia) to afford product as a white solid (0.218 g, 0.438 mmol, 48% yield). ¹H NMR (DMSO-d6) δ: 9.08 (s, 1H), 8.38 (d, J=8.4 Hz, 1H), 8.30 (s, 1H), 7.79 (dd, J=8.5, 1.8 Hz, 1H), 7.61 (s, 1H), 7.53 (dd, J=7.8, 1.6 Hz, 2H), 7.47-7.39 (m, 3H), 7.33-7.25 (m, 3H), 5.10 (dd, J=10.6, 4.4 Hz, 1H), 3.40 (dd, J=13.9, 4.5 Hz, 1H), 3.02 (dd, J=13.9, 10.6 Hz, 1H). UPLC-MS (basic 2 min) Rt=1.22 min. m/z=486.1 for [M−H]⁺

### Step 3: 3-{2-Benzenesulfonamido-2-[5-(trifluoromethyl)-1,3-benzothiazol-2-yl]ethyl}-N′-hydroxybenzene-1-carboximidamide

To a magnetically stirred solution of N-[2-(3-cyanophenyl)-1-[5-(trifluoromethyl)-1,3-benzothiazol-2-yl]ethyl]benzenesulfonamide (0.218 g, 0.447 mmol, 1.0 eq.) in EtOH (2 mL) were added hydroxylamine hydrochloride (0.062 g, 0.894 mmol, 2.0 eq.) and DIPEA (0.234 mL, 1.34 mmol, 3.0 eq.). The reaction mixture was stirred under reflux for 1 h. The reaction mixture was cooled down to RT and then concentrated to dryness to afford product as a yellow oil (0.233 g, 0.448 mmol, 100% yield) which was used in the next step without further purification. UPLC-MS (basic 2 min) Rt=1.12 min. m/z=520.9 for [M+H]⁺

### Step 4: [Amino(3-{2-benzenesulfonamido-2-[5-(trifluoromethyl)-1,3-benzothiazol-2-yl]ethyl}phenyl) methylidene]amino acetate

To a magnetically stirred solution of 3-{2-benzenesulfonamido-2-[5-(trifluoromethyl)-1,3-benzothiazol-2-yl]ethyl}-N′-hydroxybenzene-1-carboximidamide (0.236 g, 0.453 mmol, 1.0 eq) in acetic acid (2.0 mL) was added acetic anhydride (0.128 mL, 1.36 mmol, 3.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% ammonia) to afford product as a white solid (0.061 g, 0.108 mmol, 24% yield). UPLC-MS (basic 2 min): Rt=1.14 min; m/z=562.9 for [M+H]⁺

### Step 5: 3-{2-Benzenesulfonamido-2-[5-(trifluoromethyl)-1,3-benzothiazol-2-yl]ethyl}benzene-1-carboximidamide

To a magnetically stirred solution of [amino(3-{2-benzenesulfonamido-2-[5-(trifluoromethyl)-1,3-benzothiazol-2-yl]ethyl}phenyl)methylidene]amino acetate) (0.061 g, 0.108 mmol, 1.0 eq) in acetic acid (1.0 mL) was added zinc (0.071 g, 1.08 mmol, 10.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% formic acid) to afford the formate salt as a white solid. The formate salt was passed through an SCX column to afford product as a white solid (0.021 g, 0.042 mmol, 38% yield). ¹H NMR (400 MHz, DMSO-d₆): δ 8.25 (d, J=8.4 Hz, 1H), 8.20 (s, 1H), 7.71-7.66 (m, 2H), 7.52 (d, 1H), 7.47-7.42 (m, 2H), 7.31-7.17 (m, 5H), 4.85 (dd, J=8.7, 4.8 Hz, 1H), 3.25 (dd, 1H), 3.04 (dd, J=13.3, 8.7 Hz, 1H). UPLC-MS (basic 4 min): Rt=1.61 min; m/z=504.9 for [M]⁺, 100% purity. Chiral analysis performed by Reach Separations showed 50% chiral purity.

### Example 22. Synthesis of Compound 9

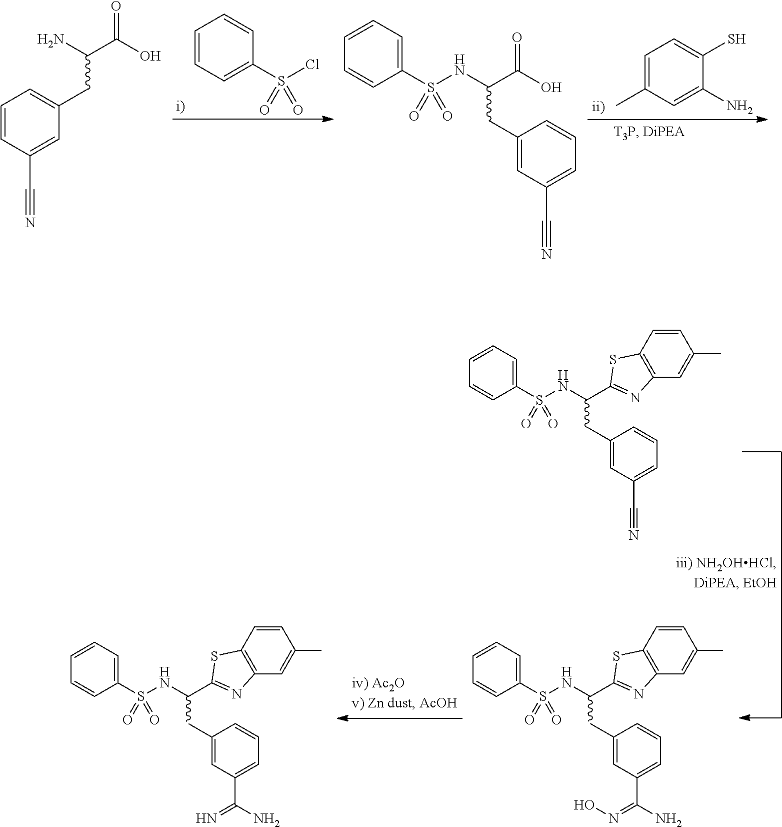

### Step 1: 2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[2-(3-Cyanophenyl)-1-(5-methyl-1,3-benzothiazol-2-yl)ethyl]benzenesulfonamide

2-Benzenesulfonamido-3-(3-cyanophenyl)propanoic acid (0.100 g, 0.303 mmol, 1.0 eq.) was dissolved in T3P (50% w/w in EtOAc) (0.36 mL, 0.605 mmol, 2.0 eq.). DIPEA (0.079 mL, 0.454 mmol, 1.5 eq.) and 2-amino-4-methylbenzene-1-thiol (0.051 g, 0.363 mmol, 1.2 eq.) were added and the reaction mixture was heated at 100° C. under microwave irradiation for 10 mins. The reaction mixture was cooled to RT and then concentrated to dryness. The residue was purified by Reverse Phase column chromatography eluting with a 5-95% H₂O:MeCN eluent (0.1% ammonia) to afford product as a white solid (0.234 g, 0.529 mmol, 87% yield). ¹H NMR (DMSO-d6) δ: 8.95 (d, J=7.9 Hz, 1H), 7.94 (d, J=8.2 Hz, 1H), 7.75 (s, 1H), 7.58 (s, 1H), 7.52 (d, 2H), 7.47-7.41 (m, 3H), 7.33-7.25 (m, 4H), 4.99 (s, 1H), 3.04-2.93 (m, 2H), 2.46 (s, 3H). UPLC-MS (basic 2 min) Rt=1.19 min. m/z=433.9 for [M−H]⁺

### Step 3: 3-[2-Benzenesulfonamido-2-(5-methyl-1,3-benzothiazol-2-yl)ethyl]-N′-hydroxybenzene-1-carboximidamide

To a magnetically stirred solution of N-[2-(3-cyanophenyl)-1-(5-methyl-1,3-benzothiazol-2-yl)ethyl]benzenesulfonamide (0.234 g, 0.540 mmol, 1.0 eq.) in EtOH (2.5 mL) were added hydroxylamine hydrochloride (0.075 g, 1.08 mmol, 2.0 eq.) and DIPEA (0.282 mL, 1.62 mmol, 3.0 eq.). The reaction mixture was stirred under reflux for 1 h. The reaction mixture was cooled down to RT and then concentrated to dryness to afford product as a yellow oil (0.252 g, 0.540 mmol, 100% yield) which was used in the next step without further purification. UPLC-MS (basic 2 min) Rt=1.06 min. m/z=467.0 for [M+H]⁺

### Step 4: [Amino({3-[2-benzenesulfonamido-2-(5-methyl-1,3-benzothiazol-2-yl)ethyl]phenyl})methylidene] amino acetate

To a magnetically stirred solution of 3-[2-benzenesulfonamido-2-(5-methyl-1,3-benzothiazol-2-yl)ethyl]-N′-hydroxybenzene-1-carboximidamide (0.252 g, 0.540 mmol, 1.0 eq) in acetic acid (3.0 mL) was added acetic anhydride (0.153 mL, 1.62 mmol, 3.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% ammonia) to afford product as a white solid (0.020 g, 0.039 mmol, 7% yield). UPLC-MS (basic 2 min): Rt=1.10 min; m/z=508.9 for [M+H]⁺

### Step 5: 3-[2-Benzenesulfonamido-2-(5-methyl-1,3-benzothiazol-2-yl)ethyl]benzene-1-carboximidamide

To a magnetically stirred solution of [amino({3-[2-benzenesulfonamido-2-(5-methyl-1,3-benzothiazol-2-yl)ethyl]phenyl})methylidene]amino acetate (0.020 g, 0.039 mmol, 1.0 eq) in acetic acid (1.0 mL) was added zinc (0.026 g, 0.393 mmol, 10.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% formic acid) to afford the formate salt as a white solid. The formate salt was passed through an SCX column to afford product as a white solid (0.005 g, 0.011 mmol, 28% yield). ¹H NMR (400 MHz, DMSO-d₆): δ 7.86 (d, J=8.1 Hz, 1H), 7.67 (d, J=11.0 Hz, 2H), 7.51 (d, J=7.6 Hz, 1H), 7.46 (d, 2H), 7.31 (t, J=7.3 Hz, 1H), 7.27-7.14 (m, 5H), 4.86 (dd, J=8.8, 5.0 Hz, 1H), 3.28-3.21 (m, 1H), 3.01 (dd, J=13.5, 8.9 Hz, 1H), 2.44 (s, 3H). UPLC-MS (basic 4 min): Rt=1.50 min; m/z=451.0 for [M]+, 98% purity. Chiral analysis performed by Reach Separations showed 50% chiral purity.

### Example 23. Synthesis of Compound 13

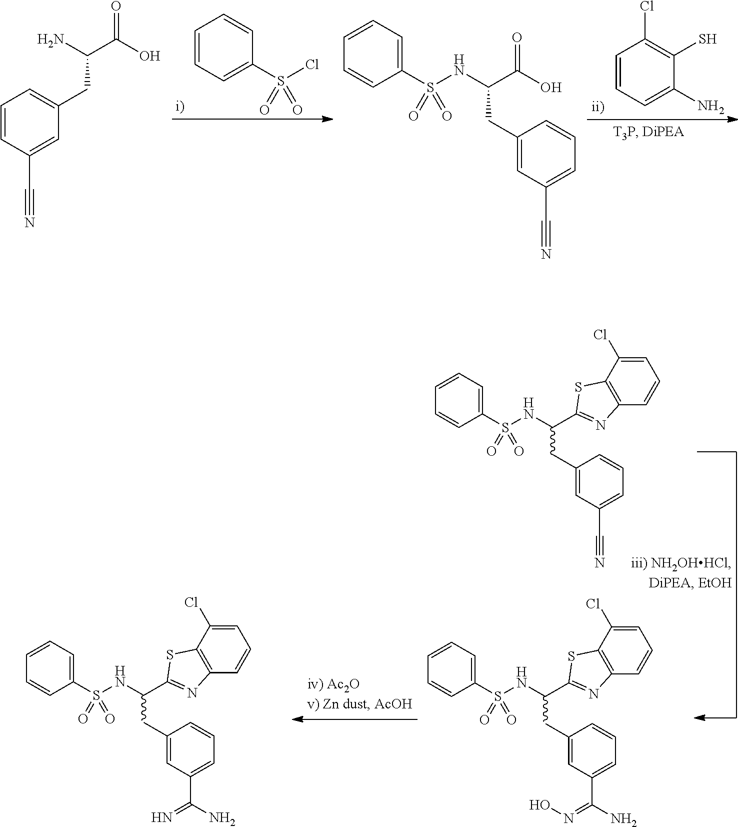

### Step 1: 2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[1-(7-Chloro-1,3-benzothiazol-2-yl)-2-(3-cyanophenyl)ethyl]benzenesulfonamide

2-Benzenesulfonamido-3-(3-cyanophenyl)propanoic acid (0.505 g, 1.53 mmol, 1.0 eq.) was dissolved in toluene (10.0 mL) and then T3P (50% w/w in EtOAc) (2.0 mL, 3.43 mmol, 2.2 eq.). DIPEA (0.800 mL, 4.59 mmol, 3.0 eq.) and 2-amino-6-chlorobenzene-1-thiol hydrochloride (0.330 g, 1.69 mmol, 1.1 eq.) were added. The reaction mixture was stirred at RT for 30 mins. and then heated at 100° C. for 1 h. The reaction mixture was cooled to RT and then aq. 1M HCl solution was added. The product was extracted with DCM (3×25 mL) and the combined organic phase was washed with aq. sat. NaHCO₃ solution (25 mL), H₂O (25 mL), dried over anhydrous sodium sulfate, filtered and then concentrated to dryness. The residue was purified by Reverse Phase column chromatography eluting with a 5-95% H₂O:MeCN eluent (0.1% ammonia) to afford product as a white solid (0.392 g, 0.864 mmol, 57% yield). ¹H NMR (DMSO-d6) δ: 9.04 (br d, J=6.4 Hz, 1H), 7.95 (dd, J=6.7, 2.3 Hz, 1H), 7.60-7.63 (m, 1H), 7.54-7.60 (m, 2H), 7.53 (dd, J=7.8, 1.5 Hz, 2H), 7.38-7.49 (m, 3H), 7.29 (q, J=8.3 Hz, 3H), 4.98-5.14 (m, 1H), 3.40 (dd, J=13.9, 4.4 Hz, 1H), 3.01 (dd, J=13.9, 10.8 Hz, 1H). UPLC-MS (basic 2 min) Rt=1.21 min. m/z=453.9 for [M]⁺

### Step 3: 3-[2-Benzenesulfonamido-2-(7-chloro-1,3-benzothiazol-2-yl)ethyl]-N′-hydroxybenzene-1-carboximidamide

To a magnetically stirred solution of N-[1-(7-chloro-1,3-benzothiazol-2-yl)-2-(3-cyanophenyl)ethyl]benzenesulfonamide (0.228 g, 0.502 mmol, 1.0 eq.) in EtOH (6.0 mL) were added hydroxylamine hydrochloride (0.105 g, 1.51 mmol, 3.0 eq.) and DIPEA (0.350 mL, 2.01 mmol, 4.0 eq.). The reaction mixture was stirred under reflux for 1 h. The reaction mixture was cooled down to RT and then concentrated to dryness to afford product as a yellow oil (0.249 g, 0.511 mmol, 100% yield) which was used in the next step without further purification. UPLC-MS (basic 2 min) Rt=1.08 min. m/z=486.9 for [M]⁺

### Step 4: [Amino({3-[2-benzenesulfonamido-2-(7-chloro-1,3-benzothiazol-2-yl)ethyl]phenyl})methylidene] amino acetate

To a magnetically stirred solution of 3-[2-benzenesulfonamido-2-(7-chloro-1,3-benzothiazol-2-yl)ethyl]-N′-hydroxybenzene-1-carboximidamide (0.249 g, 0.511 mmol, 1.0 eq) in acetic acid (5.0 mL) was added acetic anhydride (0.20 mL, 2.12 mmol, 4.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% ammonia) to afford product as a white solid (0.083 g, 0.157 mmol, 31% yield). UPLC-MS (basic 2 min): Rt=1.12 min; m/z=528.8 for [M]⁺

### Step 5: 3-[2-Benzenesulfonamido-2-(7-chloro-1,3-benzothiazol-2-yl)ethyl]benzene-1-carboximidamide

To a magnetically stirred solution of [amino({3-[2-benzenesulfonamido-2-(7-chloro-1,3-benzothiazol-2-yl)ethyl]phenyl})methylidene]amino acetate (0.083 g, 0.157 mmol, 1.0 eq) in acetic acid (2.0 mL) was added zinc (0.212 g, 3.24 mmol, 20.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% formic acid) to afford the formate salt as a white solid. The formate salt was passed through an SAX column to afford product as a white solid (0.027 g, 0.057 mmol, 37% yield). ¹H NMR (400 MHz, DMSO-d₆): δ 7.92 (dd, J=6.8, 2.2 Hz, 1H), 7.74 (s, 1H), 7.53-7.61 (m, 3H), 7.43-7.53 (m, 3H), 7.35-7.42 (m, 1H), 7.28-7.34 (m, 1H), 7.20-7.27 (m, 2H), 5.09 (br dd, J=10.0, 4.6 Hz, 1H), 3.40 (dd, J=13.9, 4.9 Hz, 1H), 3.08 (dd, J=13.8, 10.1 Hz, 1H). UPLC-MS (basic 6 min): Rt=2.69 min; m/z=470.9 for [M]⁺, 100% purity. Chiral analysis performed by Reach Separations showed 66% chiral purity.

### Example 24. Synthesis of Compound 10

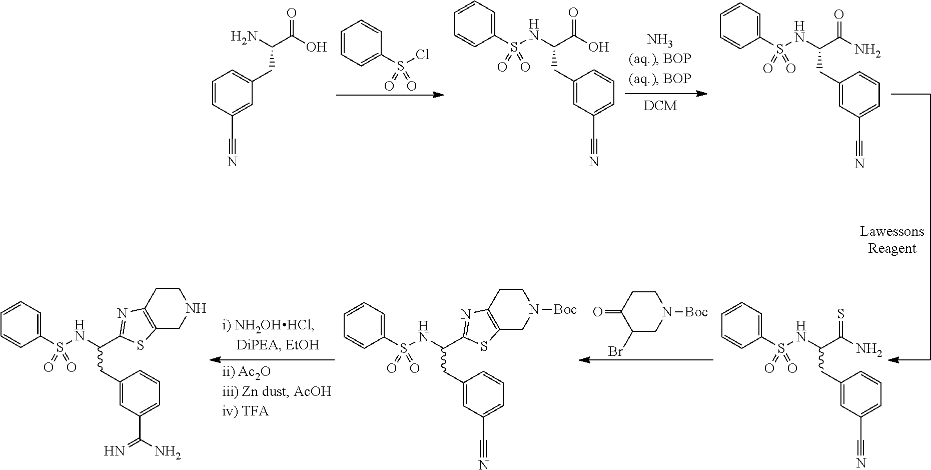

### Step 1: (2S)-2-Benzenesulfonamido-3-(3-cyanophenyl)propanoic acid

To a magnetically stirred solution of (2S)-2-amino-3-(3-cyanophenyl)propanoic acid (5.00 g, 26.3 mmol, 1.0 eq.) in water (30.0 mL) were added sodium bicarbonate (3.34 g, 31.5 mmol, 1.2 eq.) and the resulting solution was cooled to −5° C. Benzenesulfonyl chloride (4.00 mL, 31.5 mmol, 1.2 eq.) was added in 4 portions over a period of 60 mins. The resulting slurry was allowed to warm to RT and stirred for 18 h. The reaction mixture was acidified with 1M HCl and then filtered under vacuum. The solid was triturated with water (2×50 mL) and then dried to afford product as a white solid (4.26 g, 12.9 mmol, 49% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (acidic 4 min) Rt=1.31 min. m/z=328.9 for [M−H]⁺

### Step 2: (2S)-2-Benzenesulfonamido-3-(3-cyanophenyl)propanamide)

To a magnetically stirred solution of (2S)-2-benzenesulfonamido-3-(3-cyanophenyl)propanoic acid (1.00 g, 3.03 mmol, 1.0 eq.) in DCM (30.0 mL) were added DIPEA (1.60 mL, 9.08 mmol, 3.0 eq.) and BOP (1.61 g, 3.63 mmol, 1.2 eq.). The mixture was stirred at RT for 15 mins before adding conc. ammonia (0.841 mL, 15.1 mmol, 5.0 eq.). The reaction mixture was stirred at RT for 1 h and then diluted with aq. saturated NaHCO₃ solution (20 mL). The product was extracted with DCM (2×20 mL) and the organic layer dried over anhydrous sodium sulfate before filtering and concentrating to dryness. The residue was purified by Reverse Phase column chromatography eluting with a 5-95% H₂O:MeCN eluent (0.1% ammonia) to afford product as a pale yellow solid (0.530 g, 1.61 mmol, 53% yield). ¹H NMR (DMSO-d6) δ: 8.11 (d, J=9.0 Hz, 1H), 7.60 (d, J=7.4 Hz, 1H), 7.56-7.45 (m, 4H), 7.40 (dt, J=8.4, 4.0 Hz, 3H), 7.07 (s, 1H), 3.97-3.88 (m, 1H), 2.95-2.86 (m, 1H), 2.73-2.62 (m, 1H). UPLC-MS (basic 2 min) Rt=0.87 min. m/z=327.9 for [M−H]⁺

### Step 3: 2-Benzenesulfonamido-3-(3-cyanophenyl)propanethioamide

To a magnetically stirred solution of (2S)-2-benzenesulfonamido-3-(3-cyanophenyl)propenamide (0.530 g, 1.61 mmol, 1.0 eq.) in MeCN (16.0 mL) was added Lawesson's Reagent (0.781 g, 1.93 mmol, 1.2 eq.) and the reaction mixture was stirred at 100° C. for 18 h. The reaction mixture was cooled to RT and then concentrated to dryness. The residue was purified by Reverse Phase column chromatography eluting with a 5-95% H₂O:MeCN eluent (0.1% ammonia) to afford product as a pale yellow solid (0.183 g, 0.530 mmol, 33% yield). ¹H NMR (DMSO-d6) δ: 9.62 (s, 1H), 9.23 (s, 1H), 8.01 (s, 1H), 7.61-7.47 (m, 6H), 7.43-7.34 (m, 3H), 4.25 (dd, J=9.3, 4.8 Hz, 1H), 2.94 (dd, J=13.6, 4.8 Hz, 1H), 2.78 (dd, J=13.8, 9.6 Hz, 1H). UPLC-MS (basic 2 min) Rt=0.95 min. m/z=343.9 for [M−H]⁺

### Step 4: tert-Butyl 2-[1-benzenesulfonamido-2-(3-cyanophenyl)ethyl]-4H,5H,6H,7H-[1,3]thiazolo[5,4-c]pyridine-5-carboxylate

To a magnetically stirred suspension of 2-benzenesulfonamido-3-(3-cyanophenyl)propanethioamide (0.180 g, 0.521 mmol, 1.0 eq.) in EtOH (4.0 mL) was added tert-butyl 3-bromo-4-oxopiperidine-1-carboxylate (0.791 g, 1.56 mmol, 3.0 eq.) and the reaction mixture was stirred at 100° C. for 2 h and then at RT for 18 h. The reaction mixture was concentrated to dryness and the residue was purified by Reverse Phase column chromatography eluting with a 5-95% H₂O:MeCN eluent (0.1% ammonia) to afford product as a yellow solid (0.230 g, 0.438 mmol, 84% yield). ¹H NMR (DMSO-d6) δ: 8.75 (d, J=8.5 Hz, 1H), 7.58-7.38 (m, 6H), 7.34-7.28 (m, 3H), 4.80 (ddd, J=10.2, 8.4, 4.5 Hz, 1H), 3.61 (t, J=5.8 Hz, 2H), 3.22 (dd, J=13.8, 4.6 Hz, 1H), 2.91 (dd, J=13.9, 10.5 Hz, 1H), 2.72-2.59 (m, 2H), 1.63-1.58 (m, 2H), 1.44 (s, 9H). UPLC-MS (basic 2 min) Rt=1.18 min. m/z=524.9 for [M+H]⁺

### Step 5: tert-Butyl 2-{1-benzenesulfonamido-2-[3-(N′-hydroxycarbamimidoyl)phenyl]ethyl}-4H,5H,6H,7H-[1,3]thiazolo[5,4-c]pyridine-5-carboxylate

To a magnetically stirred solution of tert-butyl 2-[1-benzenesulfonamido-2-(3-cyanophenyl)ethyl]-4H,5H,6H,7H-[1,3]thiazolo[5,4-c]pyridine-5-carboxylate (0.090 g, 0.172 mmol, 1.0 eq.) in EtOH (2 mL) were added hydroxylamine hydrochloride (0.024 g, 0.343 mmol, 2.0 eq.) and DIPEA (0.090 mL, 0.515 mmol, 3.0 eq.). The reaction mixture was stirred under reflux for 1 h. The reaction mixture was cooled down to RT and then concentrated to dryness to afford product as a yellow oil (0.095 g, 0.170 mmol, 100% yield) which was used in the next step without further purification. UPLC-MS (basic 2 min) Rt=1.05 min. m/z=558.0 for [M+H]⁺

### Step 6: tert-Butyl 2-(2-{3-[N′-(acetyloxy)carbamimidoyl]phenyl}-1-benzenesulfonamidoethyl)-4H,5H,6H,7H-[1,3]thiazolo[5,4-c]pyridine-5-carboxylate

To a magnetically stirred solution of tert-butyl 2-{1-benzenesulfonamido-2-[3-(N′-hydroxycarbamimidoyl)phenyl]ethyl}-4H,5H,6H,7H-[1,3]thiazolo[5,4-c]pyridine-5-carboxylate (0.095 g, 0.170 mmol, 1.0 eq) in acetic acid (2.0 mL) was added acetic anhydride (0.052 mL, 0.511 mmol, 3.0 eq.) and the reaction mixture was stirred at RT for 1 h. The reaction mixture was concentrated to dryness and the residue was purified by Normal Phase Column Chromatography eluting with 0-10% MeOH, DCM (0.1% ammonia) to afford product as a yellow gummy solid (0.074 g, 0.123 mmol, 72% yield). ¹H NMR (400 MHz, DMSO-d₆): δ 8.72 (d, J=8.4 Hz, 1H), 7.55 (s, 1H), 7.51-7.39 (m, 4H), 7.30 (t, J=7.7 Hz, 2H), 7.24-7.09 (m, 2H), 6.73 (s, 2H), 4.82-4.73 (m, 1H), 3.55-3.42 (m, 1H), 3.16 (dd, J=13.7, 5.4 Hz, 1H), 2.93 (dd, J=13.9, 9.7 Hz, 1H), 2.60 (dd, J=13.4, 7.2 Hz, 2H), 2.35 (t, J=6.3 Hz, 3H), 1.92 (s, 9H), 1.86 (s, 3H). UPLC-MS (basic 2 min): Rt=1.09 min; m/z=600.0 for [M+H]⁺

### Step 7: 3-(2-Benzenesulfonamido-2-{4H,5H,6H,7H-[1,3]thiazolo[5,4-c]pyridin-2-yl}ethyl)benzene-1-carboximidamide

To a magnetically stirred solution of tert-butyl 2-(2-{3-[N′-(acetyloxy)carbamimidoyl]phenyl}-1-benzenesulfonamidoethyl)-4H,5H,6H,7H-[1,3]thiazolo[5,4-c]pyridine-5-carboxylate (0.074 g, 0.123 mmol, 1.0 eq.) in AcOH (2.0 mL) was added zinc (0.161 g, 2.47 mmol, 20.0 eq.). The reaction mixture was stirred at room temperature for 18 h. The reaction mixture was filtered through a plug of celite and then concentrated to dryness. The residue was dissolved in DCM (2 mL) and then TFA (1.0 mL) was added. The reaction mixture was stirred at RT for 30 mins and then concentrated to dryness. The residue was purified by Reverse Phase column chromatography on a 120 g C18 cartridge eluting with a 5-95% H₂O:MeCN eluent (0.1% ammonia) to afford product as a white solid (0.006 g, 0.013 mmol, 10% yield). ¹H NMR (400 MHz, DMSO-d₆): δ 7.61 (s, 1H), 7.54-7.38 (m, 5H), 7.35-7.23 (m, 4H), 7.19 (t, J=7.6 Hz, 1H), 4.75 (dd, J=9.3, 5.5 Hz, 1H), 3.82-3.73 (m, 2H), 3.51 (t, J=5.1 Hz, 1H), 3.16 (dd, J=13.7, 5.4 Hz, 2H), 2.97-2.83 (m, 3H). UPLC-MS (basic 6 min): Rt=1.45 min; m/z=442.0 for [M+H]+, 97% purity. Chiral analysis performed by Reach Separations showed 50% chiral purity.

### Example 25. Synthesis of Compound 14

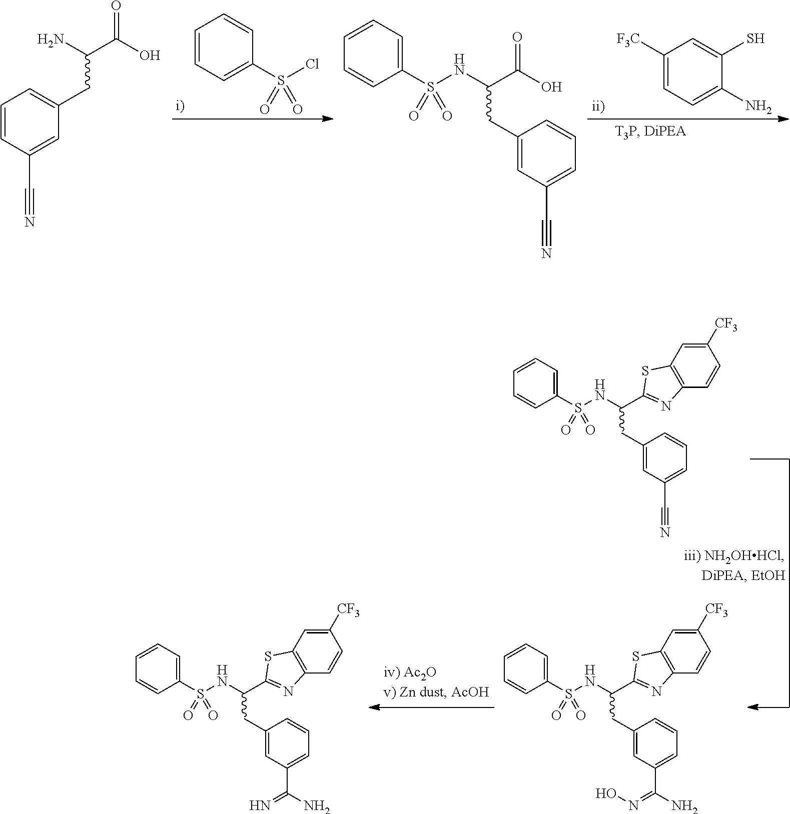

### Step 1: 2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[(1S)-2-(3-Cyanophenyl)-1-[6-(trifluoromethyl)-1,3-benzothiazol-2-yl]ethyl]benzenesulfonamide

2-benzenesulfonamido-3-(3-cyanophenyl)propanoic acid (0.300 g, 0.908 mmol, 1.0 eq.) was dissolved in T3P (50% w/w in EtOAc) (1.1 mL, 1.82 mmol, 2.0 eq.). DIPEA (0.237 mL, 1.36 mmol, 1.5 eq.) and 2-amino-5-(trifluoromethyl)benzene-1-thiol (0.210 g, 1.09 mmol, 1.2 eq.) were added and the reaction mixture was heated at 100° C. under microwave irradiation for 10 mins. The reaction mixture was cooled to RT and then concentrated to dryness. The residue was purified by Reverse Phase column chromatography eluting with a 5-95% H₂O:MeCN eluent (0.1% ammonia) to afford product as a white solid (0.325 g, 0.653 mmol, 72% yield). ¹H NMR (DMSO-d6) δ: 9.06 (s, 1H), 8.66 (t, J=1.2 Hz, 1H), 8.14 (d, J=8.5 Hz, 1H), 7.84 (dd, J=8.6, 1.9 Hz, 1H), 7.61 (t, J=1.7 Hz, 1H), 7.55-7.50 (m, 2H), 7.47-7.43 (m, 2H), 7.42-7.38 (m, 1H), 7.33-7.26 (m, 3H), 5.10 (d, J=9.4 Hz, 1H), 3.41 (dd, J=13.9, 4.5 Hz, 1H), 3.02 (dd, J=13.9, 10.6 Hz, 1H). UPLC-MS (basic 2 min) Rt=1.22 min. m/z=487.9 for [M−H]⁺

### Step 3: 3-[(2S)-2-Benzenesulfonamido-2-[6-(trifluoromethyl)-1,3-benzothiazol-2-yl]ethyl]-N′-hydroxybenzene-1-carboximidamide

To a magnetically stirred solution of N-[(1S)-2-(3-cyanophenyl)-1-[6-(trifluoromethyl)-1,3-benzothiazol-2-yl]ethyl]benzenesulfonamide (0.325 g, 0.667 mmol, 1.0 eq.) in EtOH (3.0 mL) were added hydroxylamine hydrochloride (0.093 g, 1.33 mmol, 2.0 eq.) and DIPEA (0.348 mL, 2.00 mmol, 3.0 eq.). The reaction mixture was stirred under reflux for 1 h. The reaction mixture was cooled down to RT and then concentrated to dryness to afford product as a yellow oil (0.347 g, 0.667 mmol, 100% yield) which was used in the next step without further purification. UPLC-MS (basic 2 min) Rt=1.10 min. m/z=520.9 for [M+H]⁺

### Step 4: [Amino({3-[(2S)-2-benzenesulfonamido-2-[6-(trifluoromethyl)-1,3-benzothiazol-2-yl]ethyl]phenyl})methylidene]amino acetate

To a magnetically stirred solution of 3-[(2S)-2-benzenesulfonamido-2-[6-(trifluoromethyl)-1,3-benzothiazol-2-yl]ethyl]-N′-hydroxybenzene-1-carboximidamide (0.347 g, 0.667 mmol, 1.0 eq) in acetic acid (3.0 mL) was added acetic anhydride (0.189 mL, 2.00 mmol, 3.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% ammonia) to afford product as a white solid (0.210 g, 0.373 mmol, 56% yield). UPLC-MS (basic 2 min): Rt=1.14 min; m/z=562.9 for [M+H]⁺

### Step 5: 3-[(2S)-2-Benzenesulfonamido-2-[6-(trifluoromethyl)-1,3-benzothiazol-2-yl]ethyl]benzene-1-carboximidamide

To a magnetically stirred solution of [amino({3-[(2S)-2-benzenesulfonamido-2-[6-(trifluoromethyl)-1,3-benzothiazol-2-yl]ethyl]phenyl})methylidene]amino acetate (0.210 g, 0.373 mmol, 1.0 eq) in acetic acid (3.0 mL) was added zinc (0.244 g, 3.73 mmol, 10.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford the product as a white solid (0.059 g, 0.117 mmol, 31% yield). ¹H NMR (400 MHz, DMSO-d₆): δ 8.63 (s, 1H), 8.49 (s, 1H), 8.12 (d, J=8.6 Hz, 1H), 7.85-7.80 (m, 1H), 7.72 (s, 1H), 7.55 (d, J=7.9 Hz, 1H), 7.45 (t, J=6.9 Hz, 3H), 7.37 (t, J=7.4 Hz, 1H), 7.26 (dt, J=13.3, 7.7 Hz, 3H), 5.09 (dd, J=9.9, 4.6 Hz, 1H), 3.44-3.39 (m, 1H), 3.06 (dd, J=13.9, 10.0 Hz, 1H). UPLC-MS (basic 6 min): Rt=2.87 min; m/z=504.9 for [M+H]⁺, 99% purity. Chiral analysis performed by Reach Separations showed 50% chiral purity.

### Example 26. Synthesis of Compound 11 (Enantiomer 1)

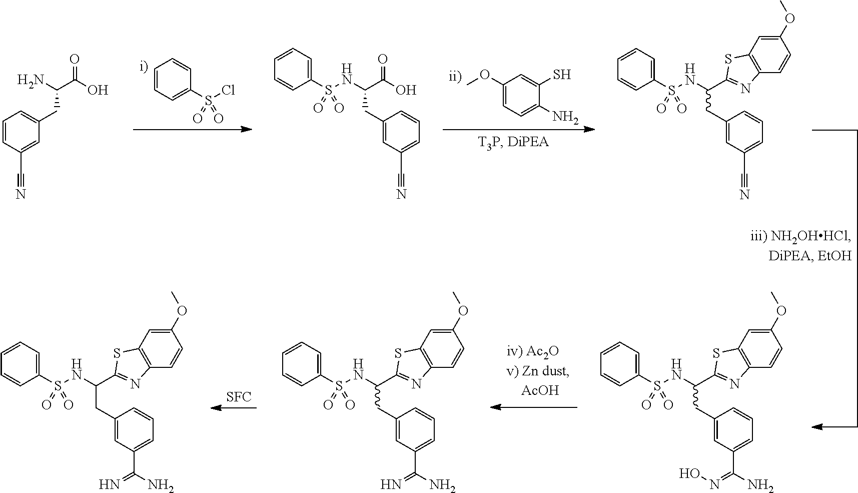

### Step 1: 2-Benzenesulfonamido-3-(3-cyanophenyl)propanoic acid)

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[2-(3-Cyanophenyl)-1-(6-methoxy-1,3-benzothiazol-2-yl)ethyl]benzenesulfonamide

2-Benzenesulfonamido-3-(3-cyanophenyl)propanoic acid (0.300 g, 0.908 mmol, 1.0 eq.) was dissolved in T3P (50% w/w in EtOAc) (1.1 mL, 1.82 mmol, 2.0 eq.). DIPEA (0.237 mL, 1.36 mmol, 1.5 eq.) and 2-amino-5-methoxybenzene-1-thiol (0.169 g, 1.09 mmol, 1.2 eq.) were added and the reaction mixture was heated at 100° C. under microwave irradiation for 10 mins. The reaction mixture was cooled to RT and then concentrated to dryness. The residue was purified by Reverse Phase column chromatography eluting with a 5-95% H₂O:MeCN eluent (0.1% ammonia) to afford product as a white solid (0.197 g, 0.429 mmol, 47% yield). ¹H NMR (DMSO-d6) δ: 8.93 (s, 1H), 7.82 (d, J=8.9 Hz, 1H), 7.65 (d, J=2.6 Hz, 1H), 7.59 (t, J=1.7 Hz, 1H), 7.52 (dq, J=8.2, 2.1, 1.7 Hz, 2H), 7.44 (td, J=7.5, 1.4 Hz, 3H), 7.33-7.26 (m, 3H), 7.10 (dd, J=8.9, 2.6 Hz, 1H), 4.97 (d, J=8.7 Hz, 1H), 2.99 (dd, J=13.9, 10.5 Hz, 1H). UPLC-MS (basic 2 min) Rt=1.10 min. m/z=448.9 for [M−H]⁺

### Step 3: 3-[2-Benzenesulfonamido-2-(6-methoxy-1,3-benzothiazol-2-yl)ethyl]-N′-hydroxybenzene-1-carboximidamide

To a magnetically stirred solution of N-[2-(3-cyanophenyl)-1-(6-methoxy-1,3-benzothiazol-2-yl)ethyl] benzenesulfonamide (0.197 g, 0.438 mmol, 1.0 eq.) in EtOH (2 mL) were added hydroxylamine hydrochloride (0.061 g, 0.876 mmol, 2.0 eq.) and DIPEA (0.229 mL, 1.32 mmol, 3.0 eq.). The reaction mixture was stirred under reflux for 1 h. The reaction mixture was cooled down to RT and then concentrated to dryness to afford product as a yellow oil (0.212 g, 0.439 mmol, 100% yield) which was used in the next step without further purification. UPLC-MS (basic 2 min) Rt=1.00 min. m/z=483.9 for [M+H]⁺

### Step 4: [Amino({3-[2-benzenesulfonamido-2-(6-methoxy-1,3-benzothiazol-2-yl)ethyl]phenyl})methylidene] amino acetate

To a magnetically stirred solution of 3-[2-benzenesulfonamido-2-(6-methoxy-1,3-benzothiazol-2-yl)ethyl]-N′-hydroxybenzene-1-carboximidamide) (0.212 g, 0.439 mmol, 1.0 eq) in acetic acid (2.0 mL) was added acetic anhydride (0.124 mL, 1.32 mmol, 3.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% ammonia) to afford product as a white solid (0.076 g, 0.145 mmol, 33% yield). UPLC-MS (basic 2 min): Rt=1.04 min; m/z=524.9 for [M+H]⁺

### Step 5: 3-[2-Benzenesulfonamido-2-(6-methoxy-1,3-benzothiazol-2-yl)ethyl]benzene-1-carboximidamide

To a magnetically stirred solution of [amino({3-[2-benzenesulfonamido-2-(6-methoxy-1,3-benzothiazol-2-yl)ethyl]phenyl})methylidene]amino acetate (0.076 g, 0.145 mmol, 1.0 eq) in acetic acid (1.0 mL) was added zinc (0.095 g, 1.45 mmol, 10.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% formic acid) to afford the formate salt as a white solid. The formate salt was passed through an SCX column to afford product as a white solid (0.023 g, 0.048 mmol, 34% yield). ¹H NMR (400 MHz, DMSO-d₆): δ 7.75 (d, J=8.9 Hz, 1H), 7.66 (s, 1H), 7.56 (d, J=2.6 Hz, 1H), 7.51 (d, J=7.7 Hz, 1H), 7.46 (d, 2H), 7.34-7.28 (m, 1H), 7.27-7.20 (m, 3H), 7.17 (t, J=7.6 Hz, 1H), 7.04 (dd, J=8.9, 2.6 Hz, 1H), 4.82 (dd, J=8.8, 5.1 Hz, 1H), 3.81 (s, 3H), 3.24 (dd, J=13.6, 5.1 Hz, 1H), 3.01 (dd, J=13.6, 8.9 Hz, 1H). UPLC-MS (basic 4 min): Rt=1.39 min; m/z=466.9 for [M]+, 100% purity. Chiral analysis performed by Reach Separations showed 50% chiral purity.

Product purified by SFC using Lux C2 (20 mm×250 mm, Sum) with a 50:50 MeOH:CO₂ (0.2% v/v NH₃) eluent to afford product as a white solid (0.005 g). ¹H NMR (400 MHz, DMSO-d₆): δ 7.75 (d, J=8.9 Hz, 1H), 7.64 (s, 1H), 7.57 (s, 1H), 7.52-7.45 (m, 2H), 7.33 (t, J=7.4 Hz, 1H), 7.27-7.20 (m, 3H), 7.16 (t, J=7.5 Hz, 1H), 7.04 (d, J=9.0 Hz, 1H), 4.84 (t, 1H), 3.81 (s, 3H), 3.05-2.96 (m, 1H). UPLC-MS (basic 4 min): Rt=1.09 min; m/z=467.2 for [M+H]⁺, 99% purity. Chiral analysis performed by Reach Separations showed 100% chiral purity. Rt=3.51 min.

### Example 27. Synthesis of Compound 11 (Enantiomer 2)

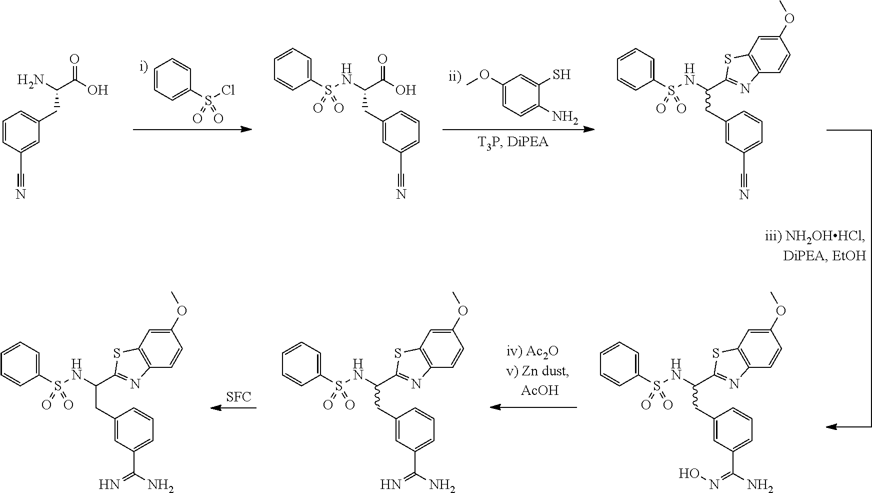

### Step 1: 2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[2-(3-Cyanophenyl)-1-(6-methoxy-1,3-benzothiazol-2-yl)ethyl]benzenesulfonamide

2-benzenesulfonamido-3-(3-cyanophenyl)propanoic acid (0.300 g, 0.908 mmol, 1.0 eq.) was dissolved in T3P (50% w/w in EtOAc) (1.1 mL, 1.82 mmol, 2.0 eq.). DIPEA (0.237 mL, 1.36 mmol, 1.5 eq.) and 2-amino-5-methoxybenzene-1-thiol (0.169 g, 1.09 mmol, 1.2 eq.) were added and the reaction mixture was heated at 100° C. under microwave irradiation for 10 mins. The reaction mixture was cooled to RT and then concentrated to dryness. The residue was purified by Reverse Phase column chromatography eluting with a 5-95% H₂O:MeCN eluent (0.1% ammonia) to afford product as a white solid (0.197 g, 0.429 mmol, 47% yield). ¹H NMR (DMSO-d6) δ: 8.93 (s, 1H), 7.82 (d, J=8.9 Hz, 1H), 7.65 (d, J=2.6 Hz, 1H), 7.59 (t, J=1.7 Hz, 1H), 7.52 (dq, J=8.2, 2.1, 1.7 Hz, 2H), 7.44 (td, J=7.5, 1.4 Hz, 3H), 7.33-7.26 (m, 3H), 7.10 (dd, J=8.9, 2.6 Hz, 1H), 4.97 (d, J=8.7 Hz, 1H), 2.99 (dd, J=13.9, 10.5 Hz, 1H). UPLC-MS (basic 2 min) Rt=1.10 min. m/z=448.9 for [M−H]⁺

### Step 3: 3-[2-Benzenesulfonamido-2-(6-methoxy-1,3-benzothiazol-2-yl)ethyl]-N′-hydroxybenzene-1-carboximidamide

To a magnetically stirred solution of N-[2-(3-cyanophenyl)-1-(6-methoxy-1,3-benzothiazol-2-yl)ethyl] benzenesulfonamide (0.197 g, 0.438 mmol, 1.0 eq.) in EtOH (2 mL) were added hydroxylamine hydrochloride (0.061 g, 0.876 mmol, 2.0 eq.) and DIPEA (0.229 mL, 1.32 mmol, 3.0 eq.). The reaction mixture was stirred under reflux for 1 h. The reaction mixture was cooled down to RT and then concentrated to dryness to afford product as a yellow oil (0.212 g, 0.439 mmol, 100% yield) which was used in the next step without further purification. UPLC-MS (basic 2 min) Rt=1.00 min. m/z=483.9 for [M+H]⁺

### Step 4: [Amino({3-[2-benzenesulfonamido-2-(6-methoxy-1,3-benzothiazol-2-yl)ethyl]phenyl})methylidene] amino acetate

To a magnetically stirred solution of 3-[2-benzenesulfonamido-2-(6-methoxy-1,3-benzothiazol-2-yl)ethyl]-N′-hydroxybenzene-1-carboximidamide) (0.212 g, 0.439 mmol, 1.0 eq) in acetic acid (2.0 mL) was added acetic anhydride (0.124 mL, 1.32 mmol, 3.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% ammonia) to afford product as a white solid (0.076 g, 0.145 mmol, 33% yield). UPLC-MS (basic 2 min): Rt=1.04 min; m/z=524.9 for [M+H]⁺

### Step 5: 3-[2-Benzenesulfonamido-2-(6-methoxy-1,3-benzothiazol-2-yl)ethyl]benzene-1-carboximidamide

To a magnetically stirred solution of [amino({3-[2-benzenesulfonamido-2-(6-methoxy-1,3-benzothiazol-2-yl)ethyl]phenyl})methylidene]amino acetate (0.076 g, 0.145 mmol, 1.0 eq) in acetic acid (1.0 mL) was added zinc (0.095 g, 1.45 mmol, 10.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% formic acid) to afford the formate salt as a white solid. The formate salt was passed through an SCX column to afford product as a white solid (0.023 g, 0.048 mmol, 34% yield). ¹H NMR (400 MHz, DMSO-d₆): δ 7.75 (d, J=8.9 Hz, 1H), 7.66 (s, 1H), 7.56 (d, J=2.6 Hz, 1H), 7.51 (d, J=7.7 Hz, 1H), 7.46 (d, 2H), 7.34-7.28 (m, 1H), 7.27-7.20 (m, 3H), 7.17 (t, J=7.6 Hz, 1H), 7.04 (dd, J=8.9, 2.6 Hz, 1H), 4.82 (dd, J=8.8, 5.1 Hz, 1H), 3.81 (s, 3H), 3.24 (dd, J=13.6, 5.1 Hz, 1H), 3.01 (dd, J=13.6, 8.9 Hz, 1H). UPLC-MS (basic 4 min): Rt=1.39 min; m/z=466.9 for [M]⁺, 100% purity. Chiral analysis performed by Reach Separations showed 50% chiral purity.

Product purified by SFC using Lux C₂ (20 mm×250 mm, Sum) with a 50:50 MeOH:CO₂ (0.2% v/v NH₃) eluent to afford product as a white solid (0.003 g). ¹H NMR (400 MHz, DMSO-d₆): δ 7.74 (d, J=8.9 Hz, 1H), 7.63 (s, 1H), 7.56 (s, 1H), 7.47 (t, J=7.4 Hz, 3H), 7.30 (d, J=7.3 Hz, 1H), 7.22 (t, J=8.3 Hz, 3H), 7.15 (t, J=7.7 Hz, 1H), 7.03 (d, J=8.8 Hz, 1H), 4.81 (t, 1H), 3.81 (s, 3H), 3.00 (dd, J=13.4, 8.5 Hz, 2H). UPLC-MS (basic 4 min): Rt=1.09 min; m/z=467.2 for [M+H]⁺, 100% purity. Chiral analysis performed by Reach Separations showed 99.8% chiral purity. Rt=7.54 min.

### Example 28. Synthesis of Compound 12 (Enantiomer 1)

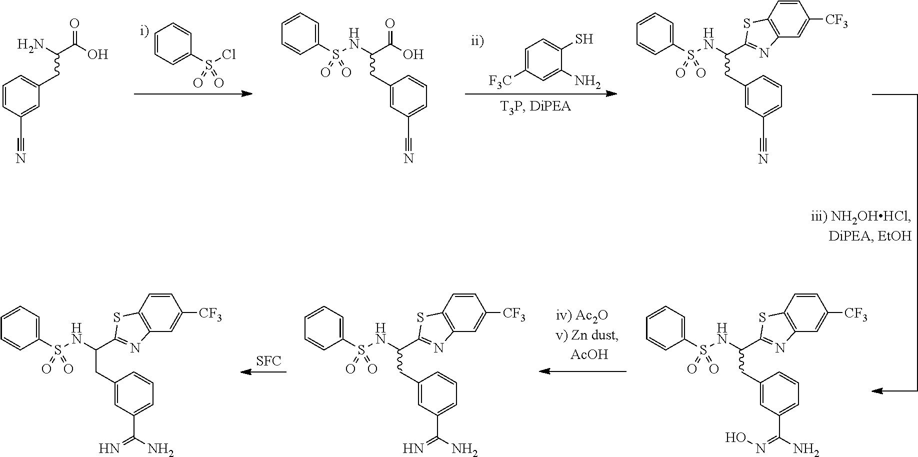

### Step 1: 2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[2-(3-Cyanophenyl)-1-[5-(trifluoromethyl)-1,3-benzothiazol-2-yl]ethyl]benzenesulfonamide

2-benzenesulfonamido-3-(3-cyanophenyl)propanoic acid (0.300 g, 0.908 mmol, 1.0 eq.) was dissolved in T3P (50% w/w in EtOAc) (1.1 mL, 1.82 mmol, 2.0 eq.). DIPEA (0.395 mL, 2.27 mmol, 2.5 eq.) and 2-amino-4-(trifluoromethyl)benzene-1-thiol hydrochloride (0.250 g, 1.09 mmol, 1.2 eq.) were added and the reaction mixture was heated at 100° C. under microwave irradiation for 10 mins. The reaction mixture was cooled to RT and then concentrated to dryness. The residue was purified by Reverse Phase column chromatography eluting with a 5-95% H₂O:MeCN eluent (0.1% ammonia) to afford product as a white solid (0.218 g, 0.438 mmol, 48% yield). ¹H NMR (DMSO-d6) δ: 9.08 (s, 1H), 8.38 (d, J=8.4 Hz, 1H), 8.30 (s, 1H), 7.79 (dd, J=8.5, 1.8 Hz, 1H), 7.61 (s, 1H), 7.53 (dd, J=7.8, 1.6 Hz, 2H), 7.47-7.39 (m, 3H), 7.33-7.25 (m, 3H), 5.10 (dd, J=10.6, 4.4 Hz, 1H), 3.40 (dd, J=13.9, 4.5 Hz, 1H), 3.02 (dd, J=13.9, 10.6 Hz, 1H). UPLC-MS (basic 2 min) Rt=1.22 min. m/z=486.1 for [M−H]⁺

### Step 3: 3-{2-Benzenesulfonamido-2-[5-(trifluoromethyl)-1,3-benzothiazol-2-yl]ethyl}-N′-hydroxybenzene-1-carboximidamide

To a magnetically stirred solution of N-[2-(3-cyanophenyl)-1-[5-(trifluoromethyl)-1,3-benzothiazol-2-yl]ethyl]benzenesulfonamide (0.218 g, 0.447 mmol, 1.0 eq.) in EtOH (2 mL) were added hydroxylamine hydrochloride (0.062 g, 0.894 mmol, 2.0 eq.) and DIPEA (0.234 mL, 1.34 mmol, 3.0 eq.). The reaction mixture was stirred under reflux for 1 h. The reaction mixture was cooled down to RT and then concentrated to dryness to afford product as a yellow oil (0.233 g, 0.448 mmol, 100% yield) which was used in the next step without further purification. UPLC-MS (basic 2 min) Rt=1.12 min. m/z=520.9 for [M+H]⁺

### Step 4: [Amino(3-{2-benzenesulfonamido-2-[5-(trifluoromethyl)-1,3-benzothiazol-2-yl]ethyl}phenyl) methylidene]amino acetate

To a magnetically stirred solution of 3-{2-benzenesulfonamido-2-[5-(trifluoromethyl)-1,3-benzothiazol-2-yl]ethyl}-N′-hydroxybenzene-1-carboximidamide (0.236 g, 0.453 mmol, 1.0 eq) in acetic acid (2.0 mL) was added acetic anhydride (0.128 mL, 1.36 mmol, 3.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% ammonia) to afford product as a white solid (0.061 g, 0.108 mmol, 24% yield). UPLC-MS (basic 2 min): Rt=1.14 min; m/z=562.9 for [M+H]⁺

### Step 5: 3-{2-Benzenesulfonamido-2-[5-(trifluoromethyl)-1,3-benzothiazol-2-yl]ethyl}benzene-1-carboximidamide

To a magnetically stirred solution of [amino(3-{2-benzenesulfonamido-2-[5-(trifluoromethyl)-1,3-benzothiazol-2-yl]ethyl}phenyl)methylidene]amino acetate) (0.061 g, 0.108 mmol, 1.0 eq) in acetic acid (1.0 mL) was added zinc (0.071 g, 1.08 mmol, 10.0 eq.). The reaction mixture was stirred at RT for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% formic acid) to afford the formate salt as a white solid. The formate salt was passed through an SCX column to afford product as a white solid (0.021 g, 0.042 mmol, 38% yield). ¹H NMR (400 MHz, DMSO-d₆): δ 8.25 (d, J=8.4 Hz, 1H), 8.20 (s, 1H), 7.71-7.66 (m, 2H), 7.52 (d, 1H), 7.47-7.42 (m, 2H), 7.31-7.17 (m, 5H), 4.85 (dd, J=8.7, 4.8 Hz, 1H), 3.25 (dd, 1H), 3.04 (dd, J=13.3, 8.7 Hz, 1H). UPLC-MS (basic 4 min): Rt=1.61 min; m/z=504.9 for [M]+, 100% purity. Chiral analysis performed by Reach Separations showed 50% chiral purity.

Product purified by SFC using Lux C2 (20 mm×250 mm, Sum) with a 50:50 MeOH:CO₂ (0.2% v/v NH₃) eluent to afford product as a white solid (0.004 g). ¹H NMR (400 MHz, DMSO-d₆): δ 8.25 (d, J=8.3 Hz, 1H), 8.19 (s, 1H), 7.68 (s, 2H), 7.51 (d, J=7.7 Hz, 1H), 7.45 (d, J=7.6 Hz, 2H), 7.27 (dd, J=14.1, 7.2 Hz, 2H), 7.20 (t, J=7.4 Hz, 2H), 4.88-4.81 (m, 1H), 3.07-2.99 (m, 2H). UPLC-MS (basic 4 min): Rt=1.24 min; m/z=505.1 for [M+H]+, 99% purity. Chiral analysis performed by Reach Separations showed 100% chiral purity. Rt=1.75 min.

### Example 29. Synthesis of Compound 12 (Enantiomer 2)

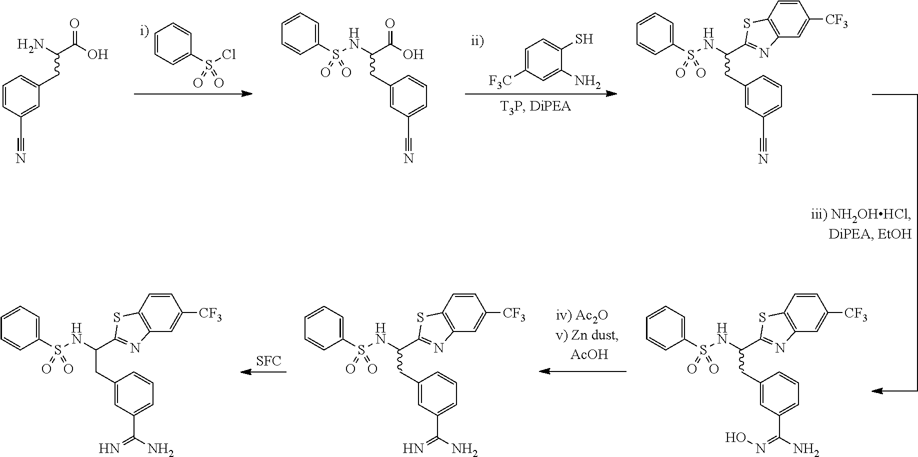

### Step 1: 2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[2-(3-Cyanophenyl)-1-[5-(trifluoromethyl)-1,3-benzothiazol-2-yl]ethyl]benzenesulfonamide

2-benzenesulfonamido-3-(3-cyanophenyl)propanoic acid (0.300 g, 0.908 mmol, 1.0 eq.) was dissolved in T3P (50% w/w in EtOAc) (1.1 mL, 1.82 mmol, 2.0 eq.). DIPEA (0.395 mL, 2.27 mmol, 2.5 eq.) and 2-amino-4-(trifluoromethyl)benzene-1-thiol hydrochloride (0.250 g, 1.09 mmol, 1.2 eq.) were added and the reaction mixture was heated at 100° C. under microwave irradiation for 10 mins. The reaction mixture was cooled to RT and then concentrated to dryness. The residue was purified by Reverse Phase column chromatography eluting with a 5-95% H₂O:MeCN eluent (0.1% ammonia) to afford product as a white solid (0.218 g, 0.438 mmol, 48% yield). ¹H NMR (DMSO-d6) δ: 9.08 (s, 1H), 8.38 (d, J=8.4 Hz, 1H), 8.30 (s, 1H), 7.79 (dd, J=8.5, 1.8 Hz, 1H), 7.61 (s, 1H), 7.53 (dd, J=7.8, 1.6 Hz, 2H), 7.47-7.39 (m, 3H), 7.33-7.25 (m, 3H), 5.10 (dd, J=10.6, 4.4 Hz, 1H), 3.40 (dd, J=13.9, 4.5 Hz, 1H), 3.02 (dd, J=13.9, 10.6 Hz, 1H). UPLC-MS (basic 2 min) Rt=1.22 min. m/z=486.1 for [M−H]⁺

### Step 3: 3-{2-Benzenesulfonamido-2-[5-(trifluoromethyl)-1,3-benzothiazol-2-yl]ethyl}-N′-hydroxybenzene-1-carboximidamide

To a magnetically stirred solution of N-[2-(3-cyanophenyl)-1-[5-(trifluoromethyl)-1,3-benzothiazol-2-yl]ethyl]benzenesulfonamide (0.218 g, 0.447 mmol, 1.0 eq.) in EtOH (2 mL) were added hydroxylamine hydrochloride (0.062 g, 0.894 mmol, 2.0 eq.) and DIPEA (0.234 mL, 1.34 mmol, 3.0 eq.). The reaction mixture was stirred under reflux for 1 h. The reaction mixture was cooled down to RT and then concentrated to dryness to afford product as a yellow oil (0.233 g, 0.448 mmol, 100% yield) which was used in the next step without further purification. UPLC-MS (basic 2 min) Rt=1.12 min. m/z=520.9 for [M+H]⁺

### Step 4: [Amino(3-{2-benzenesulfonamido-2-[5-(trifluoromethyl)-1,3-benzothiazol-2-yl]ethyl}phenyl) methylidene]amino acetate

To a magnetically stirred solution of 3-{2-benzenesulfonamido-2-[5-(trifluoromethyl)-1,3-benzothiazol-2-yl]ethyl}-N′-hydroxybenzene-1-carboximidamide (0.236 g, 0.453 mmol, 1.0 eq) in acetic acid (2.0 mL) was added acetic anhydride (0.128 mL, 1.36 mmol, 3.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% ammonia) to afford product as a white solid (0.061 g, 0.108 mmol, 24% yield). UPLC-MS (basic 2 min): Rt=1.14 min; m/z=562.9 for [M+H]⁺

### Step 5: 3-{2-Benzenesulfonamido-2-[5-(trifluoromethyl)-1,3-benzothiazol-2-yl]ethyl}benzene-1-carboximidamide

To a magnetically stirred solution of [amino(3-{2-benzenesulfonamido-2-[5-(trifluoromethyl)-1,3-benzothiazol-2-yl]ethyl}phenyl)methylidene]amino acetate) (0.061 g, 0.108 mmol, 1.0 eq) in acetic acid (1.0 mL) was added zinc (0.071 g, 1.08 mmol, 10.0 eq.). The reaction mixture was stirred at RT for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% formic acid) to afford the formate salt as a white solid. The formate salt was passed through an SCX column to afford product as a white solid (0.021 g, 0.042 mmol, 38% yield). ¹H NMR (400 MHz, DMSO-d₆): δ 8.25 (d, J=8.4 Hz, 1H), 8.20 (s, 1H), 7.71-7.66 (m, 2H), 7.52 (d, 1H), 7.47-7.42 (m, 2H), 7.31-7.17 (m, 5H), 4.85 (dd, J=8.7, 4.8 Hz, 1H), 3.25 (dd, 1H), 3.04 (dd, J=13.3, 8.7 Hz, 1H). UPLC-MS (basic 4 min): Rt=1.61 min; m/z=504.9 for [M]⁺, 100% purity. Chiral analysis performed by Reach Separations showed 50% chiral purity.

Product purified by SFC using Lux C₂ (20 mm×250 mm, Sum) with a 50:50 MeOH:CO₂ (0.2% v/v NH₃) eluent to afford product as a white solid (0.003 g). ¹H NMR (400 MHz, DMSO-d₆): δ 8.25 (d, J=8.5 Hz, 1H), 8.20 (s, 1H), 7.70-7.66 (m, 2H), 7.52 (d, J=7.7 Hz, 1H), 7.45 (d, J=7.5 Hz, 2H), 7.31-7.23 (m, 2H), 7.23-7.17 (m, 3H), 4.85 (s, 1H), 3.03 (dd, J=13.4, 8.7 Hz, 1H). UPLC-MS (basic 4 min): Rt=1.23 min; m/z=505.1 for [M+H]⁺, 100% purity. Chiral analysis performed by Reach Separations showed 99.8% chiral purity. Rt=3.53 min.

### Example 30. Synthesis of Compound 13 (Enantiomer 1)

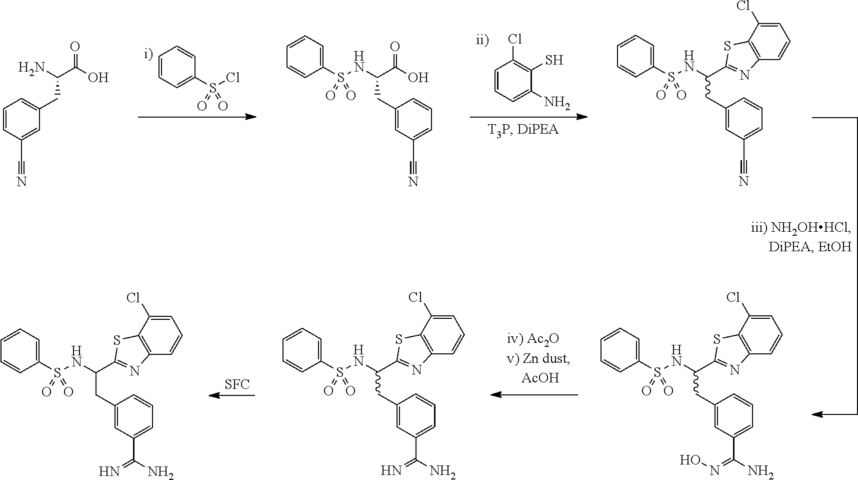

### Step 1: 2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[1-(7-Chloro-1,3-benzothiazol-2-yl)-2-(3-cyanophenyl)ethyl]benzenesulfonamide

2-benzenesulfonamido-3-(3-cyanophenyl)propanoic acid (0.505 g, 1.53 mmol, 1.0 eq.) was dissolved in toluene (10.0 mL) and then T3P (50% w/w in EtOAc) (2.0 mL, 3.43 mmol, 2.2 eq.). DIPEA (0.800 mL, 4.59 mmol, 3.0 eq.) and 2-amino-6-chlorobenzene-1-thiol hydrochloride (0.330 g, 1.69 mmol, 1.1 eq.) were added. The reaction mixture was stirred at RT for 30 mins. and then heated at 100° C. for 1 h. The reaction mixture was cooled to RT and then aq. 1M HCl solution was added. The product was extracted with DCM (3×25 mL) and the combined organic phase was washed with aq. sat. NaHCO₃ solution (25 mL), H₂O (25 mL), dried over anhydrous sodium sulfate, filtered and then concentrated to dryness. The residue was purified by Reverse Phase column chromatography eluting with a 5-95% H₂O:MeCN eluent (0.1% ammonia) to afford product as a white solid (0.392 g, 0.864 mmol, 57% yield). ¹H NMR (DMSO-d6) δ: 9.04 (br d, J=6.4 Hz, 1H), 7.95 (dd, J=6.7, 2.3 Hz, 1H), 7.60-7.63 (m, 1H), 7.54-7.60 (m, 2H), 7.53 (dd, J=7.8, 1.5 Hz, 2H), 7.38-7.49 (m, 3H), 7.29 (q, J=8.3 Hz, 3H), 4.98-5.14 (m, 1H), 3.40 (dd, J=13.9, 4.4 Hz, 1H), 3.01 (dd, J=13.9, 10.8 Hz, 1H). UPLC-MS (basic 2 min) Rt=1.21 min. m/z=453.9 for [M]⁺

### Step 3: 3-[2-Benzenesulfonamido-2-(7-chloro-1,3-benzothiazol-2-yl)ethyl]-N′-hydroxybenzene-1-carboximidamide

To a magnetically stirred solution of N-[1-(7-chloro-1,3-benzothiazol-2-yl)-2-(3-cyanophenyl)ethyl]benzenesulfonamide (0.228 g, 0.502 mmol, 1.0 eq.) in EtOH (6.0 mL) were added hydroxylamine hydrochloride (0.105 g, 1.51 mmol, 3.0 eq.) and DIPEA (0.350 mL, 2.01 mmol, 4.0 eq.). The reaction mixture was stirred under reflux for 1 h. The reaction mixture was cooled down to RT and then concentrated to dryness to afford product as a yellow oil (0.249 g, 0.511 mmol, 100% yield) which was used in the next step without further purification. UPLC-MS (basic 2 min) Rt=1.08 min. m/z=486.9 for [M]⁺

### Step 4: [Amino({3-[2-benzenesulfonamido-2-(7-chloro-1,3-benzothiazol-2-yl)ethyl]phenyl})methylidene] amino acetate

To a magnetically stirred solution of 3-[2-benzenesulfonamido-2-(7-chloro-1,3-benzothiazol-2-yl)ethyl]-N′-hydroxybenzene-1-carboximidamide (0.249 g, 0.511 mmol, 1.0 eq) in acetic acid (5.0 mL) was added acetic anhydride (0.20 mL, 2.12 mmol, 4.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% ammonia) to afford product as a white solid (0.083 g, 0.157 mmol, 31% yield). UPLC-MS (basic 2 min): Rt=1.12 min; m/z=528.8 for [M]⁺

### Step 5: 3-[2-Benzenesulfonamido-2-(7-chloro-1,3-benzothiazol-2-yl)ethyl]benzene-1-carboximidamide

To a magnetically stirred solution of [amino({3-[2-benzenesulfonamido-2-(7-chloro-1,3-benzothiazol-2-yl)ethyl]phenyl})methylidene]amino acetate (0.083 g, 0.157 mmol, 1.0 eq) in acetic acid (2.0 mL) was added zinc (0.212 g, 3.24 mmol, 20.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% formic acid) to afford the formate salt as a white solid. The formate salt was passed through an SAX column to afford product as a white solid (0.027 g, 0.057 mmol, 37% yield). ¹H NMR (400 MHz, DMSO-d₆): δ 7.92 (dd, J=6.8, 2.2 Hz, 1H), 7.74 (s, 1H), 7.53-7.61 (m, 3H), 7.43-7.53 (m, 3H), 7.35-7.42 (m, 1H), 7.28-7.34 (m, 1H), 7.20-7.27 (m, 2H), 5.09 (br dd, J=10.0, 4.6 Hz, 1H), 3.40 (dd, J=13.9, 4.9 Hz, 1H), 3.08 (dd, J=13.8, 10.1 Hz, 1H). UPLC-MS (basic 6 min): Rt=2.69 min; m/z=470.9 for [M]⁺, 100% purity. Chiral analysis performed by Reach Separations showed 66% chiral purity.

Product purified by SFC using Lux C2 (20 mm×250 mm, Sum) with a 50:50 MeOH:CO₂ (0.2% v/v NH₃) eluent to afford product as a white solid (0.013 g). 1H NMR (400 MHz, DMSO-d6): δ 7.84 (br dd, J=5.7, 2.8 Hz, 1H), 7.69 (s, 1H), 7.49-7.55 (m, 1H), 7.45-7.49 (m, 2H), 7.39-7.45 (m, 2H), 7.23-7.30 (m, 2H), 7.16-7.23 (m, 3H), 4.77 (br dd, J=8.2, 4.5 Hz, 1H), 3.19-3.23 (m, 1H), 3.01 (br dd, J=13.2, 8.8 Hz, 1H). UPLC-MS (acidic 4 min): Rt=1.19 min; m/z=471.1 for [M]⁺, 100% purity. Chiral analysis performed by Reach Separations showed 99.5% chiral purity. Rt=3.12 min.

### Example 31. Synthesis of Compound 13 (Enantiomer 2)

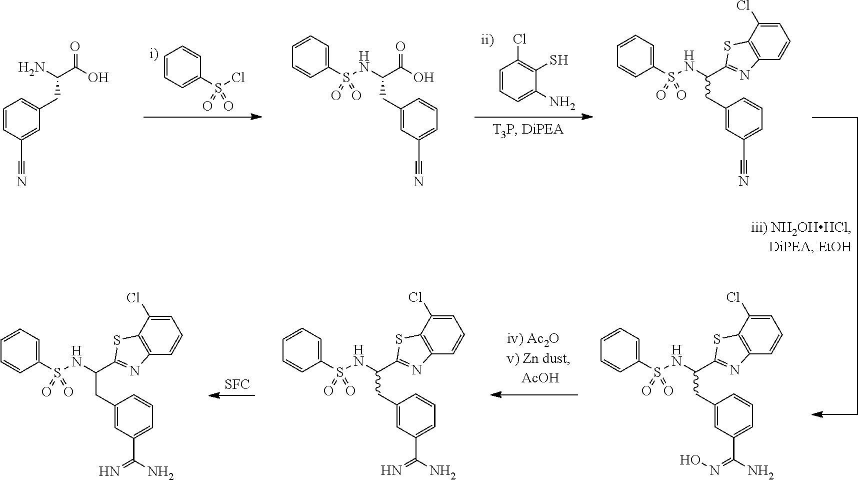

### Step 1: 2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[1-(7-Chloro-1,3-benzothiazol-2-yl)-2-(3-cyanophenyl)ethyl]benzenesulfonamide

2-benzenesulfonamido-3-(3-cyanophenyl)propanoic acid (0.505 g, 1.53 mmol, 1.0 eq.) was dissolved in toluene (10.0 mL) and then T3P (50% w/w in EtOAc) (2.0 mL, 3.43 mmol, 2.2 eq.). DIPEA (0.800 mL, 4.59 mmol, 3.0 eq.) and 2-amino-6-chlorobenzene-1-thiol hydrochloride (0.330 g, 1.69 mmol, 1.1 eq.) were added. The reaction mixture was stirred at RT for 30 mins. and then heated at 100° C. for 1 h. The reaction mixture was cooled to RT and then aq. 1M HCl solution was added. The product was extracted with DCM (3×25 mL) and the combined organic phase was washed with aq. sat. NaHCO₃ solution (25 mL), H₂O (25 mL), dried over anhydrous sodium sulfate, filtered and then concentrated to dryness. The residue was purified by Reverse Phase column chromatography eluting with a 5-95% H₂O:MeCN eluent (0.1% ammonia) to afford product as a white solid (0.392 g, 0.864 mmol, 57% yield). ¹H NMR (DMSO-d6) δ: 9.04 (br d, J=6.4 Hz, 1H), 7.95 (dd, J=6.7, 2.3 Hz, 1H), 7.60-7.63 (m, 1H), 7.54-7.60 (m, 2H), 7.53 (dd, J=7.8, 1.5 Hz, 2H), 7.38-7.49 (m, 3H), 7.29 (q, J=8.3 Hz, 3H), 4.98-5.14 (m, 1H), 3.40 (dd, J=13.9, 4.4 Hz, 1H), 3.01 (dd, J=13.9, 10.8 Hz, 1H). UPLC-MS (basic 2 min). Rt=1.21 min. m/z=453.9 for [M]⁺

### Step 3: 3-[2-Benzenesulfonamido-2-(7-chloro-1,3-benzothiazol-2-yl)ethyl]-N′-hydroxybenzene-1-carboximidamide

To a magnetically stirred solution of N-[1-(7-chloro-1,3-benzothiazol-2-yl)-2-(3-cyanophenyl)ethyl]benzenesulfonamide (0.228 g, 0.502 mmol, 1.0 eq.) in EtOH (6.0 mL) were added hydroxylamine hydrochloride (0.105 g, 1.51 mmol, 3.0 eq.) and DIPEA (0.350 mL, 2.01 mmol, 4.0 eq.). The reaction mixture was stirred under reflux for 1 h. The reaction mixture was cooled down to RT and then concentrated to dryness to afford product as a yellow oil (0.249 g, 0.511 mmol, 100% yield) which was used in the next step without further purification. UPLC-MS (basic 2 min). Rt=1.08 min. m/z=486.9 for [M]⁺

### Step 4: [Amino({3-[2-benzenesulfonamido-2-(7-chloro-1,3-benzothiazol-2-yl)ethyl]phenyl})methylidene] amino acetate

To a magnetically stirred solution of 3-[2-benzenesulfonamido-2-(7-chloro-1,3-benzothiazol-2-yl)ethyl]-N′-hydroxybenzene-1-carboximidamide (0.249 g, 0.511 mmol, 1.0 eq) in acetic acid (5.0 mL) was added acetic anhydride (0.20 mL, 2.12 mmol, 4.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% ammonia) to afford product as a white solid (0.083 g, 0.157 mmol, 31% yield). UPLC-MS (basic 2 min): Rt=1.12 min; m/z=528.8 for [M]⁺

### Step 5: 3-[2-Benzenesulfonamido-2-(7-chloro-1,3-benzothiazol-2-yl)ethyl]benzene-1-carboximidamide

To a magnetically stirred solution of [amino({3-[2-benzenesulfonamido-2-(7-chloro-1,3-benzothiazol-2-yl)ethyl]phenyl})methylidene]amino acetate (0.083 g, 0.157 mmol, 1.0 eq) in acetic acid (2.0 mL) was added zinc (0.212 g, 3.24 mmol, 20.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% formic acid) to afford the formate salt as a white solid. The formate salt was passed through an SAX column to afford product as a white solid (0.027 g, 0.057 mmol, 37% yield). ¹H NMR (400 MHz, DMSO-d₆): δ 7.92 (dd, J=6.8, 2.2 Hz, 1H), 7.74 (s, 1H), 7.53-7.61 (m, 3H), 7.43-7.53 (m, 3H), 7.35-7.42 (m, 1H), 7.28-7.34 (m, 1H), 7.20-7.27 (m, 2H), 5.09 (br dd, J=10.0, 4.6 Hz, 1H), 3.40 (dd, J=13.9, 4.9 Hz, 1H), 3.08 (dd, J=13.8, 10.1 Hz, 1H). UPLC-MS (basic 6 min): Rt=2.69 min; m/z=470.9 for [M]⁺, 100% purity. Chiral analysis performed by Reach Separations showed 66% chiral purity.

Product purified by SFC using Lux C2 (20 mm×250 mm, Sum) with a 50:50 MeOH:CO₂ (0.2% v/v NH₃) eluent to afford product as a white solid (0.008 g). 1H NMR (400 MHz, DMSO-d6): δ 7.92 (br d, J=6.8 Hz, 1H), 7.74 (s, 1H), 7.54-7.60 (m, 3H), 7.44-7.54 (m, 3H), 7.35-7.43 (m, 1H), 7.31 (s, 1H), 7.21-7.26 (m, 2H), 5.09 (dd, J=9.9, 4.5 Hz, 1H), 3.36-3.43 (m, 1H), 3.02-3.14 (m, 1H). UPLC-MS (acidic 4 min): Rt=1.19 min; m/z=471.1 for [M]+, 100% purity. Chiral analysis performed by Reach Separations showed 99.9% chiral purity. Rt=7.25 min.

### Example 32. Synthesis of Compound 14 (Enantiomer 1)

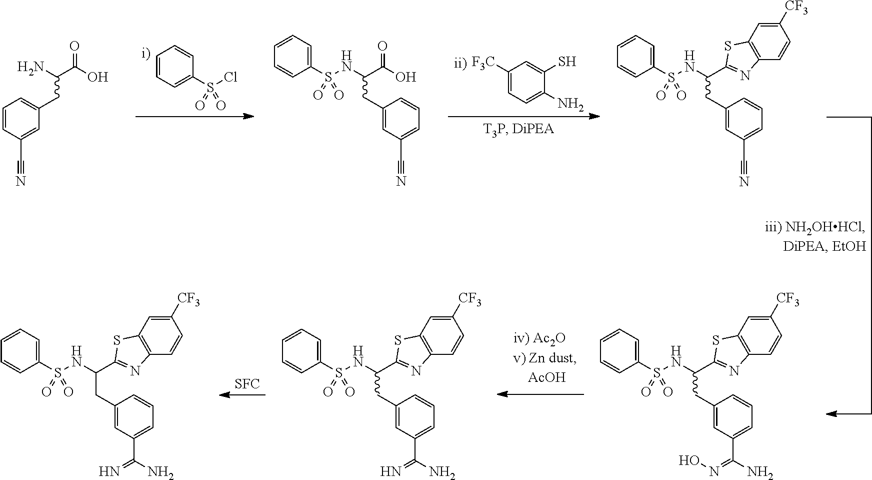

### Step 1: 2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[(1S)-2-(3-cyanophenyl)-1-[6-(trifluoromethyl)-1,3-benzothiazol-2-yl]ethyl]benzenesulfonamide

2-benzenesulfonamido-3-(3-cyanophenyl)propanoic acid (0.300 g, 0.908 mmol, 1.0 eq.) was dissolved in T3P (50% w/w in EtOAc) (1.1 mL, 1.82 mmol, 2.0 eq.). DIPEA (0.237 mL, 1.36 mmol, 1.5 eq.) and 2-amino-5-(trifluoromethyl)benzene-1-thiol (0.210 g, 1.09 mmol, 1.2 eq.) were added and the reaction mixture was heated at 100° C. under microwave irradiation for 10 mins. The reaction mixture was cooled to RT and then concentrated to dryness. The residue was purified by Reverse Phase column chromatography eluting with a 5-95% H₂O:MeCN eluent (0.1% ammonia) to afford product as a white solid (0.325 g, 0.653 mmol, 72% yield). ¹H NMR (DMSO-d6) δ: 9.06 (s, 1H), 8.66 (t, J=1.2 Hz, 1H), 8.14 (d, J=8.5 Hz, 1H), 7.84 (dd, J=8.6, 1.9 Hz, 1H), 7.61 (t, J=1.7 Hz, 1H), 7.55-7.50 (m, 2H), 7.47-7.43 (m, 2H), 7.42-7.38 (m, 1H), 7.33-7.26 (m, 3H), 5.10 (d, J=9.4 Hz, 1H), 3.41 (dd, J=13.9, 4.5 Hz, 1H), 3.02 (dd, J=13.9, 10.6 Hz, 1H). UPLC-MS (basic 2 min) Rt=1.22 min. m/z=487.9 for [M−H]⁺

### Step 3: 3-[(2S)-2-Benzenesulfonamido-2-[6-(trifluoromethyl)-1,3-benzothiazol-2-yl]ethyl]-N′-hydroxybenzene-1-carboximidamide

To a magnetically stirred solution of N-[(1S)-2-(3-cyanophenyl)-1-[6-(trifluoromethyl)-1,3-benzothiazol-2-yl]ethyl]benzenesulfonamide (0.325 g, 0.667 mmol, 1.0 eq.) in EtOH (3.0 mL) were added hydroxylamine hydrochloride (0.093 g, 1.33 mmol, 2.0 eq.) and DIPEA (0.348 mL, 2.00 mmol, 3.0 eq.). The reaction mixture was stirred under reflux for 1 h. The reaction mixture was cooled down to RT and then concentrated to dryness to afford product as a yellow oil (0.347 g, 0.667 mmol, 100% yield) which was used in the next step without further purification. UPLC-MS (basic 2 min) Rt=1.10 min. m/z=520.9 for [M+H]⁺

### Step 4: [Amino({3-[(2S)-2-benzenesulfonamido-2-[6-(trifluoromethyl)-1,3-benzothiazol-2-yl]ethyl]phenyl})methylidene]amino acetate

To a magnetically stirred solution of 3-[(2S)-2-benzenesulfonamido-2-[6-(trifluoromethyl)-1,3-benzothiazol-2-yl]ethyl]-N′-hydroxybenzene-1-carboximidamide (0.347 g, 0.667 mmol, 1.0 eq) in acetic acid (3.0 mL) was added acetic anhydride (0.189 mL, 2.00 mmol, 3.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% ammonia) to afford product as a white solid (0.210 g, 0.373 mmol, 56% yield). UPLC-MS (basic 2 min): Rt=1.14 min; m/z=562.9 for [M+H]⁺

### Step 5: 3-[(2S)-2-Benzenesulfonamido-2-[6-(trifluoromethyl)-1,3-benzothiazol-2-yl]ethyl]benzene-1-carboximidamide

To a magnetically stirred solution of [amino({3-[(2S)-2-benzenesulfonamido-2-[6-(trifluoromethyl)-1,3-benzothiazol-2-yl]ethyl]phenyl})methylidene]amino acetate (0.210 g, 0.373 mmol, 1.0 eq) in acetic acid (3.0 mL) was added zinc (0.244 g, 3.73 mmol, 10.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford the product as a white solid (0.059 g, 0.117 mmol, 31% yield). ¹H NMR (400 MHz, DMSO-d₆): δ 8.63 (s, 1H), 8.49 (s, 1H), 8.12 (d, J=8.6 Hz, 1H), 7.85-7.80 (m, 1H), 7.72 (s, 1H), 7.55 (d, J=7.9 Hz, 1H), 7.45 (t, J=6.9 Hz, 3H), 7.37 (t, J=7.4 Hz, 1H), 7.26 (dt, J=13.3, 7.7 Hz, 3H), 5.09 (dd, J=9.9, 4.6 Hz, 1H), 3.44-3.39 (m, 1H), 3.06 (dd, J=13.9, 10.0 Hz, 1H). UPLC-MS (basic 6 min): Rt=2.87 min; m/z=504.9 for [M+H]⁺, 99% purity. Chiral analysis performed by Reach Separations showed 50% chiral purity.

Product purified by SFC using Lux C₄ (21.2 mm×250 mm, Sum) with a 60:40 MeOH:CO₂ (0.2% v/v NH₃) eluent to afford product as a white solid (0.018 g). 1H NMR (400 MHz, DMSO-d6): δ 8.53 (s, 1H), 8.06 (d, J=8.5 Hz, 1H), 7.77 (d, J=8.6 Hz, 1H), 7.70 (s, 1H), 7.52 (d, J=7.8 Hz, 1H), 7.45 (d, J=7.6 Hz, 2H), 7.29 (dd, J=14.0, 7.4 Hz, 2H), 7.21 (t, J=7.4 Hz, 3H), 4.87 (s, 1H), 3.03 (dd, J=13.4, 8.8 Hz, 1H). UPLC-MS (basic 4 min): Rt=1.24 min; m/z=505.1 for [M+H]⁺, 100% purity. Chiral analysis performed by Reach Separations showed 98.4% chiral purity. Rt=1.18 min.

### Example 33. Synthesis of Compound 14 (Enantiomer 2)

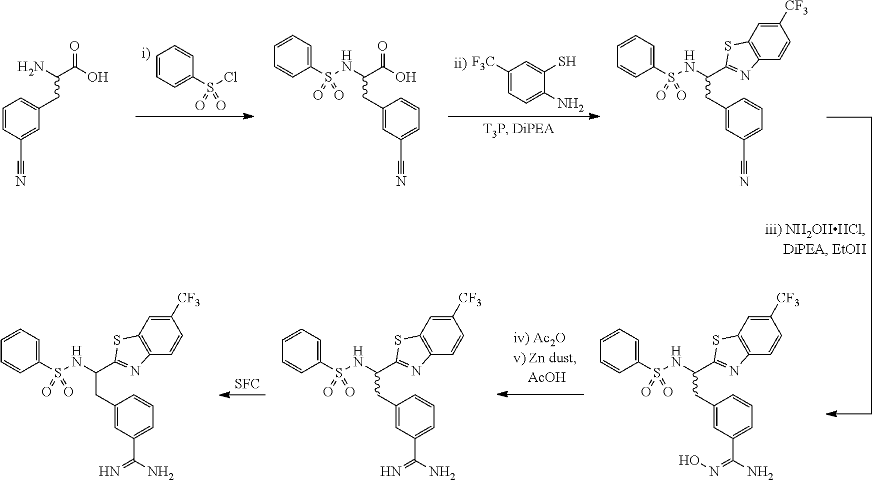

### Step 1: 2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[(1S)-2-(3-Cyanophenyl)-1-[6-(trifluoromethyl)-1,3-benzothiazol-2-yl]ethyl]benzenesulfonamide

2-Benzenesulfonamido-3-(3-cyanophenyl)propanoic acid (0.300 g, 0.908 mmol, 1.0 eq.) was dissolved in T3P (50% w/w in EtOAc) (1.1 mL, 1.82 mmol, 2.0 eq.). DIPEA (0.237 mL, 1.36 mmol, 1.5 eq.) and 2-amino-5-(trifluoromethyl)benzene-1-thiol (0.210 g, 1.09 mmol, 1.2 eq.) were added and the reaction mixture was heated at 100° C. under microwave irradiation for 10 mins. The reaction mixture was cooled to RT and then concentrated to dryness. The residue was purified by Reverse Phase column chromatography eluting with a 5-95% H₂O:MeCN eluent (0.1% ammonia) to afford product as a white solid (0.325 g, 0.653 mmol, 72% yield). ¹H NMR (DMSO-d6) δ: 9.06 (s, 1H), 8.66 (t, J=1.2 Hz, 1H), 8.14 (d, J=8.5 Hz, 1H), 7.84 (dd, J=8.6, 1.9 Hz, 1H), 7.61 (t, J=1.7 Hz, 1H), 7.55-7.50 (m, 2H), 7.47-7.43 (m, 2H), 7.42-7.38 (m, 1H), 7.33-7.26 (m, 3H), 5.10 (d, J=9.4 Hz, 1H), 3.41 (dd, J=13.9, 4.5 Hz, 1H), 3.02 (dd, J=13.9, 10.6 Hz, 1H). UPLC-MS (basic 2 min) Rt=1.22 min. m/z=487.9 for [M−H]⁺

### Step 3: 3-[(2S)-2-Benzenesulfonamido-2-[6-(trifluoromethyl)-1,3-benzothiazol-2-yl]ethyl]-N′-hydroxybenzene-1-carboximidamide

To a magnetically stirred solution of N-[(1S)-2-(3-cyanophenyl)-1-[6-(trifluoromethyl)-1,3-benzothiazol-2-yl]ethyl]benzenesulfonamide (0.325 g, 0.667 mmol, 1.0 eq.) in EtOH (3.0 mL) were added hydroxylamine hydrochloride (0.093 g, 1.33 mmol, 2.0 eq.) and DIPEA (0.348 mL, 2.00 mmol, 3.0 eq.). The reaction mixture was stirred under reflux for 1 h. The reaction mixture was cooled down to RT and then concentrated to dryness to afford product as a yellow oil (0.347 g, 0.667 mmol, 100% yield) which was used in the next step without further purification. UPLC-MS (basic 2 min) Rt=1.10 min. m/z=520.9 for [M+H]⁺

### Step 4: [Amino({3-[(2S)-2-benzenesulfonamido-2-[6-(trifluoromethyl)-1,3-benzothiazol-2-yl]ethyl]phenyl})methylidene]amino acetate

To a magnetically stirred solution of 3-[(2S)-2-benzenesulfonamido-2-[6-(trifluoromethyl)-1,3-benzothiazol-2-yl]ethyl]-N′-hydroxybenzene-1-carboximidamide (0.347 g, 0.667 mmol, 1.0 eq) in acetic acid (3.0 mL) was added acetic anhydride (0.189 mL, 2.00 mmol, 3.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% ammonia) to afford product as a white solid (0.210 g, 0.373 mmol, 56% yield). UPLC-MS (basic 2 min): Rt=1.14 min; m/z=562.9 for [M+H]⁺ Step 5: 3-[(2S)-2-Benzenesulfonamido-2-[6-(trifluoromethyl)-1,3-benzothiazol-2-yl]ethyl]benzene-1-carboximidamide

To a magnetically stirred solution of [amino({3-[(2S)-2-benzenesulfonamido-2-[6-(trifluoromethyl)-1,3-benzothiazol-2-yl]ethyl]phenyl})methylidene]amino acetate (0.210 g, 0.373 mmol, 1.0 eq) in acetic acid (3.0 mL) was added zinc (0.244 g, 3.73 mmol, 10.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford the product as a white solid (0.059 g, 0.117 mmol, 31% yield). ¹H NMR (400 MHz, DMSO-d₆): δ 8.63 (s, 1H), 8.49 (s, 1H), 8.12 (d, J=8.6 Hz, 1H), 7.85-7.80 (m, 1H), 7.72 (s, 1H), 7.55 (d, J=7.9 Hz, 1H), 7.45 (t, J=6.9 Hz, 3H), 7.37 (t, J=7.4 Hz, 1H), 7.26 (dt, J=13.3, 7.7 Hz, 3H), 5.09 (dd, J=9.9, 4.6 Hz, 1H), 3.44-3.39 (m, 1H), 3.06 (dd, J=13.9, 10.0 Hz, 1H). UPLC-MS (basic 6 min): Rt=2.87 min; m/z=504.9 for [M+H]+, 99% purity. Chiral analysis performed by Reach Separations showed 50% chiral purity.

Product purified by SFC using Lux C₄ (21.2 mm×250 mm, 5 um) with a 60:40 MeOH:CO₂ (0.2% v/v NH₃) eluent to afford product as a white solid (0.018 g). 1H NMR (400 MHz, DMSO-d6): δ 8.52 (s, 1H), 8.05 (d, J=8.5 Hz, 1H), 7.76 (d, J=8.6 Hz, 1H), 7.69 (s, 1H), 7.52 (d, J=7.7 Hz, 1H), 7.45 (d, J=7.5 Hz, 2H), 7.27 (t, J=7.8 Hz, 2H), 7.20 (t, J=7.5 Hz, 3H), 4.84 (dd, J=8.5, 4.7 Hz, 1H), 3.03 (dd, J=13.4, 8.6 Hz, 1H). UPLC-MS (basic 4 min): Rt=1.24 min; m/z=505.1 for [M+H]+, 100% purity. Chiral analysis performed by Reach Separations showed 97.6% chiral purity. Rt=1.76 min.

### Example 34. Synthesis of Compound 15

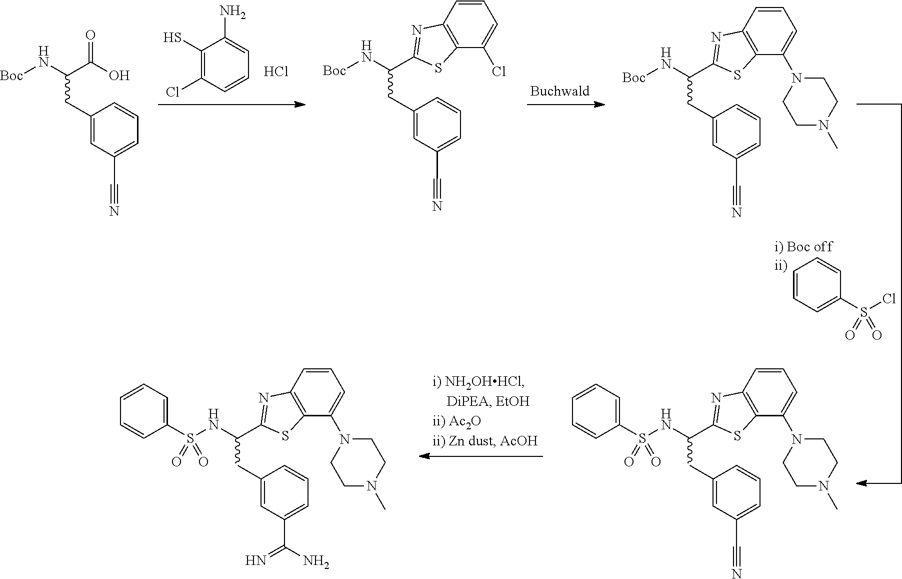

### Step 1: tert-Butyl N-[1-(7-chloro-1,3-benzothiazol-2-yl)-2-(3-cyanophenyl)ethyl]carbamate

To a magnetically stirred solution of 2-{[(tert-butoxy)carbonyl]amino}-3-(3-cyanophenyl)propanoic acid) (1.00 g, 3.44 mmol, 1.0 eq.) in toluene (23.0 mL) were added 2-amino-6-chlorobenzene-1-thiol hydrochloride (0.749 g, 3.82 mmol, 1.1 eq.), DIPEA (1.9 mL, 10.7 mmol, 3.0 eq.) and T3P (50% w/w in EtOAc) (4.2 mL, 6.99 mmol, 2.0 eq.). The reaction mixture was stirred at RT for 30 mins. and then heated at 100° C. for 1 h. The reaction mixture was cooled to RT and then aq. 1M HCl solution was added. The product was extracted with DCM (3×25 mL) and the combined organic phase was washed with aq. sat. NaHCO₃ solution (25 mL), H₂O (25 mL), dried over anhydrous sodium sulfate, filtered and then concentrated to dryness. The residue was purified by Reverse Phase column chromatography eluting with a 5-95% H₂O:MeCN eluent (0.1% ammonia) to afford product as a white solid (1.13 g, 2.73 mmol, 79% yield). ¹H NMR (DMSO-d6) δ: 7.99 (dt, J=6.2, 3.0 Hz, 2H), 7.82 (s, 1H), 7.75-7.67 (m, 2H), 7.61-7.57 (m, 2H), 7.54 (t, J=7.7 Hz, 1H), 5.20 (td, J=9.8, 4.3 Hz, 1H), 3.57 (dd, J=13.8, 4.4 Hz, 1H), 3.16-3.10 (m, 1H), 1.32 (s, 9H). UPLC-MS (basic 2 min) Rt=1.29 min. m/z=414.0 for [M]⁺

### Step 2: tert-Butyl N-[2-(3-cyanophenyl)-1-[7-(4-methylpiperazin-1-yl)-1,3-benzothiazol-2-yl]ethyl]carbamate

To a magnetically stirred solution of tert-butyl N-[1-(7-chloro-1,3-benzothiazol-2-yl)-2-(3-cyanophenyl)ethyl]carbamate (0.300 g, 0.725 mmol, 1.0 eq.) in 1,4-dioxane (3.0 mL) were added N-methylpiperazine (0.174 g, 1.74 mmol, 2.4 eq.), tripotassium phosphate (0.615 g, 2.90 mmol, 4.0 eq.), and DavePhos-Pd-G3 (0.111 g, 0.145 mmol, 0.20 eq.). The reaction mixture was heated at 100° C. for 3 h. The reaction mixture was cooled to RT and then diluted with aq. sat. NaHCO₃ solution (25 mL). The product was extracted with DCM (3×25 mL) and the combined organic phase was dried over anhydrous sodium sulfate, filtered and then concentrated to dryness. The residue was purified by Reverse Phase column chromatography eluting with a 5-95% H₂O:MeCN eluent (0.1% ammonia) to afford product as a white solid (0.136 g, 0.285 mmol, 39% yield). ¹H NMR (DMSO-d6) δ: 7.88 (d, J=8.9 Hz, 1H), 7.81 (s, 1H), 7.71 (d, 2H), 7.63 (d, J=8.0 Hz, 1H), 7.53 (t, J=7.8 Hz, 1H), 7.45 (t, J=7.9 Hz, 1H), 7.03 (d, J=7.8 Hz, 1H), 5.21-5.12 (m, 1H), 3.55 (dd, J=13.6, 4.4 Hz, 1H), 3.19-3.08 (m, 5H), 2.56-2.53 (m, 3H), 2.27 (s, 3H), 1.31 (s, 9H). UPLC-MS (basic 2 min) Rt=1.19 min. m/z=478.1 for [M+H]⁺

### Step 3: 2-(3-Cyanophenyl)-1-[7-(4-methylpiperazin-1-yl)-1,3-benzothiazol-2-yl]ethan-1-aminium chloride

To a magnetically stirred solution of tert-butyl N-[2-(3-cyanophenyl)-1-[7-(4-methylpiperazin-1-yl)-1,3-benzothiazol-2-yl]ethyl]carbamate (0.136 g, 0.285 mmol, 1.0 eq.) in diethyl ether (3.0 mL) and MeOH (1 mL) was added 4N HCl in dioxane (0.789 mL, 3.15 mmol, 11 eq.) and the reaction mixture stirred at RT for 18 h. The reaction mixture was concentrated to dryness to afford product as a white solid (0.160 g, 0.387 mmol, 100% yield) which was used in the next step without further purification. UPLC-MS (basic 2 min) Rt=0.97 min. m/z=378.0 for [M+H]⁺

### Step 4: N-[2-(3-cyanophenyl)-1-[7-(4-methylpiperazin-1-yl)-1,3-benzothiazol-2-yl]ethyl]benzene sulfonamide

To a magnetically stirred solution of 2-(3-cyanophenyl)-1-[7-(4-methylpiperazin-1-yl)-1,3-benzothiazol-2-yl]ethan-1-aminium chloride (0.136 g, 0.329 mmol, 1.0 eq.) in DMF (2.0 mL) were added triethylamine (0.101 mL, 0.723 mmol, 2.2 eq.) and then cooled to −5° C. Benzenesulfonyl chloride (0.050 mL, 0.394 mmol, 1.2 eq.) was added dropwise and the reaction mixture stirred at RT for 18 h. The reaction mixture was concentrated to dryness and the residue purified by Reverse Phase column chromatography eluting with a 5-95% H₂O:MeCN eluent (0.1% ammonia) to afford product as a white solid (0.067 g, 0.129 mmol, 39% yield). ¹H NMR (DMSO-d6) δ: 8.93 (d, J=8.2 Hz, 1H), 7.63 (s, 1H), 7.59 (d, J=8.0 Hz, 1H), 7.55 (dt, J=8.1, 2.0 Hz, 2H), 7.51-7.46 (m, 2H), 7.46-7.41 (m, 2H), 7.36-7.28 (m, 3H), 7.01 (d, J=7.8 Hz, 1H), 5.02 (s, 1H), 3.48-3.36 (m, 2H), 3.16-3.07 (m, 4H), 3.03 (dd, J=13.9, 10.5 Hz, 1H), 2.60-2.54 (m, 4H), 2.31 (s, 3H). UPLC-MS (basic 2 min) Rt=1.11 min. m/z=518.0 for [M+H]⁺

### Step 5: 3-{2-Benzenesulfonamido-2-[7-(4-methylpiperazin-1-yl)-1,3-benzothiazol-2-yl]ethyl}-N′-hydroxybenzene-1-carboximidamide

To a magnetically stirred solution of N-[2-(3-cyanophenyl)-1-[7-(4-methylpiperazin-1-yl)-1,3-benzothiazol-2-yl]ethyl]benzenesulfonamide (0.067 g, 0.129 mmol, 1.0 eq.) in EtOH (1.0 mL) were added hydroxylamine hydrochloride (0.018 g, 0.259 mmol, 2.0 eq.) and DIPEA (0.068 mL, 0.388 mmol, 3.0 eq.). The reaction mixture was stirred under reflux for 1 h. The reaction mixture was cooled down to RT and then concentrated to dryness to afford product as a yellow oil (0.127 g, 0.231 mmol, 100% yield) which was used in the next step without further purification. UPLC-MS (basic 2 min) Rt=0.97 min. m/z=551.0 for [M+H]⁺

### Step 6: [Amino(3-{2-benzenesulfonamido-2-[7-(4-methylpiperazin-1-yl)-1,3-benzothiazol-2-yl]ethyl}phenyl)methylidene]amino acetate

To a magnetically stirred solution of 3-{2-benzenesulfonamido-2-[7-(4-methylpiperazin-1-yl)-1,3-benzothiazol-2-yl]ethyl}-N′-hydroxybenzene-1-carboximidamide (0.127 g, 0.231 mmol, 1.0 eq) in acetic acid (1.0 mL) was added acetic anhydride (0.065 mL, 0.692 mmol, 3.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% ammonia) to afford product as a white solid (0.137 g, 0.231 mmol, 100% yield). UPLC-MS (basic 2 min): Rt=1.01 min; m/z=593.0 for [M]⁺

### Step 7: 3-{2-Benzenesulfonamido-2-[7-(4-methylpiperazin-1-yl)-1,3-benzothiazol-2-yl]ethyl}benzene-1-carboximidamide

To a magnetically stirred solution of [amino(3-{2-benzenesulfonamido-2-[7-(4-methylpiperazin-1-yl)-1,3-benzothiazol-2-yl]ethyl}phenyl)methylidene]amino acetate (0.137 g, 0.231 mmol, 1.0 eq) in acetic acid (2.0 mL) was added zinc (0.151 g, 2.31 mmol, 20.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford the product as a white solid (0.007 g, 0.013 mmol, 6% yield). ¹H NMR (400 MHz, DMSO-d₆): δ 7.62 (s, 1H), 7.47 (dd, J=7.9, 4.2 Hz, 4H), 7.33 (t, J=7.9 Hz, 1H), 7.19 (h, J=7.8 Hz, 5H), 6.88 (d, J=7.8 Hz, 1H), 4.73 (s, 1H), 3.10-2.95 (m, 6H), 2.27 (s, 3H), 1.25 (s, 4H). UPLC-MS (basic 6 min): Rt=2.25 min; m/z=535.0 for [M+H]+, 95% purity. Chiral analysis performed by Reach Separations showed 50% chiral purity.

### Example 35. Synthesis of Compound 16

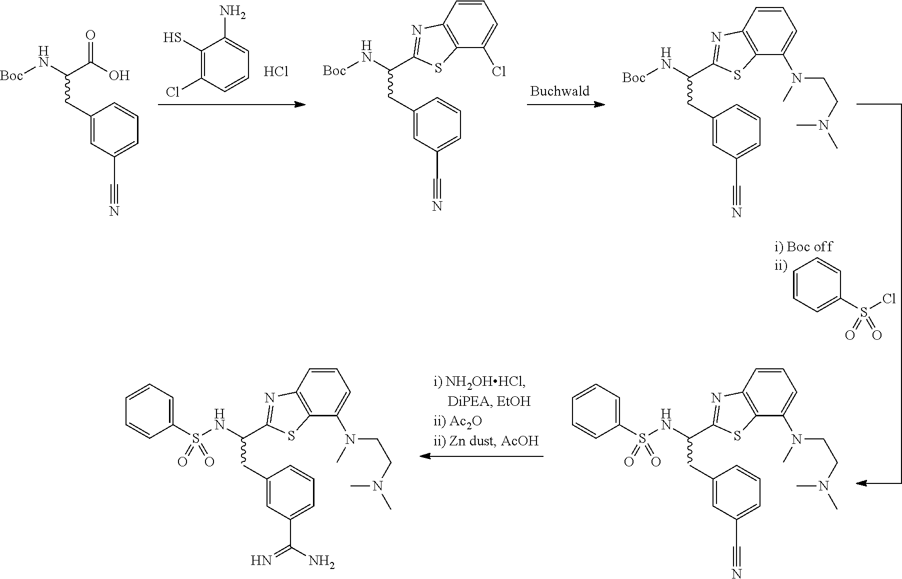

### Step 1: tert-Butyl N-[1-(7-chloro-1,3-benzothiazol-2-yl)-2-(3-cyanophenyl)ethyl]carbamate

To a magnetically stirred solution of 2-{[(tert-butoxy)carbonyl]amino}-3-(3-cyanophenyl)propanoic acid) (1.00 g, 3.44 mmol, 1.0 eq.) in toluene (23.0 mL) were added 2-amino-6-chlorobenzene-1-thiol hydrochloride (0.749 g, 3.82 mmol, 1.1 eq.), DIPEA (1.9 mL, 10.7 mmol, 3.0 eq.) and T3P (50% w/w in EtOAc) (4.2 mL, 6.99 mmol, 2.0 eq.). The reaction mixture was stirred at RT for 30 mins. and then heated at 100° C. for 1 h. The reaction mixture was cooled to RT and then aq. 1M HCl solution was added. The product was extracted with DCM (3×25 mL) and the combined organic phase was washed with aq. sat. NaHCO₃ solution (25 mL), H₂O (25 mL), dried over anhydrous sodium sulfate, filtered and then concentrated to dryness. The residue was purified by Reverse Phase column chromatography eluting with a 5-95% H₂O:MeCN eluent (0.1% ammonia) to afford product as a white solid (1.13 g, 2.73 mmol, 79% yield). ¹H NMR (DMSO-d6) δ: 7.99 (dt, J=6.2, 3.0 Hz, 2H), 7.82 (s, 1H), 7.75-7.67 (m, 2H), 7.61-7.57 (m, 2H), 7.54 (t, J=7.7 Hz, 1H), 5.20 (td, J=9.8, 4.3 Hz, 1H), 3.57 (dd, J=13.8, 4.4 Hz, 1H), 3.16-3.10 (m, 1H), 1.32 (s, 9H). UPLC-MS (basic 2 min) Rt=1.29 min. m/z=414.0 for [M]⁺

### Step 2: tert-Butyl N-[2-(3-cyanophenyl)-1-(7-{[2-(dimethylamino)ethyl](methyl)amino}-1,3-benzothiazol-2-yl)ethyl]carbamate

To a magnetically stirred solution of tert-butyl N-[1-(7-chloro-1,3-benzothiazol-2-yl)-2-(3-cyanophenyl)ethyl]carbamate (0.550 g, 1.33 mmol, 1.0 eq.) in 1,4-dioxane (3.0 mL) were added [2-(dimethylamino)ethyl](methyl)amine (0.415 mL, 3.19 mmol, 2.4 eq.), tripotassium phosphate (1.13 g, 5.32 mmol, 4.0 eq.), and DavePhos-Pd-G3 (0.203 g, 0.266 mmol, 0.20 eq.). The reaction mixture was heated at 100° C. for 2 h. The reaction mixture was cooled to RT and then diluted with aq. sat. NaHCO₃ solution (25 mL). The product was extracted with DCM (3×25 mL) and the combined organic phase was dried over anhydrous sodium sulfate, filtered and then concentrated to dryness. The residue was purified by Reverse Phase column chromatography eluting with a 5-95% H₂O:MeCN eluent (0.1% ammonia) to afford product as a white solid (0.297 g, 0.619 mmol, 47% yield). ¹H NMR (DMSO-d6) δ: 7.83 (d, J=8.8 Hz, 1H), 7.74 (s, 1H), 7.64 (d, J=7.8 Hz, 2H), 7.48-7.43 (m, 2H), 7.33 (t, J=8.0 Hz, 1H), 6.85 (d, J=7.9 Hz, 1H), 5.11-5.03 (m, 1H), 3.48 (dd, J=13.8, 4.3 Hz, 1H), 3.30 (t, J=7.3 Hz, 2H), 3.11-3.02 (m, 1H), 2.86 (s, 3H), 2.35 (t, J=7.3 Hz, 2H), 2.07 (s, 6H), 1.24 (s, 9H). UPLC-MS (basic 2 min) Rt=1.23 min. m/z=480.1 for [M+H]⁺

### Step 3: 2-(3-Cyanophenyl)-1-(7-{[2-(dimethylamino)ethyl](methyl)amino}-1,3-benzothiazol-2-yl)ethan-1-aminium chloride

tert-Butyl N-[2-(3-cyanophenyl)-1-(7-{[2-(dimethylamino)ethyl] (methyl)amino}-1,3-benzothiazol-2-yl)ethyl]carbamate (0.297 g, 0.619 mmol, 1.0 eq.) was dissolved in 4N HCl in dioxane (3 mL) and the reaction mixture stirred at RT for 18 h. The reaction mixture was concentrated to dryness to afford product as an orange solid (0.331 g, 0.796 mmol, 100% yield) which was used in the next step without further purification. UPLC-MS (basic 2 min) Rt=0.99 min. m/z=380.2 for [M+H]⁺

### Step 4: N-[2-(3-Cyanophenyl)-1-(7-{[2-(dimethylamino)ethyl](methyl)amino}-1,3-benzothiazol-2-yl)ethyl]benzenesulfonamide

To a magnetically stirred solution of 2-(3-cyanophenyl)-1-(7-{[2-(dimethylamino)ethyl](methyl)amino}-1,3-benzothiazol-2-yl)ethan-1-aminium chloride (0.331 g, 0.796 mmol, 1.0 eq.) in DMF (4.0 mL) were added triethylamine (0.243 mL, 1.75 mmol, 2.2 eq.) and then cooled to −5° C. Benzenesulfonyl chloride (0.122 mL, 0.955 mmol, 1.2 eq.) was added dropwise and the reaction mixture stirred at RT for 18 h. The reaction mixture was concentrated to dryness and the residue purified by Reverse Phase column chromatography eluting with a 5-95% H₂O:MeCN eluent (0.1% ammonia) to afford product as an orange solid (0.070 g, 0.135 mmol, 17% yield). ¹H NMR (DMSO-d6) δ: 7.61 (s, 1H), 7.54 (d, J=7.8 Hz, 2H), 7.48 (dd, J=8.0, 3.2 Hz, 3H), 7.45-7.35 (m, 3H), 7.31 (q, J=8.0 Hz, 3H), 6.92 (d, J=7.8 Hz, 1H), 5.05-4.97 (m, 1H), 3.49-3.35 (m, 3H), 3.02 (dd, J=14.0, 10.5 Hz, 1H), 2.92 (s, 3H), 2.42 (t, J=7.2 Hz, 2H), 2.17 (s, 5H). UPLC-MS (basic 2 min) Rt=1.15 min. m/z=520.0 for [M+H]⁺

### Step 5: 3-[2-Benzenesulfonamido-2-(7-{[2-(dimethylamino)ethyl](methyl)amino}-1,3-benzothiazol-2-yl)ethyl]-N′-hydroxybenzene-1-carboximidamide

To a magnetically stirred solution of N-[2-(3-cyanophenyl)-1-(7-{[2-(dimethylamino)ethyl](methyl) amino}-1,3-benzothiazol-2-yl)ethyl]benzenesulfonamide (0.100 g, 0.192 mmol, 1.0 eq.) in EtOH (1.0 mL) were added hydroxylamine hydrochloride (0.027 g, 0.385 mmol, 2.0 eq.) and DIPEA (0.101 mL, 0.577 mmol, 3.0 eq.). The reaction mixture was stirred under reflux for 1 h. The reaction mixture was cooled down to RT and then concentrated to dryness to afford product as a yellow oil (0.106 g, 0.192 mmol, 100% yield) which was used in the next step without further purification. UPLC-MS (basic 2 min) Rt=1.01 min. m/z=553.0 for [M+H]⁺

### Step 6: [Amino({3-[2-benzenesulfonamido-2-(7-{[2-(dimethylamino)ethyl](methyl)amino}-1,3-benzothiazol-2-yl)ethyl]phenyl})methylidene]amino acetate

To a magnetically stirred solution of 3-[2-benzenesulfonamido-2-(7-{[2-(dimethylamino)ethyl] (methyl)amino}-1,3-benzothiazol-2-yl)ethyl]-N′-hydroxybenzene-1-carboximidamide (0.106 g, 0.192 mmol, 1.0 eq) in acetic acid (2.0 mL) was added acetic anhydride (0.054 mL, 0.575 mmol, 3.0 eq.) and the resulting mixture was stirred at RT for 60 mins. The reaction mixture was concentrated to dryness and the residue was purified by Normal Phase column chromatography eluting with MeOH, DCM 0-15% to afford product as a clear oil (0.040 g, 0.067 mmol, 35% yield). UPLC-MS (basic 2 min): Rt=1.05 min; m/z=595.0 for [M]⁺

### Step 7: 3-[2-Benzenesulfonamido-2-(7-{[2-(dimethylamino)ethyl](methyl)amino}-1,3-benzothiazol-2-yl)ethyl]benzene-1-carboximidamide

To a magnetically stirred solution of [amino({3-[2-benzenesulfonamido-2-(7-{[2-(dimethylamino)ethyl](methyl)amino}-1,3-benzothiazol-2-yl)ethyl]phenyl})methylidene]amino acetate (0.040 g, 0.067 mmol, 1.0 eq) in acetic acid (1.0 mL) was added zinc (0.044 g, 0.673 mmol, 10.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford the product as a white solid (0.011 g, 0.020 mmol, 30% yield). ¹H NMR (400 MHz, DMSO-d₆): δ 7.61 (s, 1H), 7.47-7.39 (m, 3H), 7.36-7.27 (m, 3H), 7.24-7.14 (m, 4H), 6.85 (d, J=7.7 Hz, 1H), 4.82 (s, 1H), 3.31-3.19 (m, 3H), 3.06-2.93 (m, 2H), 2.84 (s, 3H), 2.37 (d, J=7.8 Hz, 2H), 2.11 (s, 5H). UPLC-MS (acidic 6 min): Rt=1.54 min; m/z=537.0 for [M+H]+, 84% purity. Chiral analysis performed by Reach Separations showed 52.7% chiral purity.

### Example 36. Synthesis of Compound 17

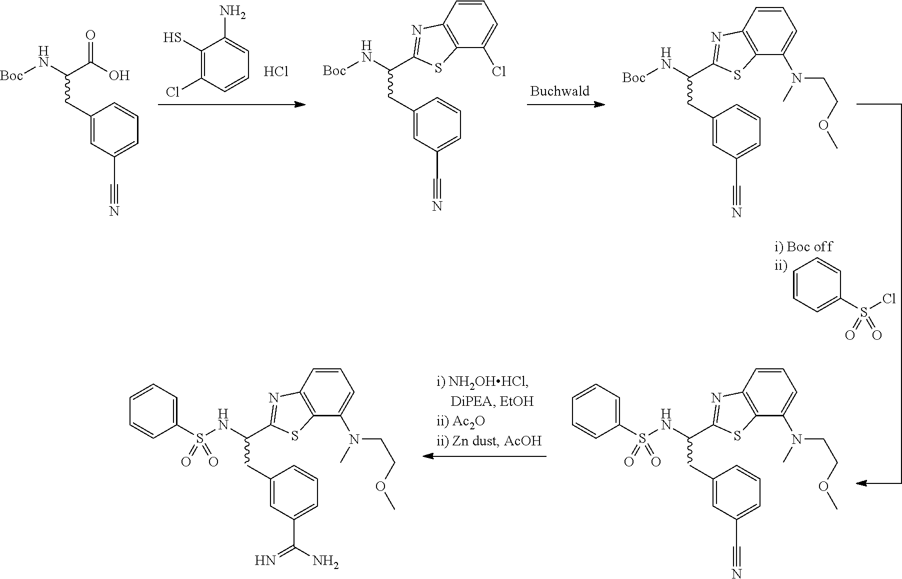

### Step 1: tert-Butyl N-[1-(7-chloro-1,3-benzothiazol-2-yl)-2-(3-cyanophenyl)ethyl]carbamate

To a magnetically stirred solution of 2-{[(tert-butoxy)carbonyl]amino}-3-(3-cyanophenyl)propanoic acid) (1.00 g, 3.44 mmol, 1.0 eq.) in toluene (23.0 mL) were added 2-amino-6-chlorobenzene-1-thiol hydrochloride (0.749 g, 3.82 mmol, 1.1 eq.), DIPEA (1.9 mL, 10.7 mmol, 3.0 eq.) and T3P (50% w/w in EtOAc) (4.2 mL, 6.99 mmol, 2.0 eq.). The reaction mixture was stirred at RT for 30 mins. and then heated at 100° C. for 1 h. The reaction mixture was cooled to RT and then aq. 1M HCl solution was added. The product was extracted with DCM (3×25 mL) and the combined organic phase was washed with aq. sat. NaHCO₃ solution (25 mL), H₂O (25 mL), dried over anhydrous sodium sulfate, filtered and then concentrated to dryness. The residue was purified by Reverse Phase column chromatography eluting with a 5-95% H₂O:MeCN eluent (0.1% ammonia) to afford product as a white solid (1.13 g, 2.73 mmol, 79% yield). ¹H NMR (DMSO-d6) δ: 7.99 (dt, J=6.2, 3.0 Hz, 2H), 7.82 (s, 1H), 7.75-7.67 (m, 2H), 7.61-7.57 (m, 2H), 7.54 (t, J=7.7 Hz, 1H), 5.20 (td, J=9.8, 4.3 Hz, 1H), 3.57 (dd, J=13.8, 4.4 Hz, 1H), 3.16-3.10 (m, 1H), 1.32 (s, 9H). UPLC-MS (basic 2 min) Rt=1.29 min. m/z=414.0 for [M]⁺

### Step 2: tert-Butyl N-[2-(3-cyanophenyl)-1-{7-[(2-methoxyethyl)(methyl)amino]-1,3-benzothiazol-2-yl}ethyl]carbamate

To a magnetically stirred solution of tert-butyl N-[1-(7-chloro-1,3-benzothiazol-2-yl)-2-(3-cyanophenyl)ethyl]carbamate (0.500 g, 1.21 mmol, 1.0 eq.) in 1,4-dioxane (5.0 mL) were added 2-methoxyethyl)(methyl)amine (0.315 mL, 2.90 mmol, 2.4 eq.), tripotassium phosphate (1.03 g, 4.83 mmol, 4.0 eq.), and DavePhos-Pd-G3 (0.184 g, 0.242 mmol, 0.20 eq.). The reaction mixture was heated at 100° C. for 2 h. The reaction mixture was cooled to RT and then diluted with aq. sat. NaHCO₃ solution (25 mL). The product was extracted with DCM (3×25 mL) and the combined organic phase was dried over anhydrous sodium sulfate, filtered and then concentrated to dryness. The residue was purified by Reverse Phase column chromatography eluting with a 5-95% H₂O:MeCN eluent (0.1% ammonia) to afford product as a white solid (0.231 g, 0.495 mmol, 41% yield). ¹H NMR (DMSO-d6) δ: 7.91 (t, J=8.5 Hz, 1H), 7.81 (s, 1H), 7.72 (d, J=7.8 Hz, 2H), 7.55-7.51 (m, 2H), 7.41 (t, J=7.8 Hz, 1H), 6.95 (d, J=7.8 Hz, 1H), 5.19-5.11 (m, 1H), 3.51 (d, J=5.5 Hz, 2H), 3.47-3.43 (m, 2H), 3.24 (s, 3H), 3.19-3.08 (m, 2H), 2.96 (s, 3H), 1.32 (s, 9H). UPLC-MS (basic 2 min) Rt=1.30 min. m/z=467.0 for [M+H]⁺

### Step 3: 2-(3-Cyanophenyl)-1-{7-[(2-methoxyethyl)(methyl)amino]-1,3-benzothiazol-2-yl}ethan-1-aminium chloride

tert-Butyl N-[2-(3-cyanophenyl)-1-{7-[(2-methoxyethyl)(methyl)amino]-1,3-benzothiazol-2-yl}ethyl]carbamate (0.231 g, 0.495 mmol, 1.0 eq.) was dissolved in 4N HCl in dioxane (3 mL) and the reaction mixture stirred at RT for 18 h. The reaction mixture was concentrated to dryness to afford product as an orange solid (0.226 g, 0.561 mmol, 100% yield) which was used in the next step without further purification. UPLC-MS (basic 2 min) Rt=1.02 min. m/z=367.0 for [M+H]⁺

### Step 4: N-[2-(3-Cyanophenyl)-1-{7-[(2-methoxyethyl)(methyl)amino]-1,3-benzothiazol-2-yl}ethyl]benzenesulfonamide

To a magnetically stirred solution of 2-(3-cyanophenyl)-1-{7-[(2-methoxyethyl)(methyl)amino]-1,3-benzothiazol-2-yl}ethan-1-aminium chloride (0.226 g, 0.561 mmol, 1.0 eq.) in DMF (4.0 mL) were added triethylamine (0.172 mL, 1.23 mmol, 2.2 eq.) and then cooled to −5° C. Benzenesulfonyl chloride (0.086 mL, 0.673 mmol, 1.2 eq.) was added dropwise and the reaction mixture stirred at RT for 18 h. The reaction mixture was concentrated to dryness and the residue purified by Reverse Phase column chromatography eluting with a 5-95% H₂O:MeCN eluent (0.1% ammonia) to afford product as an orange solid (0.085 g, 0.168 mmol, 30% yield). ¹H NMR (DMSO-d6) δ: 7.62 (s, 1H), 7.56-7.53 (m, 2H), 7.50-7.46 (m, 3H), 7.42 (dd, J=14.8, 7.0 Hz, 3H), 7.33-7.30 (m, 3H), 6.94 (d, J=7.9 Hz, 1H), 5.09-4.97 (m, 1H), 3.51 (t, J=5.9 Hz, 2H), 3.47-3.35 (m, 4H), 3.26 (s, 3H), 3.09-2.97 (m, 2H), 2.94 (s, 3H). UPLC-MS (basic 2 min) Rt=1.18 min. m/z=507.0 for [M+H]⁺

### Step 5: 3-(2-Benzenesulfonamido-2-{7-[(2-methoxyethyl)(methyl)amino]-1,3-benzothiazol-2-yl}ethyl)-N′-hydroxybenzene-1-carboximidamide

To a magnetically stirred solution of N-[2-(3-cyanophenyl)-1-{7-[(2-methoxyethyl)(methyl)amino]-1,3-benzothiazol-2-yl}ethyl]benzenesulfonamide (0.103 g, 0.203 mmol, 1.0 eq.) in EtOH (1.0 mL) were added hydroxylamine hydrochloride (0.028 g, 0.407 mmol, 2.0 eq.) and DIPEA (0.106 mL, 0.610 mmol, 3.0 eq.). The reaction mixture was stirred under reflux for 1 h. The reaction mixture was cooled down to RT and then concentrated to dryness to afford product as a yellow oil (0.110 g, 0.204 mmol, 100% yield) which was used in the next step without further purification. UPLC-MS (basic 2 min) Rt=1.05 min. m/z=540.0 for [M+H]⁺

### Step 6: {Amino[3-(2-benzenesulfonamido-2-{7-[(2-methoxyethyl)(methyl)amino]-1,3-benzothiazol-2-yl}ethyl)phenyl]methylidene}amino acetate

To a magnetically stirred solution of 3-(2-benzenesulfonamido-2-{7-[(2-methoxyethyl)(methyl)amino]-1,3-benzothiazol-2-yl}ethyl)-N′-hydroxybenzene-1-carboximidamide (0.110 g, 0.204 mmol, 1.0 eq) in acetic acid (2.0 mL) was added acetic anhydride (0.058 mL, 0.611 mmol, 3.0 eq.) and the resulting mixture was stirred at RT for 60 mins. The reaction mixture was concentrated to dryness and the residue was purified by Normal Phase column chromatography eluting with MeOH, DCM 0-15% to afford product as a clear oil (0.067 g, 0.115 mmol, 57% yield). UPLC-MS (basic 2 min): Rt=1.09 min; m/z=582.0 for [M]⁺

### Step 7: 3-(2-Benzenesulfonamido-2-{7-[(2-methoxyethyl)(methyl)amino]-1,3-benzothiazol-2-yl}ethyl)benzene-1-carboximidamide

To a magnetically stirred solution of {amino[3-(2-benzenesulfonamido-2-{7-[(2-methoxyethyl)(methyl) amino]-1,3-benzothiazol-2-yl}ethyl)phenyl]methylidene}amino acetate (0.067 g, 0.115 mmol, 1.0 eq) in acetic acid (1.0 mL) was added zinc (0.075 g, 1.15 mmol, 10.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford the product as a white solid (0.004 g, 0.007 mmol, 6% yield). ¹H NMR (400 MHz, DMSO-d₆): δ 7.60 (s, 1H), 7.46-7.38 (m, 3H), 7.35 (d, J=8.0 Hz, 1H), 7.29-7.08 (m, 6H), 6.79 (d, J=7.8 Hz, 1H), 4.78 (dd, J=8.9, 5.1 Hz, 1H), 2.94 (dd, J=13.5, 8.9 Hz, 2H), 2.84 (s, 3H). UPLC-MS (basic 4 min): Rt=0.97 min; m/z=524.2 for [M+H]⁺, 90% purity. Chiral analysis performed by Reach Separations showed 51% chiral purity.

### Example 37. Synthesis of Compound 18

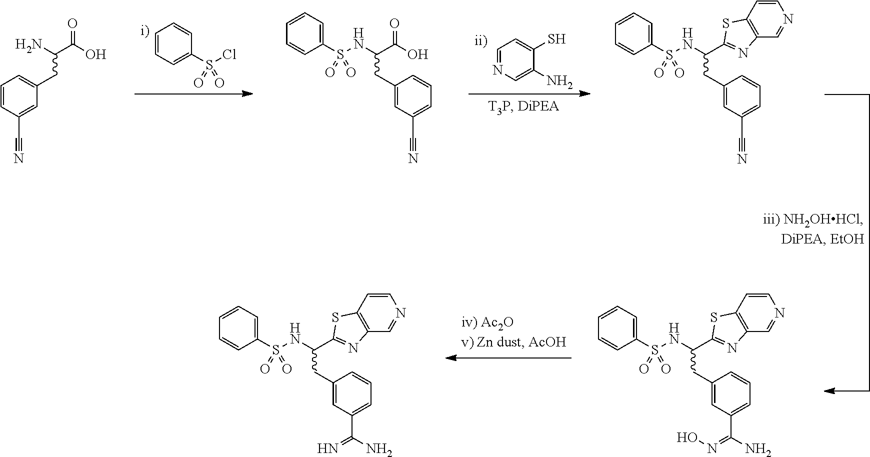

### Step 1: 2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[2-(3-Cyanophenyl)-1-{[1,3]thiazolo[4,5-c]pyridin-2-yl}ethyl]benzenesulfonamide

To a magnetically stirred solution of 2-benzenesulfonamido-3-(3-cyanophenyl)propanoic acid (0.200 g, 0.605 mmol, 1.0 eq.) in toluene (5.0 mL) were added T3P (50% w/w in EtOAc) (0.75 mL, 1.28 mmol, 2.0 eq.), DIPEA (0.35 mL, 2.00 mmol, 3.0 eq.) and 3-aminopyridine-2-thiol (0.092 g, 0.726 mmol, 1.2 eq.). The reaction mixture was heated at 100° C. for 24 h. The reaction mixture was cooled to RT and then concentrated to dryness. The residue was purified by Reverse Phase column chromatography eluting with a 5-95% H₂O:MeCN eluent (0.1% ammonia) to afford product as a white solid (0.096 g, 0.228 mmol, 38% yield). ¹H NMR (DMSO-d6) δ: 9.20 (s, 1H), 8.51 (br d, J=5.1 Hz, 1H), 8.17 (br d, J=5.1 Hz, 1H), 7.61 (s, 1H), 7.53 (br d, J=7.8 Hz, 2H), 7.42-7.50 (m, 3H), 7.37-7.42 (m, 1H), 7.24-7.33 (m, 3H), 5.10 (br dd, J=10.4, 4.0 Hz, 1H), 3.40 (br dd, J=13.9, 4.2 Hz, 1H), 2.96-3.12 (m, 1H). UPLC-MS (basic 2 min) Rt=0.99 min. m/z=421.0 for [M−H]⁺

### Step 3: 3-(2-Benzenesulfonamido-2-{[1,3]thiazolo[4,5-c]pyridin-2-yl}ethyl)-N′-hydroxybenzene-1-carboximidamide

To a magnetically stirred solution of N-[2-(3-cyanophenyl)-1-{[1,3]thiazolo[4,5-c]pyridin-2-yl}ethyl]benzenesulfonamide (0.096 g, 0.228 mmol, 1.0 eq.) in EtOH (3.0 mL) were added hydroxylamine hydrochloride (0.036 g, 0.518 mmol, 2.0 eq.) and DIPEA (0.120 mL, 0.689 mmol, 3.0 eq.). The reaction mixture was stirred under reflux for 1 h. The reaction mixture was cooled down to RT and then concentrated to dryness to afford product as a yellow oil (0.119 g, 0.262 mmol, 100% yield) which was used in the next step without further purification. UPLC-MS (basic 2 min) Rt=0.81 min. m/z=454.0 for [M+H]⁺

### Step 4: {Amino[3-(2-benzenesulfonamido-2-{[1,3]thiazolo[4,5-c]pyridin-2-yl}ethyl)phenyl]methylidene} amino acetate)

To a magnetically stirred solution of 3-(2-benzenesulfonamido-2-{[1,3]thiazolo[4,5-c]pyridin-2-yl}ethyl)-N′-hydroxybenzene-1-carboximidamide (0.119 g, 0.262 mmol, 1.0 eq) in acetic acid (3.0 mL) was added acetic anhydride (0.080 mL, 0.848 mmol, 3.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% formic acid) to afford product as a white solid (0.046 g, 0.093 mmol, 35% yield). ¹H NMR (400 MHz, DMSO-d₆): δ 9.09-9.24 (m, 1H), 8.93-9.09 (m, 1H), 8.49 (br d, J=4.6 Hz, 1H), 8.12-8.16 (m, 1H), 7.60 (s, 1H), 7.42-7.49 (m, 3H), 7.32-7.39 (m, 1H), 7.24 (t, J=7.6 Hz, 3H), 7.09-7.17 (m, 1H), 6.73 (s, 2H), 5.03 (br s, 1H), 3.33 (br d, J=5.1 Hz, 1H), 3.04 (br dd, J=13.8, 9.7 Hz, 1H), 2.15 (s, 3H). UPLC-MS (basic 2 min): Rt=0.95 min; m/z=496.0 for [M+H]⁺

### Step 5: 3-(2-Benzenesulfonamido-2-{[1,3]thiazolo[4,5-c]pyridin-2-yl}ethyl)benzene-1-carboximidamide

To a magnetically stirred solution of {amino[3-(2-benzenesulfonamido-2-{[1,3]thiazolo[4,5-c]pyridin-2-yl}ethyl)phenyl]methylidene}amino acetate (0.046 g, 0.093 mmol, 1.0 eq) in acetic acid (2.0 mL) was added zinc (0.289 g, 4.42 mmol, 48.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford the product as a white solid (0.005 g, 0.012 mmol, 13% yield). ¹H NMR (400 MHz, DMSO-d₆): δ 9.11 (s, 1H), 8.43 (d, J=5.4 Hz, 1H), 8.06 (d, J=5.4 Hz, 1H), 7.67 (s, 1H), 7.51 (br d, J=7.6 Hz, 1H), 7.45 (br d, J=7.3 Hz, 2H), 7.31 (br d, J=7.3 Hz, 1H), 7.14-7.28 (m, 4H), 4.87 (br s, 1H), 3.03 (br dd, J=13.3, 8.7 Hz, 1H). UPLC-MS (basic 6 min): Rt=1.56 min; m/z=438.0 for [M+H]+, 96% purity. Chiral analysis performed by Reach Separations showed 50% chiral purity.

### Example 38. Synthesis of Compound 19

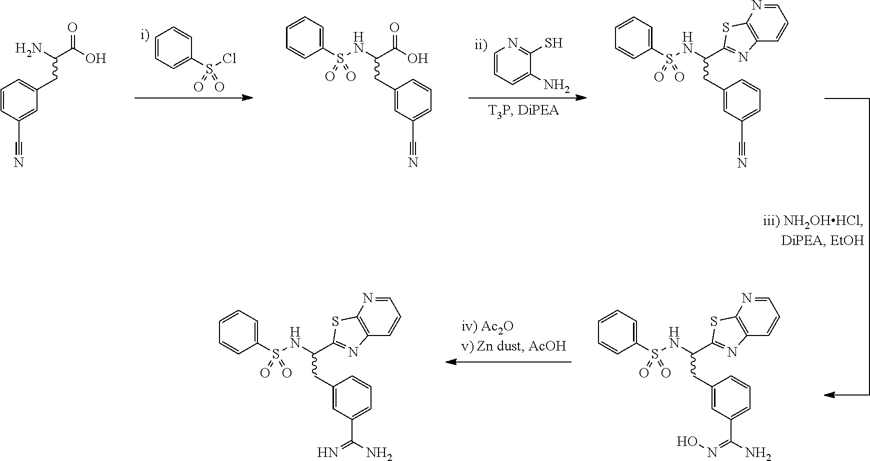

### Step 1: 2-Benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[2-(3-Cyanophenyl)-1-{[1,3]thiazolo[5,4-b]pyridin-2-yl}ethyl]benzenesulfonamide

To a magnetically stirred solution of 2-benzenesulfonamido-3-(3-cyanophenyl)propanoic acid (0.200 g, 0.605 mmol, 1.0 eq.) in toluene (5.0 mL) were added T3P (50% w/w in EtOAc) (0.75 mL, 1.28 mmol, 2.0 eq.), DIPEA (0.35 mL, 2.00 mmol, 3.0 eq.) and 3-aminopyridine-2-thiol (0.092 g, 0.726 mmol, 1.2 eq.). The reaction mixture was heated at 100° C. for 24 h. The reaction mixture was cooled to RT and then concentrated to dryness. The residue was purified by Reverse Phase column chromatography eluting with a 5-95% H₂O:MeCN eluent (0.1% ammonia) to afford product as a white solid (0.154 g, 0.366 mmol, 61% yield). ¹H NMR (DMSO-d6) δ: 9.01 (br s, 1H), 8.60 (d, J=4.4 Hz, 1H), 8.33 (d, J=8.1 Hz, 1H), 7.62 (s, 1H), 7.56-7.61 (m, 1H), 7.54 (br d, J=7.6 Hz, 2H), 7.47 (d, J=7.6 Hz, 2H), 7.39-7.44 (m, 1H), 7.30 (dt, J=15.6, 7.7 Hz, 3H), 5.04 (br d, J=5.6 Hz, 1H), 3.39 (br dd, J=14.1, 4.5 Hz, 1H), 3.04 (dd, J=13.6, 10.9 Hz, 1H). UPLC-MS (basic 2 min) Rt=1.03 min. m/z=421.0 for [M−H]⁺

### Step 3: 3-(2-Benzenesulfonamido-2-{[1,3]thiazolo[5,4-b]pyridin-2-yl}ethyl)-N′-hydroxybenzene-1-carboximidamide)

To a magnetically stirred solution of N-[2-(3-cyanophenyl)-1-{[1,3]thiazolo[5,4-b]pyridin-2-yl}ethyl]benzenesulfonamide (0.154 g, 0.366 mmol, 1.0 eq.) in EtOH (4.0 mL) were added hydroxylamine hydrochloride (0.055 g, 0.791 mmol, 2.0 eq.) and DIPEA (0.200 mL, 1.15 mmol, 3.0 eq.). The reaction mixture was stirred under reflux for 1 h. The reaction mixture was cooled down to RT and then concentrated to dryness to afford product as a yellow oil (0.165 g, 0.366 mmol, 100% yield) which was used in the next step without further purification. UPLC-MS (basic 2 min) Rt=0.87 min. m/z=454.0 for [M+H]⁺

### Step 4: {Amino[3-(2-benzenesulfonamido-2-{[1,3]thiazolo[5,4-b]pyridin-2-yl}ethyl)phenyl]methylidene} amino acetate)

To a magnetically stirred solution of 3-(2-benzenesulfonamido-2-{[1,3]thiazolo[5,4-b]pyridin-2-yl}ethyl)-N′-hydroxybenzene-1-carboximidamide (0.201 g, 0.443 mmol, 1.0 eq) in acetic acid (4.0 mL) was added acetic anhydride (0.130 mL, 1.38 mmol, 3.0 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% formic acid) to afford product as a white solid (0.085 g, 0.72 mmol, 39% yield). ¹H NMR (400 MHz, DMSO-d₆): δ 8.98 (d, J=8.1 Hz, 1H), 8.58 (d, J=4.4 Hz, 1H), 8.30 (d, J=8.1 Hz, 1H), 7.60 (s, 1H), 7.56 (dd, J=8.1, 4.6 Hz, 1H), 7.47 (d, J=7.8 Hz, 3H), 7.32-7.38 (m, 1H), 7.21-7.28 (m, 3H), 7.13-7.19 (m, 1H), 6.73 (s, 2H), 4.92-5.03 (m, 1H), 3.29 (br s, 1H), 3.05 (dd, J=13.9, 9.5 Hz, 1H), 2.15 (s, 3H). UPLC-MS (basic 2 min): Rt=0.95 min; m/z=496.0 for [M+H]⁺

### Step 5: 3-(2-Benzenesulfonamido-2-{[1,3]thiazolo[5,4-b]pyridin-2-yl}ethyl)benzene-1-carboximidamide

To a magnetically stirred solution of {amino[3-(2-benzenesulfonamido-2-{[1,3]thiazolo[5,4-b]pyridin-2-yl}ethyl)phenyl]methylidene}amino acetate (0.085 g, 0.172 mmol, 1.0 eq) in acetic acid (2.0 mL) was added zinc (0.428 g, 6.55 mmol, 38.0 eq.). The reaction mixture was stirred at room temperature for 18 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% ammonia) to afford the product as a white solid (0.014 g, 0.031 mmol, 18% yield). ¹H NMR (400 MHz, DMSO-d₆): δ 8.48 (d, J=4.6 Hz, 1H), 8.18 (d, J=8.3 Hz, 1H), 7.59 (s, 1H), 7.44-7.52 (m, 2H), 7.40 (d, J=7.3 Hz, 2H), 7.31 (br d, J=7.6 Hz, 1H), 7.19-7.27 (m, 2H), 7.11-7.18 (m, 2H), 4.73 (br dd, J=7.9, 4.3 Hz, 1H), 3.13-3.24 (m, 1H), 3.01 (br dd, J=13.3, 8.9 Hz, 1H). UPLC-MS (basic 6 min): Rt=1.79 min; m/z=438.0 for [M+H]⁺, 95% purity. Chiral analysis performed by Reach Separations showed 50% chiral purity.

### Example 39. Synthesis of Compound 20

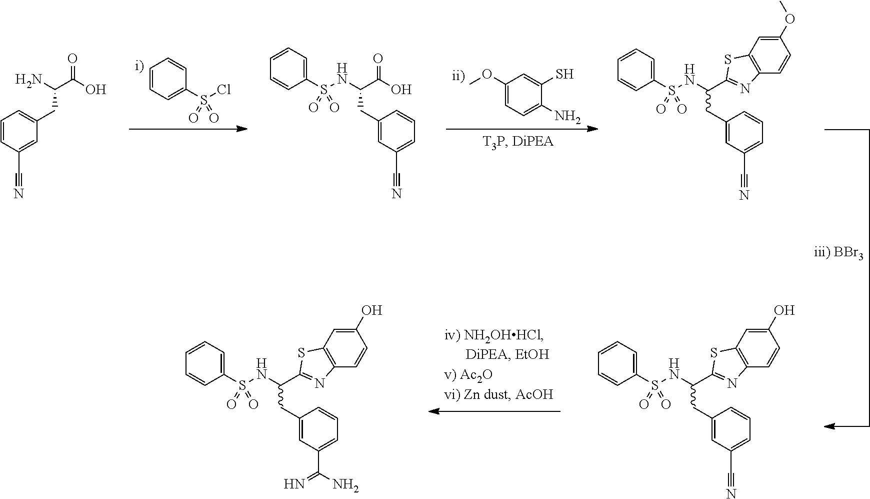

### Step 1: 2-benzenesulfonamido-3-(3-cyanophenyl)propanoic acid

To a magnetically stirred solution of sodium bicarbonate (0.669 g, 6.31 mmol, 1.2 eq.) in water (6.3 mL) was added benzenesulfonyl chloride (0.805 mL, 6.31 mmol, 1.2 eq.) and the resulting solution was cooled to −5° C. (2S)-2-amino-3-(3-cyanophenyl)propanoic acid (1.00 g, 5.28 mmol, 1.0 eq.) was added in 4 portions over a period of 70 mins. The resulting slurry was allowed to warm to RT and stirred for 3 h. The reaction mixture was acidified with 20% HCl and then filtered under vacuum. The solid was triturated with water (2×50 mL) and then dried to afford product as a white solid (0.697 g, 1.71 mmol, 33% yield). ¹H NMR (DMSO-d6) δ: 8.37 (d, J=9.1 Hz, 1H), 7.87-7.68 (m, 1H), 7.68-7.43 (m, 9H), 7.43-7.32 (m, 1H), 4.02 (td, J=9.6, 4.8 Hz, 1H), 3.11 (dd, J=13.8, 4.9 Hz, 1H), 2.83 (dd, J=13.8, 10.0 Hz, 1H). UPLC-MS (basic 2 min) Rt=0.75 min. m/z=329.0 for [M−H]⁺

### Step 2: N-[2-(3-cyanophenyl)-1-(6-methoxy-1,3-benzothiazol-2-yl)ethyl]benzenesulfonamide

2-benzenesulfonamido-3-(3-cyanophenyl)propanoic acid (0.300 g, 0.908 mmol, 1.0 eq.) was dissolved in T3P (50% w/w in EtOAc) (1.1 mL, 1.82 mmol, 2.0 eq.). DIPEA (0.237 mL, 1.36 mmol, 1.5 eq.) and 2-amino-5-methoxybenzene-1-thiol (0.169 g, 1.09 mmol, 1.2 eq.) were added and the reaction mixture was heated at 100° C. under microwave irradiation for 10 mins. The reaction mixture was cooled to RT and then concentrated to dryness. The residue was purified by Reverse Phase column chromatography eluting with a 5-95% H₂O:MeCN eluent (0.1% ammonia) to afford product as a white solid (0.197 g, 0.429 mmol, 47% yield). ¹H NMR (DMSO-d6) δ: 8.93 (s, 1H), 7.82 (d, J=8.9 Hz, 1H), 7.65 (d, J=2.6 Hz, 1H), 7.59 (t, J=1.7 Hz, 1H), 7.52 (dq, J=8.2, 2.1, 1.7 Hz, 2H), 7.44 (td, J=7.5, 1.4 Hz, 3H), 7.33-7.26 (m, 3H), 7.10 (dd, J=8.9, 2.6 Hz, 1H), 4.97 (d, J=8.7 Hz, 1H), 2.99 (dd, J=13.9, 10.5 Hz, 1H). UPLC-MS (basic 2 min) Rt=1.10 min. m/z=448.9 for [M−H]⁺

### Step 3: N-[2-(3-cyanophenyl)-1-(6-hydroxy-1,3-benzothiazol-2-yl)ethyl]benzenesulfonamide

To a magnetically stirred solution of N-[2-(3-cyanophenyl)-1-(6-methoxy-1,3-benzothiazol-2-yl)ethyl]benzenesulfonamide (0.250 g, 0.556 mmol, 1.0 eq.) in DCM (5.0 mL) was added tribromoborane (1.2 mL, 1.20 mmol, 2.2 eq.) at −78° C. The reaction mixture was warmed to RT and stirred for 24 h. Additional tribromoborane (1.2 mL, 1.20 mmol, 2.2 eq.) was added and the reaction mixture stirred at RT for 1 h. The mixture was quenched with IPA, and then concentrated to dryness. The residue was purified by Reverse Phase column chromatography eluting with a 5-95% H₂O:MeCN eluent (0.1% formic acid) to afford product as a white solid (0.154 g, 0.354 mmol, 64% yield). ¹H NMR (DMSO-d6) δ: 9.77 (br s, 1H), 8.58-9.05 (m, 1H), 7.71 (d, J=8.8 Hz, 1H), 7.57 (s, 1H), 7.47-7.54 (m, 2H), 7.40-7.47 (m, 3H), 7.24-7.36 (m, 4H), 6.94 (br d, J=8.8 Hz, 1H), 4.92 (dd, J=10.3, 4.6 Hz, 1H), 3.36 (br d, J=4.2 Hz, 1H), 2.97 (dd, J=13.6, 10.6 Hz, 1H). UPLC-MS (basic 2 min) Rt=1.01 min. m/z=434.0 for [M+H]⁺

### Step 4: 3-[2-benzenesulfonamido-2-(6-hydroxy-1,3-benzothiazol-2-yl)ethyl]-N′-hydroxybenzene-1-carboximidamide)

To a magnetically stirred solution of N-[2-(3-cyanophenyl)-1-(6-hydroxy-1,3-benzothiazol-2-yl)ethyl]benzenesulfonamide) (0.154 g, 0.354 mmol, 1.0 eq.) in EtOH (4 mL) were added hydroxylamine hydrochloride (0.067 g, 0.964 mmol, 2.7 eq.) and DIPEA (0.20 mL, 1.15 mmol, 3.2 eq.). The reaction mixture was stirred under reflux for 1 h. The reaction mixture was cooled down to RT and then concentrated to dryness to afford product as a brown glass (0.190 g, 0.354 mmol, 100% yield) which was used in the next step without further purification. UPLC-MS (acidic 2 min) Rt=0.85 min. m/z=469.0 for [M+H]⁺

### Step 5: [amino({3-[2-benzenesulfonamido-2-(6-hydroxy-1,3-benzothiazole-2-yl)ethyl]phenyl}) methylidene]amino acetate)

To a magnetically stirred solution of 3-[2-benzenesulfonamido-2-(6-hydroxy-1,3-benzothiazol-2-yl)ethyl]-N′-hydroxybenzene-1-carboximidamide) (0.190 g, 0.406 mmol, 1.0 eq) in acetic acid (4.0 mL) was added acetic anhydride (0.120 mL, 1.27 mmol, 3.1 eq.) and the resulting mixture was stirred at RT for 60 mins. The reaction mixture was concentrated to dryness and the residue was purified by Reverse Phase column chromatography eluting with a 5-95% H₂O:MeCN eluent (0.1% formic acid) to afford product as a pale brown solid (0.076 g, 0.145 mmol, 33% yield). ¹H NMR (400 MHz, DMSO-d₆): δ 9.75 (s, 1H), 8.68-8.94 (m, 1H), 7.69 (d, J=8.8 Hz, 1H), 7.57 (s, 1H), 7.46 (br d, J=8.1 Hz, 3H), 7.34-7.41 (m, 1H), 7.30 (d, J=2.0 Hz, 1H), 7.22-7.28 (m, 2H), 7.20 (br d, J=7.6 Hz, 1H), 7.08-7.16 (m, 1H), 6.92 (dd, J=8.9, 2.3 Hz, 1H), 6.72 (s, 2H), 4.81-4.95 (m, 1H), 3.25 (br d, J=5.1 Hz, 1H), 2.97 (br dd, J=13.6, 9.9 Hz, 1H), 2.15 (s, 3H). UPLC-MS (basic 2 min): Rt=0.91 min; m/z=509.0 for [M+H]⁺

### Step 6: 3-[2-(benzenesulfonamido)-2-(6-hydroxy-1,3-benzothiazol-2-yl)ethyl]benzamidine

To a magnetically stirred solution of [amino({3-[2-benzenesulfonamido-2-(6-hydroxy-1,3-benzothiazole-2-yl)ethyl]phenyl}) methylidene]amino acetate) (0.083 g, 0.163 mmol, 1.0 eq) in acetic acid (2.0 mL) was added zinc (0.438 g, 6.70 mmol, 40.0 eq.). The reaction mixture was stirred at room temperature for 48 h. The reaction mixture was filtered through a plug of celite and then concentrated to dryness. The residue was purified by Reverse Phase column chromatography on a 120 g C18 cartridge eluting with a 5-95% H₂O:MeCN eluent (0.1% formic acid) to afford the formate salt as a white solid. The formate salt was passed through an SCX column to afford product as a white solid (0.005 g, 0.011 mmol, 7% yield). ¹H NMR (400 MHz, DMSO-d₆): δ 9.78 (s, 1H), 9.20 (br s, 2H), 8.91 (s, 2H), 8.83 (d, J=8.3 Hz, 1H), 7.67-7.71 (m, 2H), 7.56 (br d, J=7.8 Hz, 1H), 7.47 (d, J=7.6 Hz, 3H), 7.35-7.42 (m, 1H), 7.28-7.34 (m, 2H), 7.22-7.27 (m, 2H), 6.91-6.97 (m, 1H), 4.93-5.03 (m, 1H), 3.36 (br d, J=4.6 Hz, 1H), 2.99-3.06 (m, 1H). UPLC-MS (basic 4 min): Rt=1.61 min; m/z=451.1 for [M−H]f, 98% purity

### Example 40. Exemplary Compounds

The compounds of the disclosure are identified in Table 2. The compounds may be prepared using the preparation methods described above.

TABLE 2
Exemplary compounds

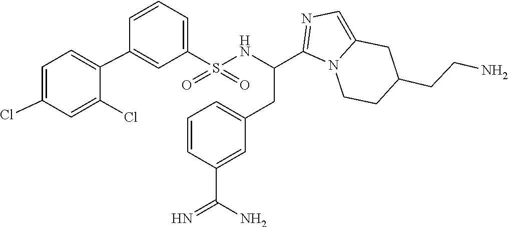 [SMILES: N=C(N)c1cccc(CC(NS(=O)(=O)c2cccc(-c3ccc(Cl)cc3Cl)c2)c2ncc3n2CCC(CCN)C3)c1]

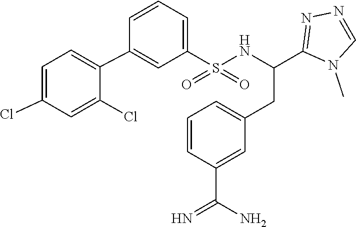 [SMILES: Cn1cnnc1C(Cc1cccc(C(=N)N)c1)NS(=O)(=O)c1cccc(-c2ccc(Cl)cc2Cl)c1]

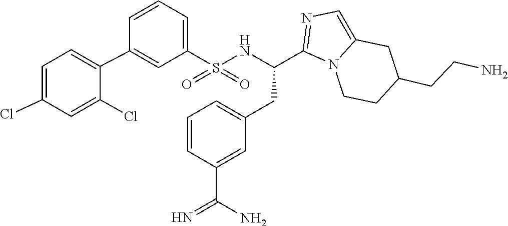 [SMILES: N=C(N)c1cccc(C[C@H](NS(=O)(=O)c2cccc(-c3ccc(Cl)cc3Cl)c2)c2ncc3n2CCC(CCN)C3)c1]

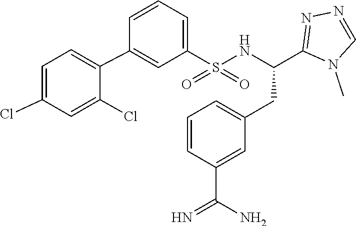 [SMILES: Cn1cnnc1[C@H](Cc1cccc(C(=N)N)c1)NS(=O)(=O)c1cccc(-c2ccc(Cl)cc2Cl)c1]

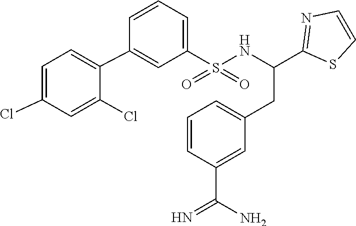 [SMILES: N=C(N)c1cccc(CC(NS(=O)(=O)c2cccc(-c3ccc(Cl)cc3Cl)c2)c2nccs2)c1]

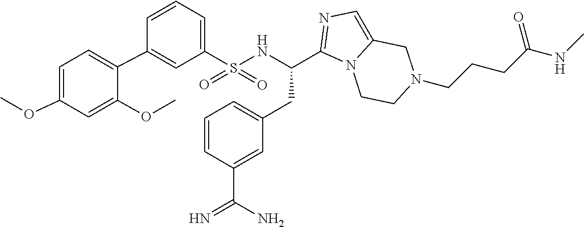

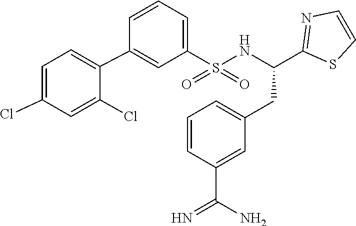 [SMILES: N=C(N)c1cccc(C[C@H](NS(=O)(=O)c2cccc(-c3ccc(Cl)cc3Cl)c2)c2nccs2)c1]

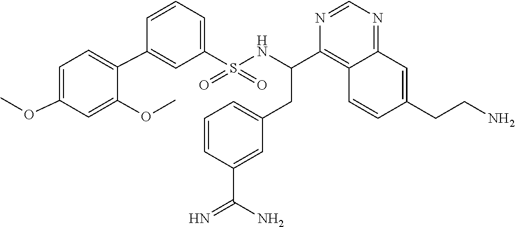 [SMILES: COc1ccc(-c2cccc(S(=O)(=O)NC(Cc3cccc(C(=N)N)c3)c3ncnc4cc(CCN)ccc34)c2)c(OC)c1]

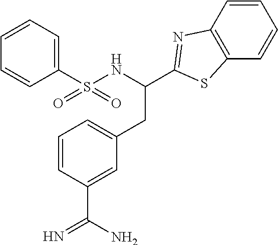 [SMILES: N=C(N)c1cccc(CC(NS(=O)(=O)c2ccccc2)c2nc3ccccc3s2)c1]

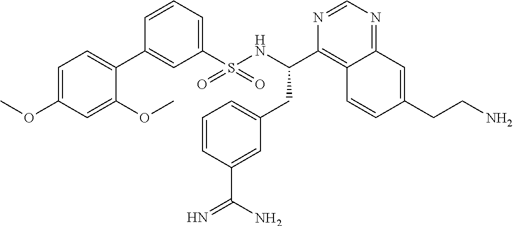 [SMILES: COc1ccc(-c2cccc(S(=O)(=O)N[C@@H](Cc3cccc(C(=N)N)c3)c3ncnc4cc(CCN)ccc34)c2)c(OC)c1]

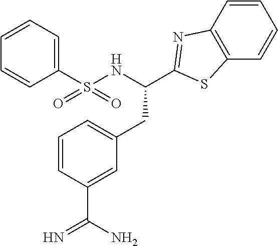 [SMILES: N=C(N)c1cccc(C[C@H](NS(=O)(=O)c2ccccc2)c2nc3ccccc3s2)c1]

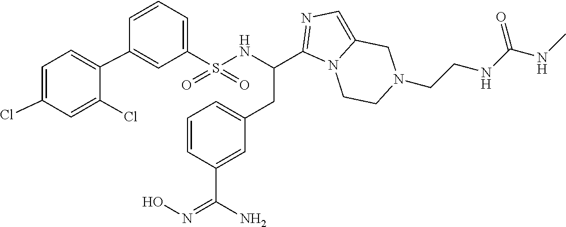 [SMILES: CNC(=O)NCCN1CCn2c(cnc2C(Cc2cccc(/C(N)=N\O)c2)NS(=O)(=O)c2cccc(-c3ccc(Cl)cc3Cl)c2)C1]

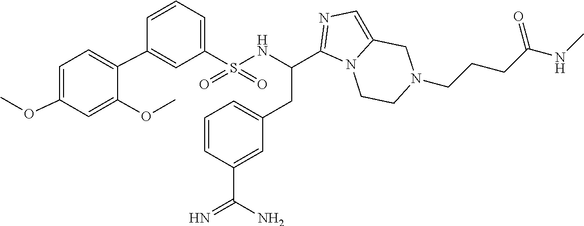

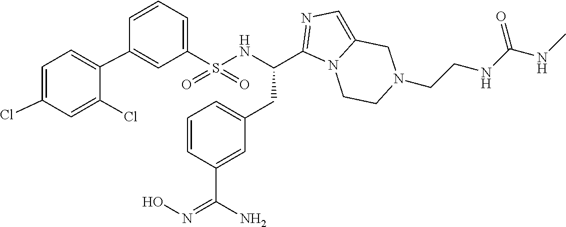 [SMILES: CNC(=O)NCCN1CCn2c(cnc2[C@H](Cc2cccc(/C(N)=N\O)c2)NS(=O)(=O)c2cccc(-c3ccc(Cl)cc3Cl)c2)C1]

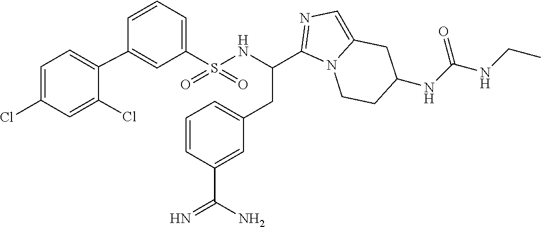 [SMILES: CCNC(=O)NC1CCn2c(cnc2C(Cc2cccc(C(=N)N)c2)NS(=O)(=O)c2cccc(-c3ccc(Cl)cc3Cl)c2)C1]

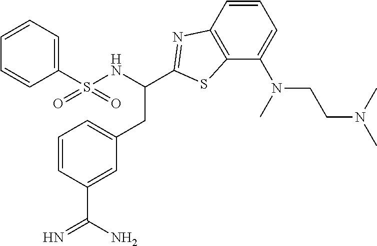 [SMILES: CN(C)CCN(C)c1cccc2nc(C(Cc3cccc(C(=N)N)c3)NS(=O)(=O)c3ccccc3)sc12]

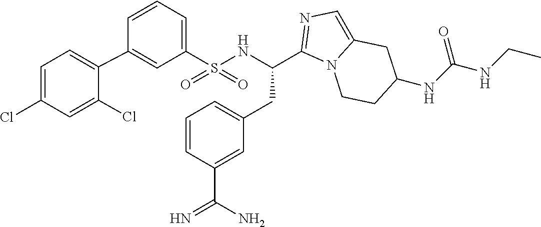 [SMILES: CCNC(=O)NC1CCn2c(cnc2[C@H](Cc2cccc(C(=N)N)c2)NS(=O)(=O)c2cccc(-c3ccc(Cl)cc3Cl)c2)C1]

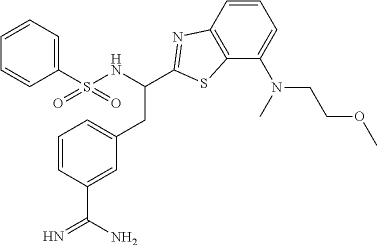 [SMILES: COCCN(C)c1cccc2nc(C(Cc3cccc(C(=N)N)c3)NS(=O)(=O)c3ccccc3)sc12]

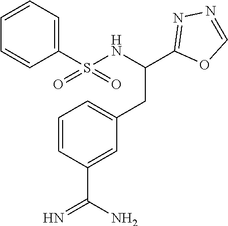 [SMILES: N=C(N)c1cccc(CC(NS(=O)(=O)c2ccccc2)c2nnco2)c1]

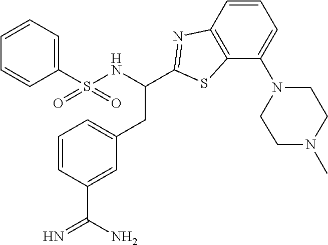 [SMILES: CN1CCN(c2cccc3nc(C(Cc4cccc(C(=N)N)c4)NS(=O)(=O)c4ccccc4)sc23)CC1]

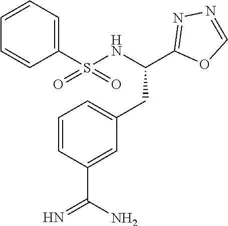 [SMILES: N=C(N)c1cccc(C[C@H](NS(=O)(=O)c2ccccc2)c2nnco2)c1]

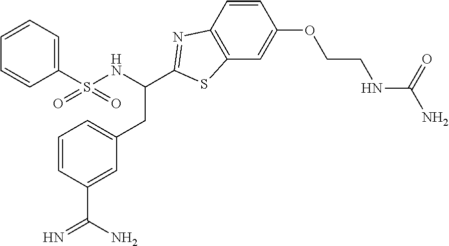 [SMILES: N=C(N)c1cccc(CC(NS(=O)(=O)c2ccccc2)c2nc3ccc(OCCNC(N)=O)cc3s2)c1]

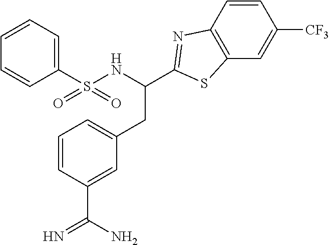 [SMILES: N=C(N)c1cccc(CC(NS(=O)(=O)c2ccccc2)c2nc3ccc(C(F)(F)F)cc3s2)c1]

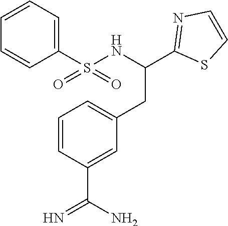 [SMILES: N=C(N)c1cccc(CC(NS(=O)(=O)c2ccccc2)c2nccs2)c1]

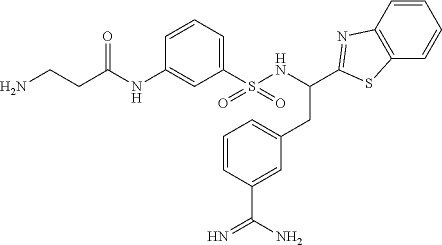 [SMILES: N=C(N)c1cccc(CC(NS(=O)(=O)c2cccc(NC(=O)CCN)c2)c2nc3ccccc3s2)c1]

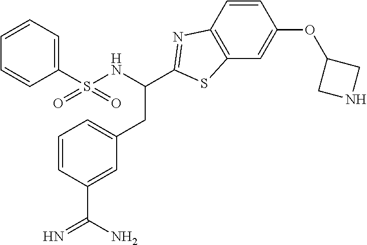 [SMILES: N=C(N)c1cccc(CC(NS(=O)(=O)c2ccccc2)c2nc3ccc(OC4CNC4)cc3s2)c1]

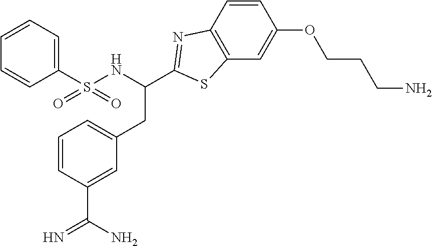 [SMILES: N=C(N)c1cccc(CC(NS(=O)(=O)c2ccccc2)c2nc3ccc(OCCCN)cc3s2)c1]

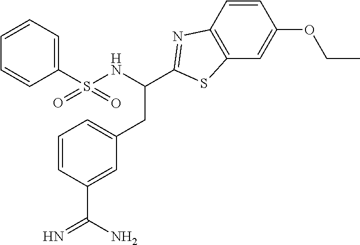 [SMILES: CCOc1ccc2nc(C(Cc3cccc(C(=N)N)c3)NS(=O)(=O)c3ccccc3)sc2c1]

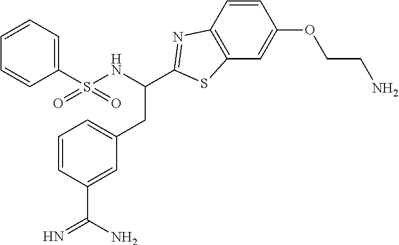 [SMILES: N=C(N)c1cccc(CC(NS(=O)(=O)c2ccccc2)c2nc3ccc(OCCN)cc3s2)c1]

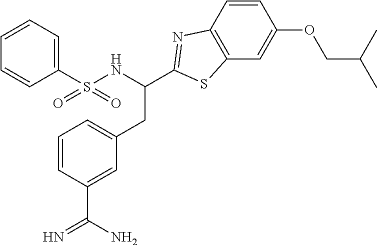 [SMILES: CC(C)COc1ccc2nc(C(Cc3cccc(C(=N)N)c3)NS(=O)(=O)c3ccccc3)sc2c1]

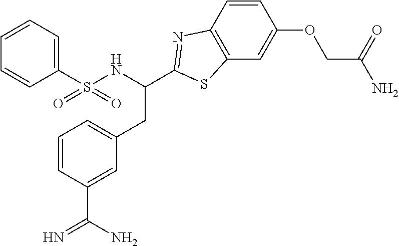 [SMILES: N=C(N)c1cccc(CC(NS(=O)(=O)c2ccccc2)c2nc3ccc(OCC(N)=O)cc3s2)c1]

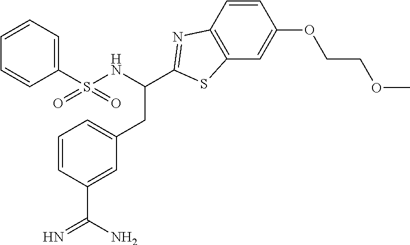 [SMILES: COCCOc1ccc2nc(C(Cc3cccc(C(=N)N)c3)NS(=O)(=O)c3ccccc3)sc2c1]

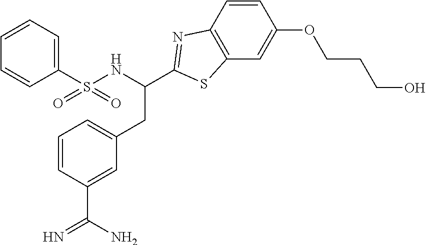 [SMILES: N=C(N)c1cccc(CC(NS(=O)(=O)c2ccccc2)c2nc3ccc(OCCCO)cc3s2)c1]

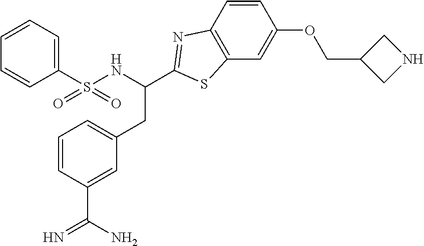 [SMILES: N=C(N)c1cccc(CC(NS(=O)(=O)c2ccccc2)c2nc3ccc(OCC4CNC4)cc3s2)c1]

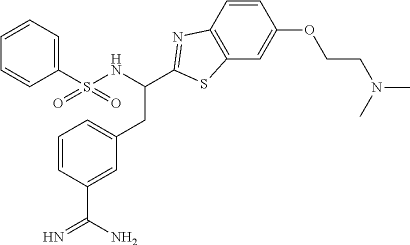 [SMILES: CN(C)CCOc1ccc2nc(C(Cc3cccc(C(=N)N)c3)NS(=O)(=O)c3ccccc3)sc2c1]

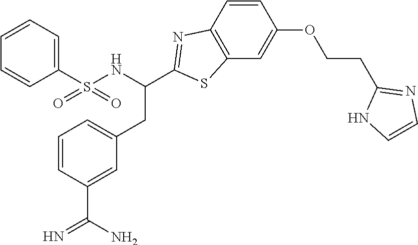 [SMILES: N=C(N)c1cccc(CC(NS(=O)(=O)c2ccccc2)c2nc3ccc(OCCc4ncc[nH]4)cc3s2)c1]

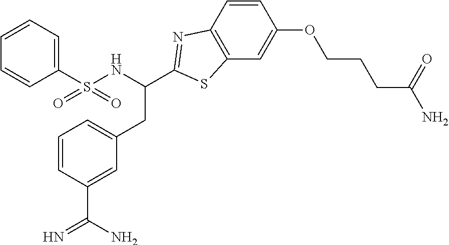 [SMILES: N=C(N)c1cccc(CC(NS(=O)(=O)c2ccccc2)c2nc3ccc(OCCCC(N)=O)cc3s2)c1]

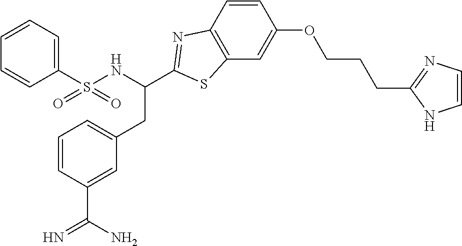 [SMILES: N=C(N)c1cccc(CC(NS(=O)(=O)c2ccccc2)c2nc3ccc(OCCCc4ncc[nH]4)cc3s2)c1]

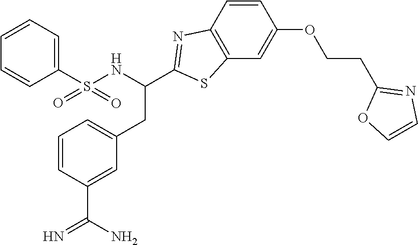 [SMILES: N=C(N)c1cccc(CC(NS(=O)(=O)c2ccccc2)c2nc3ccc(OCCc4ncco4)cc3s2)c1]

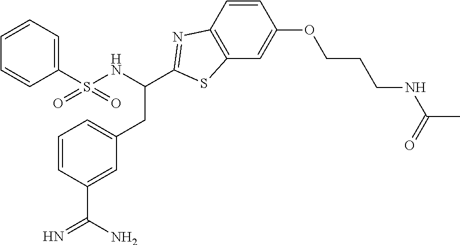 [SMILES: CC(=O)NCCCOc1ccc2nc(C(Cc3cccc(C(=N)N)c3)NS(=O)(=O)c3ccccc3)sc2c1]

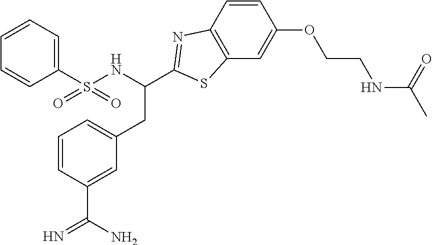 [SMILES: CC(=O)NCCOc1ccc2nc(C(Cc3cccc(C(=N)N)c3)NS(=O)(=O)c3ccccc3)sc2c1]

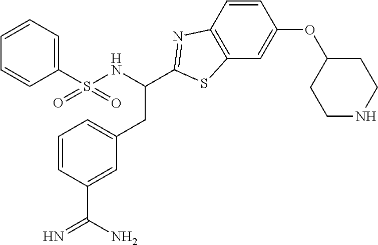 [SMILES: N=C(N)c1cccc(CC(NS(=O)(=O)c2ccccc2)c2nc3ccc(OC4CCNCC4)cc3s2)c1]

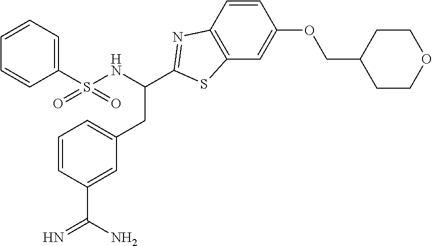 [SMILES: N=C(N)c1cccc(CC(NS(=O)(=O)c2ccccc2)c2nc3ccc(OCC4CCOCC4)cc3s2)c1]

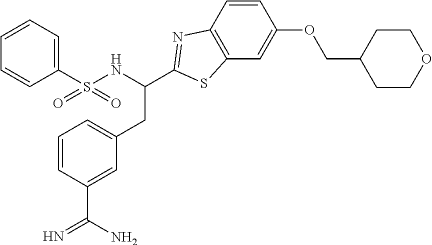 [SMILES: N=C(N)c1cccc(CC(NS(=O)(=O)c2ccccc2)c2nc3ccc(OCC4CCOCC4)cc3s2)c1]

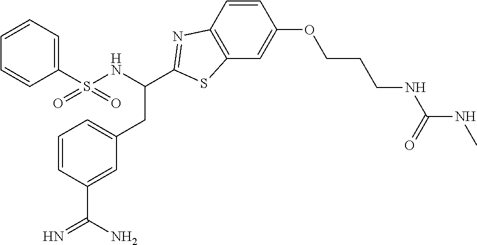 [SMILES: CNC(=O)NCCCOc1ccc2nc(C(Cc3cccc(C(=N)N)c3)NS(=O)(=O)c3ccccc3)sc2c1]

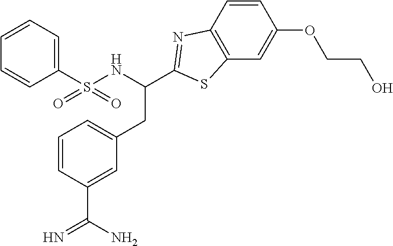 [SMILES: N=C(N)c1cccc(CC(NS(=O)(=O)c2ccccc2)c2nc3ccc(OCCO)cc3s2)c1]

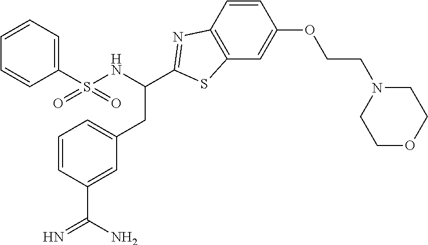 [SMILES: N=C(N)c1cccc(CC(NS(=O)(=O)c2ccccc2)c2nc3ccc(OCCN4CCOCC4)cc3s2)c1]

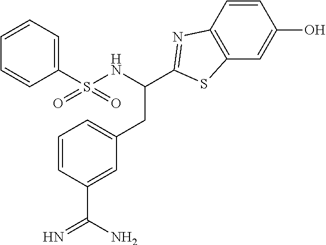 [SMILES: N=C(N)c1cccc(CC(NS(=O)(=O)c2ccccc2)c2nc3ccc(O)cc3s2)c1]

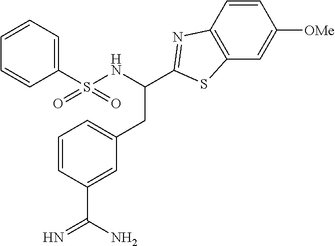 [SMILES: COc1ccc2nc(C(Cc3cccc(C(=N)N)c3)NS(=O)(=O)c3ccccc3)sc2c1]

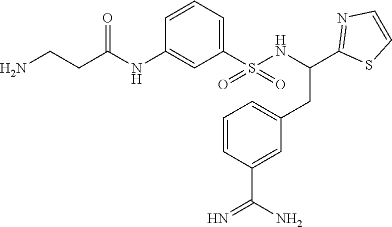 [SMILES: N=C(N)c1cccc(CC(NS(=O)(=O)c2cccc(NC(=O)CCN)c2)c2nccs2)c1]

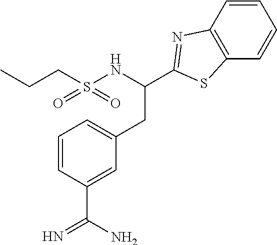 [SMILES: CCCS(=O)(=O)NC(Cc1cccc(C(=N)N)c1)c1nc2ccccc2s1]

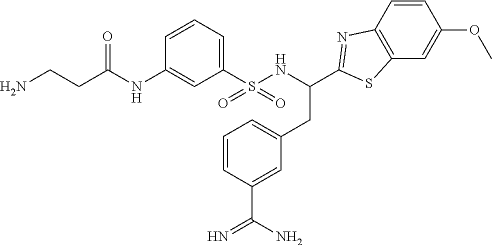 [SMILES: COc1ccc2nc(C(Cc3cccc(C(=N)N)c3)NS(=O)(=O)c3cccc(NC(=O)CCN)c3)sc2c1]

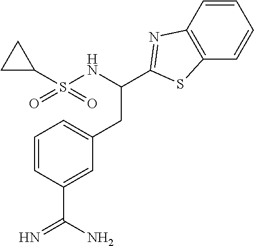 [SMILES: N=C(N)c1cccc(CC(NS(=O)(=O)C2CC2)c2nc3ccccc3s2)c1]

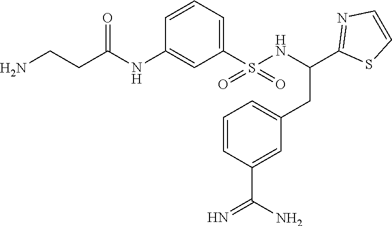 [SMILES: N=C(N)c1cccc(CC(NS(=O)(=O)c2cccc(NC(=O)CCN)c2)c2nccs2)c1]

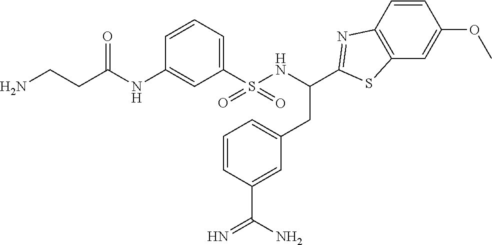 [SMILES: COc1ccc2nc(C(Cc3cccc(C(=N)N)c3)NS(=O)(=O)c3cccc(NC(=O)CCN)c3)sc2c1]

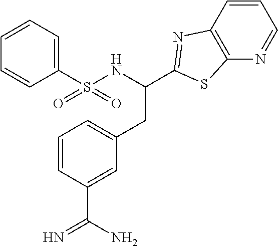 [SMILES: N=C(N)c1cccc(CC(NS(=O)(=O)c2ccccc2)c2nc3cccnc3s2)c1]

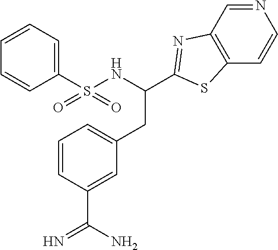 [SMILES: N=C(N)c1cccc(CC(NS(=O)(=O)c2ccccc2)c2nc3cnccc3s2)c1]

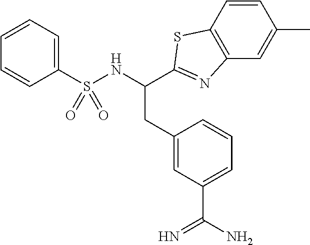 [SMILES: Cc1ccc2sc(C(Cc3cccc(C(=N)N)c3)NS(=O)(=O)c3ccccc3)nc2c1]

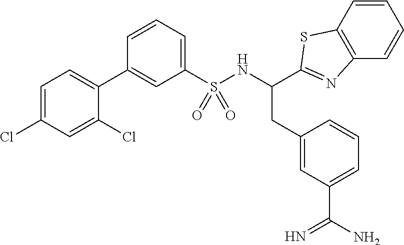 [SMILES: N=C(N)c1cccc(CC(NS(=O)(=O)c2cccc(-c3ccc(Cl)cc3Cl)c2)c2nc3ccccc3s2)c1]

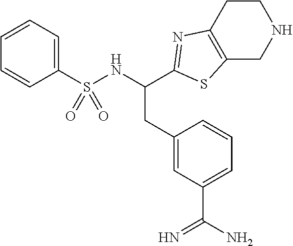 [SMILES: N=C(N)c1cccc(CC(NS(=O)(=O)c2ccccc2)c2nc3c(s2)CNCC3)c1]

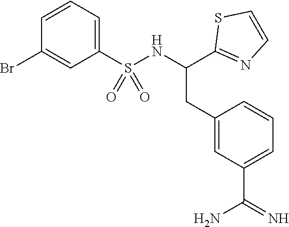 [SMILES: N=C(N)c1cccc(CC(NS(=O)(=O)c2cccc(Br)c2)c2nccs2)c1]

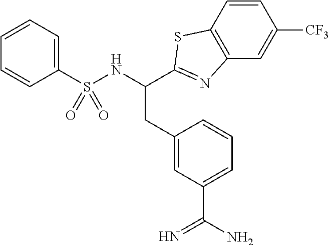 [SMILES: N=C(N)c1cccc(CC(NS(=O)(=O)c2ccccc2)c2nc3cc(C(F)(F)F)ccc3s2)c1]

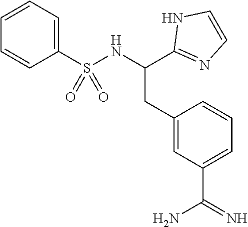 [SMILES: N=C(N)c1cccc(CC(NS(=O)(=O)c2ccccc2)c2ncc[nH]2)c1]

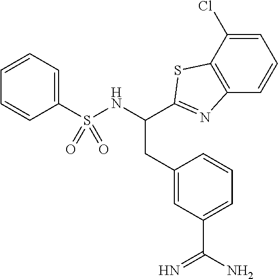 [SMILES: N=C(N)c1cccc(CC(NS(=O)(=O)c2ccccc2)c2nc3cccc(Cl)c3s2)c1]

### Example 41. Evaluation of Activity of Compounds in the TMPRSS2 Assay 1

The enzyme (TMPRSS2, CusaBio cat #CSB-YP023924HU. Recombinant human TMPRSS2 protein (106-492 aa)N-terminal 6×His-tagged (“6×His” disclosed as SEQ ID NO: 4), expressed in yeast cells; MW=44.8 kDa, lyophilized from 20 mM: Tris HCl, 0.5 M NaCl, 6% Trehalose, pH 8.0, >85% purity as determined by SDS-PAGE. Uniprot: 015393; 10 nM in the reaction) and substrate (Boc-Gln-Ala-Arg-AMC; Enzo Cat #BML-P237-0005, MW=581.76 Da. 25 μM in the reaction) were prepared in Reaction Buffer (50 mM Tris-HCl pH 8, 150 mM NaCl, 0.005% Brij35, 1% DMSO). After the enzyme solution was delivered into the reaction well, the compounds in DMSO were delivered to the reaction mixture by using Acoustic Technology (Echo 550, LabCyte Inc. Sunnyvale, CA) in nanoliter range. After 20 min pre-incubation, the substrate solution was delivered into the reaction well to initiate the reaction. The enzyme activities were monitored every 5 min as a time-course measurement of the increase in fluorescence signal from fluorescently-labeled peptide substrate for 120 min. at room temperature. Data was analyzed by taking slope*(signal/time) of linear portion of measurement. The slope was calculated using Excel, and the curve fits were performed using Prism software.

### Example 42. Evaluation of Activity of Compounds in the TMPRSS2 Assay 2

The concentration-response testing of compounds was in primary, readout counter as 11-point compound dilution series in 384-well assay plate format in triplicates (n=3). The compound dilutions was prepared in an intermediate plate (spot volume=150 nL), and then the assay reagents were prepared. After preparing the intermediate plates containing the assay reagents for the transfer into the assay plate using the Selma 384/25 μl pipettor, the assay reagents were transferred into the assay plate.

| Primary assay | Readout counter assay |
|---|---|
| 5 μl assay buffer | 15 μl coumarin dilution |
| (Multichannel pipet) | (Multichannel pipet) |
| 5 μl 3X substrate dilution (Selma) | |
| 5 μl 3X enzyme dilution (Selma) | |

The resulting plate was centrifuged at g=290 for 30 s, and then the plate was incubated at room temperature for 150 minutes. 10 μl 2.5× of stop reagent (50 mM acetic acid) (Selma) was added, and then the plate was read on Safire e.

The materials, reagents, and equipment used for the assay is described in Table 3, Table 4, Table 5, and Table 6.

TABLE 3
Materials for the TMPRSS2 assay

| Name | Supplier/Batch No | Preparation/Description | Storage |
|---|---|---|---|
| 1M Trizma ® | Sigma Aldrich | ready-to-use | RT |
| hydrochloride solution | cat# T3038-1L | (0.2 μm filtered) | |
| | lot# SLCf1681 | | |
| 5M NaCl | Sigma Aldrich | ready-to-use | RT |
| | cat# 59222C-500ML | | |
| | lot# SLCF8319 | | |
| 5% glycerol/dH₂O | Sigma Aldrich | 2.5 g glycerol (99%) are diluted in | 4° C. |
| (w/w) | cat# G55161 | 47.5 g dH₂O (0.2 μm filtered) | |
| | batch# 07734DH | (for reconstitution of TMPRSS2) | |
| 10% Tween ® 20 | Sigma Aldrich | 0.5 ml of Tween ® 20 are diluted in 4.5 | 4° C. |
| | cat# P2287 | ml dH₂O. | |
| | lot# MKBG0511V | | |
| 100% DMSO | Sigma Aldrich | ready-to-use | RT |
| | cat# D8418 | | |
| | lot# SHBK2703 | | |
| 125 mM Acetic Acid | Sigma | MW 60.05 g/mol | RT |
| stop reagent | cat# 320099 | Add 1.5 ml of the 99.7% (~16.65M) | |
| | lot# STBD5733V | acetic acid to 198.5 ml dH₂O | |

TABLE 4
Specific assay reagents for the TMPRSS2 assay

| Name | Supplier/Batch No | Preparation | Storage |
|---|---|---|---|
| 22.32 μM | CUSABIO | Human TMPRSS2-(AA 106-492)- | −80° C. |
| TMPRSS2 | cat# CSB- | O15393-Partial Protein | Do not repeat |
| | YP023924HU | MW (g/mol) 44800 | freezing and |
| | batch# DA04624a3g0 | Reconstitution in 0.1 ml sterile dH₂O, | thawing |
| | | 5% glycerol to a concentration of 1 | |
| | | mg/ml per vial -> 22.32 μM | |
| 10 mM Boc-Gln- | Bachem | MW (g/mol) 667.16 | Aliquots |
| Ala-Arg-AMC | cat# I-1550 | 5 mg was dissolved in 749 μl 100% | at −20° C. |
| | lot# 100015378 | DMSO | |
| | product# | | |
| | 4017019.0005 | | |
| 10 mM Coumarin | Sigma | MW (g/mol) 175.18 | Aliquots |
| (7-Amino-4- | cat# A9891-250MG | 5.9 mg was dissolved in 3.368 ml | at −20° C. |
| Methylcoumarin) | lot# SLBX7741 | 100% DMSO | |

TABLE 5
Other reagents for the TMPRSS2 assay

| Name | Supplier/Batch No | Preparation |
|---|---|---|
| 10 mM | Evotec Abingdon | STBD5733Vdissolved in 100% |
| MI-461 | EOAI10017641 | DMSO by Compound |
| | EV-HDK001-170-006 | Management |
| 10 mM | Evotec Abingdon | MW (g/mol) 539.58 |
| Nafamostat | EOAI3936573 | dissolved in 100% DMSO by |
| | EV-HDK001-170-002 | Compound Management |

TABLE 6
Equipment for the TMPRSS2 assay

| Name | Source | Description | Additional information |
|---|---|---|---|
| Safire2 | Tecan | Multilabel Reader | Firmware: V 1.60 07/2006 |
| | | | Safire2 |
| Selma, 384/25 μl | analytikjena | Bench top pipettor | System ID 307001-16-2006 |
| Intermediate plate | Axygen (Corning) | PP, 384 well, deep | |
| | (cat # P-384-120SQ- | well, with square | |
| | C) | wells (“Diamond”) | |
| Intermediate plate | Greiner bio-one | 384-well plate pp, | Internal Evotec name: |
| | (cat # 784201) | small volume, deep | “greiner-dilution” |
| | | well, v-shape | |
| Assay plate | Greiner bio-one | 384 well plate black, | |
| | (cat# 781900) | PS, non-binding | |
| | | surface | |

Preparation of Assay Reagents

For the primary assay, the assay buffer (50 mM Tris-HCL, 150 mM NaCl, 0.01% Tween®20, pH8.0) was prepared using the stocks as described in Table 7.

TABLE 7
Preparation of Assay Buffer

| | | | |
|---|---|---|---|
| Required | Volume | Concentration of stock | Final assay |
| stocks | (ml) | solution | concentration |

Preparation of Assay Buffer (500 ml)

| | | | |
|---|---|---|---|
| Trizma | 25 | 1M | 50 mM |
| NaCl | 15 | 5M | 150 mM |
| Tween20 | 0.5 | 10% | 0.10% |
| Distilled Water | 480 | — | — |

Adjust pH to 8.0, fill up to 500 ml, filter through a 0.22 μm membrane

The enzyme dilution was prepared as described in Table 8.

TABLE 8
Preparation of the enzyme dilution

| | | | |
|---|---|---|---|
| | Volume | Concentration of | Final assay |
| Required stocks | (μl) | working solution | concentration |

Preparation of the enzyme dilution (6 ml,
sufficient for a test of three half plates)

| | | | |
|---|---|---|---|
| Assay Buffer | 5983.9 | — | — |
| TMPRSS2, 22.32 μM | 16.1 | 60 nM | 20 nM |

The final assay concentration was referred to 15 μl assay volume. Using a “greiner-dilution” as intermediate plate, the according wells were filled (23 l). The volume was adjusted using another plate type. Substrate dilution was prepared as described in Table 9.

Preparation of the Substrate Dilution

TABLE 9
Preparation of the enzyme dilution

| | Volume | Concentration of | Final assay |
|---|---|---|---|
| Required stocks | (μl) | working solution | concentration |
| Preparation of the enzyme dilution (6 ml, | | | |
| sufficient for a test of three half plates) | | | |
| Assay Buffer | — | — | — |
| Boc-Gln-Ala- | 72 | 120 μM | 40 μM |
| Arg-AMC, 10 mM | | | |

The final assay concentration was referred to 15 μl assay volume. The DMSO concentration of the substrate working dilution was 1.2%. Using a “greiner-dilution” as intermediate plate, the according wells were filled (23 μl). Using another plate type, the volume was adjusted.

Readout Counter Assay

The coumarin dilution was prepared as described in Table 10.

TABLE 10
Preparation of coumarin dilution

| | Volume | Concentration of | Final |
|---|---|---|---|
| Required stocks | (μl) | working solution | assay conc. |
| Preparation of the coumarin dilution (12 ml, | | | |
| sufficient for a test of three half plates) | | | |
| Assay Buffer | 11856 | | |
| DMSO | 48 | 0.4% | 1.4% |
| Coumarin, 10 mM | 2.4 | 2 μM | 2 μM |

The final assay concentration was referred to 15 μl assay volume. Using a “greiner-dilution” as intermediate plate, 35 μl was filled into the according wells. Using another plate type, the volume was adjusted.

Compound Handling:

Table 11 demonstrates the calculation for 1 well of the intermediate plate. The compounds were dissolved in DM50. 150 nl of the compound were transferred into the assay plate prior to addition of the assay components (final assay volume of 15 μl).

Top concentration for the dose response of control compound MI-461 was 2.187 μM. The compound was tested in threefold dilution up to 11I concentrations (3.7037·10⁻⁵ μM-2.187 μM).

TABLE 11
MI-461 compound titration

| | Stock conc. intermediate plate (μM) | Final assay conc. |
|---|---|---|
| | (150 nl) | (nM) |
| 1 | 218.7 | 2187 |
| 2 | 72.9 | 729 |
| 3 | 24.3 | 243 |
| 4 | 8.1 | 81 |
| 5 | 2.7 | 27 |
| 6 | 0.9 | 9 |
| 7 | 0.3 | 3 |
| 8 | 0.1 | 1 |
| 9 | 0.033333 | 0.3333 |
| 10 | 0.011111 | 0.1111 |
| 11 | 0.003704 | 0.0370 |

TABLE 12
Titration of test compounds

| | Stock conc. intermediate plate (mM) | Final assay conc. |
|---|---|---|
| | (0.15 μl) | (μM) |
| 1 | 10 | 100 |
| 2 | 3.3333 | 33.3333 |
| 3 | 1.1111 | 11.1111 |
| 4 | 0.3704 | 3.7037 |
| 5 | 0.1235 | 1.2345 |
| 6 | 0.0412 | 0.4115 |
| 7 | 0.0137 | 0.1372 |
| 8 | 0.0046 | 0.0457 |
| 9 | 0.015 | 0.0152 |
| 10 | 0.005 | 0.0051 |
| 11 | 0.002 | 0.0017 |

Preparation of the Control Plate

Table 13 describes pipetting scheme for the Low control (0.8 μM MI-461).

TABLE 13
Pipetting scheme

| | | | | | Transfer conc. |
|---|---|---|---|---|---|
| | | | | | of 0.080 |
| | Low | Volume | Volume | | mM to |
| | control | of | of | Total | intermediate |
| Stock | conc. | Stock | DMSO | Volume | plate column |
| (mM) | (mM) | (μl) | (μl) | (μl) | 18 (μl) |
| 10 | 0.08 | 1 | 199 | 120 | 5 |

The final concentration of the tool compound (low control) in the assay (assay volume l) was 0.8 μM. For the High control 5 μl/well 10000 DMSO was filled in column 6. The readout settings are described in Table 14. The readout was performed utilizing Safire² Microplate Reader.

TABLE 14
Readout settings

| Measurement mode | Fluorescence Top | |
|---|---|---|
| Excitation wavelength | 380 | nm |
| Emission wavelength | 460 | nm |
| Excitation bandwidth | 20 | nm |
| Emission bandwidth | 20 | nm |
| Gain (Manual) | 75 | |
| Number of reads | 10 | |
| FlashMode | High sensitivity | |
| Integration time | 40 | μs |
| Lag time | 0 | μs |
| Plate definition file | GRE384fb.pdf | |
| Z-Position (Manual) | 6900 | μm |
| Time between move and flash | 80 | ms |

Typical results in the assay development is described in Table 15a and Table 15b.

TABLE 15a
Typical readout in 384-well format

| | |
|---|---|
| Negative control = high ctrl | ~1400-2200 RFU |
| (DMSO) | |
| Positive control = lo ctrl | ~75-160 RFU |
| (50 nM MI-461) | |
| Without enzyme control = lo ctrl_2 | ~75-160 RFU |

TABLE 15b
Statistics

| | |
|---|---|
| Z' in 384-well format (High ctrl/lo ctrl) | ~0.8 |
| S/B in 384-well format (High ctrl/lo ctrl) | ~7-8 |
| Z' in 384-well format (High ctrl/lo ctrl_2) | ~0.8 |
| S/B in 384-well format (High ctrl/lo ctrl_2) | ~7-8 |

The Kₘ was determined by preparing a curve of 20 nM TMPRSS2 (CUSABIO) against Boc-Gln-Ala-Arg-AMC (slope v. substrate (μM)). The Michaelis-Menten best-fit values based on the curve were Vmax=6.127, and Km=42.93. The buffer was optimized by measuring the effect of detergents additives (+nth, +0.2% Tween20, +0.01% Tween20, +0.1% Triton X-100, and +0.01% Triton-X-100) to the TMPRSS2 catalyzed cleavage of Boc-Gln-Ala-Arg-AMC, and it was determined that 0.01% Tween-20 was beneficial for the reaction. The DMSO tolerance was measured using the following assay conditions: 20 nM TMPRSS2 (CUSABIO) (Hi), without enzyme (Lo), 40 μM substrate (Boc-Gln-Ala-Arg-AMC). As a result, it was determined that the assay window decreased slightly with increasing DMSO concentration. Typical IC₅₀ values for control inhibitor MI-461 was IC₅₀ in assay development 384 well format=26 nM.

Procedure: Volumes and Concentrations AD

The description of hardware and plates used for the assay development is described in Table 16.

TABLE 16
Hardware and plates for the assay development

| Hardware | Instrument |
|---|---|
| Compound handling (CM) | 150 nl into the assay plates |
| Assay plate | greiner bio-one #781900 384-well PS flat-bottom, black, non-binding |
| Control plate | Greiner bio-one #784201: 384 well plate pp, small volume, deep well, “greiner |
| | dilution” |
| Intermediate plate | Greiner bio-one #784201: 384 well plate pp, small volume, deep well, “greiner |
| | dilution” |
| Transfer of assay reagents | Assay Buffer: Multichannel pipette |
| | Substrate and enzyme dilution: SELMA 383/25 μl, analytikjena |
| | Coumarin dilution: Multichannel pipette |
| | Stop reagent: SELMA 383/25 μl, analytikjena |
| Measurement | Safire 2, Tecan |

Volumes and Incubations Steps/Assay Development in 384-Well Format

The volumes and incubation steps for the assay development is described in Table 17 and Table 18.

TABLE 17
Volumes and incubation steps for primary assay

| Solutions pipetted/ | Negative Control = | Positive Control = | | |
|---|---|---|---|---|
| dispensed in this | High Ctrl | Low Ctrl | | |
| order | (column 6) | (column 18) | Samples | Instrument |
| Compound | 0.15 μl | 0.15 μl | 0.15 μl | Mosquito(CM) |
| Assay buffer | 5 μl | 5 μl | 5 μl | Multichannel pipette |
| Substrate dilution | 5 μl | 5 μl | 5 μl | SELMA 384/25 μl |
| Enzyme dilution | 5 μl | 5 μl | 5 μl | SELMA 384/25 μl |
| Total volume | 15.15 μl | 15.15 μl | 15.15 μl | |
| Incubation | 150 min at room temperature | | | |
| Stop reagent | 10 μl | 10 μl | 10 μl | SELMA 384/25 μl |
| Read on Safire² | | | | |

TABLE 18
Readout counter assay

| Solutions pipetted/ | Negative Control = | Positive Control = | | |
|---|---|---|---|---|
| dispensed in this | High Ctrl | Low Ctrl | | |
| order | (column 6) | (column 18) | Samples | Instrument |
| Compound | 0.15 μl | 0.15 μl | 0.15 μl | Multidrop |
| | | | | (CM) |
| Coumarin dilution | 15 μl | 15 μl | 15 μl | Multichannel pipette |
| Total volume | 15.15 μl | 15.15 μl | 15.15 μl | |
| Incubation | 150 min at room temperature | | | |
| Stop reagent | 10 μl | 10 μl | 10 μl | SELMA 384/25 μl |
| Read on Safire² | | | | |

Table 19 and Table 20 below show activity data.

TABLE 19
In Vitro TMPRSS2 biochemical activity of compounds
measured using assay described in Example 41.

| Compound No. | Activity (IC₅₀) |
|---|---|
| 1 (racemic mixture, batch 1) | A |
| 1 (racemic mixture, batch 3) | A |
| 2 (enantiomer 1, batch 1) | B |
| 1 (enantiomer 1, batch 1) | B |
| 1 (enantiomer 1, batch 2) | B |
| 1 (enantiomer 2, batch 1) | A |
| 1 (enantiomer 2, batch 2) | A |
| 3 (racemic) | A |
| 4 | B |
| 5 (Isomer 1, batch 1) | A |
| 6 | B |
| 2 (enantiomer 1, batch 2) | B |
| 3 (enantiomer 1) | B |
| 3 (enantiomer 2) | A |
| 5 (Isomer 2) | A |
| 5 (Isomer 3) | A |
| 7 | B |
| 8 | A |
| 12 | A |
| 9 | A |
| 13 | A |
| 10 | B |
| 14 (batch 1) | A |
| 11 (enantiomer 1) | B |
| 11 (enantiomer 2) | A |
| 12 (enantiomer 1) | B |
| 12 (enantiomer 2) | A |
| 13 (enantiomer 1) | B |
| 13 (enantiomer 2) | A |
| 14 (enantiomer 1) | B |
| 14 (enantiomer 2) | A |
| 15 | A |
| 16 | B |
| 17 | A |
| 18 | A |
| 19 | A |
| 20 | A |

A = IC₅₀ < 10 μM;
B = IC₅₀ > 10 μM (or minimal or no inhibition and/or compound activity that cannot be fit to an IC₅₀ curve)

TABLE 20
In Vitro TMPRSS2 biochemical activity of compounds
measured using assay described in Example 42.

| Compound No. | Activity (IC₅₀) |
|---|---|
| 1 (racemic mixture, batch 1) | X |
| 2 (enantiomer 1, batch 1) | Y |
| 3 (racemic) | Y |

X = IC₅₀ < 30 μM;
Y = IC₅₀ > 30 μM

### Example 43. Evaluation of Activity of Compounds Against Non-TMPRSS2 Proteases

Biochemical activity of compound 1 (racemic mixture, batch 1), compound 3 (racemic), compound 11, and compound 2 (enantiomer 1, batch 1) in proteases indicated in Table 21, Table 22, Table 23, and Table 24, respectively, were measured by testing the compounds in a 10-dose IC₅₀ with a 3-fold series dilution starting at 10 μM against the proteases. The protease activities were monitored as a time-course measurement of the increase in fluorescence signal from fluorescently-labeled peptide substrate, and initial linear portion of slope (signal/min) was analyzed. The curve fits were performed when the activities at the highest concentration of compounds were less than 65%.

TABLE 21
In Vitro protease biochemical activity of
compound 1 (racemic mixture, batch 1)

| Protease | Activity (IC₅₀) |
|---|---|
| ACE2 | B |
| Cathepsin B | B |
| Cathepsin L | B |
| Elastase | B |
| FVIIa | B |
| FXa | B |
| FXIa | B |
| Furin | B |
| Kallikrein 1 | A |
| Kallikrein 5 | B |
| Kallikrein 7 | B |
| Kallikrein 12 | B |
| Kallikrein 13 | B |
| Kallikrein 14 | B |
| Matriptase 2 | B |
| MMP 1 | B |
| MMP 2 | B |
| MMP 7 | B |
| MMP 10 | B |
| MMP 13 | B |
| MMP 14 | B |
| Mpro | B |
| Plasma Kallikrein | B |
| Plasmin | B |
| PLpro | B |
| TACE | B |
| Thrombin a | B |
| Trypsin | B |
| Tryptase b2 | B |
| Tryptase g1 | B |
| Urokinase | B |

A = IC₅₀ < 10 μM, and
B = IC₅₀ > 10 μM (or minimal or no inhibition and/or compound activity that cannot be fit to an IC₅₀ curve)

TABLE 22
In Vitro protease biochemical
activity of compound 3 (racemic)

| Protease | Activity (IC₅₀) |
|---|---|
| ACE2 | B |
| Cathepsin B | B |
| Cathepsin L | B |
| Elastase | B |
| FVIIa | B |
| FXa | A |
| FXIa | B |
| Furin | B |
| Kallikrein 1 | B |
| Kallikrein 5 | B |
| Kallikrein 7 | B |
| Kallikrein 12 | B |
| Kallikrein 13 | B |
| Kallikrein 14 | B |
| Matriptase 2 | B |
| MMP 1 | B |
| MMP 2 | A |
| MMP 7 | B |
| MMP 10 | B |
| MMP 13 | B |
| MMP 14 | A |
| Mpro | B |
| Plasma Kallikrein | B |
| Plasmin | B |
| PLpro | B |
| TACE | B |
| Thrombin a | A |
| Trypsin | A |
| Tryptase b2 | A |
| Tryptase g1 | A |
| Urokinase | B |

A = IC₅₀ < 10 μM, and
B = IC₅₀ > 10 μM (or minimal or no inhibition and/or compound activity that cannot be fit to an IC₅₀ curve)

TABLE 23
In Vitro protease biochemical activity of compound 11

| Protease | Activity (IC₅₀) |
|---|---|
| ACE2 | B |
| Cathepsin B | B |
| Cathepsin L | B |
| Elastase | B |
| Factor VIIa | B |
| Factor Xa | B |
| Factor XIa | B |
| Furin | B |
| Kallikrein 1 | A |
| Kallikrein 5 | B |
| Kallikrein 7 | B |
| Kallikrein 12 | B |
| Kallikrein 13 | B |
| Kallikrein 14 | B |
| Matriptase 2 | A |
| MMP 1 | B |
| MMP 2 | B |
| MMP 7 | B |
| MMP 10 | B |
| MMP 13 | B |
| MMP 14 | B |
| Mpro | B |
| Plasma Kallikrein | B |
| Plasmin | B |
| PLpro | B |
| TACE | B |
| Thrombin a | B |
| Trypsin | B |
| Tryptase b2 | B |
| Tryptase g1 | B |
| Urokinase | A |

A = IC₅₀ < 10 μM, and
B = IC₅₀ > 10 μM (or minimal or no inhibition and/or compound activity that cannot be fit to an IC₅₀ curve)

TABLE 24
In Vitro protease biochemical activity of
compound 2 (enantiomer 1, batch 1)

| Protease | Activity (IC₅₀) |
|---|---|
| ACE2 | B |
| Cathepsin B | B |
| Cathepsin L | B |
| Elastase | B |
| FVIIa | B |
| FXa | B |
| FXIa | B |
| Furin | B |
| Kallikrein 1 | B |
| Kallikrein 5 | B |
| Kallikrein 7 | B |
| Kallikrein 12 | B |
| Kallikrein 13 | B |
| Kallikrein 14 | B |
| Matriptase 2 | B |
| MMP 1 | B |
| MMP 2 | B |
| MMP 7 | B |
| MMP 10 | B |
| MMP 13 | B |
| MMP 14 | B |
| Mpro | B |
| Plasma Kallikrein | B |
| Plasmin | B |
| PLpro | B |
| TACE | B |
| Thrombin a | B |
| Trypsin | B |
| Tryptase b2 | B |
| Tryptase g1 | B |
| Urokinase | B |

A = IC₅₀ < 10 μM, and
B = IC₅₀ > 10 μM (or minimal or no inhibition and/or compound activity that cannot be fit to an IC₅₀ curve)

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

What is claimed is:

1-18. (canceled)

19. A compound represented by: 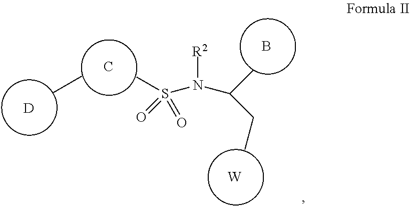
wherein:
C and D are independently selected, for each occurrence, from the group consisting of phenyl and 5-6 membered monocyclic heteroaryl, wherein the phenyl or 5-6 membered monocyclic heteroaryl may optionally be substituted by one or more substituents each selected from R^{g};
R² is selected from hydrogen and C₁₋₆ alkyl;
B is selected from the group consisting of 5-6 membered monocyclic heteroaryl, 8-10 membered bicyclic heteroaryl, and 8-10 membered bicyclic heterocyclyl, wherein B contains at least one nitrogen and B is optionally substituted on one, two, or three carbons by a substituent each independently selected from R^{B}; wherein when said heterocyclyl contains a —NH moiety, the nitrogen of —NH may optionally be substituted by one or more substituents each selected from R^{h};
W is selected from phenyl and heteroaryl, wherein W is substituted on a carbon by a warhead moiety R^{w}, wherein R^{w} is selected from the group consisting of amidine, amidoxime, guanidine, and N-hydroxyguanidine;
R^{B} is selected from the group consisting of: halogen, cyano, hydroxyl, —N(RⁱR^{j}), phenyl, 5-6 membered heteroaryl, 5-6 membered heterocyclyl, —OR^{k}, C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}-(wherein w is 0, 1 or 2), C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆ alkoxycarbonyl-N(R^{a})—, C₁₋₆alkylcarbonyl-N(R^{a})—C₁₋₆alkyl-, C₁₋₆alkyl-N(R^{a})-carbonyl-C₁₋₆alkyl-C₁₋₆alkoxyC₁₋₆alkyl-; wherein the C₁₋₆alkyl, C₂₋₆alkenyl, C₂₋₆alkynyl, C₃₋₆cycloalkyl, C₁₋₆alkoxy, C₃₋₆alkenyloxy, C₃₋₆alkynyloxy, C₃₋₆cycloalkoxy, C₁₋₆alkyl-S(O)_{w}—, C₁₋₆alkyl-N(R^{a})—, C₁₋₆alkyl-N(R^{a})-carbonyl-, C₁₋₆alkylcarbonyl-N(R^{a})—, C₁₋₆alkyl-N(R^{a})-carbonyl-N(R^{a})—, C₁₋₆alkyl-N(R^{a})—SO₂—, C₁₋₆alkyl-SO₂—N(R^{a})—, C₁₋₆alkoxycarbonyl-N(R^{a})—, C₁₋₆alkylcarbonyl-N(R^{a})C₁₋₆alkyl-, C₁₋₆ alkyl-N(R^{a})-carbonyl-C₁₋₆alkyl-, and C₁₋₆alkoxy-C₁₋₆alkyl may optionally be substituted by one, two, or three R^{P}, phenyl, phenoxy, 5-6 membered heteroaryl, heteroaryloxy, 5-6 membered heteroaryl-(NR^{a})—, 4-6 membered heterocyclyl, 4-6 membered heterocyclyloxy or 4-6 membered heterocyclyl-N(R^{a})—; and wherein said heteroaryl or phenyl may optionally be substituted with one or more substituents each selected from R^{f}; and wherein said heterocyclyl may optionally be substituted by one or more substituents each selected from R^{g}; and wherein when said heterocyclyl contains a —NH moiety, the nitrogen of —NH may optionally be substituted by one or more substituents each selected from R^{h};
R^{a} and R^{b} are independently selected, for each occurrence, from the group consisting of hydrogen and C₁₋₃alkyl, wherein the C₁₋₃alkyl may optionally be substituted by one or more substituents each selected from the group consisting of halogen, cyano, oxo, and hydroxyl;
or R^{a} and R^{b}, together with the nitrogen to which they are attached, may form a 4-6 membered heterocyclic ring, which may have an additional heteroatom selected from 0, S, and N; wherein the 4-6 membered heterocyclic ring may optionally be substituted by one or more substituents each selected from the group consisting of halogen, cyano, oxo, and hydroxyl;
R^{g} is independently selected, for each occurrence, from the group consisting of R^{P}, hydrogen, oxo, C₁₋₆alkyl, C₂₋₆alkenyl, C₂₋₆alkynyl, C₃₋₆cycloalkyl, C₁₋₆alkoxy, C₁₋₆alkyl-S(O)_{w}—, C₁₋₆alkylcarbonyl-N(R^{a})—, and C₁₋₆alkoxycarbonyl-N(R^{a})—; wherein the C₁₋₆alkyl, C₂₋₆alkenyl, C₂-6alkynyl, C₃₋₆cycloalkyl, C₁₋₆alkoxy, C₁₋₆alkyl-S(O)_{w}—, C₁₋₆alkylcarbonyl-N(R^{a})—, or C₁₋₆ alkoxycarbonyl-N(R^{a})— may optionally be substituted by one or more substituents each selected from R^{p};
R^{h} is independently selected, for each occurrence, from the group consisting of hydrogen, halogen, hydroxyl, cyano, C₁₋₆alkyl, C₃₋₆alkenyl, C₃₋₆alkynyl, C₃₋₆cycloalkyl, C₁₋₆alkoxy, (RⁱR^{j})N—, C₁₋₆alkyl-S(O)₂—, C₁₋₆alkoxycarbonyl-, (RⁱR^{j})N-carbonyl-, and (RⁱR^{j})N—SO₂—; wherein the C₁₋₆alkyl, C₁₋₆alkoxy, C₃₋₆alkenyl, C₃₋₆alkynyl C₃₋₆cycloalkyl, C₁₋₆alkyl-S(O)₂—, or C₁₋₆alkylcarbonyl- may optionally be substituted by one or more substituents each selected from R^{p};
Rⁱ and R^{j} are independently selected, for each occurrence, from the group consisting of hydrogen, C₁₋₆alkyl, and C₃₋₆cycloalkyl; wherein the C₁₋₆alkyl and C₃₋₆cycloalkyl may be optionally substituted by one or more substituents each selected from halogen, hydroxyl, cyano, (R^{a}R^{b})N—, (R^{a}R^{b})N-carbonyl-, (R^{a}R^{b})N-carbonyl-(R^{a})N—, and C₁₋₃alkoxy;
or Rⁱ and R^{j} taken together with the nitrogen to which they are attached form a 4-7 membered heterocyclic ring, which may have an additional heteroatom selected from 0, S, and N; wherein the 4-7 membered heterocyclic ring may optionally be substituted on carbon by one or more substituents each selected from the group consisting of halogen, hydroxyl, oxo, cyano, C₁₋₆alkyl, C₁₋₆alkoxy, (R^{a}R^{b})N—, (R^{a}R^{b})N—SO₂—, and (R^{a}R^{b})N-carbonyl-; wherein said C₁₋₆alkyl or C₁₋₆alkoxy may optionally be substituted by halogen, hydroxyl or cyano; wherein the 4-7 membered heterocyclic ring may be optionally substituted on nitrogen by one or more substituents each selected from the group consisting of C₁₋₆alkyl and (R^{a}R^{b})N-carbonyl-; and wherein said C₁₋₆ alkyl may be optionally substituted by one or more substituents each selected from the group consisting of halogen, hydroxyl, and cyano;
R^{p} is independently selected, for each occurrence, from the group consisting of halogen, hydroxyl, cyano, C₁₋₆alkoxy, (RⁱⁱR^{jj})N—, (RⁱⁱR^{jj})N-carbonyl-, (RⁱⁱR^{jj})N—SO₂—, (RⁱⁱR^{jj})N-carbonyl-N(R¹)—, (CH₃)C(O)N(Rⁱⁱ)—, —C(O)OH, and —C(O)N(RⁱⁱR^{jj});
Rⁱⁱ and R^{jj} are independently selected, for each occurrence, from the group consisting of hydrogen, C₁₋₆alkyl, and C₃₋₆cycloalkyl;
R^{k} is selected from the group consisting of C₁₋₆alkyl, C₃₋₆cycloalkyl, 4-6 membered heterocyclyl, C₁₋₆alkyl-(4-6 membered heterocyclyl), and C₁₋₆alkyl-(5-6 membered heteroaryl), wherein the C₁₋₆alkyl may optionally be substituted by one, two, or three R^{p};
w 0, 1, or 2; and
pharmaceutically acceptable salts, stereoisomers, esters and prodrugs thereof.

20. The compound of claim 19, wherein C is a phenyl optionally substituted by one, two, or three substituents each selected from the group consisting of halogen, hydroxyl, cyano, C₁₋₆alkyl, C₁₋₆alkoxy, and C₃₋₆cycloalkyl.

21. The compound of claim 19, wherein D is a phenyl optionally substituted by one, two, or three substituents each selected from the group consisting of halogen, hydroxyl, cyano, C₁₋₆alkyl, C₁₋₆alkoxy, and C₃₋₆cycloalkyl.

22. The compound of claim 19, wherein R^{w} is selected from the group consisting of: 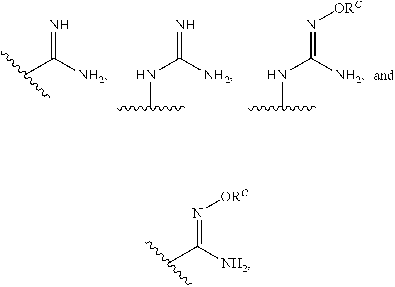
wherein R^{C} is hydrogen or C₁₋₆alkyl.

23. The compound of claim 19, wherein W is phenyl.

24. The compound of claim 19, wherein the compound is represented by: 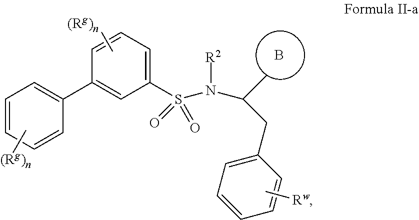
wherein n is independently, for each occurrence, 1, 2, or 3.

25. The compound of claim 24, wherein n is independently, for each occurrence, 1 or 2.

26. The compound of claim 24, wherein R^{g} is selected from the group consisting of hydrogen, —Cl, —OCH₃, and —OCH₂CH₃.

27. The compound of claim 19, wherein B is selected from the group consisting of 5-6 membered monocyclic heteroaryl, 8-10 membered bicyclic heteroaryl, and 8-10 membered bicyclic heterocyclyl, wherein B contains at least one nitrogen and B is optionally substituted by one, two or three substituents each independently selected from the group consisting of halogen, hydroxyl, C₁₋₆haloalkyl, C₁₋₆alkoxy, C₁₋₆alkyl, (R^{a}R^{b})N—, N(R^{a}R^{b})—C₁₋₆alkyl-, (R^{a}R^{b})N—C(O)—C₁₋₆alkyl-, N(R^{a}R^{b})—C(O)—N(R^{a})—C₁₋₆alkyl-, C₁₋₆alkyl-N(R^{a})—C(O)—C₁₋₆alkyl-, C₁₋₆alkyl-C(O)—N(R^{a})—C₁₋₆alkyl-, C(O)OH—C₁₋₆alkyl-, N(R^{a}R^{b})—C₁₋₆alkyl-N(R^{a})—, C₁₋₆alkoxy-C₁₋₆alkyl-N(R^{a})—, N(R^{a}R^{b})—C(O)—C₁₋₆alkyl-N(R^{a})—, N(R^{a}R^{b})—C(O)—N(R^{a})—C₁₋₆alkyl-N(R^{a})—, C₁₋₆alkyl-N(R^{a})—C(O)—N(R^{a})—, N(R^{a}R^{b})—C₁₋₆alkyl-C(O)—N(R^{a})—, N(R^{a}R^{b})—C₁₋₆alkyl-O—, (R^{a})O—C₁₋₆alkyl-O—, N(R^{a}R^{b})—C(O)—N(R^{a})—C₁₋₆alkyl-O—, N(R^{a}R^{b})—C(O)—C₁₋₆alkyl-O—, (5-6 membered heterocyclyl)-C₁₋₆alkyl-O—, (5-6 membered heterocyclyl)-O—, (5-6 membered heteroaryl)-C₁₋₆alkyl-O—, (R^{a})C(O)—N(R^{a})—C₁₋₆alkyl-O—, and —N(RⁱR^{j}), wherein Rⁱ and R^{j} are taken together with the nitrogen to which they are attached form a 4-7 membered heterocyclic ring optionally be substituted by one, two or three substituent each independently selected from the group consisting of C₁₋₆alkyl, —NH₂, —C(O)NH(C₁₋₃alkyl), and R^{a} and R^{b} are independently, for each occurrence, hydrogen or C₁₋₃alkyl.

28. (canceled)

29. The compound of claim 19, wherein B is selected from the group consisting of 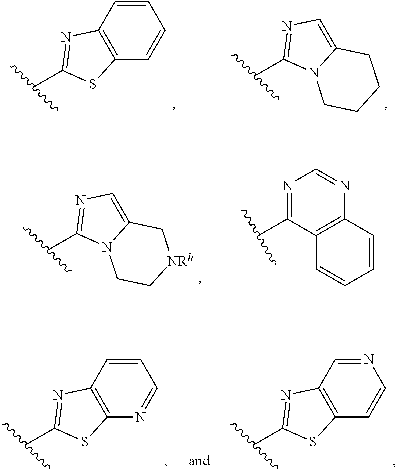
wherein B may optionally substituted on one, two, or three carbons by a substituent each independently selected from the group consisting of halogen, hydroxyl, C₁₋₆haloalkyl, C₁₋₆alkoxy, C₁₋₆alkyl, (R^{a}R^{b})N—, N(R^{a}R^{b})—C₁₋₆alkyl-, (R^{a}R^{b})N—C(O)—C₁₋₆alkyl-, N(R^{a}R^{b})—C(O)—N(R^{a})—C₁₋₆alkyl-, C₁₋₆alkyl-N(R^{a})—C(O)—C₁₋₆alkyl-, C₁₋₆alkyl-C(O)—N(R^{a})—C₁₋₆alkyl-, C(O)OH—C₁₋₆alkyl-, N(R^{a}R^{b})—C₁₋₆alkyl-N(R^{a})—, C₁₋₆alkoxy-C₁₋₆alkyl-N(R^{a})—, N(R^{a}R^{b})—C(O)—C₁₋₆alkyl-N(R^{a})—, N(R^{a}R^{b})—C(O)—N(R^{a})—C₁₋₆alkyl-N(R^{a})—, C₁₋₆alkyl-N(R^{a})—C(O)—N(R^{a})—, N(R^{a}R^{b})—C₁₋₆alkyl-C(O)—N(R^{a})—, N(R^{a}R^{b})—C₁₋₆alkyl-O—, (R^{a})O—C₁₋₆alkyl-O—, N(R^{a}R^{b})—C(O)—N(R^{a})—C₁₋₆alkyl-O—, N(R^{a}R^{b})—C(O)—C₁₋₆alkyl-O—, (5-6 membered heterocyclyl)-C₁₋₆alkyl-O—, (5-6 membered heterocyclyl)-O—, (5-6 membered heteroaryl)-C₁₋₆alkyl-O—, (R^{a})C(O)—N(R^{a})—C₁₋₆alkyl-O—, and —N(RⁱR^{j}), wherein Rⁱ and R^{j} are taken together with the nitrogen to which they are attached form a 4-7 membered heterocyclic ring optionally be substituted by one, two or three substituent each independently selected from the group consisting of C₁₋₆alkyl, —NH₂, —C(O)NH(C₁₋₃alkyl), and R^{a} and R^{b} are independently, for each occurrence, hydrogen or C₁₋₃alkyl.

30. The compound of claim 19, wherein B is selected from the group consisting of:

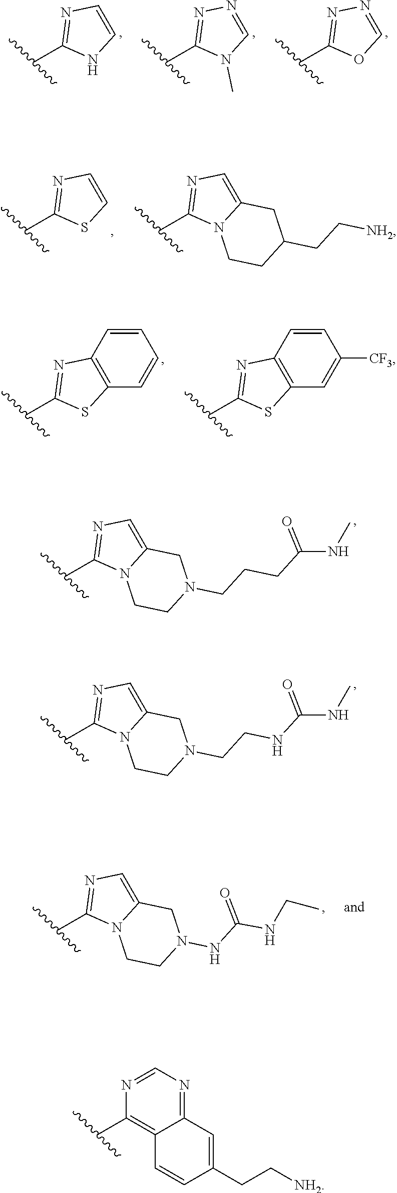

31. The compound of claim 19, wherein R² is hydrogen.

32. A compound represented by: 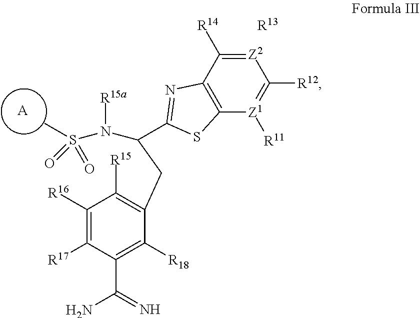
wherein:
A is selected from the group consisting of phenyl, naphthyl, and 5-6 membered heteroaryl, wherein A is optionally substituted by one, two, or three substituents each independently selected from the group consisting of R^{A};
R¹¹, R¹², R¹³, and R¹⁴ are independently selected, for each occurrence, from the group consisting of H, —CN, —OH, —NO₂, —NH₂, halogen, C₁₋₆haloalkyl, C₁₋₆alkyl, C₁₋₆alkoxy, and C₃₋₆cycloalkyl;
R^{15a} is selected from the group consisting of H, C₁₋₆alkyl, and C₃₋₆cycloalkyl;
R¹⁵, R¹⁶, R¹⁷, and R¹⁸ are independently selected, for each occurrence, from the group consisting of H, —NO₂, —NH₂, cyano, hydroxyl, halogen, C₁₋₆haloalkyl, C₁₋₆alkyl, C₁₋₆alkoxy, and C₃₋₆cycloalkyl;
R^{A} is selected from the group consisting of —NO₂, —NH₂, cyano, hydroxyl, halogen, C₁₋₆haloalkyl, C₁₋₆alkyl, C₁₋₆alkoxy, and C₃₋₆cycloalkyl;
Z¹ is C or N, wherein when Z¹ is N then R¹¹ is absent;
Z² is C or N, wherein when Z² is N then R¹³ is absent; and
pharmaceutically acceptable salts, stereoisomers, esters and prodrugs thereof.

33. The compound of claim 32, wherein A is a phenyl substituted by one, two or three substituents each independently selected from the group consisting of halogen and phenyl, wherein the phenyl is optionally substituted by one, two or three halogen.

34. The compound of claim 32, wherein A is a phenyl optionally substituted by one, two or three halogen or one

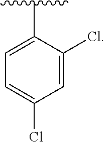 [SMILES: *c1ccc(Cl)cc1Cl]

35. The compound of claim 32, wherein R¹¹ is selected from the group consisting of H, Cl,

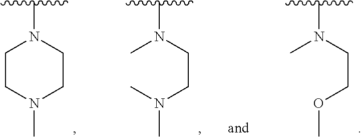

36. The compound of claim 32, wherein R¹² is selected from the group consisting of H, —OH, —OCH₃, and CF₃.

37. The compound of claim 32, wherein R¹³ is selected from the group consisting of H, CF₃, and CH₃.

38. The compound of claim 32, wherein R¹⁴ is H.

39. (canceled)

40. The compound of claim 32, wherein R¹⁵, R¹⁶, R¹⁷, and R¹⁸ are H.

41. The compound of claim 32, wherein Z¹ is C and Z² is N.

42. The compound of claim 32, wherein Z¹ is N and Z² is C.

43. A compound selected from the group consisting of: 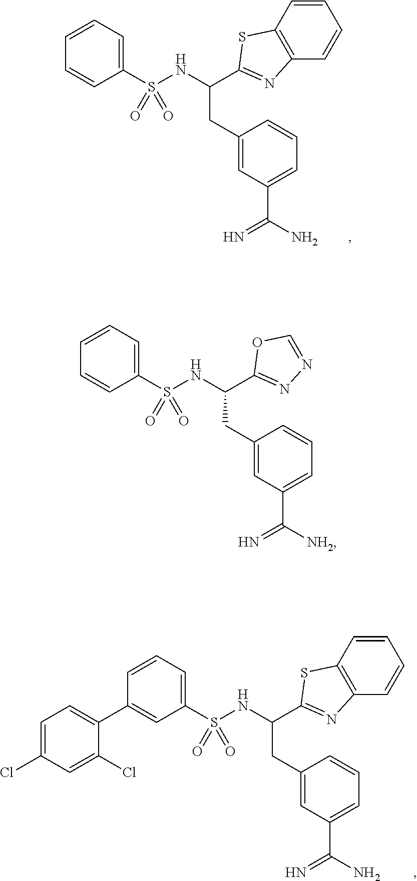 [SMILES: N=C(N)c1cccc(CC(NS(=O)(=O)c2ccccc2)c2nc3ccccc3s2)c1 ; N=C(N)c1cccc(C[C@H](NS(=O)(=O)c2ccccc2)c2nnco2)c1 ; N=C(N)c1cccc(CC(NS(=O)(=O)c2cccc(-c3ccc(Cl)cc3Cl)c2)c2nc3ccccc3s2)c1] 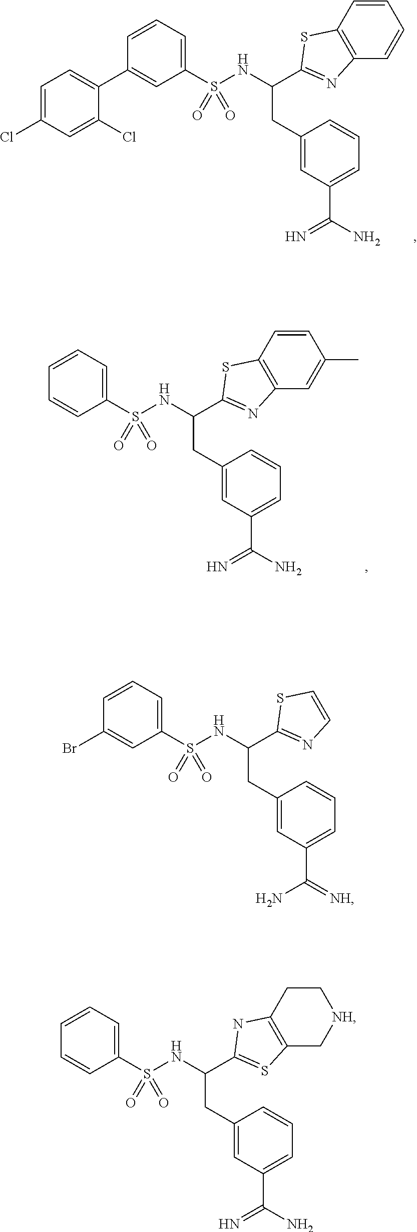 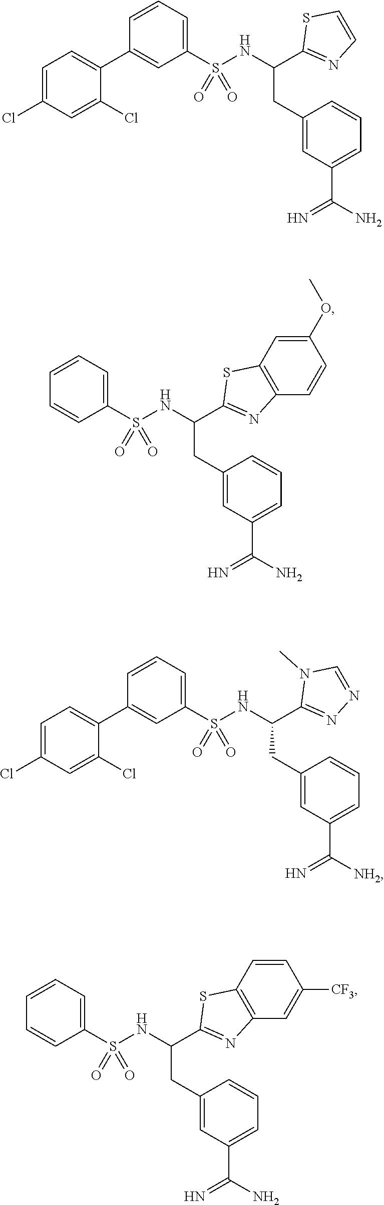 [SMILES: N=C(N)c1cccc(CC(NS(=O)(=O)c2cccc(-c3ccc(Cl)cc3Cl)c2)c2nccs2)c1 ; COc1ccc2nc(C(Cc3cccc(C(=N)N)c3)NS(=O)(=O)c3ccccc3)sc2c1 ; Cn1cnnc1[C@H](Cc1cccc(C(=N)N)c1)NS(=O)(=O)c1cccc(-c2ccc(Cl)cc2Cl)c1 ; N=C(N)c1cccc(CC(NS(=O)(=O)c2ccccc2)c2nc3cc(C(F)(F)F)ccc3s2)c1] 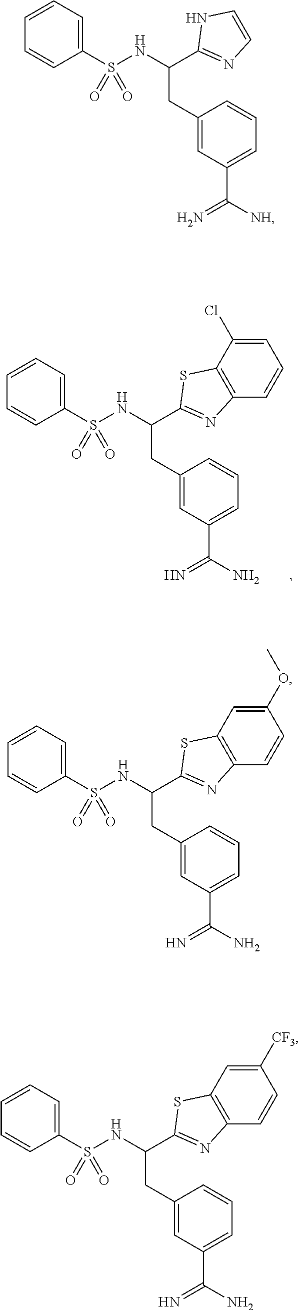 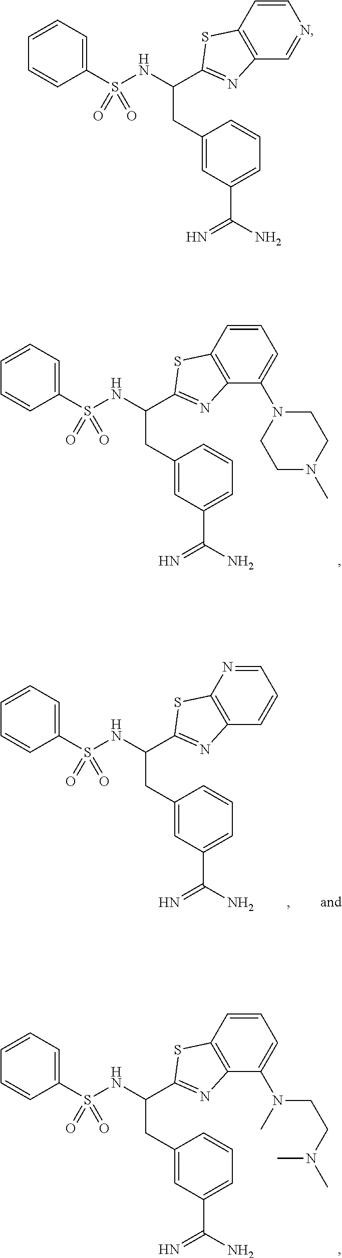 [SMILES: N=C(N)c1cccc(CC(NS(=O)(=O)c2ccccc2)c2nc3cnccc3s2)c1 ; CN1CCN(c2cccc3sc(C(Cc4cccc(C(=N)N)c4)NS(=O)(=O)c4ccccc4)nc23)CC1 ; N=C(N)c1cccc(CC(NS(=O)(=O)c2ccccc2)c2nc3cccnc3s2)c1 ; CN(C)CCN(C)c1cccc2sc(C(Cc3cccc(C(=N)N)c3)NS(=O)(=O)c3ccccc3)nc12] 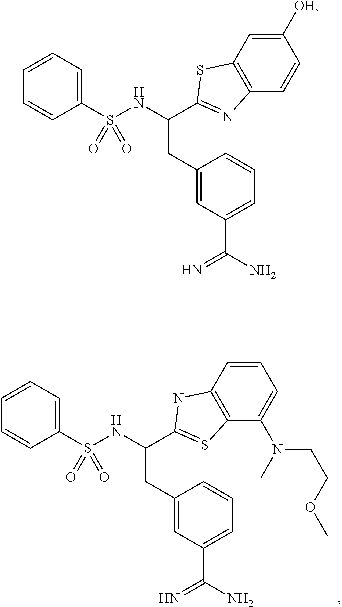
or a pharmaceutically acceptable salt thereof.

44. A method of treating a viral infection in a patient in need thereof, comprising administering to the patient an effective amount of a compound of claim 19.

45-51. (canceled)

52. A method of treating a viral infection in a patient in need thereof, comprising administering to the patient an effective amount of claim 32.
